(19) United States
(12) Patent Application Publication
(10) Pub. No.: US 2024/0093234 A1
(43) Pub. Date: Mar. 21, 2024

(54) CHIMERIC ADENOVIRAL VECTORS

(71) Applicant: Vaxart, Inc., South San Francisco, CA (US)

(72) Inventors: Sean Tucker, South San Francisco, CA (US); Emery Dora, South San Francisco, CA (US)

(21) Appl. No.: 18/263,462

(22) PCT Filed: Jan. 31, 2022
(86) PCT No.: PCT/US2022/014599

§ 371 Date: Jul. 28, 2023

Related U.S. Application Data
(63) Continuation-in-part of application No. PCT/US2021/035930, filed on Jun. 4, 2021.
(60) Provisional application No. 63/144,339, filed on Feb. 1, 2021.

(51) Int. Cl. C12N 15/86; A61K 39/215; A61P 37/04; C07K 14/005

(52) U.S. Cl. CPC C12N 15/86; A61K 39/215; A61P 37/04; C07K 14/005; A61K 2039/5256; C12N 2710/10044; C12N 2710/10071; C12N 2770/20022; C12N 2830/50

(57) ABSTRACT

The present disclosure provides expression vectors, e.g., chimeric adenoviral vectors, comprising a nucleic acid encoding a coronavirus disease 2019 (COVID-19) N protein, e.g., an N protein from SARS-CoV-2, and a heterologous antigenic polypeptide and methods employing such expression vectors for using the vectors to elicit an immune response.

### CROSS-REFERENCE TO RELATED APPLICATIONS

This applications claims priority benefit of U.S. Provisional Application No. 63/144,339, filed Feb. 1, 2021, and is a continuation-in-part of International Application No. PCT/US2021/035930, filed Jun. 4, 2021, which claims priority benefit of U.S. Provisional Application No. 63/144,339, filed Feb. 1, 2021, U.S. Provisional Application No. 63/074,954, filed Sep. 4, 2020; U.S. Provisional Application No. 63/045,710, filed Jun. 29, 2020; and U.S. Provisional Application No. 63/035,490, filed Jun. 5, 2020. Each application is incorporated by reference for all purposes.

### BACKGROUND OF THE DISCLOSURE

Coronavirus disease 2019 (COVID-19) is an infectious disease caused by severe acute respiratory syndrome coronavirus 2 (SARS-CoV-2). Some symptoms of the disease include, for example, fever, cough, shortness of breath, muscle pain, sputum production, diarrhea, sore throat, loss of smell, and abdominal pain. While the majority of cases result in mild symptoms, some progress to viral pneumonia and multi-organ failure. The disease currently has no cure and has spread rapidly across several continents, with community outbreaks throughout the world.

### SUMMARY OF THE DISCLOSURE

In one aspect, described herein is a chimeric adenoviral expression vector, comprising an expression cassette comprising: a nucleic acid encoding an antigenic polypeptide; and a nucleic acid encoding a SARS-CoV-2 N protein, wherein the antigenic polypeptide is not a SARS-CoV2 protein. In some embodiments, the antigenic polypeptide is not a coronavirus protein. In some embodiments, the SARS-CoV-2 N protein comprises an amino acid sequence having at least 95%, 96%, 97%, 98%, 99%, or 100% identity to the amino acid sequence of SEQ ID NO:2. In some embodiments, the nucleic acid encoding the SARS-CoV-2 N protein comprises a sequence having at least 85%, 90%, 95%, 97%, 99%, or 100% identity to the sequence of SEQ ID NO:4. In some embodiments, the antigenic polypeptide is a cancer antigen. In other embodiments, the antigenic polypeptide is from a pathogen, e.g., a virus, bacteria, fungus, or parasite. In some embodiments, the expression cassette comprises a bicistronic or multicistronic construct comprising the nucleic acid encoding the antigenic polypeptide and the nucleic acid encoding the SARS-CoV-2 N protein operably linked to a promoter. In some embodiments, the nucleic acid encoding the antigenic protein is positioned 5′ of the nucleic acid encoding the SARS-CoV2-N protein. In other embodiments, the nucleic acid encoding the SARS-CoV2-N protein is positioned 5′ of the nucleic acid encoding the antigenic polypeptide. In some embodiments, the expression cassette comprises an internal ribosome entry site (IRES), a ribosome skipping element, or a furin cleavage site positioned between the nucleic acid encoding the antigenic polypeptide and the nucleic acid encoding the SARS-CoV-2 N protein. In some embodiments, the expression cassette comprises a ribosomal skipping element encoding a peptide selected from the group consisting of a 2A peptide (T2A), a porcine teschovirus-1 2A peptide (P2A), a foot-and-mouth disease virus 2A peptide (F2A), equine rhinitis A virus 2A peptide (E2A), a cytoplasmic polyhedrosis virus 2A peptide (BmCPV 2A), and a flacherie virus of B. mori 2A peptide (BmIFV 2A). In some embodiments, the ribosomal skipping element is a sequence encoding a T2A peptide. In some embodiments, the promoter is a CMV promoter. In some embodiments, the nucleic acid encoding the antigenic polypeptide is operably linked to a first promoter and the nucleic acid encoding the SARS-CoV-2 N protein is operably linked to a second promoter. In some embodiments, the first promoter and the second promoter are each a CMV promoter. In some embodiments, the first promoter is a CMV promoter and is a beta-actin promoter; or the first promoter is a beta-actin promoter and the second promoter is a CMV promoter. In many embodiments, the expression cassette comprises a polyadenylation signal, such as a bovine growth hormone polyadenylation signal. In some embodiments, the chimeric adenoviral expression vector further comprises a nucleic acid encoding a toll-like receptor-3 (TLR-3). In some embodiments, the TLR-3 agonist comprises a nucleic acid encoding a dsRNA. In some embodiments, the nucleic acid encoding the TLR-3 agonist comprises a sequence selected from the group consisting of: SEQ ID NOS: 11-18. In other aspects, the disclosure further provides a host comprising a chimeric adenoviral vector as described herein, e.g., in this paragraph, an immunogenic composition comprising the chimeric adenoviral expression vector as described herein, e.g., in this paragraph and a pharmaceutically acceptable carrier; and methods for eliciting an immune response towards an antigenic polypeptide in a subject, comprising administering to the subject an immunogenically effective amount of the chimeric adenoviral expression vector as described herein, e.g., in this paragraph, to a mammalian subject. In some embodiments, the route of administration is oral, intranasal, or mucosal. In some embodiments, the route of administration is oral delivery by swallowing a tablet. In some embodiments, the immune response is elicited in an alveolar cell, an absorptive enterocyte, a ciliated cell, a goblet cell, a club cells, and/or an airway basal cell of the subject. In some embodiments, the subject is a human.

In an additional aspect, the disclosure provides a chimeric polynucleotide, comprising an expression cassette comprising: a nucleic acid encoding an antigenic polypeptide, with the proviso that the antigenic polypeptide is not a SARS-CoV-2 protein; and a nucleic acid encoding a SARS-CoV-2 N protein. In some embodiments, the antigenic polypeptide is not a coronavirus polypeptide. In some embodiments, the SARS-CoV-2 N protein comprises an amino acid sequence having at least 95%, 96%, 97%, 98%, 99%, or 100% identity to the amino acid sequence of SEQ ID NO:2. In some embodiments, the SARS-CoV-2 N protein comprises a sequence having at least 85%, 90%, 95%, 97%, 99%, or 100% identity to the sequence of SEQ ID NO:4. In some embodiments, the antigenic polypeptide is from a pathogen, such as a virus, bacteria, fungus, or parasite. In some embodiments, the expression cassette comprises a bicistronic or multicistronic construct comprising the nucleic acid encoding the antigenic polypeptide and the nucleic acid encoding the SARS-CoV-2 N protein operably linked to a promoter. In some embodiments, the nucleic acid encoding the antigenic protein is positioned 5′ of the nucleic acid encoding the SARS-CoV2-N protein. In other embodiments, the nucleic acid encoding the SARS-CoV2-N protein is positioned 5′ of the nucleic acid encoding the antigenic polypeptide. In some embodiments, the expression cassette comprises an internal ribosome entry site (IRES), a ribosome skipping element, or a furin cleavage site positioned between the nucleic acid encoding the antigenic polypeptide and the nucleic acid encoding the SARS-CoV-2 N protein. In some embodiments, the ribosomal skipping element is a sequence encoding a virus polypeptide selected from the group consisting of a 2A peptide (T2A), a porcine teschovirus-1 2A peptide (P2A), a foot-and-mouth disease virus 2A peptide (F2A), equine rhinitis A virus 2A peptide (E2A), a cytoplasmic polyhedrosis virus 2A peptide (BmCPV 2A), and a flacherie virus of B. mori 2A peptide (BmIFV 2A. In some embodiments, the promoter is a CMV promoter. In some embodiments, the nucleic acid encoding the antigenic polypeptide is operably linked to a first promoter and the nucleic acid encoding the SARS-CoV-2 N protein is operably linked to a second promoter. In some embodiments, the first promoter and the second promoter are each a CMV promoter. In some embodiments, the first promoter is a CMV promoter and is a beta-actin promoter; or the first promoter is a beta-actin promoter and the second promoter is a CMV promoter. In some embodiments, the expression cassette comprises a polyadenylation signal. In some embodiments, the polyadenylation signal is a bovine growth hormone polyadenylation signal. In some embodiments, the chimeric polynucleotide comprises a sequence encoding a TLR-3 agonist. In some embodiments, the TLR-3 agonist comprises a nucleic acid encoding a dsRNA. In some embodiments, the TLR-3 agonist comprises a sequence selected from the group consisting of SEQ ID NOS. 1-18. In further aspects, the disclosure also provides an expression construct comprising the chimeric polynucleotide as described herein, e.g., in this paragraph; a method of inducing an immune response in a subject comprising administering the expression construct; and a host cell comprising the chimeric polynucleotide or the expression construct. In some embodiments, the host cell is a mammalian host cell.

In a further aspect, provided herein is a chimeric adenoviral expression vector, comprising a bicistronic or multicistronic expression construct comprising: a nucleic acid encoding a SARS-CoV-2 S protein; and a nucleic acid encoding a SARS-CoV-2 N protein. In some embodiments, the SARS-CoV-2 N protein comprises an amino acid sequence having at least 95%, 96%, 97%, 98%, 99%, or 100% identity to the amino acid sequence of SEQ ID NO:2. In some embodiments, the nucleic acid encoding the SARS-CoV-2 N protein comprises a sequence having at least 85%, 90%, 95%, 97%, 99%, or 100% identity to the sequence of SEQ ID NO:4. In some embodiments, the SARS-CoV-2 S protein comprises a sequence having at least 90% identity to SEQ ID NO:1. In some embodiments, the nucleic acid encoding the SARS-CoV-2 S protein comprises a sequence having at least 85%, 90%, 95%, 97%, 99%, or 100% identity to the sequence of SEQ ID NO:3. In some embodiments, the bicistronic construct is operably linked to a promoter. In some embodiments, the nucleic acid encoding the SARS-CoV-2 protein is positioned 5′ of the nucleic acid encoding the SARS-CoV2-N protein. In other embodiments, the nucleic acid encoding the SARS-CoV2-N protein is positioned 5′ of the nucleic acid encoding the SARS-CoV-2 S protein. In some embodiments, the expression cassette comprises an internal ribosome entry site (IRES), a ribosome skipping element, or a furin cleavage site positioned between the nucleic acid encoding the SARS-CoV-2 S protein and the nucleic acid encoding the SARS-CoV-2 N protein. In some embodiments, the ribosomal skipping element is a sequence encoding a peptide selected from the group consisting of a 2A peptide (T2A), a porcine teschovirus-1 2A peptide (P2A), a foot-and-mouth disease virus 2A peptide (F2A), equine rhinitis A virus 2A peptide (E2A), a cytoplasmic polyhedrosis virus 2A peptide (BmCPV 2A), and a flacherie virus of B. mori 2A peptide (BmIFV 2A). In some embodiments, the ribosomal skipping element is a sequence encoding a T2A peptide. In some embodiments, the promoter is a CMV promoter. In some embodiments, the expression cassette comprises a polyadenylation signal. In some embodiments, the polyadenylation signal is a bovine growth hormone polyadenylation signal. In some embodiments, the chimeric adenoviral expression vector further comprises a nucleic acid encoding a a toll-like receptor-3 (TLR-3). In some embodiments, the TLR-3 agonist comprises a nucleic acid encoding a dsRNA. In some embodiments, the nucleic acid encoding the TLR-3 agonist comprises a sequence selected from the group consisting of: SEQ ID NOS:11-18.

In another aspect, the disclosure provides a chimeric adenoviral expression vector, comprising an expression cassette comprising the following elements: (a) a first promoter operably linked to a nucleic acid encoding a first severe acute respiratory syndrome coronavirus 2 (SARS-CoV-2) protein; and (b) a second promoter operably linked to a nucleic acid encoding a toll-like receptor-3 (TLR-3) agonist. In some embodiments, the chimeric adenoviral expression vector comprises additional element (c): a third promoter operably linked to a nucleic acid encoding a second SARS-CoV-2 protein. In some embodiments, element (c) is placed between elements (a) and (b) in the expression cassette. In certain embodiments, the first SARS-CoV-2 protein in (a) and the second SARS-CoV-2 protein in (c) are different. In other embodiments, the SARS-CoV-2 protein in (a) and the SARS-CoV-2 protein in (c) are the same.

In some embodiments of this aspect, the nucleic acid encoding the first SARS-CoV-2 protein in element (a) and/or the nucleic acid encoding the second SARS-CoV-2 protein in element (c) comprises a sequence having at least 85%, 90%, 95%, 97%, 99%, or 100% identity to the sequence of SEQ ID NO:3. In some embodiments, the first and/or second SARS-CoV-2 protein comprises a SARS-CoV-2 S protein having a sequence with at least 85%, 90%, 95%, 97%, 99%, or 100% identity to the sequence of SEQ ID NO:1 or SEQ ID NO:20 or SEQ ID NO:20.

In some embodiments, the nucleic acid encoding the first SARS-CoV-2 protein in element (a) and/or the nucleic acid encoding the second SARS-CoV-2 protein in element (c) comprises a sequence having at least 85%, 90%, 95%, 97%, 99/o, or 100% identity to the sequence of SEQ ID NO:4. In some embodiments, the first and/or the second SARS-CoV-2 protein comprises a SARS-CoV-2 N protein having a sequence with at least 85%, 90%, 95%, 97%, 99%, or 100% identity to the sequence of SEQ ID NO:2.

In some embodiments of this aspect, the nucleic acid encoding the first SARS-CoV-2 protein in element (a) and/or the nucleic acid encoding the second SARS-CoV-2 protein in element (c) comprises a sequence having at least 85%, 90%, 95%, 97%, 99%, or 100% identity to the sequence of SEQ ID NO:5. In some embodiments, the first and/or second SARS-CoV-2 protein comprises a fusion protein comprising a S1 region of a SARS-CoV-2 S protein, a furin site, and a SARS-CoV-2 N protein, and wherein the fusion protein comprises a sequence having at least 85% identity to the sequence of SEQ ID NO:10.

Moreover, the first promoter and the second promoter in the chimeric adenoviral vector can be identical or different. For example, the first promoter and the second promoter each can be a CMV promoter.

In some embodiments of the aspect, when all three elements (a)-(c) are present, the first promoter can be a CMV promoter, the second promoter can be a CMV promoter, and the third promoter can be a beta-actin promoter (e.g., a human beta-actin promoter).

In another aspect, the disclosure features a chimeric adenoviral expression vector, comprising an expression cassette comprising the following elements: (a) a first promoter operably linked to a nucleic acid encoding a SARS-CoV-2 S protein; and (b) a second promoter operably linked to a nucleic acid encoding a toll-like receptor-3 (TLR-3) agonist.

In some embodiments of this aspect, the nucleic acid encoding the SARS-CoV-2 S protein comprises the sequence of SEQ ID NO:3. In some embodiments, the SARS-CoV-2 S protein comprises the sequence of SEQ ID NO:1 or SEQ ID NO: 19 or SEQ ID NO:20.

In some embodiments, the first promoter and the second promoter are each a CMV promoter.

In another aspect, the disclosure features a chimeric adenoviral expression vector, comprising an expression cassette comprising the following elements: (a) a first promoter operably linked to a nucleic acid encoding a SARS-CoV-2 S protein; (b) a second promoter operably linked to a nucleic acid encoding a toll-like receptor-3 (TLR-3) agonist; and (c) a third promoter operably linked to a nucleic acid encoding a SARS-CoV-2 N protein, optionally in which the order of the elements in the expression cassette from the N-terminus to the C-terminus is: element (a), element (c), and element (b).

In some embodiments of this aspect, the nucleic acid encoding the SARS-CoV-2 S protein comprises the sequence of SEQ ID NO:3. In some embodiments, the SARS-CoV-2 S protein comprises the sequence of SEQ ID NO:1 or SEQ ID NO:19 or SEQ ID NO:20.

In some embodiments of this aspect, the nucleic acid encoding the SARS-CoV-2 N protein comprises the sequence of SEQ ID NO:4. In some embodiments, the SARS-CoV-2 N protein comprises the sequence of SEQ ID NO:2.

Further, in some embodiments of this aspect, the first promoter in element (a) is a CMV promoter, the second promoter in element (b) is a CMV promoter, and the third promoter in element (c) is a beta-actin promoter (e.g., a human beta-actin promoter).

In some embodiments, the elements (a), (b), and (c) together are encoded by the sequence of SEQ ID NO:6. Further, the chimeric adenoviral expression vector of this aspect is encoded by the sequence of SEQ ID NO:8.

In another aspect, the disclosure features a chimeric adenoviral expression vector, comprising an expression cassette comprising the following elements: (a) a first promoter operably linked to a nucleic acid encoding a SARS-CoV-2 fusion protein, wherein the SARS-CoV-2 fusion protein comprises a S1 region of a SARS-CoV-2 S protein, a furin site, and a SARS-CoV-2 N protein; and (b) a second promoter operably linked to a nucleic acid encoding a toll-like receptor-3 (TLR-3) agonist.

In some embodiments of this aspect, the nucleic acid encoding the SARS-CoV-2 fusion protein comprises the sequence of SEQ ID NO:5. In some embodiments, the SARS-CoV-2 fusion protein comprises the sequence of SEQ ID NO:10.

In some embodiments of this aspect, the first promoter and the second promoter are each a CMV promoter.

In some embodiments of this aspect, the elements (a) and (b) together are encoded by the sequence of SEQ ID NO:7. Further, the chimeric adenoviral expression vector of this aspect is encoded by the sequence of SEQ ID NO:9.

In another aspect, the disclosure features an immunogenic composition comprising a chimeric adenoviral expression vector described herein and a pharmaceutically acceptable carrier.

In a further aspect, the disclosure additionally features a chimeric adenoviral expression vector, comprising an expression cassette comprising the following elements: (a) a first promoter operably linked to a nucleic acid encoding a first severe acute respiratory syndrome coronavirus 2 (SARS-CoV-2) protein; (b) a second promoter operably linked to a nucleic acid encoding a toll-like receptor-3 (TLR-3) agonist; and (c) a third promoter operably linked to a nucleic acid encoding a SARS-CoV-2 N protein. In some embodiments, the SARS-CoV-2 N protein comprises an amino acid sequence having at least 95%, 96%, 97%, 98%, 99%, or 100% identity to the amino acid sequence of SEQ ID NO:2. In some embodiments, element (c) is situated between elements (a) and (b) in the expression cassette. In some embodiments, the first SARS-CoV-2 protein comprises a SARS-CoV-2 S protein having a sequence with at least 95%, 96%, 97%, 98%, 99%, or 100% identity to the sequence of SEQ ID NO:1 or SEQ ID NO:19 or SEQ ID NO:20. In some embodiments, the nucleic acid encoding the TLR-3 agonist comprises a nucleic acid encoding a dsRNA. In some embodiments, the nucleic acid encoding the TLR-3 agonist comprises a sequence selected from the group consisting of: SEQ ID NOS:11-18. In some embodiments, the nucleic acid encoding the first SARS-CoV-2 protein in element (a) comprises a sequence having at least 85%, 90%, 95%, 97%, 99%, or 100% identity to the sequence of SEQ ID NO:3. In some embodiments, the nucleic acid encoding the SARS-CoV-2 N protein comprises a sequence having at least 85%, 90%, 95%, 97%, 99%, or 100% identity to the sequence of SEQ ID NO:4. In some embodiments, the first promoter and the second promoter are identical. In some embodiments, the first promoter and the second promoter are each a CMV promoter. In some embodiments, the first promoter is a CMV promoter, the second promoter is a CMV promoter, and the third promoter is a beta-actin promoter. In some embodiments, element (c) is situated between elements (a) and (b), and elements (a), (c), and (b) together are encoded by a sequence having at least 95% identity to SEQ ID NO:6 or is encoded by the sequence of SEQ ID NO:6. In some embodiments, the chimeric adenoviral expression vector comprises a sequence having at least 95% identity to SEQ ID NO:8 or comprises the sequence of SEQ ID NO:8.

In another aspect, the disclosure provides a method for eliciting an immune response towards a SARS-CoV-2 protein (e.g., a SARS-CoV-2 protein having the sequence of SEQ ID NOS:1, 2, or 10, or a variant thereof as described herein (e.g., having at least 90% or at least 95% identity to SEQ ID NO:1, 2, or 10) in a subject, comprising administering to the subject an immunogenically effective amount of a chimeric adenoviral expression vector described herein or an immunogenic composition described herein. In some embodiments, the route of administration is oral, intranasal, or mucosal (e.g., oral). In certain embodiments, the route of administration is oral delivery by swallowing a tablet.

In some embodiments of the method, the immune response is elicited in an alveolar cell, an absorptive enterocyte, a ciliated cell, a goblet cell, a club cells, and/or an airway basal cell of the subject. In certain embodiments, the subject is a human.

Also provided is a chimeric polynucleotide (which can be used to induce an immune response in a subject, including but not limited to a CD8 T-cell response), comprising an expression cassette comprising the following elements: (a) a first promoter operably linked to a nucleic acid encoding a first severe acute respiratory syndrome coronavirus 2 (SARS-CoV-2) protein; and (b) a second promoter operably linked to a nucleic acid encoding a toll-like receptor-3 (TLR-3) agonist; and (c) a third promoter operably linked to a nucleic acid encoding a SARS-CoV-2 protein or a non-SARS-CoV-2 antigenic protein.

In some embodiments, the chimeric polynucleotide is a chimeric adenoviral expression vector. In some embodiments, the nucleic acid encoding the TLR-3 agonist comprises a nucleic acid encoding a dsRNA. In some embodiments, the nucleic acid encoding the TLR-3 agonist comprises a sequence selected from the group consisting of: SEQ ID NOS. 11-18 In some embodiments, element (c) is placed between elements (a) and (b) in the expression cassette.

In a further aspect, the disclosure provides a chimeric polynucleotide, comprising an expression cassette comprising the following elements: (a) a first promoter operably linked to a nucleic acid encoding an antigenic protein; (b) a second promoter operably linked to a nucleic acid encoding a toll-like receptor-3 (TLR-3) agonist; and (c) a third promoter operably linked to a nucleic acid encoding a SARS-CoV-2 N-protein. In some embodiments, the SARS-CoV-2 N protein has at least 95%, 96%, 97%, 98%, 99%, or 100% identity to SEQ ID NO:2. In some embodiments, the chimeric polynucleotide is a chimeric adenoviral expression vector. In some embodiments, the nucleic acid encoding the TLR-3 agonist comprises a nucleic acid encoding a dsRNA. In some embodiments, the nucleic acid encoding the TLR-3 agonist comprises a sequence selected from the group consisting of: SEQ ID NOS:11-18. In some embodiments, element (c) is placed between elements (a) and (b) in the expression cassette. In some embodiments, the antigenic protein is from a bacteria, fungus, virus, or parasite. In some embodiments, the antigenic protein is a cancer antigen.

In a further aspect, the disclosure provides a method of inducing an immune response in a subject, the method comprising administering a chimeric polynucleotide as seat forth in the preceding paragraph to a subject.

### BRIEF DESCRIPTION OF THE DRAWINGS

FIG. 1 shows the expression of the antigens in human cells post infection.

FIG. 2 shows the IgG antibody titers to S1 following immunization of mice on days 0 and 14. Titers measured by standard ELISA.

FIGS. 3A and 3B show the IgG antibody titers to S1 and S2 following immunization of mice on days 0 and 14. MSD was used to measure the binding signal at multiple time points for both antigens. There were no significant differences in the signal at early timepoints, but more antibody responses were detected at the higher dose groups at later time points.

FIG. 4A-4D. Transgene inserts developed to test vaccine specific responses. Recombinant adenoviruses were made using these inserts a. rAd-S b. rAd-S-N c. rAd-S1-N d. rAd-S(fixed)-N.

FIGS. 5A-5D. Immunization with candidate rAd vaccines induce serum IgG and lung IgA responses. Antibody titers to S following immunization of Balb/c mice on days 0 and 14 with 1×108 IU rAd expressing full-length S (rAd-S), co-expressing full length S and N (rAd-S-N) or co-expressing a fusion protein comprising the S1 domain and N (rAd-S1-N). (FIG. 5A) IgG serum IgG endpoint titers to S1 were measured by standard ELISA (n=6 per vaccinated group, n=3 for PBS administered group). Symbols represent mean titers and bars represent the standard error) (FIG. 5B) Neutralizing antibody responses comparing rAd-S-N and rAd-S1-N using two different methods, surrogate VNT (sVNT) and cell-based VNT (cVNT). (FIG. 5C) IgA lung antibody titers to S1 and S2 in immunized mice. Endpoint titers were measured by standard ELISA (n=10 per group). Lines represent the median and inter-quartile range. ** p<0.0.01, *** p<0.001 defined by Mann-Whitney t-test. FIG. 5D. Neutralizing antibodies measured in the lungs post immunization.

FIGS. 6A-6B. Immunization with rAd co-expressing full length S and N vaccines induce IgG responses in a dose-dependent manner. FIGS. 6A and 6B. Balb/c mice were immunized, IN, on days 0 and 14 with 1×10⁷ IU, 1×10⁸ IU or 7.2×10⁸ IU of rAd co-expressing full length S and N (rAd-S-N). The amount of IgG specific for S1 (FIG. 6A) and S2 in serum diluted 1/4000, was evaluated using a Mesoscale binding assay. Points represent the mean and lines represent the standard deviation.

FIGS. 7A-7C. Immunization with rAd co-expressing full length S and N vaccines induce polyfunctional T cell responses in a dose-dependent manner. (FIG. 7A) Balb/c mice were immunized, IN, on days 0 and 14 with 1×10⁸ IU (Ad-S-N high), 1×10⁷ IU (Ad-S-N low) of rAd-S-N. The frequency of CD4+(top panel) or CD8+ T cells (bottom panel) that produced only IFN-γ, TNF-α, IL-2 or IL-4 after stimulation of spleen cells with 1 μg/ml (CD4+) or 5 μg/ml (CD8+) of the S peptide pools, as determined by ICS-FACS. (B) The frequency of polyfunctional CD4+(top panel) or CD8+ T cells (bottom panel) that produced more than one cytokine after stimulation of spleen cells with 1 μg/ml (CD4+) or 5 μg/ml (CD8+) S peptide pools, Bars represent the mean and the lines represent the standard error of the mean. (C) IFN-γ T cell responses to S protein 4 weeks following immunization on weeks 0 and 4 with 1×10⁶ IU, 1×10⁷ IU, 1×10⁸ IU doses of rAd-S-N were measured by ELISPOT. Bars represent the mean and the lines represent the standard deviation. * p<0.05; one-way non-parametric ANOVA with multiple comparisons.

FIGS. 8A-8B: Antibodies to S were superior when the S protein expressed in the wild-type configuration compared to the fixed version. Balb/c mice were immunized on weeks 0 and 4 with 1e8 IU per mouse (n=6), and antibody titers were measured. (FIG. 8A) IgG antibody titers over time. (FIG. 8B) Neutralizing antibody responses were measured at week 6. Note that 1:1000 was the maximum dilution performed.

FIGS. 9A-9F: (FIG. 9A) (left) Frequency of CD27++CD38++ plasmablasts in peripheral blood before (day 1) and after (day 8) vaccination as measured by flow cytometry. Bars represent median values, while error bars correspond to 95% confidence intervals. Wilcoxon test was used to compare frequencies before and after vaccination; (right) Representative flow cytometry plot showing pre- and day 8 post-vaccination CD27++CD38++ plasmablasts for one vaccine; (FIG. 9B) Fold change (day 8/day 1) in plasmablast frequencies. A total of 24/35 subjects (69%) showed a 2-fold or higher increase (with a 3.3 median fold change increase overall); (FIG. 9C) Fold change (day 8/day 1) of IgA- and B7-expressing plasmablasts in low and high dose vaccine cohorts. Mann-Whitney test was used to compare frequencies between the two different dose groups; (FIG. 9D) Fold change (day 8/day 1) in the number of IgA-positive antibody-secreting cells (ASC) reactive against the S1 domain of the Sars-CoV-2 spike antigen; (FIG. 9E) Fold change (day 29/day 1) in S-, N-, or RBD-specific IgA antibodies in the serum as measured by MSD platform. Red dotted lines represent median values. Mann-Whitney test was used to compare frequencies between the two different dose groups; (FIG. 9F) Fold change (day 29/day 1) in S-, N-, or RBD-specific IgA antibodies in nasal and saliva samples as measured by MSD platform.

FIG. 10A-E provides data illustrating that VXA-CoV2-1 elicits anti-viral T cells of high magnitude. PBMCs pre- and post-immunization were restimulated with SARS-CoV-2 peptides, surface stained for CD8 and degranulation marker CD107a, and intracellularly stained for cytokines. (A) Percentage of IFNγ, TNFα, and CD107a CD8 T cells pre (d0) and 7 days post (d7) immunization in response to SARS-CoV-2 Spike peptides. (B) Dual IFNγ⁺ TNFα⁺CD8⁺ T cells as a percent of CD8 T cells, pre (d0) and post (d7) immunization in response to SARS-CoV-2 Spike peptides. C) Pie-chart representing the % of subjects that had anti-viral T cell responses of various types. (D) Representative facs plots of IFNγ after stimulation with either CEF or S peptides. (E) IFNγ, percent of CD8+ T cells post immunization increase over d0 in response to S&N peptides from 4 endemic coronaviruses.

FIG. 11A-B provides data illustrating that oral VXA-CoV-2 elicits anti-viral CD8 T cells of higher magnitude than intramuscular mRNA vaccines. PBMCs pre- and post-immunization were re-stimulated with SARS-CoV-2 peptides, surface stained for CD8 and degranulation marker CD107a, and intracellularly stained for cytokines. PBMCs from all 3 vaccines were analyzed at the same time. (A) Graph shows IFNγ, TNFα, and CD107a percent of CD8⁺ T cells increase over background post immunization in response to SARS-CoV-2 Spike protein. (B) IFNγ data from (A) is plotted alongside vaxart cohort and convalescents. Convalescent subjects are not day 1 subtracted due to no pre-infection samples obtained. (C) Time course of Pfizer and Moderna T cell responses.

FIG. 12A-E provides data illustrating that PBMCs pre- and post-immunization were restimulated with either SARS-CoV-2 Nucleocapsid or Spike peptides, surface stained for CD4, CD8 and degranulation marker CD107a, and intracellularly stained for cytokines. (A) Dose stratification of data in FIG. 10A. (B) Time course of sentinel subjects showing maintenance of CD8⁺IFNγ⁺ T cell responses post boost. (C) CD4 T cell responses to spike. (D-E) VXA-CoV2-1 induces anti-viral T cells in response to nucleocapsid Graph shows IFNγ, TNFα, and CD107a percent of CD8⁺ T cells (D) or CD4⁺ T cells (E) increase over background post immunization in response to SARS-CoV-2 nucleocapsid peptides.

FIG. 13A-B: (A) Human antibody titers (IgG) against SARS-CoV-2 spike (S1) in individuals fully vaccinated (two doses) with Moderna or Pfizer COVID-19 vaccine. The titers were measured at day 7 post second dose using a standardized SARS-CoV-2 spike (S1) human IgG ELISA kit. *One subject was measured at day 29 post first dose due to sample loss, two individuals did not have serum taken prior to vaccination (B) CD4 responses in comparator experiment: PBMCs pre- and post-immunization were restimulated with SARS-CoV-2 peptides, surface stained for CD4 and degranulation marker CD107a, and intracellularly stained for cytokines. Graph shows IFNγ, TNFα, and CD107a percent of CD4 T cells increase over background post immunization in response to SARS-CoV-2 spike peptides.

FIG. 14 provides data illustrating that intranasal administration of a vaccine construct that expresses HPV E6 and E7 proteins and a SARS-CoV-2 N protein resulted in enhanced ability of T cells to response to HPV compared to a comparison construct that lacked the SARS-CoV-2 N protein.

FIG. 15 provides data illustrating that a vaccine construct administered intranasally that expressed SARS-CoV-2 S and N proteins elicited a cytotoxic anti-spike T cells response that was higher than a comparable vaccine that expressed S alone.

### DETAILED DESCRIPTION OF THE DISCLOSURE

### I. Introduction

Coronavirus disease 2019 (COVID-19) is an infectious disease caused by severe acute respiratory syndrome coronavirus 2 (SARS-CoV-2). SARS-CoV-2 is a mucosal viral pathogen that infects the epithelial cells of the lungs and possibly even the intestine (9). Some symptoms of the disease include, for example, fever, cough, shortness of breath, muscle pain, sputum production, diarrhea, sore throat, loss of smell, and abdominal pain. While the majority of cases result in mild symptoms, some progress to viral pneumonia and multi-organ failure.

The virus is spread mainly through close contact and via respiratory droplets produced when people cough or sneeze. People may also contract COVID-19 by touching a contaminated surface and then their face. The infection is most contagious when people are symptomatic, although spread may be possible before symptoms appear. Currently, there is no vaccine or specific antiviral treatment for COVID-19. Managing the disease involves treatment of symptoms, supportive care, isolation, and some experimental measures.

The genome of SARS-CoV-2 virus encodes four major structural proteins including spike (S), nucleocapsid (N), membrane (M), and envelope (E), which are required to make a complete virus particle. After viral entry, 16 non-structural proteins are formed from two large precursor proteins. These viruses have a relatively large positive sense RNA strand (26-32 kb), and without erroneous editing, the RNA can mutate, evolve, and undergo homologous recombination with other family members to create new viral species (6). The S protein is believed to be the major antibody target for coronavirus vaccines, as the protein is responsible for receptor binding, membrane fusion, and tissue tropism. When comparing SARS-CoV-2 Wu-1 (GenBank Accession No. QHD43416.1) to SARS-CoV (GenBank Accession No. AY525636.1), the S protein was found to have 76.2% identity, 87.2% similarity, and 2% gaps in 1273 positions (7). Both SARS-CoV and SARS-CoV-2 are believed to use the same receptor for cell entry: the angiotensin-converting enzyme 2 receptor (ACE2), which is expressed on some human cell types (8). As discussed in the article by Xu, et al., high expression levels of ACE2 are present in type II alveolar cells the lungs, absorptive enterocytes of the ileum and colon, and possibly even in oral tissues such as the tongue (32).

Provided herein are vaccines, immunogenic compositions, and methods for treating COVID-19 that involve the use of chimeric adenoviral vectors that contain one or more nucleic acids encoding one or more SARS-CoV-2 proteins and a nucleic acid encoding a TLR-3 agonist.

### II. Definitions

The term “chimeric” or “recombinant” as used herein with reference, e.g., to a nucleic acid, protein, or vector, indicates that the nucleic acid, protein or vector, has been modified by the introduction of a heterologous nucleic acid or protein or the alteration of a native nucleic acid or protein. Thus, for example, chimeric and and recombinant vectors include nucleic acid sequences that are not found within the native (non-chimeric or non-recombinant) form of the vector. A chimeric adenoviral expression vector refers to an adenoviral expression vector comprising a nucleic acid sequence encoding a heterologous polypeptide, such as a SARS-CoV-2 protein.

The term “expression vector” refers to a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The term “promoter” refers to an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as many as several thousand base pairs from the start site of transcription. Promoters include constitutive and inducible promoters. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “SARS-CoV-2” or “severe acute respiratory syndrome coronavirus 2” refers to a coronavirus within a large genus of betacoronaviruses from the viral family of Coronaviridae. Genbank Accession No. MN908947.3 is a published DNA sequence of SARS-CoV-2. The virus is spread mainly through close contact and via respiratory droplets produced when people cough or sneeze.

The term “SARS-CoV-2 protein” refers to a protein encoded by the nucleic acid of SARS-CoV-2 (e.g., Genbank Accession No. MN908947.3) or a fragment of the protein. In some embodiments, a fragment of the SARS-CoV-2 protein comprises at least 10, 20, or more contiguous amino acids from the full-length protein encoded by the sequence of Genbank Accession No. MN908947.3. For example, a SARS-CoV-2 protein can be a structural protein of the full-length protein encoded by the nucleic acid of the SARS-CoV-2 virus, such as a SARS-CoV-2 S protein (surface glycoprotein; e.g., SEQ ID NO:1 or SEQ ID NO:19 or SEQ ID NO:20, or variants thereof, e.g., that are at least 90%, 95%, 97%, 98%, or 99% identical to SEQ ID NO:1 or or SEQ ID NO:19 or SEQ ID NO:20) or a SARS-CoV-2 N protein (nucleocapsid phosphoprotein; SEQ ID NO:2). A SARS-CoV-2 protein can also be a fusion protein that contains different portions of the full-length protein encoded by the nucleic acid of the SARS-CoV-2 virus. For example, a SARS-CoV-2 fusion protein can contain a S1 region of a SARS-CoV-2 S protein, a furin site, and a SARS-CoV-2 N protein (e.g., SEQ ID NO:10).

The term “COVID-19” or “coronavirus disease 2019” refers to an infectious disease caused by the SARS-CoV-2 virus.

The term “TLR agonist” or “Toll-like receptor agonist” as used herein refers to a compound that binds and stimulates a Toll-like receptor including, e.g., TLR-2, TLR-3, TLR-6, TLR-7, or TLR-8. TLR agonists are reviewed in MacKichan, IAVI Report. 9.1-5 (2005) and Abreu et al., J Immunol, 174(8), 4453-4460 (2005). Agonists induce signal transduction following binding to their receptor.

The term “TLR-3 agonist” or “Toll-like receptor 3 agonist” as used herein refers to a compound that binds and stimulates the TLR-3. TLR-3 agonists have been identified including double-stranded RNA, virally derived dsRNA, several chemically synthesized analogs to double-stranded RNA including polyinosine-polycytidylic acid (poly I:C)-polyadenylic-polyuridylic acid (poly A:U) and poly I:poly C, and antibodies (or cross-linking of antibodies) to TLR-3 that lead to IFN-beta production (Matsumoto, M, et al, Biochem Biophys Res Commun 24:1364 (2002), de Bouteiller, et al, J Biol Chem 18:38133-45 (2005)). In some embodiments, a TLR-3 agonist comprises a sequence of any one of SEQ ID NOS: 11-18. In some embodiments, a TLR-3 agonist is a dsRNA (e.g., dsRNA encoded by a nucleic acid comprising a sequence set forth in SEQ ID NO: 11).

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The terms “nucleic acid” and “polynucleotide” are used interchangeably herein to refer to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

The term “antigen” refers to a protein or part of a polypeptide chain that can be recognized by T cell receptors and/or antibodies. Typically, antigens are derived from bacterial, viral, or fungal proteins.

The term “immunogenically effective dose or amount” of the compositions of the present disclosure is an amount that elicits or modulates an immune response specific for the SARS-CoV-2 protein. Immune responses include humoral immune responses and cell-mediated immune responses. An immunogenic composition can be used therapeutically or prophylactically to treat or prevent disease at any stage. Humoral immune responses are generally mediated by cell free components of the blood, i.e., plasma or serum; transfer of the serum or plasma from one individual to another transfers immunity. Cell mediated immune responses are generally mediated by antigen specific lymphocytes; transfer of the antigen specific lymphocytes from one individual to another transfers immunity.

The term “therapeutic dose” or “therapeutically effective amount” or “effective amount” of a chimeric adenoviral vector or a composition comprising a chimeric adenoviral vector refers to an amount of the vector or composition comprising the vector which prevents, alleviates, abates, or reduces the severity of symptoms of diseases and disorders associated with the source of the SARS-CoV-2 protein (e.g., a SARS-CoV-2 virus).

The term “adjuvant” refers to a non-specific immune response enhancer. Suitable adjuvants include, for example, cholera toxin, monophosphoryl lipid A (MPL), Freund's Complete Adjuvant, Freund's Incomplete Adjuvant, Quil A, and Al(OH). Adjuvants can also be those substances that cause antigen-presenting cell activation and enhanced presentation of T cells through secondary signaling molecules like Toll-like receptors. Examples of Toll-like receptors include the receptors that recognize double-stranded RNA, bacterial flagella, LPS, CpG DNA, and bacterial lipopeptide (Reviewed recently in Abreu et al., J Immunol, 174(8), 4453-4460 (2005)).

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline and 0-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, the term “percent identity” or “percent identical,” used in the context of nucleic acids or polypeptides, refers to a sequence that has at least 50% sequence identity with a reference sequence. Alternatively, percent identity can be any integer from 50% to 100%. In some embodiments, a sequence is substantially identical to a reference sequence if the sequence has at least 50%, 55%, 60%, 65%, 70%, 75%, 80%, 85%, 90%, 91%, 92%, 93%, 94%, 95%, 96%, 97%, 98%, or 99% sequence identity to the reference sequence as determined using the methods described herein; preferably BLAST using standard parameters, as described below. Percent identity may also be determined by manual alignment.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A comparison window includes reference to a segment of any one of the number of contiguous positions, e.g., a segment of at least 10 residues. In some embodiments, the comparison window has from 10 to 600 residues, e.g., about 10 to about 30 residues, about 10 to about 20 residues, about 50 to about 200 residues, or about 100 to about 150 residues, in which a sequence may be compared to a reference sequence of the same number of contiguous positions after the two sequences are optimally aligned.

Algorithms that are suitable for determining percent sequence identity and sequence similarity are the BLAST and BLAST 2.0 algorithms, which are described in Altschul el al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul el al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff& Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, an amino acid sequence is considered similar to a reference sequence if the smallest sum probability in a comparison of the test amino acid sequence to the reference amino acid sequence is less than about 0.01, more preferably less than about 10⁻⁵, and most preferably less than about 10⁻²⁰.

### II. Compositions and Methods of the Present Disclosure

The disclosure provides compositions comprising chimeric adenoviral vectors. The chimeric adenoviral vectors can include one or more nucleic acids encoding one or more SARS-CoV-2 proteins. The chimeric adenoviral vectors can also include a nucleic acid encoding a toll-like receptor (TLR) agonist (e.g., a TLR-3 agonist), which can serve as an effective adjuvant when administered in conjunction with viral vectors.

In some embodiments, the chimeric adenoviral vectors of the disclosure comprise an expression cassette comprising the following elements: (a) a first promoter operably linked to a nucleic acid encoding a first SARS-CoV-2 protein; and (b) a second promoter operably linked to a nucleic acid encoding a toll-like receptor-3 (TLR-3) agonist. The first SARS-CoV-2 protein can be a full-length protein (or a substantially identical protein thereof) encoded by the nucleic acid of SARS-CoV-2 (e.g., Genbank Accession No. MN908947.3) or a fragment of the protein. For example, a first SARS-CoV-2 protein can be a structural protein of the full-length protein encoded by the nucleic acid of the SARS-CoV-2 virus, such as a SARS-CoV-2 S protein (surface glycoprotein; e.g., SEQ ID NO:1 or a substantially identical protein thereof, e.g., SEQ ID NO:19 or SEQ ID NO:20, or variants thereof, e.g., that are at least 90%, or at least 95%, 96%, 97%, 98%, or 99% identical to SEQ ID NO:1 or SEQ ID NO:19 or SEQ ID NO:20); or a SARS-CoV-2 N protein (nucleocapsid phosphoprotein; SEQ ID NO:2 or a substantially identical protein thereof, e.g., a variant thereof, e.g., that has at least 90%, or at least 95%, 96%, 97%, 98%, or 99% identity to SEQ ID NO:2). In other embodiments, a first SARS-CoV-2 protein can be a protein encoded by other parts of the nucleic acid of the SARS-CoV-2 virus, such as a protein encoded by the ORF1ab gene, a protein encoded by the ORF3a gene, a protein encoded by the E gene (encoding an envelope protein), a protein encoded by the M gene (encoding a membrane glycoprotein), a protein encoded by the ORF6 gene, a protein encoded by the ORF7a gene, a protein encoded by the ORF8 gene, or a protein encoded by the ORF10 gene.

In further embodiments, a first SARS-CoV-2 protein can be a fusion protein that contains different portions of the full-length protein encoded by the nucleic acid of the SARS-CoV-2 virus. For example, a SARS-CoV-2 fusion protein can contain a S1 region of a SARS-CoV-2 S protein, a furin site, and a SARS-CoV-2 N protein (e.g., SEQ ID NO:10).

A nucleic acid that encodes a first SARS-CoV-2 protein in element (a) can comprise a sequence having at least 85%, 90%, 95%, 96%, 97%, 99%, or 100% (e.g., 85%, 87%, 89%, 91%, 93%, 95%, 97%, 99% c, or 100%) identity to the sequence of SEQ ID NO:3, which encodes the amino acid sequence of the SARS-CoV-2 S protein (SEQ ID NO:1). In some embodiments, a first SARS-CoV-2 protein in element (a) can comprise a sequence having at least 85%, 90%, 95%, 96%, 97%, 99%, or 100% (e.g., 85%, 87%, 89%, 91%, 93%, 95%, 97%, 99%, or 100%) identity to the sequence of SEQ ID NO:3 and encode a SARS-CoV-2 S protein of SEQ ID NO:19 or SEQ ID NO:20. In some embodiments, a first SARS-CoV-2 protein in element (a) can comprise a sequence having at least 85%, 90%, 95%, 96%, 97%, 99%, or 100% (e.g., 85%, 87%, 89%, 91%, 93%, 95%, 97%, or 99%) identity to the sequence of SEQ ID NO:3 and encodes a SARS-CoV-2 S protein variant at least 90%, 95%, 97%, 98%, or 99% identical to SEQ ID NO:1 or or SEQ ID NO:19 or SEQ ID NO:20. In other embodiments, a nucleic acid that encodes a first SARS-CoV-2 protein in element (a) can comprises a sequence having at least 85%, 90%, 95%, 97%, 99%, or 100% (e.g., 85%, 87%, 89%, 91%, 93%, 95%, 97%, 99%, or 100%) identity to the sequence of SEQ ID NO:4, which encodes the amino acid sequence of the SARS-CoV-2 N protein (SEQ ID NO:2). In some embodiments, a first SARS-CoV-2 protein in element (a) can comprise a sequence having at least 85%, 90%, 95%, 96%, 97%, 99%, or 100% (e.g., 85%, 87%, 89%, 91%, 93%, 95%, 97%, or 99%) identity to the sequence of SEQ ID NO:4 and encodes a SARS-CoV-2 N protein variant at least 90% identical, or at least 95%, 96%, 97%, 98%, or 99% identical to SEQ ID NO:2. In further embodiments, a nucleic acid that encodes a first SARS-CoV-2 protein in element (a) can comprises a sequence having at least 85%, 90%, 95%, 97%, 99%, or 100% (e.g., 85%, 87%, 89%, 91%, 93%, 95%, 97%, 99%, or 100%) identity to the sequence of SEQ ID NO:5, which encodes the amino acid sequence of the SARS-CoV-2 fusion protein that contains a S1 region of a SARS-CoV-2 S protein, a furin site, and a SARS-CoV-2 N protein (SEQ ID NO:10).

In addition to a first SARS-CoV-2 protein, the chimeric adenoviral vectors of the disclosure can further comprise element (c) a third promoter operably linked to a nucleic acid encoding a second SARS-CoV-2 protein. In particular embodiments, the order of the elements in the expression cassette from the N-terminus to the C-terminus is: element (a), element (c), and element (b). In some embodiments, the first and second SARS-CoV-2 proteins encoded by their respective nucleic acids in elements (a) and (c) in the expression cassette are the same. In some embodiments, the first and second SARS-CoV-2 proteins encoded by their respective nucleic acids in elements (a) and (c) in the expression cassette are different.

For example, the first SARS-CoV-2 protein can be a SARS-CoV-2 S protein (e.g., SEQ ID NO:1 or SEQ ID NO:19 or SEQ ID NO:20, or variants thereof, e.g. that are at least 90%, 95%, 96%, 97%, 98%, or 99% identical to SEQ ID NO:1 or SEQ ID NO:19 or SEQ ID NO:20, which is encoded by a nucleic acid sequence having at least 85%, 90%, 95%, 97%, 99%, or 100% (e.g., 85%, 87%, 89%, 91%, 93%, 95%, 97%, 99%, or 100%) identity to the sequence of SEQ ID NO:3) and the second SARS-CoV-2 protein can be a SARS-CoV-2 N protein (e.g., SEQ ID NO:2, which is encoded by a nucleic acid sequence having at least 85%, 90%, 95%, 96%, 97%, 99%, or 100% (e.g., 85%, 87%, 89%, 91%, 93%, 95%, 97%, 99%, or 100%) identity to the sequence of SEQ ID NO:4). In another example, the first SARS-CoV-2 protein can be a SARS-CoV-2 N protein (e.g., SEQ ID NO:2, which is encoded by a nucleic acid sequence having at least 85%, 90%, 95%, 97%, 99%, or 100% (e.g., 85%, 87%, 89%, 91%, 93%, 95%, 97%, 99%, or 100%) identity to the sequence of SEQ ID NO:4) and the second SARS-CoV-2 protein can be a SARS-CoV-2 S protein (e.g., SEQ ID NO:1 or SEQ ID NO:19 or SEQ ID NO:20, or variants thereof, e.g that are at least 90%, 95%, 97%, 98%, or 99% identical to SEQ ID NO:1 or or SEQ ID NO:19 or SEQ ID NO:20, which is encoded by a nucleic acid sequence having at least 85%, 90%, 95%, 97%, 99%, or 100% (e.g., 85%, 87%, 89%, 91%, 93%, 95%, 97%, 99%, or 100%) identity to the sequence of SEQ ID NO:3).

In another example, the first SARS-CoV-2 protein can be a SARS-CoV-2 N protein (e.g., SEQ ID NO:2; or a variant thereof, e.g., having at least 95%, 96%, 97%, 98%, or 99% identity to SEQ ID NO:2) or a SARS-CoV-2 S protein (e.g., SEQ ID NO:1 or SEQ ID NO:19 or SEQ ID NO:20, or variants thereof, e.g that are at least 90%, 95%, 97%, 98%, or 99% identical to SEQ ID NO:1 or or SEQ ID NO:19 or SEQ ID NO:20) and the second SARS-CoV-2 protein can be a SARS-CoV-2 fusion protein (e.g., SEQ ID NO:10, which is encoded by a nucleic acid sequence having at least 85%, 90%, 95%, 97%, 99%, or 100% (e.g., 85%, 87%, 89%, 91%, 93%, 95%, 97%, 99%, or 100%) identity to the sequence of SEQ ID NO:5).

In yet another example, the first SARS-CoV-2 protein can be a SARS-CoV-2 fusion protein (e.g., SEQ ID NO:10, which is encoded by a nucleic acid sequence having at least 85%, 90%, 95%, 97%, 99%, or 100% (e.g., 85%, 87%, 89%, 91%, 93%, 95%, 97%, 99%, or 100%) identity to the sequence of SEQ ID NO:5) and the second SARS-CoV-2 protein can be a SARS-CoV-2 N protein (e.g., SEQ ID NO:2; or a variant at least 95%, 96%, 97%, 98%, or 99% identical to SEQ ID NO:2) or a SARS-CoV-2 S protein (e.g., SEQ ID NO:1 or SEQ ID NO:19 or SEQ ID NO:20, or variants thereof, e.g., that are at least 90%, 95%, 97%, 98%, or 99% identical to SEQ ID NO:1 or or SEQ ID NO:19 or SEQ ID NO:20).

One of skill understands that variants of SARS-CoV-2 proteins, e.g., variants of the SARS-CoV-2 S protein, emerge rapidly. Examples of two variant S protein sequences, UK B.1.1.1.7 variant and South African B.1.351 501Y.V2 variant, are provided in SEQ ID NOS:19 and 20, respectively. Other S protein variants are known, including a Brazil variant, P.1 (L18F, T20N, P26S, D138Y, R190S, K417T, E484K, N501Y, D614G, H655Y, T1027I); an Indian variant B.1.617 (L452R, E484Q, D614G), and an Omicron variant, among others. Thus, in some embodiments, the SARS-CoV-2 S protein sequence is a variant sequence identified in a patient population.

In addition to the above-described vectors triggering an immune response to SARS-CoV-2 protein, in view of the data shown in Example 6, provided herein are embodiments in which co-introduction of a coronavirus N protein, typically a SARS-CoV-2 N protein, with any second antigen, which can be from a non-SARS-CoV-2 antigen source, can be used to stimulate a CD8 T-cell immune response to the second antigen.

Accordingly the disclosure also provides for polynucleotides encoding a SARS-CoV-2 N protein (e.g., SEQ ID NO:2 or a variant thereof having at least 90% identity, or at least 95% identity, to SEQ ID NO:2, or a fragment thereof) and encoding a second antigenic protein from any source. For example the second antigenic protein can be from a non-SARS-CoV-2 virus, a bacterium, other pathogen or cancer. For example, in some embodiments, the second antigen is a protein or fragment thereof from Herpes simplex, type 1; Herpes simplex, type 2; encephalitis virus, papillomavirus, Varicella-zoster virus; Epstein-barr virus; Human cytomegalovirus; Human herpes virus, type 8; Human papillomavirus; BK virus, JC virus; Smallpox; polio virus; Hepatitis B virus; Human bocavirus; Parvovirus B19; Human astrovirus; Norwalk virus; coxsackievirus; hepatitis A virus; poliovirus; rhinovirus; Severe acute respiratory syndrome virus; Hepatitis C virus; Yellow Fever virus; Dengue virus; West Nile virus; Rubella virus; Hepatitis E virus; Human Immunodeficiency virus (HIV); Influenza virus: Guanarito virus; Junin virus; Lassa virus; Machupo virus; Sabia virus; Crimean-Congo hemorrhagic fever virus; Ebola virus; Marburg virus; Measles virus; Mumps virus; Parainfluenza virus; Respiratory syncytialvirus; Human metapneumovirus; Hendra virus; Nipah virus; Rabies virus; Hepatitis D; Rotavirus; Orbivirus; Coltivirus; Banna virus; Human Enterovirus; Hanta virus; West Nile virus; Middle East Respiratory Syndrome Corona Virus; Japanese encephalitis virus; Vesicular exanthernavirus; or Eastern equine encephalitis. See, also, U.S. Pat. No. 8,222,224 for a list of antigens that can be used.

Particular examples of second antigens that can be used as described herein in combination with a SARS-CoV-2 N protein include but are not limited to those derived from norovirus (e.g., VP1), Respiratory syncytial virus (RSV), the influenza virus (e.g., HA, NA, M1, NP), human immunodeficiency virus (HIV, e.g., gag, pol, env, etc.), human papilloma virus (HPV, e.g., capsid proteins such as L1), Venezuelan Equine Encephalomyelitis (VEE) virus, Epstein Barr virus, herpes simplex virus (HSV), human herpes virus, rhinoviruses, cocksackieviruses, enteroviruses, hepatitis A, B, C, E, and G (HAV, HBV, HCV, HEV, HGV e.g., surface antigen), mumps virus, rubella virus, measles virus, poliovirus, smallpox virus, rabies virus, and Varicella-zoster virus.

Suitable viral antigens useful as second antigens as described herein also include viral nonstructural proteins, e.g., proteins encoded by viral nucleic acid that do not encode for structural polypeptides, in contrast to those that make capsid or the protein surrounding a virus. Non-structural proteins include those proteins that promote viral nucleic acid replication, viral gene expression, or post-translational processing, such as, for example, Nonstructural proteins 1, 2, 3, and 4 (NS1, NS2, NS3, and NS4, respectively) from Venezuelan Equine encephalitis (VEE), Eastern Equine Encephalitis (EEE), or Semliki Forest.

Bacterial antigens useful as second antigens as described herein can be derived from, for example, Staphylococcus aureus, Staphylococcus epidermis, Helicobacter pylori, Streptococcus bovis, Streptococcus pyogenes, Streptococcus pneumoniae, Listeria monocytogenes, Mycobacterium tuberculosis, Mycobacterium leprae, Corynebacterium diphtheriae, Borrelia burgdorferi, Bacillus anthracis, Bacillus cereus, Clostridium botulinum, Clostridium difficile, Salmonella typhi, Vibrio chloerae, Haemophilus influenzae, Bordetella pertussis, Yersinia pestis, Neisseria gonorrhoeae, Treponema pallidum, Mycoplasm sp., Legionella pneumophila, Rickettsia typhi, Chlamydia trachomatis, and Shigella dysenteriae, Vibrio cholera (e.g., Cholera toxin subunit B, cholera toxin-coregulated pilus (TCP)); Helicobacter pylorii (e.g., VacA, CagA, NAP, Hsp, catalase, urease); E. coli (e.g., heat-labile enterotoxin, fimbrial antigens).

Parasite antigens useful as second antigens as described herein can be derived from, for example, Giardia lamblia, Leishmania sp., Trypanosoma sp., Trichomonas sp., Plasmodium sp. (e.g., P. falciparum surface protein antigens such as pfs25, pfs28, pfs45, pfs84, pfs 48/45, pfs 230, Pvs25, and Pvs28); Schistosoma sp.; Mycobacterium tuberculosis (e.g., Ag85, MPT64, ESAT-6, CFP10, R8307, MTB-32 MTB-39, CSP, LSA-1, LSA-3, EXP1, SSP-2, SALSA, STARP, GLURP, MSP-1, MSP-2, MSP-3, MSP-4, MSP-5, MSP-8, MSP-9, AMA-1, Type 1 integral membrane protein, RESA, EBA-175, and DBA).

Fungal antigens useful as second antigens as described herein can be derived from, for example, Tinea pedis, Tinea corporus, Tinea cruris, Tinea unguium, Cladosporium carionii, Coccidioides immitis, Candida sp., Aspergillus fumigatus, and Pneumocystis carinii.

Cancer antigens useful as second antigens as described herein include, for example, antigens expressed or over-expressed in colon cancer, stomach cancer, pancreatic cancer, lung cancer, ovarian cancer, prostate cancer, breast cancer, skin cancer (e.g., melanoma), leukemia, or lymphoma. Exemplary cancer antigens include, for example, HPV L1, HPV L2, HPV E1, HPV E2, placental alkaline phosphatase, AFP, BRCA1, Her2/neu, CA 15-3, CA 19-9, CA-125, CEA, Hcg, urokinase-type plasminogen activator (Upa), plasminogen activator inhibitor, CD53, CD30, CD25, C5, CD11a, CD33, CD20, ErbB2, CTLA-4. See Sliwkowski & Mellman (2013) Science 341:6151 for additional cancer targets.

While an attenuated adenovirus can be used to express a SARS-CoV-2 N protein and second antigenic protein (e.g., to generate a CD8 T-cell response), other polynucleotides or vectors can also be used. Expression vectors can include, for example, virally-derived vectors, e.g., recombinant adeno-associated virus (AAV) vectors, retroviral vectors, adenoviral vectors, modified vaccinia Ankara (MVA) vectors, and lentiviral (e.g., HSV-1-derived) vectors (see, e.g., Brouard et al. (2009) British J. Pharm. 157:153). In other embodiments, the SARS-CoV-2 N protein (e.g., SEQ ID NO:2) and second antigenic protein can be encoded by a polynucleotide, e.g., naked or encapsulated DNA or RNA, e.g., mRNA (see, e.g., U.S. Patent Publication No. 2020/0254086 for details of various aspects for RNA-based vaccines).

In some embodiments a vector that comprises a region encoding a SAR-CoV-2 N protein and a region encoding a second antigenic protein, further comprises a nucleic acid encoding a TLR agonist (e.g., a TLR-3 agonist), which can serve as an effective adjuvant when administered in conjunction with vectors, such as viral vectors.

In some embodiments, the vector comprises a ribosomal skipping element situated between the region of the nucleic acid that encode the N protein and the region encoding the second antigenic protein. In some embodiments, the vector comprises an IRES situated between the N protein and second antigenic protein to produce a bicistronic transcript. In some embodiments, the ribosomal skipping element is a sequence encoding a virus 2A peptide (T2A), a porcine teschovirus-1 2A peptide (P2A), a foot-and-mouth disease virus 2A peptide (F2A), equine rhinitis A virus 2A peptide (E2A), a cytoplasmic polyhedrosis virus 2A peptide (BmCPV 2A), or a flacherie virus of B. mori 2A peptide (BmIFV 2A); situated between the N protein and the second antigenic protein. In some embodiments, the construct further encodes a TLR agonist.

In some embodiments, a vector, e.g., a viral vector, encodes a SARS-Co-V2 N protein (e.g., an N protein sequence of SEQ ID NO:2, or a variant thereof, e.g., at least 90% identical, or at least 95% identical to SEQ ID NO:2) and a second antigenic protein, in which expression of the N protein and second antigenic protein is driven by different promoters.

In some embodiments, the vector comprises a first promoter operably linked to polynucleotide sequence encoding a SARS-CoV-2 N protein and a second promoter operably linked to the second antigenic protein. In some embodiments, the vector, e.g., a viral vector, can further comprise a third promoter operably linked to a TLR agonist, e.g., a TLR-3 agonist.

In particular embodiments, the order of the elements in the expression cassette from the N-terminus to the C-terminus is: a sequence encoding an antigenic protein, a sequence encoding a SARS-Co-V2 N protein and a sequence encoding a TLR agonist, e.g., a TLR 3 agonist.

In further embodiments, an antigenic protein can be fused to the N protein sequence For example, a fusion protein can contain an antigenic protein, a furin site, and a SARS-CoV-2 N protein, or variant thereof, e.g., at least 90% identical, or at least 95% identical to SEQ ID NO:2.

In some embodiments, a SARS-CoV-2 N protein encoded by a vector has at least 90% identity to SEQ ID NO:2. In some embodiments, the N protein encoded by the vector has at least 95%, 96%, 97%, 98%, or 99% identity to SEQ ID NO:2.

In some embodiments, the vector comprises an expression cassette as described herein in which a second antigenic protein replaces a SARS-CoV-2 S protein in the constructs provided herein that encode both the N protein and SARS-CoV-2 S proteins. Thus, for example, in some embodiments, the vector comprises sequences as follows (5′-3):
CMV-second antigenic protein-BGH-βActin-N protein-SPA-BGH-CMV-dsRNA-SPA
   in which “CMV” is a CMV promoter; “second antigenic protein” is a nucleic acid sequence encoding a second antigenic protein, e.g., from an infectious disease agent or a cancer antigen as described herein, “BGH” is a bovine growth hormone polyadenylation signal sequence”; “βActin” is a beta-actin promoter, e.g., a human beta-actin promoter; “N-protein” is a nucleic acid sequence encoding a SARS-CoV2 N protein as described herein, e.g., SEQ ID NO:2, or a protein having at least 90% identity or at least 95% identity to SEQ ID NO:2, “SPA” is a synthetic polyA sequence, and “dsRNA” is a nucleic acid sequence encoding a TLR agonist, e.g., a TLR-3 agonist.

In some embodiments, an N protein from an alternative coronavirus is employed in place of the SARS-CoV-2 N protein in constructs comprising an N protein and an antigenic protein, such as an infection disease antigen or cancer antigen. Thus, for example, in some embodiments, such a construct can comprise a SARS-CoV or MERS N protein.

In some embodiments, the vector is an adenoviral vector, e.g., an adenovirus 5 (Ad5) vector as described below.

Suitable Adenoviral Vectors

In some embodiments, an adenoviral vector as described herein is adenovirus 5 (Ad5), which can include, for example, Ad5 with deletions of the E1/E3 regions and Ad5 with a deletion of the E4 region. Other suitable adenoviral vectors include strains 2, orally tested strains 4 and 7, enteric adenoviruses 40 and 41, and other strains (e.g. Ad34) that are sufficient for delivering an antigen and eliciting an adaptive immune response to the transgene antigen (Lubeck et al., Proc Natl Acad Sci USA, 86(17), 6763-6767 (1989); Shen et al., J Virol, 75(9), 4297-4307 (2001); Bailey et al., Virology, 202(2), 695-706 (1994)). In some embodiments, the adenoviral vector is a live, replication incompetent adenoviral vector (such as E1 and E3 deleted rAd5), live and attenuated adenoviral vector (such as the EIB55K deletion viruses), or a live adenoviral vector with wild-type replication.

The transcriptional and translational control sequences in expression vectors to be used in transforming vertebrate cells in vivo may be provided by viral sources. For example, commonly used promoters and enhancers are derived, e.g., from beta-actin, adenovirus, simian virus (SV40), and human cytomegalovirus (CMV). For example, vectors allowing expression of proteins under the direction of the CMV promoter, beta-actin promoter, SV40 early promoter, SV40 later promoter, metallothionein promoter, murine mammary tumor virus promoter, Rous sarcoma virus promoter, transducer promoter, or other promoters shown effective for expression in mammalian cells are suitable. Further viral genomic promoter, control and/or signal sequences may be used, provided such control sequences are compatible with the host cell chosen.

Various promoters can be used in the chimeric adenoviral vectors described herein. In some embodiments, when the chimeric adenoviral vector expresses two or more nucleic acids, the promoters used to express the nucleic acids can be identical or different. For example, in some embodiments, a first promoter used in to express an element (a) and a second promoter used to express an element (b) can both be a CMV promoter, or the two promoters may be different, e.g., one promoter is a CMV promoter and the other promoter is a beta-actin promoter. In other embodiments, when an element (c) is included, a third promoter can be identical or different from the first and/or second promoter. For example, the first promoter and the second promoter can both be a CMV promoter and the third promoter can be a beta-actin promoter (e.g., a human beta-actin promoter).

One of skill additionally understands that expression cassettes to express polypeptides as described herein can contain additional regulatory elements such as a polyadenylation signal, e.g., bovine growth hormone polyadenylation signal, and other sequences to regulate expression, such as terminator sequences or RNA stability elements.

TLR Agonists

The chimeric adenoviral vectors described herein can also include a nucleic acid encoding a toll-like receptor (TLR) agonist, which can serve as an effective adjuvant when administered in conjunction with viral vectors. TLR agonists can be used to enhance the immune response to the SARS-CoV-2 protein. In some embodiments, TLR-3 agonists are used. In some embodiments, the TLR agonists described herein can be delivered simultaneously with the expression vector encoding an antigen of interest (e.g., a SARS-CoV-2 protein). In other embodiments, the TLR agonists can be delivered separately (i.e., temporally or spatially) from the expression vector encoding an antigen of interest (e.g., a SARS-CoV-2 protein). For example, the expression vector can be administered via a non-parenteral route (e.g., orally, intranasally, or mucosally), while the TLR agonist can be delivered by a parenteral route (e.g., intramuscularly, intraperitoneally, or subcutaneously).

In particular embodiments, a TLR-3 agonist is can be used to stimulate immune recognition of an antigen of interest. TLR-3 agonists include, for example, short hairpin RNA, virally derived RNA, short segments of RNA that can form double-strands or short hairpin RNA, and short interfering RNA (siRNA). In one embodiment of the disclosure, the TLR-3 agonist is virally derived dsRNA, such as for example, a dsRNA derived from a Sindbis virus or dsRNA viral intermediates (Alexopoulou et al, Nature 413:732-8 (2001)). In some embodiments, the TLR-3 agonist is a short hairpin RNA. Short hairpin RNA sequences typically comprise two complementary sequences joined by a linker sequence. The particular linker sequence is not a critical aspect of the disclosure. Any appropriate linker sequence can be used so long as it does not interfere with the binding of the two complementary sequences to form a dsRNA.

In some embodiments, the TLR-3 agonist can comprise a sequence having at least 85%, 90%, 95%, 97%, 99%, or 100% (e.g., 85%, 87%, 89%, 91%, 93%, 95%, 97%, 99/o, or 100%) identity to a sequence set forth in SEQ ID NOS:11-18. In particular embodiments, the TLR-3 agonists comprises the sequence of SEQ ID NO:11. In certain embodiments, dsRNA that is a TLR-3 agonist does not encode a particular polypeptide, but produces a pro-inflammatory cytokine (e.g. IL-6, IL-8, TNF-alpha, IFN-alpha, IFN-beta) when contacted with a responder cell (e.g., a dendritic cell, a peripheral blood mononuclear cell, or a macrophage) in vitro or in-vivo.

In particular embodiments, the TLR agonist (e.g., TLR-3 agonist) described herein can be delivered simultaneously within the same the expression vector that encodes a SARS-CoV-2 protein. In other embodiments, the TLR agonist (e.g., TLR-3 agonist) can be delivered separately (i.e., temporally or spatially) from the expression vector that encodes a SARS-CoV-2 protein. In some cases when the TLR-3 agonist is delivered separately from the expression vector, the nucleic acid encoding the TLR-3 agonist (e.g., an expressed dsRNA) and the chimeric adenoviral vector comprising a nucleic acid encoding a SARS-CoV-2 protein can be administered in the same formulation. In other cases the nucleic acid encoding the TLR-3 agonist and the chimeric adenoviral vector comprising a nucleic acid encoding a SARS-CoV-2 protein can be administered in different formulations. When the nucleic acid encoding the TLR-3 agonist and the adenoviral vector comprising a nucleic acid encoding a SARS-CoV-2 protein are administered in different formulations, their administration may be simultaneous or sequential. For example, the nucleic acid encoding the TLR-3 agonist may be administered first, followed by the chimeric adenoviral vector (e.g., 1, 2, 4, 8, 12, 16, 20, or 24 hours, 2, 4, 6, 8, or 10 days later). Alternatively, the adenoviral vector may be administered first, followed by the nucleic acid encoding the TLR-3 agonist (e.g., 1, 2, 4, 8, 12, 16, 20, or 24 hours, 2, 4, 6, 8, or 10 days later). In some embodiment, the nucleic acid encoding the TLR-3 agonist and the nucleic acid encoding the SARS-CoV-2 protein are under the control of the same promoter. In other embodiments, the nucleic acid encoding the TLR-3 agonist and the nucleic acid encoding the SARS-CoV-2 protein are under the control of different promoters.

### IV. Pharmaceutical Compositions and Routes of Administration

An immunogenic pharmaceutical composition can contain a chimeric adenoviral vector described herein and a pharmaceutically acceptable carrier. Suitable carriers include, for example, water, saline, alcohol, a fat, a wax, a buffer, a solid carrier, such as mannitol, lactose, starch, magnesium stearate, sodium saccharine, talcum, cellulose, glucose, sucrose, and magnesium carbonate, or biodegradable microspheres (e.g., polylactate polyglycolate). Suitable biodegradable microspheres are disclosed, for example, in U.S. Pat. Nos. 4,897,268; 5,075,109; 5,928,647; 5,811,128; 5,820,883. The immunogenic polypeptide and/or carrier expression vector can be encapsulated within the biodegradable microsphere or associated with the surface of the microsphere.

The ingredients in an immunogenic pharmaceutical composition are closely related to factors such as, but are not limited to, the route of administration of the immunogenic pharmaceutical composition, the timeline and/or duration of drug release, and the targeted delivery site. In some embodiments, a delayed release coating or an additional coating of the formulation can contain other film-forming polymers being non-sensitive to luminal conditions for technical reasons or chronographic control of the drug release. Materials to be used for such purpose includes, but are not limited to; sugar, polyethylene glycol, polyvinylpyrrolidone, polyvinyl alcohol, polyvinyl acetate, hydroxypropyl cellulose, methylcellulose, ethylcellulose, hydroxypropyl methylcellulose, carboxymethylcellulose sodium and others, used alone or in mixtures.

Additives such as dispersants, colorants, pigments, additional polymers, e.g., poly(ethylacrylate, methylmethacrylate), anti-tacking and anti-foaming agents can be included into a coating layer. Other compounds may be added to increase film thickness and to decrease diffusion of acidic gastric juices into the core material. The coating layers can also contain pharmaceutically acceptable plasticizers to obtain desired mechanical properties. Such plasticizers are for instance, but not restricted to, triacetin, citric acid esters, phthalic acid esters, dibutyl sebacate, cetyl alcohol, polyethylene glycols, glycerol monoesters, polysorbates or other plasticizers and mixtures thereof. The amount of plasticizer can be optimised for each formula, and in relation to the selected polymer(s), selected plasticizer(s) and the applied amount of said polymer(s).

Such immunogenic pharmaceutical compositions can also comprise non-immunogenic buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), suspending agents, thickening agents and/or preservatives. Alternatively, compositions of the present disclosure may be formulated as a lyophilate. Compounds may also be encapsulated within liposomes using well known technology.

Further, pharmaceutical compositions can be prepared to protect against stomach degradation such that the administered immunogenic biological agent reach the desired location. Methods for microencapsulation of DNA and drugs for oral delivery are described, e.g., in US2004043952. For the oral environment, several of these are available including the Eudragit and the TimeClock release systems as well as other methods specifically designed for adenovirus (Lubeck et al., Proc Natl Acad Sci USA, 86(17), 6763-6767 (1989); Chourasia and Jain, J Pharm Pharm Sci, 6(1), 33-66 (2003)). In some embodiments, the Eudragit system can be used to to deliver the chimeric adenoviral vector to the lower small intestine.

In particular embodiments, the immunogenic composition is in the form of a tablet or capsule, e.g., in the form of a compressed tablet covered by enteric coating. In some embodiments, the immunogenic composition is encapsulated in a polymeric capsule comprising gelatin, hydroxypropylmethylcellulose, starch, or pullulan. In some embodiments, the immunogenic composition is in the form of microparticles less than 2 mm in diameter, e.g., each microparticle covered with enteric coating as described herein. In particular embodiments, the immunogenic composition in the form of a tablet, a capsule, or a microparticle can be orally administered. In some embodiments, site-specific delivery can be achieved via tablets or capsules that release upon an externally generated signal. Early models released for a high-frequency (HF) signal, as disclosed in Digenis et al. (1998) Pharm. Sci. Tech. Today 1:160. The original HF capsule concept has since been updated and the result marketed as InteliSite®. The updated capsule is a radio-frequency activated, non-disintegrating delivery system. Radiolabeling of the capsule permits the determination of the capsule location within a specific region of the GI tract via gamma scintigraphy. When the capsule reaches the desired location in the GI tract, external activation opens a series of windows to the capsule drug reservoir.

In some embodiments, the immunogenic composition can be enclosed in a radio-controlled capsule, so that the capsule is tracked and signaled once it reaches the delivery site. In some embodiments, the capsule is signaled at a given time after administration that corresponds to when the capsule is expected to arrive at the delivery site, with or without detecting.

The compositions described herein may be administered as part of a sustained release formulation (i.e., a formulation such as a capsule or sponge that effects a slow release of compound following administration). Such formulations may generally be prepared using well known technology (see, e.g., Coombes el al. (1996) Vaccine 14:1429-1438). Sustained-release formulations may contain a polypeptide, polynucleotide or antibody dispersed in a carrier matrix and/or contained within a reservoir surrounded by a rate controlling membrane.

Carriers for use within such formulations are biocompatible and may also be biodegradable; preferably the formulation provides a relatively constant level of active component release. Such carriers include microparticles of poly(lactide-co-glycolide), as well as polyacrylate, latex, starch, cellulose and dextran. Other delayed-release carriers include supramolecular biovectors, which comprise a non-liquid hydrophilic core (e.g., a cross-linked polysaccharide or oligosaccharide) and, optionally, an external layer comprising an amphiphilic compound (see, e.g., WO 94/20078; WO 94/23701; and WO 96/06638). The amount of active compound contained within a sustained release formulation depends upon the site of implantation, the rate and expected duration of release and the nature of the condition to be treated or prevented.

In some embodiments, the immunogenic compositions are presented in unit-dose or multi-dose containers, such as sealed ampoules or vials. Such containers are preferably hermetically sealed to preserve sterility of the formulation until use. In general, formulations can be stored as suspensions, solutions, or emulsions in oily or aqueous vehicles. Alternatively, an immunogenic composition may be stored in a freeze-dried condition requiring only the addition of a sterile liquid carrier immediately prior to use.

Compositions for Targeted Delivery

In some embodiments of targeted delivery, enteric coatings are used to shield substances from the low pH environment of the stomach and delay release of the enclosed substance until it reaches a desired target later in the digestive tract. Enteric coatings are known, and commercially available. Examples include pH-sensitive polymers, bio-degradable polymers, hydrogels, time-release systems, and osmotic delivery systems (see, e.g., Chourasia & Jain (2003) J. Pharm. Pharmaceutical Sci. 6:33).

In some embodiments, the targeted delivery site is the ileum. The pH of the gastrointestinal tract (GIT) progresses from very acidic in the stomach (pH ˜2), to more neutral in the ileum (pH˜5.8-7.0). pH sensitive coatings can be used that dissolve in the ileum or just before the ileum. Examples include Eudragit® L and S polymers (threshold pH's ranging from 5.5-7.0); polyvinyl acetate phthalate (pH 5.0), hydroxypropyl methylcellulose phthalate 50 and 55 (pH 5.2 and 5.4, respectively), and cellulose acetate phthalate (pH 5.0). Thakral et al. (2013) Expert Opin. Drug Deliv. 10:131 review Euragit® formulations for ileal delivery, in particular, combinations of L and S that ensure delivery at pH≤7.0. Crotts et al. (2001) Eur. J Pharm. Biol. 51:71 describe Eudragit® formulations with appropriate disintegration properties. Vijay et al. (2010). J. Mater. Sci. Mater. Med. 21:2583 review acrylic acid (AA)-methyl methacrylate (MMA) based copolymers for ileal delivery at pH 6.8.

For ileal delivery, the polymer coating typically dissolves at about pH 6.8 and allows complete release within about 40 min (see, e.g., Huyghebaert et al. (2005) Int. J. Pharm. 298:26). To accomplish this, a therapeutic substance can be covered in layers of different coatings, e.g., so that the outermost layer protects the substance through low pH conditions and is dissolved when the tablet leaves the stomach, and at least one inner layer that dissolves as the tablet passes into increasing pH. Examples of layered coatings for delivery to the distal ileum are described, e.g., in WO 2015/127278, WO 2016/200951, and WO 2013/148258.

Biodegradable polymers (e.g., pectin, azo polymers) typically rely on the enzymatic activity of microflora living in the GIT. The ileum harbors larger numbers of bacteria than earlier stages, including lactobacilli and enterobacteria.

Osmotic-controlled Release Oral delivery Systems (OROS®; Alza) is an example of an osmotic system that degrades over time in aqueous conditions. Such materials can be manipulated with other coatings, or in varying thicknesses, to deliver specifically to the ileum (see. e.g., Conley et al. (2006) Curr. Med. Res. Opin. 22:1879).

Combination polymers for delivery to the ileum are reported in WO2000062820. Examples include Eudragit® L100-55 (25 mg/capsule) with triethyl citrate (2.4 mg/capsule), and Povidone K-25 (20 mg/tablet) followed by Eudragit® FS30D (30 mg/tablet). pH sensitive polymers can be applied to effect delivery to the ileum, as described above and, e.g., methacrylic acid copolymers (e.g., poly(methacylic acid-co-methyl methacrylate) 1:1), cellulose acetate phthalate, hydroxypropyl methylcellulose phthalate, hydroxypropyl methylcellulose acetate succinate, polyvinyl acetate phthalate, cellulose acetate trimellitate, carboxymethyl ethyl-cellulose, shellac or other suitable polymer(s) The coating layer can also be composed of film-forming polymers being sensitive to other luminal components than pH, such as bacterial degradation or a component that has such a sensitivity when it is mixed with another film-forming polymer. Examples of such components providing delayed release to the ileum are polymers comprising azo bond(s), polysaccharides such as pectin and its salts, galactomannans, amylose and chondroitin, disulphide polymers and glycosides.

Components with varying pH, water, and enzymatic sensitivities can be used in combination to target a therapeutic composition to the ileum. The thickness of the coating can also be used to control release. The components can also be used to form a matrix, in which the therapeutic composition is embedded. See generally, Frontiers in Drug Design & Discovery (Bentham Science Pub. 2009) vol. 4.

Adjuvant

In some embodiments of the present disclosure, in addition to the TLR agonist (e.g., TLR-3 agonist) encoded in the chimeric adenoviral vector, the compositions can further comprise additional adjuvants. Suitable adjuvants include, for example, the lipids and non-lipid compounds, cholera toxin (CT), CT subunit B, CT derivative CTK63, E. coli heat labile enterotoxin (LT), LT derivative LTK63, Al(OH)₃, and polyionic organic acids as described in e.g., WO 04/020592, Anderson and Crowle, Infect. Immun. 31(1):413-418 (1981), Roterman et al., J. Physiol. Pharmacol., 44(3):213-32 (1993), Arora and Crowle, J. Reticuloendothel. 24(3):271-86 (1978), and Crowle and May, Infect. Immun. 38(3):932-7 (1982)). Suitable polyionic organic acids include for example, 6,6′-[3,3′-demithyl[1,1′-biphenyl]-4,4′-diyl]bis(azo)bis[4-amino-5-hydroxy-1,3-naphthalene-disulfonic acid] (Evans Blue) and 3,3′-[1,1′biphenyl]-4,4′-diylbis(azo)bis[4-amino-1-naphthalenesulfonic acid] (Congo Red). It will be appreciated by those of skill in the art that the polyionic organic acids may be used for any genetic vaccination method in conjunction with any type of administration.

Other suitable adjuvants include topical immunomodulators such as, members of the imidazoquinoline family such as, for example, imiquimod and resiquimod (see, e.g., Hengge et al., Lancet Infect. Dis. 1(3):189-98 (2001).

Additional suitable adjuvants are commercially available as, for example, additional alum-based adjuvants (e.g., Alhydrogel, Rehydragel, aluminum phosphate, Algammulin); oil based adjuvants (Freund's Incomplete Adjuvant and Complete Adjuvant (Difco Laboratories, Detroit, Mich.), Specol, RIBI, TiterMax, Montanide ISA50 or Seppic MONTANIDE ISA 720); nonionic block copolymer-based adjuvants, cytokines (e.g., GM-CSF or Flat3-ligand); Merck Adjuvant 65 (Merck and Company, Inc., Rahway, N.J.); AS-2 (SmithKline Beecham, Philadelphia, Pa.); salts of calcium, iron or zinc; an insoluble suspension of acylated tyrosine; acylated sugars; cationically or anionically derivatized polysaccharides; polyphosphazenes; biodegradable microspheres; monophosphoryl lipid A and Quil A. Cytokines, such as GM-CSF or interleukin-2, -7, or -12, are also suitable adjuvants. Hemocyanins (e.g., keyhole limpet hemocyanin) and hemoerythrins may also be used in the disclosure. Polysaccharide adjuvants such as, for example, chitin, chitosan, and deacetylated chitin are also suitable as adjuvants. Other suitable adjuvants include muramyl dipeptide (MDP, N acetylmuramyl L alanyl D isoglutamine) bacterial peptidoglycans and their derivatives (e.g., threonyl-MDP, and MTPPE). BCG and BCG cell wall skeleton (CWS) may also be used as adjuvants in the disclosure, with or without trehalose dimycolate. Trehalose dimycolate may be used itself (see, e.g., U.S. Pat. No. 4,579,945). Detoxified endotoxins are also useful as adjuvants alone or in combination with other adjuvants (see, e.g., U.S. Pat. Nos. 4,866,034; 4,435,386; 4,505,899; 4,436,727; 4,436,728; 4,505,900; and 4,520,019. The saponins QS21, QS17, QS7 are also useful as adjuvants (see, e.g., U.S. Pat. No. 5,057,540; EP 0362279; WO 96/33739; and WO 96/11711). Other suitable adjuvants include Montanide ISA 720 (Seppic, France), SAF (Chiron, Calif., United States), ISCOMS (CSL), MF-59 (Chiron), the SBAS series of adjuvants (e.g., SBAS-2, SBAS-4 or SBAS-6 or variants thereof, available from SmithKline Beecham, Rixensart, Belgium), Detox (Corixa, Hamilton, Mont.), and RC-529 (Corixa, Hamilton, Mont.).

Within the pharmaceutical compositions provided herein, the adjuvant composition can be designed to induce, e.g., an immune response predominantly of the Th1 or Th2 type. High levels of Th1-type cytokines (e.g., IFN-gamma, TNF-alpha, IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Following oral delivery of a composition comprising an immunogenic polypeptide as provided herein, an immune response that includes Th1- and Th2-type responses will typically be elicited.

Routes of Administration

A composition comprising the chimeric adenoviral vector can be administered by any non-parenteral route (e.g., orally, intranasally, or mucosally via, for example, the vagina, lungs, salivary glands, nasal cavities, small intestine, colon, rectum, tonsils, or Peyer's patches). The composition may be administered alone or with an adjuvant as described above. In particular embodiments, the immunogenic composition is administered orally in the form of a tablet or capsule. In further embodiments, the immunogenic composition is administered orally for targeted delivery in the ileum in the form of a tablet or capsule.

### V. Therapeutic Uses

One aspect of the present disclosure involves using the immunogenic compositions described herein to elicit an antigen specific immune response towards a SARS-CoV-2 protein (e.g., a SARS-CoV-2 protein having the sequence of SEQ ID NOS:1, 2, or 10) in a subject. In some embodiments, the immune response is elicited in an alveolar cell, an absorptive enterocyte, a ciliated cell, a goblet cell, a club cells, and/or an airway basal cell of the subject. As used herein, a “subject” refers to any warm-blooded animal, such as, for example, a rodent, a feline, a canine, or a primate, preferably a human. The immunogenic compositions can be used before the subject developed COVID-19 to prevent disease. The disease can be diagnosed using criteria generally accepted in the art. For example, viral infection can be diagnosed by the measurement of viral titer in a biological sample (e.g., a nostril swab or mucosal sample) from the subject.

As shown in the examples, vaccines described herein can be notably effective in triggering CD4+ and CD8⁺ T-cell immune response. In some embodiments, this significant T-cell response, e.g., CD8+ T cell response, may be triggered by the presence of the SARS-CoV-2 N protein (e.g., SEQ ID NO:2 or substantially identical variants thereof), which acts to stimulate a T cell response, including a CD8⁺ T-cell response, to a second antigenic protein (which in the example was SARS-CoV-2 S protein, but which could be a different SARS-CoV-2 protein, or as discussed in more detail below, a non-SARS-CoV-2 protein). Accordingly, in some embodiments, a vaccine as described herein resulting in expression of a SARS-CoV-2 N protein as well as a second antigenic protein, can be used to trigger an immune response, which includes a CD8′ T-cell response, in a subject, e.g., a human subject. In some embodiments, the human subject is a subject with less ability to develop an antibody-based immune response or would otherwise benefit from a CD8⁺ T-cell immune response. Exemplary subjects can include, but are not limited to: elderly humans, e.g., at least 50, at least 60 or at least 70 years old, or that has an antibody deficiency disorder (see, e.g., Angel A. Justiz Vaillant; Kamleshun Ramphul, ANTIBODY DEFICIENCY DISORDER (Treasure Island (FL): StatPearls Publishing; 2020) for a description thereof), which can include but is not limited to subjects with X-linked agammaglobulinemia (Bruton disease), transient hypogammaglobulinemia of newborn, Selective Ig immunodeficiencies, for example, IgA selective deficiency, Super IgM syndrome, and common variable immunodeficiency discord.

Immunotherapy is typically active immunotherapy, in which treatment relies on the in vivo stimulation of the endogenous host immune system to react against, e.g., virally infected cells, with the administration of immunogenic composition comprising the chimeric adenoviral vectors described herein.

Frequency of administration of the immunogenic composition described herein, as well as dosage, will vary from individual to individual, and may be readily established using standard techniques. In some embodiments, between 1 and 10 (e.g., between 2 and 10, between 3 and 10, between 4 and 10, between 5 and 10, between 6 and 10, between 7 and 10, between 8 and 10, between 9 and 10, between 1 and 9, between 1 and 8, between 1 and 7, between 1 and 6, between 1 and 5, between 1 and 4, between 1 and 3, or between 1 and 2) doses may be administered over a 52 week period. In some embodiments, 2 or 3 doses are administered at intervals of 1 month; or for example, 2-3 doses are administered every 2-3 months. It is possible that the intervals will be once a year for certain therapies. Booster vaccinations may be given periodically thereafter.

A suitable dose is an amount of a compound for example that, when administered as described above, is capable of promoting an anti-viral immune response, and is at least 10-50% above the basal (i.e., untreated) level. Such response can be monitored by measuring the anti-viral antibodies in a patient or by vaccine-dependent generation of cytolytic T cells capable of killing, e.g., the patient's virus-infected cells in vitro. Immunogenic responses can also be measured by detecting immunocomplexes formed between the immunogenic polypeptides and antibodies in body fluid which are specific for the immunogenic polypeptides. Samples of body fluid taken from an individual prior to and subsequent to initiation of therapy may be analyzed for the immunocomplexes. Briefly, the number of immunocomplexes detected in both samples can be compared. A substantial change in the number of immunocomplexes in the second sample (post-therapy initiation) relative to the first sample (pre-therapy) reflects successful therapy. Such vaccines should also be capable of causing an immune response that leads to prevention of the COVID-19 disease in vaccinated patients as compared to non-vaccinated patients.

Exemplary dosages can be measured in infectious units (I.U.). A replication-deficient recombinant Ad5 vector can be tittered and quantified using I.U. units. This is accomplished through performance of an IU assay in the adherent human embryonic kidney (HEK) 293 cell line, which is permissive for growth of replication-deficient Ad5. HEK293 cells are plated in a 24-well sterile tissue culture plate and allowed to adhere. The viral material is diluted in sequential 10-fold dilutions and infected into individual wells of plated HEK293 cells in an appropriate number of replicates, usually in duplicate or triplicate. Infection is allowed to proceed via incubation for ˜40-42 hours at 37 C, 5% CO2. Cells are then fixed with methanol to allow permeability, washed, and blocked with a buffer solution containing bovine serum albumin (BSA). Cells are then incubated with a rabbit-derived primary antibody against the Ad5 hexon surface protein, washed, and probed again with an HRP-conjugated anti-rabbit secondary antibody. Infected cells are then stained via incubation with 3,3′-diaminobenzidine tetrahydrochloride (DAB) and hydrogen peroxide. Infected cells are visualized using phase-contrast microscopy and a dilution is chosen that exhibits discreet individual infection events—these are visible as darkly stained cells that are highly visible against the semi-transparent monolayer of uninfected cells. Total infected cells are counted per field-of-vision in at least ten fields-of-vision of the appropriate dilution. Viral titer can be calculated using the average number of these counts in conjunction with the total number of fields-of-vision for the objective lens/eyepiece magnification used and multiplying by the dilution factor used in the counts.

In some embodiments, the vaccines administered can have a dosage of 10⁷-10¹¹, e.g., 10⁸-10¹¹, 10⁹-10¹¹, 5×10⁹-5×10¹⁰ I.U. Suitable dose sizes will vary with the size of the patient, but will typically range from about 0.01 ml to about 10 ml for an injected vaccine, more typically from about 0.025 to about 7.5 ml, most typically from about 0.05 to about 5 ml. For a tablet or capsule final product, the size would be between 10 mg to 1000 mg, most typically between 100-400 mg. Those of skill in the art will appreciate that the dose size may be adjusted based on the particular patient or the particular disease or disorder being treated.

### EXAMPLES

The following examples are intended to illustrate, but not to limit the present disclosure.

### Example 1. Generation of Recombinant Adenoviral Constructs

Several different recombinant adenoviral (rAd) constructs to prevent SARS-CoV-2 infection were developed, using the same vector platform that was previously evaluated clinically (14, 15), with the exception that different antigens were used. Several rAd SARS-CoV-2 vaccines were generated by standard methods (e.g., as described by He, et al (50)).

Three vaccine constructs were created based on the published DNA sequence of SARS-CoV-2 publicly available as Genbank Accession No. MN908947.3. Specifically, the published amino acid sequence of the SARS-CoV-2 S protein (or surface glycoprotein; SEQ 1 below) and the SARS-CoV-2 N protein (or nucleocapsid phosphoprotein; SEQ 2 below) were used to synthesize nucleic acid sequences codon optimized for expression in Homo sapiens cells. Codon optimized nucleic acid sequences for the SARS-CoV-2 S gene and SARS-CoV-2 N gene are shown in SEQ ID NOS:3 and 4, respectively. These sequences were used to create recombinant plasmids containing transgenes cloned into the E1 region of Adenovirus Type 5 (pAd).

Two recombinant pAd plasmids were constructed using sequences from SARS-CoV-2:
1. ED81.4.1: pAd-CMV-SARS-CoV-2-S-BGH-CMV-dsRNA-SPA. Recombinant Ad5 vector containing SEQ ID NO:3 under control of the CMV promoter. S
2. ED84A6.4.1: pAd-CMV-SARS-CoV-2-S-BGH-bActin-SARS-CoV-2-N-SPA-BGH-CMV-dsRNA-SPA. Recombinant Ad5 vector containing SEQ ID NO:3 under control of the CMV promoter and SEQ ID NO:4 under control of the beta-actin promoter. Sequence of the entire transgene cassette from initial CMV promoter through the SPA following the dsRNA adjuvant is included as SEQ ID NO:6. Sequence of the entire recombinant adenoviral genome containing this transgene construct is included as SEQ ID NO: 9

In addition, a third pAd plasmid was constructed using a fusion sequence (SEQ ID NO:5) combining the S1 region of SARS-CoV-2 S gene (including the native furin site between S1 and S2) with the full-length SARS-CoV-2 N gene:
3. ST05.1.3.3: pAd-CMV-SARS-CoV-2-S1-Furin-N-BGH-CMV-dsRNA-SPA. Recombinant Ad5 vector containing SEQ ID NO:5 under control of the CMV promoter. Sequence of the entire transgene cassette from initial CMV promoter through the SPA following the dsRNA adjuvant is included as SEQ ID NO:7. Sequence of the entire recombinant adenoviral genome containing this transgene construct is included as SEQ ID NO:9.

Sequences were cloned into a shuttle plasmid using the restriction sites (e.g., Sthl and Sgfl). The shuttle plasmid was used to lock the transgenes onto a plasmid (pAd) containing the full sequence of Adenovirus Type 5 deleted for the E1 gene (pAd). The pAd plasmid was transfected into human cells providing the E1 gene product in trans to allow replication and purification of recombinant adenovirus to be used as API in vaccines.

### Example 2. Expression of the Antigen Proteins

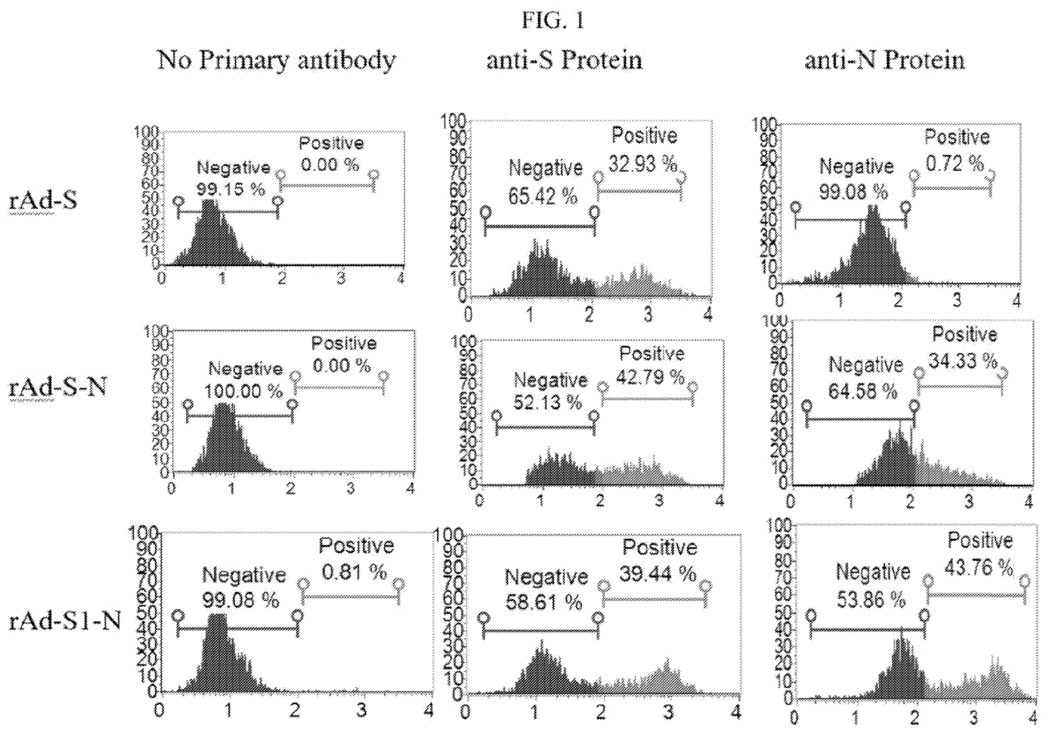

Three different candidates were evaluated for expression by intracellular staining/flow cytometry. HEK293 cells were placed in tissue culture at 3e5 cells/well in a 24-well plate. Four hrs later, the cells were infected with the various constructs at a MOI of 1. Cells were harvested 40 hours later, and human monoclonal antibodies that recognize the S1 or N proteins (Genscript) were used to stain separate wells. An anti-human IgG PE secondary antibody was used to visualize expression on the fixed cells. The candidate (rAd-S; plasmid pAd-CMV-SARS-CoV-2-S-BGH-CMV-dsRNA-SPA described above) that expressed full length SARS-CoV-2 S protein, but not the N protein clearly showed such expression patterns. The candidate (rAd-S1-N; plasmid pAd-CMV-SARS-CoV-2-S1-Furin-N-BGH-CMV-dsRNA-SPA as described above) that expressed a fusion protein of S1-N expressed both S and N proteins, as did the candidate (rAd-S-N; plasmid pAd-CMV-SARS-CoV-2-S-BGH-bActin-SARS-CoV-2-N-SPA-BGH-CMV-dsRNA-SPA as described above) that expressed S and N off separate promoters (FIG. 1).

### Example 3. Immunogenicity in Mice

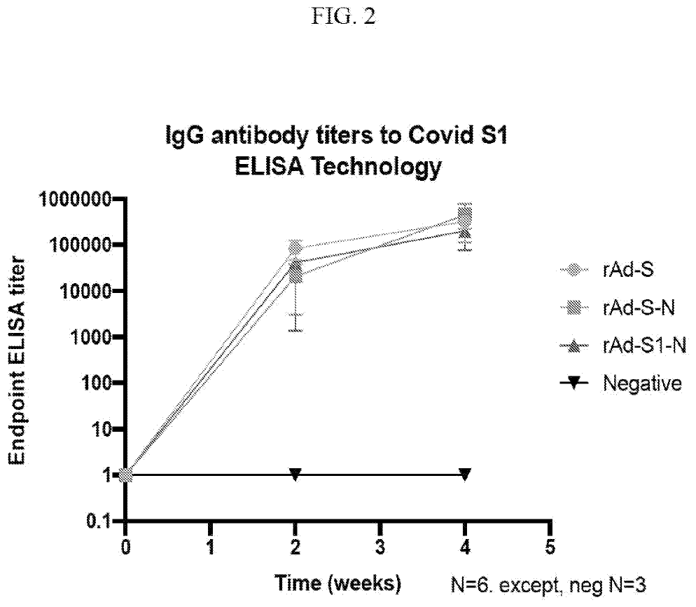

The primary objective of the initial mouse immunogenicity studies was to determine which of the rAd vectors induced significant antibody responses. The results were used to determine which candidate vaccine would be selected for GMP manufacturing. Animals were immunized by i.n. (N=6) and the antibody titers were measured over time. The rAd vector expressing both S and N off separate promoters (plasmid pAd-CMV-SARS-CoV-2-S-BGH-bActin-SARS-CoV-2-N-SPA-BGH-CMV-dsRNA-SPA as described above) produced equivalent titers to the S1 component of the S protein from SARS-CoV-2. The rAd-S-N vector had slightly higher S1 antibody responses than the fusion protein expressing rAd-S1-N (FIG. 2).

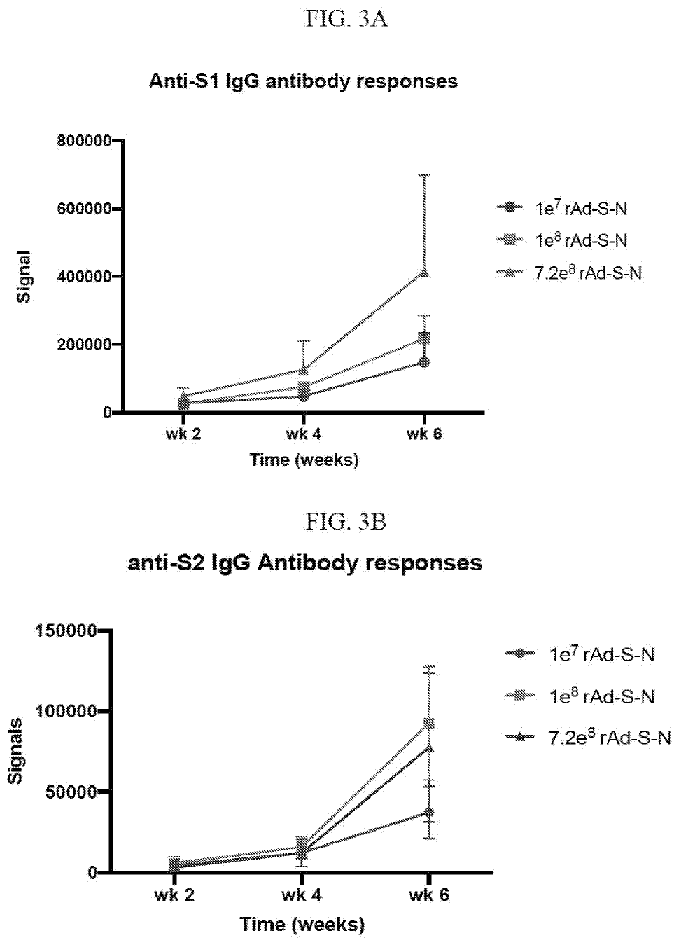

A dose response of the chosen vaccine rAd-S-N was then performed to test immunogenicity. Three different dose levels were tested, and the antibody responses to both S1 and S2 were measured using the Mesoscale device. Similar responses were seen at all three dose levels at early timepoints, but the higher dose groups had improved antibody responses at later time points (FIGS. 3A and 3B).

### Example 4. Immunogenicity in Humans

The rAd-S-N plasmid (pAd-CMV-SARS-CoV-2-S-BGH-bActin-SARS-CoV-2-N-SPA-BGH-CMV-dsRNA-SPA as described above) will be manufactured in a GMP facility, dried, and placed into tablets. A human trial will evaluate the ability of the rAd-S-N to elicit immune responses in humans at different dose levels.

### Example 5. Pre-Clinical Studies of a Recombinant Adenoviral Mucosal Vaccine to Prevent Sars-CoV-2 Infection

### INTRODUCTION

The emergence of a novel coronavirus, severe acute respiratory syndrome coronavirus 2 (SARS-CoV-2), the causative agent of COVID-19 disease, in 2019, has led to a global pandemic and significant morbidity, mortality and socio-economic disruption not seen in a century. Coronavirus disease 2019 (COVID-19) is a respiratory illness of variably severity; ranging from asymptomatic infection to mild infection, with fever and cough to severe pneumonia and acute respiratory distress1. Current reports suggest that asymptomatic spread is substantial (2), and SARS-CoV-2 infection induces a transient antibody response in most individuals (3). Therefore, development of successful interventions is an immediate requirement to protect the global population against infection and transmission of this virus and its associated clinical and societal consequences. Mass immunization with efficacious vaccines has been highly successful to prevent the spread of many other infectious diseases and can also prevent disease in the vulnerable through the induction of herd immunity. Significant effort and resources are being invested in urgently identifying efficacious SARS-CoV-2 vaccines. A number of different vaccine platforms have demonstrated pre-clinical immunogenicity and efficacy against pneumonia (4, 5); and several vaccines have demonstrated phase I or phase II safety and immunogenicity (6-8). Efficacy in the field has also been established for some platforms.

The most advanced SARS-CoV-2 vaccine candidates are all given by the intramuscular (IM) route, with some requiring −80° C. storage. This is a major barrier for vaccine dissemination and deployment during a pandemic in which people are asked to practice social distancing and avoid congregation. The ultimate goal of any vaccine campaign is to protect against disease by providing enough herd immunity to inhibit viral spread, not to make a set number of doses of vaccine. An injected solution takes a long period of time to administer and distribute and requires costly logistics, which means dose availability does not immediately translate to immunity. Further, systemic immunization can induce immunity in the periphery and lower respiratory tract. However, these vaccines cannot induce mucosal immunity in the upper respiratory tract, as evidenced by the poor mucosal IgA reported from van Doremalen, et al., 4 Mucosal IgA (with the polymeric structure and addition of the secretory component), creates more potent viral neutralization (9), can block viral transmission (10, 11), and in general, is more likely to create sterilizing immunity given that this is the first line of defense for a respiratory pathogen.

Mucosal vaccines can induce mucosal immune responses, antibodies and T cells at wet surfaces. We are developing oral vaccines for multiple indications, including influenza and noroviruses, delivered in a tablet form for people. Our vaccine platform is a replication-defective adenovirus type-5 vectored vaccine that expresses antigen along with a novel toll-like receptor 3 agonist as an adjuvant. These vaccines have been well tolerated, and able to generate robust humoral and cellular immune responses to the expressed antigens (12-14). Protective efficacy in humans was demonstrated against a respiratory virus 90 days or more post vaccination, as shown in a well characterized experimental influenza infection model (15). Furthermore, the vaccine also has the advantage of room temperature stability and needle-free, ease of administration, providing several advantages over injected vaccine approaches with respect to vaccine deployment and access.

Here, we describe the pre-clinical development of a SARS-CoV-2 vaccine based on Vaxart's oral adenovirus platform. The key approach was to develop several vaccine candidates in parallel, in order to create premanufacturing seeds while initial immunogenicity experiments were in progress. Given that the vaccines were made during the pandemic, rapid decisions were required to keep the manufacturing and regulatory timelines from slipping. We assessed the relative immunogenicity of four candidate vaccines that expressed antigens based on the spike (S) and nucleocapsid (N) SARS-CoV-2 proteins. These proteins have been well characterized as antigens for related coronaviruses, such as SARS-CoV and MERS (reviewed in Yong, et al., (16)) and, increasingly, for SARS-CoV-2 spike. The aim of our vaccine is to induce immunogenicity on three levels; firstly, to induce potent serum neutralizing antibodies to S, secondly to induce mucosal immune responses, and thirdly to induce T cell responses to both vaccine antigens. This three-fold approach aims to induce robust and broad immunity capable of protecting the individual from virus infection as well as disease, promote rapid dissemination of vaccine during a pandemic, and to protect the population from virus transmission through herd immunity.

Here, we report the induction of neutralizing antibody (Nab), IgG and IgA antibody responses, and T cell responses in mice following immunization of rAd vectors expressing one or more SARS-CoV-2 antigens.

### Results

### Vector Construction

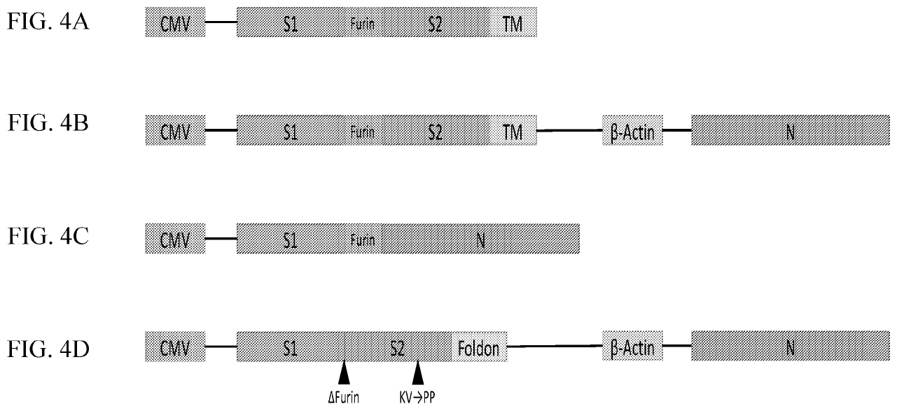

Initially, three different rAd vectors were constructed to express different SARS-CoV-2 antigens. These were a vector expressing the full-length S protein (rAd-S), a vector expressing the S protein and the N protein (rAd-S-N), and a vector expressing a fusion protein of the S1 domain with the N protein (rAd-S1-N). The N protein of rAd-S-N was expressed under control of the human beta actin promoter, which is much more potent in human cells than mouse cells. An additional construct where the expressed S protein was fixed in a prefusion conformation (rAd-S(fixed)-N) was constructed at a later date as a control for exploring neutralizing antibody responses. These are described in FIG. 4. Expression of the various transgenes was confirmed following infection of 293 cells using flow cytometry and monoclonal antibodies to the S or N protein.

### Immunogenicity of rAd Vectors Expressing S and N Antigens

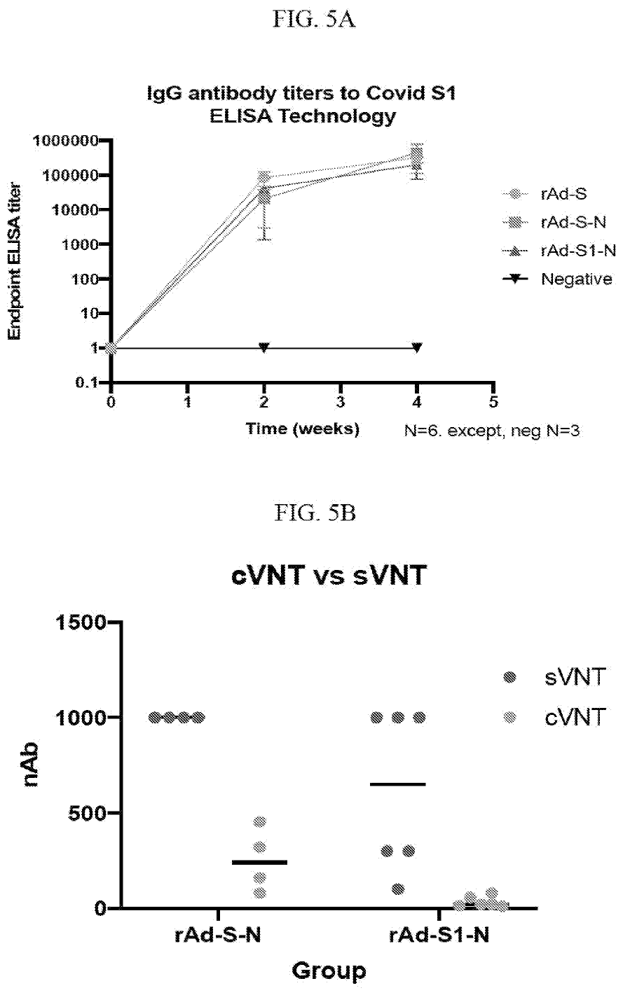

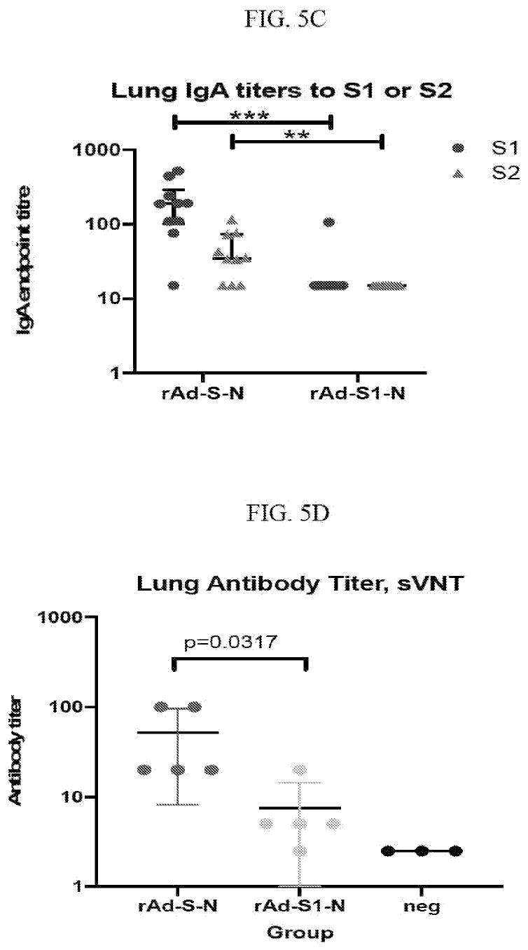

The primary objective of the initial mouse immunogenicity studies was to determine which of the rAd vectors induced significant antibody responses to S, and to obtain those results rapidly enough to provide a GMP seed in time for manufacturing. We and others (17) have observed that transgene expression by vaccine vectors orally administered to mice can be suppressed in their intestinal environment, so immunogenicity was assessed following intranasal (i.n.) immunization. Animals were immunized i.n. and the antibody titers were measured over time by IgG ELISA. All three rAd vectors induced nearly equivalent anti-S1 IgG titers, at weeks 2 and 4 and the IgG titer in all animals was significantly boosted by the second immunization (p<0.05 Mann Whitney t-tests) (FIG. 5A). However, the vector expressing full-length S (rAd-S-N) induced higher neutralizing titers compared to the vector expressing only S1 (FIG. 5B). This was measured by two different neutralizing assays, one based on SARS-CoV-2 infection of Vero cells (cVNT) and one based on a surrogate neutralizing assay (sVNT). Furthermore, rAd-S-N induced higher lung IgA responses to S1 and unsurprisingly, to S2 (FIG. 5C) compared to rAd-S1-N two weeks after the final immunization. Notably, neutralizing titers in the lung were also significantly higher when rAd-S-N was used compared to the S1-containing vaccine (rAd-S1-N) (FIG. 5D). This demonstrated that the rAd-S-N candidate induced greater functional responses (NAb and IgA) compared to the vaccine containing the just the S1 domain. Because the N protein is much more highly conserved than the S protein, and is a target of long term T cell responses induced by infection (18), the vector rAd-S-N was chosen for GMP manufacturing.

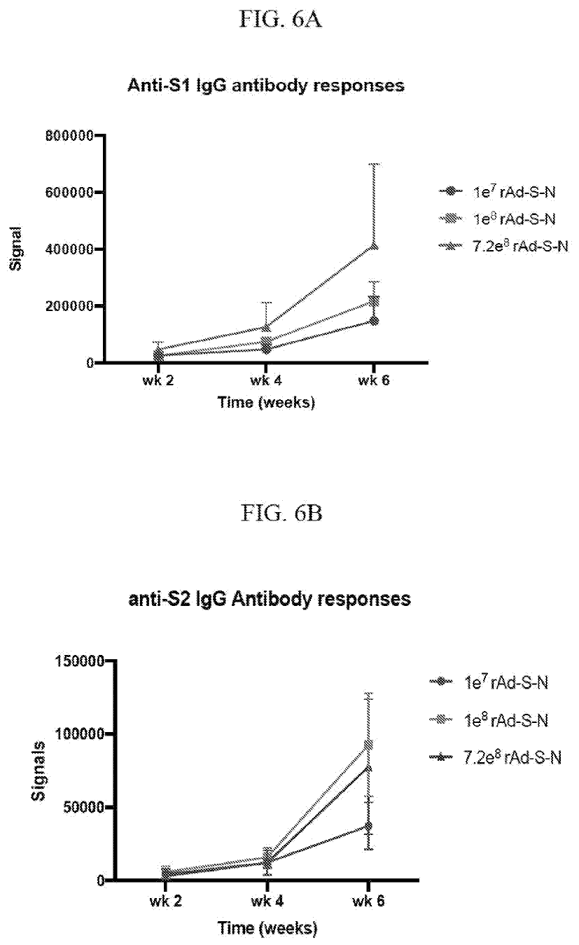

Three dose levels of rAd-S-N were then tested to understand the dose responsiveness of this vaccine. The antibody responses to both S1 (FIG. 6A) and S2 (FIG. 6B) were measured. Similar responses were seen at all three dose levels at all timepoints. Responses to S1 and S2 were significantly increased at week 6 compared to earlier times, in all groups.

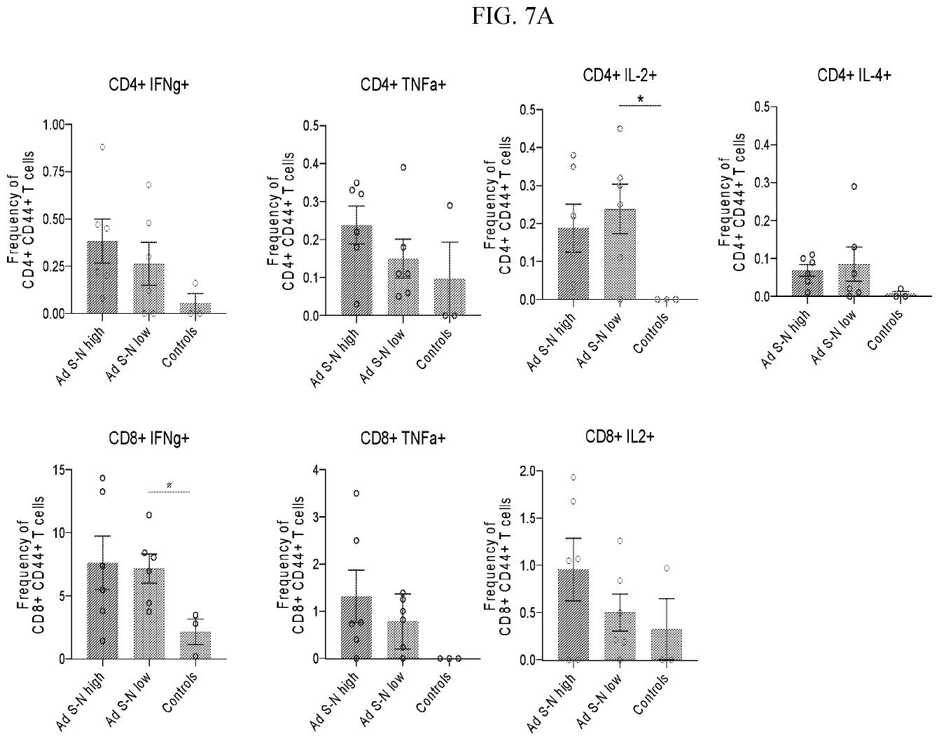

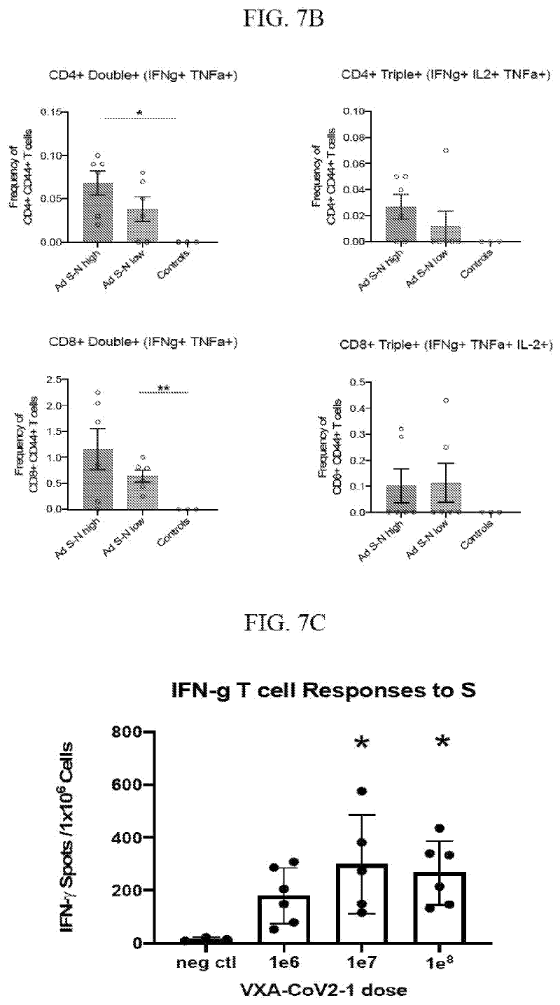

The induction of S-specific T cells by rAd-S-N at different doses was then assessed. Induction of antigen-specific CD4+ and CD8+ T cells that produced effector cytokines such as IFN-γ, TNF-α and IL-2 was observed two weeks after 2 immunizations (FIG. 7A). Notably, little IL-4 was induced by this vaccine and only in CD4+ T cells; providing a level of assurance that the risk for vaccine dependent enhancement of disease was very low. Furthermore, immunization with rAd-S-N induced double and triple positive, multi-functional IFN-γ, TNF-α and IL-2 CD4+ T cells (FIG. 7B). A second dose response experiment was performed to focus on T cell responses to the S protein, 4 weeks after the final immunization (week 8 of the study). Splenocytes were stimulated overnight with a peptide library to the S protein, divided in two separate peptide pools. T cell responses in the two pools were summed and plotted (FIG. 7C). Animals administered the 1e7 IU and the 1e8 IU dose levels had significantly higher T cell responses compared to the untreated animals but produced a similar number of IFN-γ secreting cells to each other, demonstrating a dose plateau at the 1e7 IU dose. Notably, this T cell analysis was conducted 4 weeks after the second immunization, potentially after the peak of T cell responses.

rAd-Expressed Wild-Type S Induces a Superior Neutralizing Response Compared to Stabilized Pre-Fusion S.

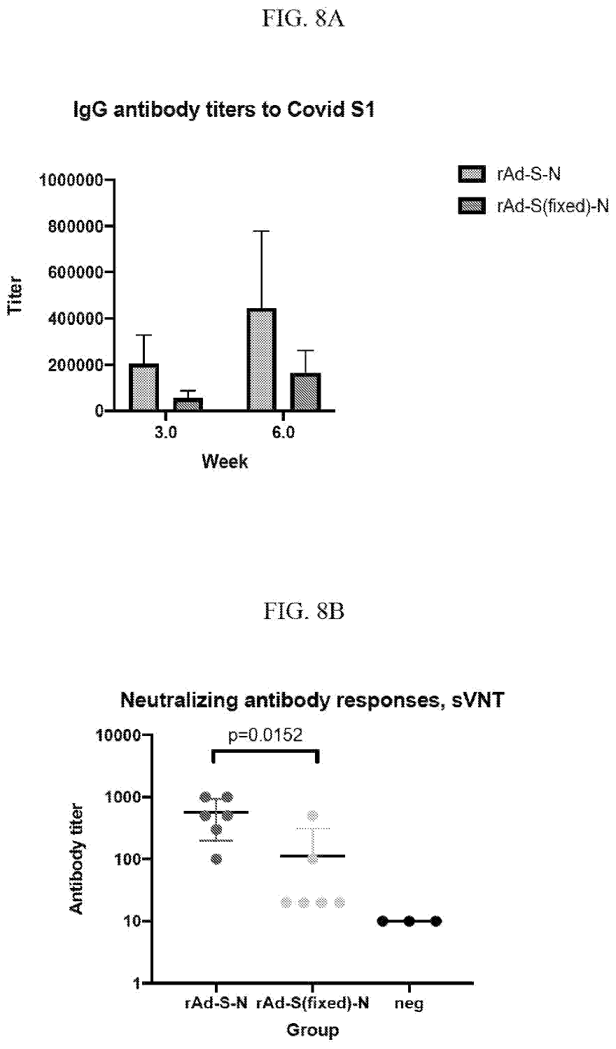

An additional study was performed to compare rAd-S-N to a vaccine candidate with the S-protein stabilized and with the transmembrane region removed (rAd-S(fixed)-N). A stabilized version of the S protein has been proposed as a way to improve neutralizing antibody responses and produce less non-neutralizing antibodies. The S protein was stabilized through modifications as described by Amanat et al., (19). rAd-S-N induced higher serum IgG titers to S1 (FIG. 8A) at both timepoints tested, although these were not statistically significant at week 6 by Mann-Whitney (p=0.067). However, rAd-S-N induced significantly higher neutralizing antibody responses (FIG. 8B) than the stabilized version (p=0.0152). These results suggest that a wild-type version of the S protein is superior for a rAd based vaccine in mice.

### DISCUSSION

The endgame to the COVID-19 pandemic requires the identification and manufacture of a safe and effective vaccine and a subsequent global immunization campaign. A number of vaccine candidates have accelerated to phase III global efficacy testing and, if sufficiently successful in these trials, may form the first generation of an immunization campaign.

However, all of these advanced candidates are S-based vaccines that are injected. Such an approach will unlikely prevent virus transmission, but should prevent pneumonia and virus growth and damage in the lower respiratory tract and periphery, as evidenced in macaque challenge studies (4, 5).

One key constraint in a global COVID-19 immunization campaign will be the cold chain distribution logistics and a bottleneck of requiring suitably trained health care workers (HCWs) to inject the vaccine. Current logistics costs, including cold chain and training, can double the cost of fully immunizing an individual in a low-middle income country (LMIC) (20). Implementing a mass immunization campaign, requiring trained HCWs for injection-based vaccines, will have a significant impact on healthcare resources in all countries. The need for cold chain, biohazardous sharps waste disposal and training will result in increased cost, inequitable vaccine access, delayed vaccine uptake and prolongation of this pandemic. These costs will be magnified if vaccines are unable to provide long-term protection (natural immunity to other beta-coronaviruses is short-lived (21)), and annual injection-based campaigns are needed. Vaxart's oral tablet vaccine platform provides a solution to these immunological as well as logistic, economic, access and acceptability problems. In this study we demonstrate, in an animal pre-clinical model, the immunogenicity of a SARS-CoV-2 vaccine using Vaxart's vaccine platform; namely the induction of serum and mucosal neutralising antibodies and poly-functional T cells.

Mouse studies were designed to test immunogenicity of candidate vaccines rapidly in the spring of 2020, before moving onto manufacturing and clinical studies critical to addressing the pandemic. Vaxart's oral tablet vaccine platform has previously proven to be able to create reliable mucosal (respiratory and intestinal), T cell, and antibody responses against several different pathogens in humans (12, 14, 22, 23). We know from our prior human influenza virus challenge study that oral immunization was able to induce protective efficacy 90 days post immunization; on par with the commercial quadrivalent inactivated vaccine (15). These features provide confidence that the adoption of the platform to COVID-19 could translate to efficacy against this pathogenic coronavirus and could provide durable protection against virus infection. Finally, a tablet vaccine campaign is much easier because qualified medical support is not needed to administer it. This ease of administration will result in increased vaccine access and potentially, acceptability, as has been evidenced by the success of easy-to-administer, oral polio vaccine, in the elimination of polio virus (24). These features could be even more important during SARS-CoV-2 immunization campaigns compared to other vaccines, as substantially more resources may be required to ensure uptake of this vaccine, given the global levels of COVID-19 denialism, mistrust and increased vaccine hesitancy (25, 26). The tablet vaccine does not need refrigerators or freezers, does not require needles or vials, and can potentially be shipped via standard mail or by a delivery drone. These qualities significantly enhance deployment and distribution logistics, even permitting access to isolated regions with fewer technical resources. Finally, from an immunological perspective, oral administration of this adenovirus is not compromised by pre-existing immunity to adenoviruses or creates substantial anti-vector immunity (12, 13), issues that have been shown to cause significantly decreased vaccine potency in an rAd5 based SARS-CoV2 vaccine (27) and can prevent durable increased immunity when the same adenovirus platform is re-administered by the IM route (28).

The choice of antigen can be difficult during a novel pandemic, a time in which key decisions are needed quickly. The S protein is believed to be the major neutralizing antibody target for coronavirus vaccines, as the protein is responsible for receptor binding, membrane fusion, and tissue tropism. When comparing SARS-CoV-2 Wu-1 to SARS-CoV, the S protein was found to have 76.2% identity (29). Both SARS-CoV and SARS-CoV-2 are believed to use the same receptor for cell entry: the angiotensin-converting enzyme 2 receptor (ACE2), which is expressed on some human cell types30. Thus, SARS-CoV-2 S protein is being used as the leading target antigen in vaccine development so far and is an ideal target given that it functions as the key mechanism for viral binding to target cells. However, the overall reliance on the S protein and an IgG serum response in the vaccines could eventually lead to viral escape. For influenza, small changes in the hemagglutinin binding protein, including a single glycosylation site, can greatly affect the ability of injected vaccines to protect (31). SARS-CoV-2 appears to be more stable than most RNA viruses, but S protein mutations have already been observed without the selective pressure of a widely distributed vaccine. Once vaccine pressure begins, escape mutations might emerge. We took two approaches to address this issue; firstly to include the more conserved N protein in the vaccine and secondly to induce broader immune responses, namely through mucosal IgA.

High expression levels of ACE2 are present in type II alveolar cells of the lungs, absorptive enterocytes of the ileum and colon, and possibly even in oral tissues such as the tongue (32). Transmission of the virus is believed to occur primarily through respiratory droplets and fomites between unprotected individuals in close contact (33), although there is some evidence of transmission via the oral-fecal route as seen with both SARS-CoV and MERS-CoV viruses where coronaviruses can be secreted in fecal samples from infected humans (34). There is also evidence that a subset of individuals exist that have gastrointestinal symptoms, rather than respiratory symptoms, are more likely to shed virus longer (35). Driving immune mucosal immune responses to S at both the respiratory and the intestinal tract may be able provide broader immunity and a greater ability to block transmission, than simply targeting one mucosal site alone. Blocking transmission, rather than just disease, will be essential to reducing infection rates and eventually eradicating SARS-CoV-2. We have previously demonstrated that an oral, tableted rAd-based vaccine can induce protection against respiratory infection and shedding following influenza virus challenge (15) as well as intestinal immunity to norovirus antigens in humans (12). Furthermore, mucosal IgA is more likely to be able address any heterogeneity of the S proteins in circulating viruses than a monomeric IgG response. mIgA has also been found to be more potent at cross reactivity than IgG for other respiratory pathogens (36). IgA may also be a more neutralizing isotype than IgG in COVID-19 infection, and in fact neutralizing IgA dominates the early immune response (37). Notably, we saw a higher ratio between neutralizing to non-neutralizing antibodies in our lung versus serum antibody results in our mouse study as well, which supports the concept that IgA may have more potency compared to IgG. Polymeric IgA, through multiple binding interactions to the antigen and to Fc receptors can turn a weak single interaction into a higher overall affinity binding and activation signal, creating more cross-protection against heterologous viruses (38).

Our second strategy to mitigate this potential vaccine-driven escape problem was to include the N protein in the vaccine construct. The N protein is highly conserved among β-coronaviruses, (greater than 90% identical) contains several immunodominant T cell epitopes, and long-term memory to N can be found in SARS-CoV recovered subjects as well as people with no known exposure to SARS-CoV or SARS-CoV-2 (18, 39). In an infection setting, T cell responses to the N protein seem to correlate to increased neutralizing antibody responses (40). All of these reasons led us to add N to our vaccine approach. The protein was expressed in 293A cells. However, as the human beta actin promoter is more active in human cells than mice, we did not explore immune responses in Balb/c mice, but will examine them more carefully in future NHP and human studies.

The optimum sequence and structure of the S protein to be included in a SARS-CoV-2 vaccine is a subject of debate. Several labs have suggested that reducing the S protein to the key neutralizing domains within the receptor binding domain (RBD) would promote higher neutralizing antibody responses, and fewer non-neutralizing antibodies (41, 42). We made a vaccine candidate composed of the S1 domain, which includes the RBD, in an attempt to promote this approach. Although the S1-based vaccine produced similar IgG binding titers to S1, neutralizing antibody responses were significantly lower compared to the full-length S antigen. Other gene-based vaccines have also shown the reductionist approach to S does not work so well, demonstrating that the DNA vaccine expressing the full-length S-protein produced higher neutralizing antibodies than shorter S segments (5). In agreement with these macaque studies, we observed that the sequence of the Ad-encoded antigen had a significant impact on antibody function, here with respect to neutralization. While reducing the potential for exposing non-neutralizing antibody epitopes seems reasonable in theory, this might reduce the T cell help that allows for greater neutralizing antibodies to develop. Indeed, of the spike protein T cell responses, which make up 54% of the responses to SARS-CoV-2, only 11% map to receptor binding domain (43). Stabilizing the S protein might be important for a protein vaccine, but not necessarily for a gene-based vaccine. The former is produced in vitro and it is produced to retain a homogenous, defined structure, ready for injection. In contrast, the latter, is expressed on the surface of a cell, in vivo, like natural infection, substantially in a prefusion form, and the additional stabilization may be unnecessary for B cells to create antibodies against the key neutralizing epitopes. We directly compared a stabilized version of S to the wild-type version in construct encoding the S and N proteins as described in this example. The wild-type version was significantly better at inducing neutralizing antibody responses. Of interest, this was also observed in a DNA vaccine study in NHPs, where the stabilized version appeared to induce lower neutralizing antibody (NAb) titers compared to the wild-type S5. A slightly different result was observed in studies of rAd26 vectors by Mercado, et al in NHPs, where expressing a stabilized version of the S protein appeared to improve NAb but lower T cell responses (44). In summary, stabilization doesn't universally improve the immune responses in gene-based or vector-based vaccines.

Multiple vaccine candidates are in, or are about to begin, clinical testing. Due to known safety and immunogenicity for epidemic pathogens such as Ebola virus, two leading candidate vaccines are based on recombinant adenovirus vectors; University of Oxford's ChAdOx1-nCov and Janssen Pharmaceutical's AdVac platforms (45-48). We saw stronger serum IgG and NAb titers in our study compared to a ChAdOx1-nCov in Balb/c mice. (4) However, this might reflect differences in assay components. An rAd36 vaccine study was performed by Hassan, et al., where doses of 1e10 VP were given by intranasal delivery (49). The results were significant from the standpoint of blocking lung infection in a mouse SARS-CoV-2 challenge model. They reported titers of serum antibody titers of 1e4 above the background titers, similar to our results, despite using doses 2- to 3-log fold higher compared to our study. Indeed, in our study, equivalently strong T cell and antibody responses were observed using 1e7 IU and 1e8 IU by the intranasal route. Using these doses, we observed high percentages of CD8+ T cell responses (up to 14%) secreting IFN-γ and TNF-α and strong CD4+ T cells after peptide restimulation. Although we did not evaluate the trafficking properties of these antigen-specific T cells, we know that oral administration of this Ad-based vaccine in humans induces high levels of mucosal homing lymphocytes (12, 15). A proportion of the antigen-specific CD4+ and CD8+ T cells were polyfunctional in this mouse study. Vaccine-induced T cells possessing multiple functions may provide more effective elimination of virus subsequent to infection and therefore could be involved in the prevention of disease, however it is uncertain at this time what is the optimum T cell phenotype required for protection against disease.

In summary, these studies in mice represent were our first step in creating a vaccine candidate, demonstrating the immunogenicity of the construct at even low vaccine doses and the elucidation of the full-length spike protein as a leading candidate antigen to induce T cell responses and superior systemic and mucosal neutralizing antibody. Future work will focus on the immune responses in humans.

### Methods

### Vaccine Constructs

For this study, four recombinant adenoviral vaccine constructs were created based on the published DNA sequence of SARS-CoV-2 publicly available as Genbank Accession No. MN908947.3. Specifically, the published amino acid sequences of the SARS-CoV-2 spike protein (S protein) and the SARS-CoV-2 Nucleocapsid protein (N protein) were used to synthesize nucleic acid sequences codon optimized for expression in Homo sapiens cells (Blue Heron Biotechnology, Bothell, WA). These sequences were used to create recombinant plasmids containing transgenes cloned into the E1 region of Adenovirus Type 5 (rAd5), as described by He, et al. (50), using the same vector backbone used in prior clinical trials for oral rAd tablets (12, 15). As shown in FIG. 4, the following four constructs were created:
a. rAd-S: rAd5 vector containing full-length SARS-CoV-2 S gene under control of the CMV promoter.
b. rAd-S-N: rAd5 vector containing full-length SARS-CoV-2 S gene under control of the CMV promoter and full-length SARS-CoV-2 N gene under control of the human beta-actin promoter.
c. rAd-S1-N: rAd5 vector using a fusion sequence combining the S1 region of SARS-CoV-2 S gene (including the native furin site between S1 and S2) with the full-length SARS-CoV-2 N gene.
d. rAd-S(fixed)-N: rAd5 vector containing a stabilized S gene with the transmembrane region removed under the control of the CMV promoter and full-length SARS-CoV-2 N gene under control of the human beta-actin promoter. The S gene is stabilized through the following modifications:
a) Arginine residues at aa positions 682, 683, 685 were deleted to remove the native furin cleavage site
b) Two stabilizing mutations were introduced: K986P and V987P
c) Transmembrane region was removed following P1213 and replaced with bacteriophage T4 fibritin trimerization foldon domain sequence (51) (GYIPEAPRDGQAYVRKDGEWVLLSTFL)

All vaccines were grown in the Expi293F suspension cell-line (Thermo Fisher Scientific), purified by CsCl density centrifugation and provided in a liquid form for animal experiments.

### Animal Experiments

Studies were approved for ethics by the Animal Care and Use Committees (IACUC). All of the procedures were carried out in accordance with local, state and federal guidelines and regulations. Female 6-8 week old Balb/c mice were purchased from Jackson labs (Bar Harbor, ME). Because mice do not swallow pills, liquid formulations were instilled intranasally in 10 μl per nostril, 20 μl per mouse in order to test immunogenicity of the various constructs. Serum was acquired by cheek puncture at various timepoints.

### Antibody Assessment

### ELISAs.

Specific antibody titers to proteins were measured similarly to methods described previously (52). Briefly, microtiter plates (MaxiSorp: Nunc) were coated in 1 carbonate buffer (0.1 M at pH 9.6) with 1.0 ug/ml S1 protein (GenScript). The plates were incubated overnight at 4° C. in a humidified chamber and then blocked in PBS plus 0.05% Tween 20 (PBST) plus 1% BSA solution for 1 h before washing. Plasma samples were serially diluted in PBST. After a 2-h incubation, the plates were washed with PBST at least 5 times. Antibodies were then added as a mixture of anti-mouse IgGI-horseradish peroxidase (HRP) and anti-mouse IgG2a-HRP (Bethyl Laboratories, Montgomery, TX). Each secondary antibody was used at a 1:5,000 dilution. The plates were washed at least 5 times after a 1-h incubation. Antigen-specific mouse antibodies were detected with 3,3=,5,5=-tetramethyl-benzidine (TMB) substrate (Rockland, Gilbertsville, PA) and H2SO4 was used as a stop solution. The plates were read at 450 nm on a Spectra Max M2 Microplate Reader. Average antibody titers were reported as the reciprocal dilution giving an absorbance value greater than the average background plus 2 standard deviations, unless otherwise stated.

### Antibody Binding Antibodies

To measure responses to both S1 and S2 simultaneously, A MULTI-SPOT® 96-well, 2-Spot Plate (Mesoscale Devices; MSD) was coated with SARS CoV-2 antigens. Proteins were commercially acquired from a source (Native Antigen Company) that produced them in mammalian cells (293 cells). These were biotinylated and adhered to their respective spots by their individual U-PLEX linkers. To measure IgG antibodies, plates were blocked with MSD Blocker B for 1 hour with shaking, then washed three times prior to the addition of samples, diluted 1:4000. After incubation for 2 hours with shaking, the plates were washed three times. The plates were then incubated for 1 hour with the detection antibody at 1 μg/mL (MSD SULFO-TAG™ Anti-mouse IgG). After washing 3 times, the Read Buffer was added and the plates were read on the Meso QuickPlex SQ 120.

### SARS-CoV-2 Neutralization Assays

Neutralizing antibodies were routinely detected based on the SARS-CoV-2 Surrogate Virus Neutralization Test (sVNT) kit (GenScript). This ELISA-based kit detects antibodies that hinder the interaction between the receptor binding domain (RBD) of the SARS-CoV-2 spike glycoprotein and the ACE2 receptor on host cells, and is highly correlated to conventional virus neutralizing titers for SARS-CoV-2 infection of Vero cells (53). The advantage of this approach is that the assay can be done in a BSL-2 laboratory. Sera from mice immunized with the candidate vaccines was diluted at 1:20, 1:100, 1:300, 1:500, 1:750 and 1:1000 using the provided sample dilution buffer. Sera from non-immunized mice was diluted 1:20. Lung samples were diluted 1:5, 1:20, and 1:100. Positive and negative controls were prepared at a 1:9 volume ratio following the provided protocol. After dilution, sera or lung samples were individually incubated at a 1:1 ratio with HRP-RBD solution for 30 minutes at 37° C. Following incubation, 100 μl of the each HRP-RBD and sample or control mixture was added to the corresponding wells in the hACE2-precoated capture plate and once again incubated at 37° C. for 15 minutes. Afterwards, wells were thoroughly washed and 100 μl of the provided TMB (3,3=,5,5=-tetramethyl-benzidine) solution was added to each well and left to incubate for 15 minutes at room temperature (20-25° C.). Lastly 50 μl of Stop Solution was added to each well, and the plate was read on a Spectra Max M2 Microplate Reader at 450 nm. The absorbance of a given sample is inversely related on the titer of anti-SARS-CoV-2 RBD neutralizing antibody in a given sample. Per test kit protocol, a cut-off of 20% inhibition when comparing the OD of the sample versus the OD of the negative control was determined to be positive for the presence of neutralizing antibodies. Samples that were negative at the lowest dilution were set equal to ½ of the lowest dilution tested, either 10 for sera or 2.5 for lung samples.

Additional neutralizing antibodies responses were measured in some studies using a cVNT assay at Visimederi under BSL3 conditions. The cVNT assay has a readout of Cytopathic Effect (CPE) to detect specific neutralizing antibodies against live SARS-COV-2 in animal or human samples. The cVNT/CPE assay permits the virus to makes multiples cycles of infection and release from cells; its exponential grow in few days (usually 72 hours of incubation) causes the partial or complete cell monolayer detachment from the surface of the support, clearly identifiable as CPE. Serum samples are heat inactivated for 30 min at 56°; two-fold dilutions, starting from 1:10 are performed then mixed with an equal volume of viral solution containing 100 TCID50 of SARS-CoV-2. The serum-virus mixture is incubated for 1 hour at 370 in humidified atmosphere with 5% CO2. After incubation, 100 μL of the mixture at each dilution are added in duplicate to a cell plate containing a semiconfluent Vero E6 monolayer. After 72 hours of incubation the plates are inspected by an inverted optical microscope. The highest serum dilution that protect more than 50% of cells from CPE is taken as the neutralization titer.

### Lung IgA ELISAs.

Two weeks after the final immunization (day 28 of the study), mice were sacrificed and bled via cardiac puncture. Lungs were removed and snap frozen at −80° C. On thawing, lungs were weighed. Lungs were homogenized in 150 μl DPBS using pellet pestles (Sigma Z359947). Homogenates were centrifuged at 1300 rpm for 3 minutes and supernatants were frozen. The total protein content in lung homogenate was evaluated using a Bradford assay to ensure equivalent amounts of tissue in all samples before evaluation of IgA content. Antigen-specific IgA titers in lungs were detected using a mouse IgA ELISA kit (Mabtech) and pNPP substrate (Mabtech). Briefly, MaxiSorp plates (Nunc) were coated with S1 or S2 (The Native Antigen Company; 50 ng/well) in PBS for overnight adsorption at 4° C. and then blocked in PBS plus 0.05% Tween 20 (PBST) plus 0.1% BSA (PBS/T/B) solution for 1 h before washing. Lung homogenates were serially diluted in PBS/TB, starting at a 1:30 dilution. After 2 hours incubation and washing, bound IgA was detected using MT39A-ALP conjugated antibody (1:1000), according to the manufacturer's protocol. Plates were read at 415 nm. Endpoint titers were taken as the x-axis intercept of the dilution curve at an absorbance value 3× standard deviations greater than the absorbance for naïve mouse serum. Non-responding animals were set a titer of 15 or 2 the value of the lowest dilution tested.

### T Cell Responses

Spleens were removed and placed in 5 ml Hanks Balanced Salt Solution (with 1 M HEPES and 5% FBS) before pushing through a sterile strainer with a 5 ml syringe. After RBC lysis (Ebiosolutions), resuspension, and counting, the cells were ready for analysis. Cells were cultured at 5e5 cells/well with two peptide pools representing the full-length S protein at 1 μg/ml (Genscript) overnight in order to stimulate the cells. The culture media consisted of RPMI media (Lonza) with 0.01M HEPES, 1×1-glutamine, 1×MEM basic amino acids, 1× penstrep, 10% FBS, and 5.5e-5 mole/l beta-mercaptoethanol. Antigen specific IFN-γ ELISPOTs were measured using a Mabtech kit. Flow cytometric analysis was performed using an Attune Flow cytometer and Flow Jo version 10.7.1, after staining with the appropriate antibodies. For flow cytometry, 2e6 splenocytes per well were incubated for 18 hours at 37° C. with peptide pools representing full-length S at either I or 5 ug/ml, adding Brefeldin A (ThermoFisher) for the last 4 hours of incubation. The antibodies used were APC-H7 conjugated CD4, FITC conjugated CD8, BV650 conjugated CD3, PerCP-Cy5.5 conjugated IFN-γ, BV421 conjugated IL-2, PE-Cy7 conjugated TNFa, APC conjugated IL-4, Alexa Fluor conjugated CD44, and PE conjugated CD62L (BD biosciences).

### REFERENCES CITED IN EXAMPLES 1-5

1. Huang C, Wang Y, Li X, et al. Clinical features of patients infected with 2019 novel coronavirus in Wuhan, China. Lancet 2020; 395(10223): 497-506.
2. Tan J, Liu S, Zhuang L, et al. Transmission and clinical characteristics of asymptomatic patients with SARS-CoV-2 infection. Future Virol 2020: 10.2217/fvl-020-0087.
3. Long Q X, Tang X J, Shi Q L, et al. Clinical and immunological assessment of asymptomatic SARS-CoV-2 infections. Nat Med 2020; 26(8): 1200-4.
4. van Doremalen N, Lambe T, Spencer A, et al. ChAdOx1 nCoV-19 vaccine prevents SARS-CoV-2 pneumonia in rhesus macaques. Nature 2020.
5. Yu J, Tostanoski L H, Peter L, et al. DNA vaccine protection against SARS-CoV-2 in rhesus macaques. Science 2020.
6. Folegatti P M, Ewer K J, Aley P K, et al. Safety and immunogenicity of the ChAdOx1 nCoV-19 vaccine against SARS-CoV-2: a preliminary report of a phase ½, single-blind, randomised controlled trial. Lancet 2020.
7. Zhu F C, Guan X H, Li Y H, et al. Immunogenicity and safety of a recombinant adenovirus type-5-vectored COVID-19 vaccine in healthy adults aged 18 years or older; a randomised, double-blind, placebo-controlled, phase 2 trial. Lancet 2020; 396(10249): 479-88.
8. Jackson L A, Anderson E J, Rouphael N G, et al. An mRNA Vaccine against SARS-CoV-2—Preliminary Report. N Engl J Med 2020.
9. Renegar K B, Jackson G D, Mestecky J. In vitro comparison of the biologic activities of monoclonal monomeric IgA, polymeric IgA, and secretory IgA. Journal of immunology 1998; 160(3): 1219-23.
10. Seibert C W, Rahmat S, Krause J C, et al. Recombinant IgA is sufficient to prevent influenza virus transmission in guinea pigs. Journal of virology 2013; 87(14): 7793-804.
11. Lowen A C, Steel J, Mubareka S, Carnero E, Garcia-Sastre A, Palese P. Blocking interhost transmission of influenza virus by vaccination in the guinea pig model. J Virol 2009; 83(7): 2803-18.
12. Kim L, Liebowitz D, Lin K, et al. Safety and immunogenicity of an oral tablet norovirus vaccine, a phase I randomized, placebo-controlled trial. JCI Insight 2018; 3(13): e121077.
13. Liebowitz D, Lindbloom J D, Brandl J R, Garg S J, Tucker S N. High titre neutralising antibodies to influenza after oral tablet immunisation: a phase 1, randomised, placebo-controlled trial. The Lancet infectious diseases 2015; 15(9): 1041-8.
14. Peters W, Brandl J R, Lindbloom J D, et al. Oral administration of an adenovirus vector encoding both an avian influenza A hemagglutinin and a TLR3 ligand induces antigen specific granzyme B and IFN-gamma T cell responses in humans. Vaccine 2013; 31: 1752-8.
15. Liebowitz D, Gottlieb K, Kolhatkar N S, et al. Efficacy and immune correlates of protection induced by an oral influenza vaccine evaluated in a phase 2, placebo-controlled human experimental infection study. The Lancet inrfectious diseases 2020; 20: 435-44.
16. Yong C Y, Ong H K, Yeap S K, Ho K L, Tan W S. Recent Advances in the Vaccine Development Against Middle East Respiratory Syndrome-Coronavirus. Front Microbiol 2019; 10: 1781.
17. Revaud J, Unterfinger Y, Rol N, et al. Firewalls Prevent Systemic Dissemination of Vectors Derived from Human Adenovirus Type 5 and Suppress Production of Transgene-Encoded Antigen in a Murine Model of Oral Vaccination. Front Cell Infect Microbiol 2018; 8: 6.
18. Peng H, Yang L T, Wang L Y, et al. Long-lived memory T lymphocyte responses against SARS coronavirus nucleocapsid protein in SARS-recovered patients. Virology 2006; 351(2): 466-75.
19. Amanat F, Stadlbauer D, Strohmeier S, et al. A serological assay to detect SARS-CoV-2 seroconversion in humans. medRxiv 2020.
20. Gandhi G, Lydon P, Cornejo S, Brenzel L, Wrobel S, Chang H. Projections of costs, financing, and additional resource requirements for low- and lower middle-income country immunization programs over the decade, 2011-2020. Vaccine 2013; 31 Suppl 2: B137-48.
21. Kissler S M, Tedijanto C, Goldstein E, Grad Y H, Lipsitch M. Projecting the transmission dynamics of SARS-CoV-2 through the postpandemic period. Science 2020; 368(6493): 860-8.
22. Kim L, Martinez C J, Hodgson K A, et al. Systemic and mucosal immune responses following oral adenoviral delivery of influenza vaccine to the human intestine by radio controlled capsule. Scientific Reports 2016; 6(1): 37295.
23. Liebowitz D, Lindbloom J D, Brandl J R, Garg S J, Tucker S N. High Titer Neutralizing Antibodies to Influenza Following Oral Tablet Immunization: A Randomized, Placebo-controlled Trial The Lancet infectious diseases 2015, 15: 1041-8.
24. Hird T R, Grassly N C. Systematic review of mucosal immunity induced by oral and inactivated poliovirus vaccines against virus shedding following oral poliovirus challenge. PLoS pathogens 2012; 8(4): e1002599.
25. Jaiswal J, LoSchiavo C, Perlman D C. Disinformation, Misinformation and Inequality-Driven Mistrust in the Time of COVID-19: Lessons Unlearned from AIDS Denialism. AIDS Behav 2020.
26. Palamenghi L, Barello S, Boccia S, Graffigna G. Mistrust in biomedical research and vaccine hesitancy: the forefront challenge in the battle against COVID-19 in Italy. Eur J Epidemiol 2020.
27. Zhu F C, Li Y H, Guan X H, et al. Safety, tolerability, and immunogenicity of a recombinant adenovirus type-5 vectored COVID-19 vaccine: a dose-escalation, open-label, non-randomised, first-in-human trial. Lancet 2020; 395(10240): 1845-54.
28. Baden L R, Walsh S R, Seaman M S, et al. First-in-Human Evaluation of the Safety and Immunogenicity of a Recombinant Adenovirus Serotype 26 HIV-1 Env Vaccine (IPCAVD 001). The Journal of infectious diseases 2012; 207(2): 240-7.
29. Kumar S, Maurya V K, Prasad A K, Bhatt M L B, Saxena S K. Structural, glycosylation and antigenic variation between 2019 novel coronavirus (2019-nCoV) and SARS coronavirus (SARS-CoV). Virus disease 2020; 31(1): 13-21.
30. Wan Y, Shang J, Graham R, Baric R S, Li F. Receptor Recognition by the Novel Coronavirus from Wuhan: an Analysis Based on Decade-Long Structural Studies of SARS Coronavirus. Journal of virology 2020; 94(7).
31. Zost S J, Parkhouse K, Gumina M E, et al. Contemporary H3N2 influenza viruses have a glycosylation site that alters binding of antibodies elicited by egg-adapted vaccine strains. Proceedings of the National Academy of Sciences of the United States of America 2017; 114(47): 12578-83.
32. Xu H, Zhong L, Deng J, et al. High expression of ACE2 receptor of 2019-nCoV on the epithelial cells of oral mucosa. Int J Oral Sci 2020; 12(1): 8.
33. Aylward B, Liang W. Report of the WHO-China Joint Mission on Coronavirus Disease 2019 (COVID-19). WHO Report 2020.
34. Yeo C, Kaushal S, Yeo D. Enteric involvement of coronaviruses: is faecal-oral transmission of SARS-CoV-2 possible? Lancet Gastroenterol Hepatol 2020.
35. Han C, Duan C, Zhang S, et al. Digestive Symptoms in COVID-19 Patients with Mild Disease Severity: Clinical Presentation, Stool Viral RNA Testing, and Outcomes. American Journal of Gastroenterology 2020.
36. Muramatsu M, Yoshida R, Yokoyama A, et al. Comparison of antiviral activity between IgA and IgG specific to influenza virus hemagglutinin: increased potential of IgA for heterosubtypic immunity. PLoS One 2014; 9(1): e85582.
37. Sterlin D, Mathian A, Miyara M, et al. IgA dominates the early neutralizing antibody response to SARS-CoV-2. medRxiv 2020: 2020.06.10.20126532.
38. Tamura S, Funato H, Hirabayashi Y, et al. Cross-protection against influenza A virus infection by passively transferred respiratory tract IgA antibodies to different hemagglutinin molecules. Eur J Immunol 1991; 21(6): 1337-44.
39. Le Bert N, Tan A T, Kunasegaran K, et al. SARS-CoV-2-specific T cell immunity in cases of COVID-19 and SARS, and uninfected controls. Nature 2020; 584(7821): 457-62.
40. Ni L, Ye F, Cheng M L, et al. Detection of SARS-CoV-2-Specific Humoral and Cellular Immunity in COVID-19 Convalescent Individuals. Immunity 2020; 52(6): 971-7 e3.
41. Tai W, Zhang X, Drelich A, et al. A novel receptor-binding domain (RBD)-based mRNA vaccine against SARS-CoV-2. Cell Res 2020.
42. Zha L, Zhao H, Mohsen M O, et al. Development of a COVID-19 vaccine based on the receptor binding domain displayed on virus-like particles. bioRriv 2020: 2020.05.06.079830.
43. Mateus J, Grifoni A, Tarke A, et al. Selective and cross-reactive SARS-CoV-2 T cell epitopes in unexposed humans. Science 2020: eabd3871.
44. Mercado N B, Zahn R, Wegmann F, et al. Single-shot Ad26 vaccine protects against SARS-CoV-2 in rhesus macaques. Nature 2020.
45. Mutua G, Anzala O, Luhn K, et al. Safety and Immunogenicity of a 2-Dose Heterologous Vaccine Regimen With Ad26.ZEBOV and MVA-BN-Filo Ebola Vaccines: 12-Month Data From a Phase 1 Randomized Clinical Trial in Nairobi, Kenya. J Infect Dis 2019; 220(1): 57-67.
46. Anywaine Z, Whitworth H, Kaleebu P, et al. Safety and Immunogenicity of a 2-Dose Heterologous Vaccination Regimen With Ad26.ZEBOV and MVA-BN-Filo Ebola Vaccines: 12-Month Data From a Phase 1 Randomized Clinical Trial in Uganda and Tanzania. J Infect Dis 2019; 220(1): 46-56.
47. Antrobus R D, Coughlan L, Berthoud T K, et al. Clinical assessment of a novel recombinant simian adenovirus ChAdOx1 as a vectored vaccine expressing conserved Influenza A antigens. Mol Ther 2014; 22(3): 668-74.
48. van Doremalen N, Haddock E, Feldmann F, et al. A single dose of ChAdOx1 MERS provides broad protective immunity against a variety of MERS-CoV strains. bioRxiv 2020: 2020.04.13.036293.
49. Hassan A O, Kafai N M, Dmitriev I P, et al. A single intranasal dose of chimpanzee adenovirus-vectored vaccine confers sterilizing immunity against SARS-CoV-2 infection. bioRxiv 2020: 2020.07.16.205088.
50. He T C, Zhou S, da Costa L T, Yu J, Kinzler K W, Vogelstein B. A simplified system for generating recombinant adenoviruses. Proceedings of the National Academy of Sciences of the United States of America 1998: 95(5): 2509-14.
51. Guthe S, Kapinos L, Moglich A, Meier S, Grzesiek S, Kiefhaber T. Very fast folding and association of a trimerization domain from bacteriophage T4 fibritin. J Mol Biol 2004; 337(4): 905-15.
52. Tucker S N, Lin K, Stevens S, Scollay R, Bennett M J, Olson D C. Systemic and mucosal antibody responses following retroductal gene transfer to the salivary gland. Mol Therapy 2003: 8: 392-9.
53. Tan C W, Chia W N, Qin X, et al. A SARS-CoV-2 surrogate virus neutralization test based on antibody-mediated blockage of ACE2-spike protein-protein interaction. Nat Biotechnol 2020.

### Example 6

Study VXA-COV2-101 was a Phase 1 open-label, dose-ranging trial to evaluate the safety and immunogenicity of a SARS-CoV-2 oral tableted vaccine (rAd-S-N, SEQ ID NO:8), which is referred to in Examples 6 and 7 as VXA-CoV2-1, administered to healthy adult subjects 18-55 years of age.

The objectives of this study were to evaluate the safety and immunogenicity of VXA-CoV2-1 oral vaccine delivered by enteric tablet.

Subjects were enrolled at a single phase I unit in Southern California. Following completion of screening assessment and confirmation of eligibility 35 subjects were enrolled into this trial; Cohort 1 sentinel subjects (n=5) were vaccinated at Day 1 and have a repeat dose at Day 29. Cohort 2 and 3 subjects received a single vaccination at Day 1. The study design is shown in the table below.

VXA-COV2-101 Study Design

| | | Dose | No. of | No of |
|---|---|---|---|---|
| Group | Vaccine | (IU ± 0.5 log)* | Doses | Subjects |
| Cohort 1 | VXA-CoV2-1 | 1 × 10¹⁰ | 2 | 5 |
| (Sentinels) | | | | |
| | SMC Review of Safety Data through Day 8 Visit | | | |
| Cohort 2 | VXA-CoV2-1 | 1 × 10¹⁰ | 1 | 15 |
| Cohort 3 | VXA-CoV2-1 | 5 × 10¹⁰ | 1 | 15 |

| | |
|---|---|
| Total | 35 |

Cohort 1 sentinel subjects received a second dose (boost) at same dose level as the first at Day 29

### B Cell/Antibody Analysis

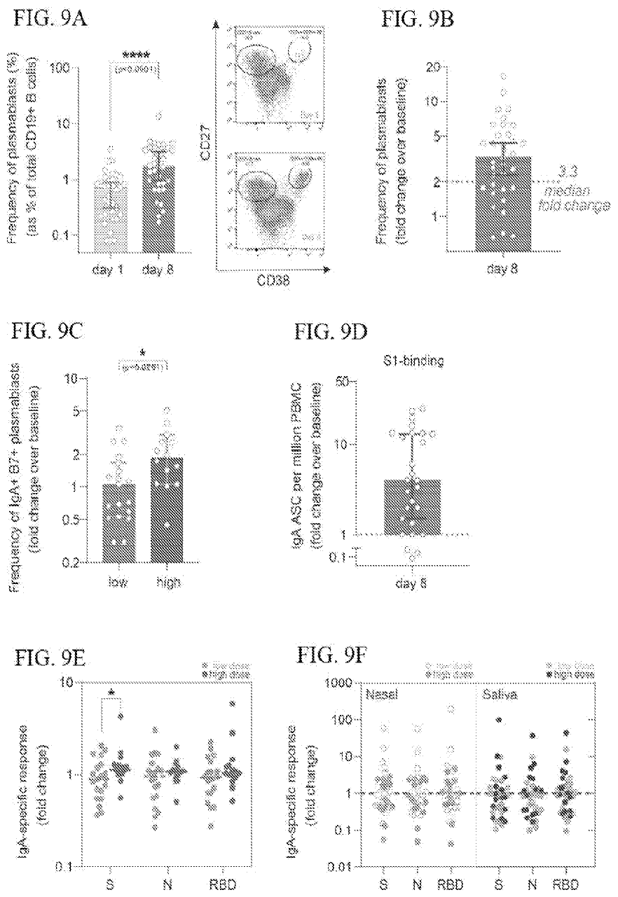

The ability of VXA-CoV2-1 in promoting B cells with high antibody-making potential was assessed using both flow cytometry-based measurements and an antibody-secreting cell (ASC) assay by ELISPOT. It has been previously well established that B cells responding to vaccination become activated at the site of administration and in local draining lymph nodes, where they differentiate into plasmablasts following germinal center reactions. Between 6 and 8 days after immunization, a significant proportion of plasmablasts leave the germinal centers and appear transiently in the peripheral circulation, where they can be found highly enriched for vaccine antigen-specific antibody-secreting cells (ASC). Accordingly, flow cytometry analysis of fixed whole blood samples collected pre- and post-vaccination in the VXA-COV2-101 study revealed a significant expansion in the overall CD27++ CD38++ plasmablast population 8 days following vaccination, with roughly 70% ( 24/35) of the vaccines showing a 2-fold or higher increase in plasmablast frequencies compared to baseline levels (FIG. 9A-B). Further investigation indicated upregulation of both IgA and the mucosal homing receptor α4β7 on the surface of circulating plasmablasts post vaccination, particularly in the cohort who received VXA-CoV2-1 at a higher dose level (FIG. 9C), thus suggesting vaccine-induced migration of this IgA-producing B cell population to mucosal tissues (Mora and von Andrian, 2008). Overall, these results are consistent with previous data published by the company in the context of a phase 2 Influenza A challenge study in humans, where generation of IgA plasmablasts with similar mucosal features following oral influenza vaccination was found to be a strong indicator of vaccine-induced protection (Liebowitz et al., 2020).

Additionally, an ELISpot assay was used to measure the ability of VXA-CoV2-1 to induce circulating antibody-secreting B cells that could recognize and bind the S1 domain of the SARS-CoV-2 spike (S) antigen. This analysis indicated significant vaccine-induced generation of S1-reactive, IgA-secreting ASC on day 8 post first immunization (p=0.0002 by Wilcoxon test), with an overall median 4-fold increase over baseline levels (FIG. 9D). More specifically, 8/12 (67%) subjects in the lower dose vaccine group for which both day 1 and day 8 ASC measurements were available were here classified as “responders”, as indicated by a median 2-fold or higher increase in day 8 post-vaccination IgA-secreting ASC numbers per million cells versus pre-vaccination levels (median fold increase of 2.67; 95% CI: 1.0-13.32). A slightly higher percentage of responders ( 11/15 subjects, 73%) was recorded in the higher vaccine dose cohort (median fold increase of 4; 95% CI: 1.3-13.32).

Levels of IgA antibodies specific to different SARS-CoV-2 antigens were measured in serum, saliva, and nasal samples pre- and post-immunization using the Meso Scale Discovery (MSD) platform. In agreement with the mucosal features of the B cell responses observed via flow cytometry and ELISPOT, IgA antibodies targeting SARS-CoV-2 spike (S), Nucleoprotein (N), and the spike receptor binding domain (RBD) could be found in both serum and mucosal compartments. Overall, 23% ( 8/35) of the vaccines showed a 50% or higher vaccine-specific IgA increase in the serum by day 29, with 6/8 of these subjects producing IgA targeting all three SARS-CoV-2 antigens in analysis. Consistent with the IgA+ B7+ plasmablast measurements, subjects in the higher dose cohort showed higher IgA antibody responses specific to S in the serum (FIG. 9). As we expected, given the unique characteristics of the VXA-CoV2-1 oral vaccine candidate, a higher percentage of vaccines mounted SARS-COV-2-specific IgA antibody responses in the mucosal compartments versus serum, with 54% of vaccines ( 19/35) reaching a 2-fold or higher increase in mucosal IgA in either saliva or nasal samples (FIG. 9F. More specifically, 10/35 (29%) of vaccines had a 2-fold or higher increase in IgA antibodies in their saliva, while 12/35 (35%) reached the same threshold in their nasal compartment by d29 post vaccination. No significant differences in vaccine-specific IgA responses in the saliva or nasal samples between the two dose groups could be observed (FIG. 9F). Measuring the ability of IgA to neutralize is difficult with the limitations of mucosal samples, but preliminary findings suggest that the subjects that had a two-fold increase in specific nasal IgA also had the ability to neutralize in a surrogate neutralization assay that measured ACE2 binding to spike protein (FIG. 9F). Secretory IgA has been shown to have higher neutralizing ability then IgG to SARS-CoV-2 as reported by Sterlin, et al, (Sterlin et al., 2021).

These findings are promising since several reports have highlighted the potential of mucosal immunity and generation of IgA antibodies in contributing to protection against COVID-19 (Ejemel et al., 2020; Russell et al., 2020; Sterlin et al., 2021). Notably, while injectable vaccines are not designed to effectively induce IgA antibodies in the respiratory mucosa, oral vaccination strategies might offer this advantage (Jeyanathan et al., 2020). Induction of SARS-CoV-2-specific IgA responses at key mucosal surfaces might also elicit sterlizing immunity and have a greater ability to block viral transmission, highly desirable features, particularly in a scenario where novel SARS-CoV-2 variants can replicate in vaccinated subjects undetected.

Analysis of IgG antibodies in the serum post-vaccination indicated an absence of an increase in SARS-CoV-2 specific antibody responses. Similarly, no significant SARS-CoV-2 antibody-mediated neutralization in the serum was observed. While the underlying reasons for the lack of vaccine-specific IgG and antibody neutralization in the serum are not defined at present, it is possible that a single oral dose of VXA-CoV2-1 at the dose levels used in this study were not sufficient to elicit robust vaccine-specific IgG neutralizing responses. Additionally, it cannot be excluded that the presence of a gene encoding for N in the VXA-CoV2-1 construct may have skewed the immunogenicity profile of this vaccine candidate away from serum neutralizing antibodies towards T cell-mediated immunity.

### T Cell Analysis

To determine if VXA-CoV2-1 elicited anti-viral CD4 and CD8 T cells, PBMCs were stimulated with SARS-CoV-2 overlapping peptide pools of the full-length sequence of the S and N proteins, and the release of the anti-viral cytokines interferon gamma (IFNγ) and tumor necrosis factor alpha (TNFα) was measured. Importantly IFNγ and TNFα have been identified as correlates of anti-viral protection (Madedonas et al., Springer Semin Immmunopathol 28:209-219, 2006; Precopio et al. J. Exp Med 204:1405-1416, 2007). Similarly, as the combination of IFNγ and CD107a has been identified as a signature of potent cytotoxic cells (Soghoian, et al. Sci Transl Med 4:123ra125, 2012), the degranulation marker CD107a was additionally assessed.

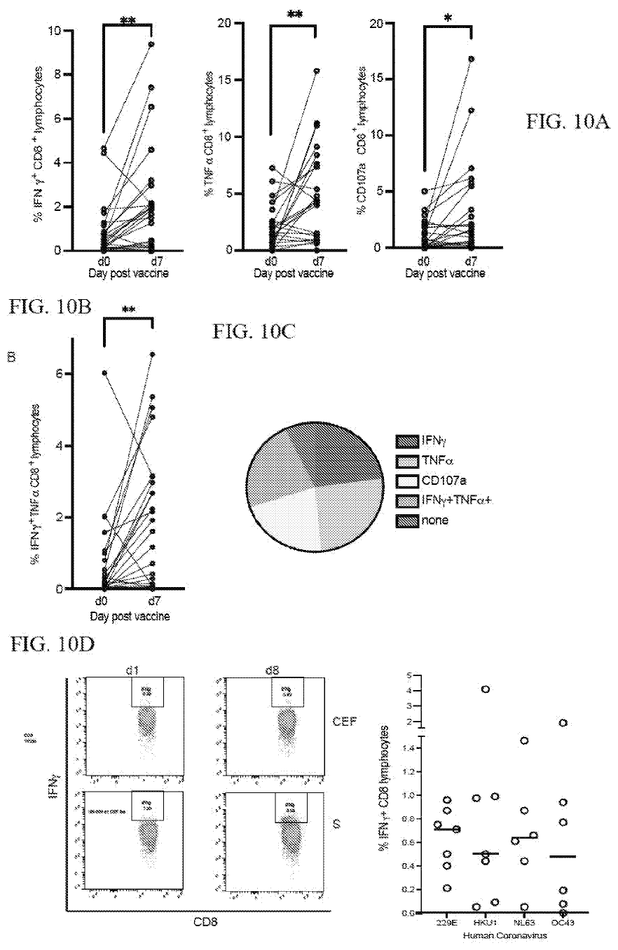

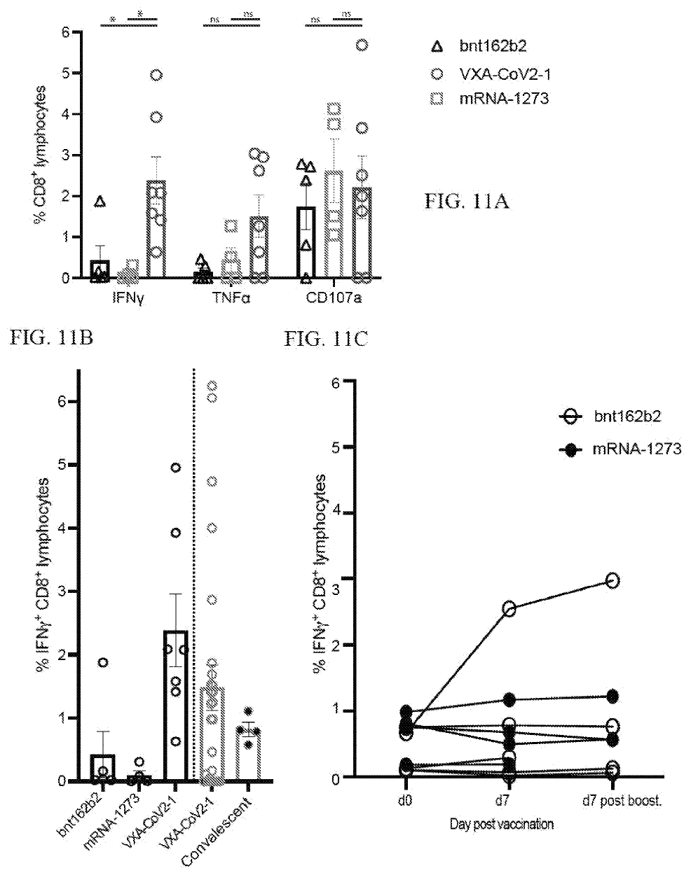

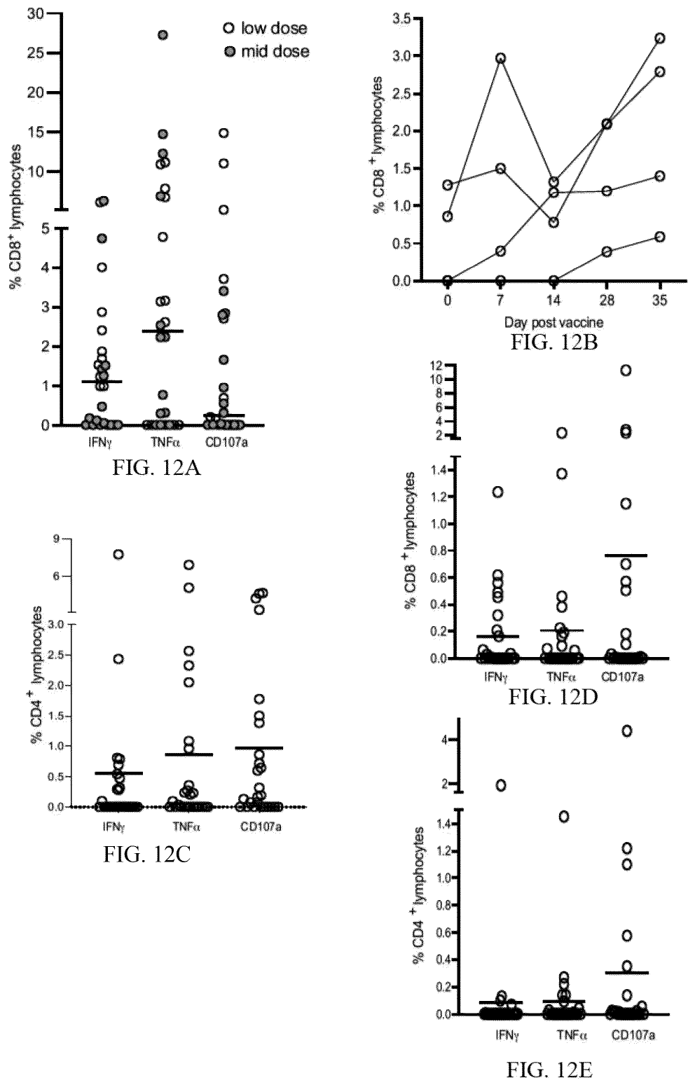

We found that vaccination with a single oral dose of VXA-CoV2-1 induced a statistically significant increase in CD8+S-specific T cells expressing IFNγ, TNFα, and CD107a at day 7 in response to S, compared with day 0 baseline levels (FIG. 10A). A dose response plot of the same data is shown as well (FIG. 12A).

Further, polyfunctionality was assessed by measuring the S-specific dual expression of IFNγ and TNFα, and we observed a significant increase in the amount of T cells that produced both IFNγ/TNFα producing cells at day 7 vs. day 0 (FIG. 10B). Polyfunctionality is seen as correlate of protection, particularly in vaccination (Makedonas et al, 2006; Precopio et al, 2007, both supra). Therefore the significant increase in the dual IFNγ and TNFα secreting CD8 T cells represents a meaningful and significant advancement to generating an anti-viral response. Approximately 25% of subjects developed a polyfunctional CD8 S-specific T cell response 7 days post vaccination, consistent with a robust anti-viral response (FIG. 10C). To show that the strong increases in cytokine expressing CD8 T were S-specific and not the result of a generalized inflammation post vaccination, cells were stimulated and parallel with a peptide pool of EBV, CMV and influenza peptides (CEF), and measured by flow cytometry for IFNγ expression (FIG. 10D). Unlike the S peptide responses, CEF peptide stimulated IFNγ responses remained unchanged before and after vaccination.

VXA-CoV2-1 induced CD8 T cell responses showed no trend towards a dose effect with the narrow dose range measured in this study (FIG. 12A) so subjects from both dose levels of VXA-CoV2-1 are combined for statistical analysis of the CD8 responses. The 4 subjects that were boosted and had PBMCs available for analysis were monitored for IFN-γ responses over time (FIG. 12B), demonstrating that T cell responses either were maintained or boosted with the second immunization.

T cell responses to N were also increased in several individuals, though to a lesser extent than to S (FIG. 12D, E). IFNγ⁺ CD107a⁺ cytotoxic CD4 T cells have the capability to augment CD8 T cells in viral control. Although not significant, vaccines also showed an increase in S-specific CD4 T cells that had cytotoxic abilities (FIG. 12C). It has previously been shown that IFNγ⁺ CD107a⁺ cytotoxic CD4 T cells have the capability to augment CD8 T cells in viral control (Johnson et al, J. Virol 89:7494-7505, 2015).

Anti-Viral T Cells are Cross-Reactive with Human Endemic Coronaviruses

To evaluate the cross reactivity of the VXA-CoV2-1-induced immune response towards endemic coronaviruses, PBMCs from nine VXA-CoV2-1 vaccinated subjects were stimulated with peptide libraries from the S and N proteins of four endemic human coronaviruses (HCoV) (229E, HKU1, OC43, and NL63) with IFNγ release measured via intracellular staining. PBMC samples were selected for evaluation based on availability and previous T cell responses to the wild type SARS-CoV-2 spike protein. An increase in IFNγ secreting CD8 T cells was detected compared to pre-vaccination levels for all four endemic HCoV (FIG. 10E), suggesting that the VXA-CoV2-1 induced T cells are cross-reactive with circulating endemic HCoV.

### Example 7

### Oral Vaccination Induces T Cells of Higher Magnitude

To compare the responses induced by VXA-CoV2-1 with the intramuscular (IM) mRNA covid vaccines, we recruited volunteers that were due to be vaccinated with the mRNA vaccines under EUA to provide blood samples. PBMCs were taken at the same timepoints as our vaccines, pre vaccination and 7 days post vaccination, and T cell activity was measured in the same in vitro assay alongside PBMCs from the VXA-CoV2-101 trial and subject to the same analysis to control for assay variability.

Subjects that took the VXA-CoV2-1 tablets had T cell responses that were several fold higher than those that were vaccinated IM with either the Pfizer (bnt162b1) or Moderna (mRNA-1273) vaccines. IFNγ release from CD8⁺ T cells was significantly increased, p=0.0283 (bnt162b1) and p=0.061(mRNA-1273), (FIG. 11A), with TNFα and CD107a showing a smaller increase over pre-vaccination baseline. The average percent increase above day 0 of IFNγ from CD8⁺ T cells for the vaccines was 0.4/0.09/2.3 for bnt162b1/mRNA-1273/VXA-CoV2-1 vaccines respectively. This amounts to a >5 fold increase of those that took VXA-CoV2-1 tablets versus the IM vaccines.

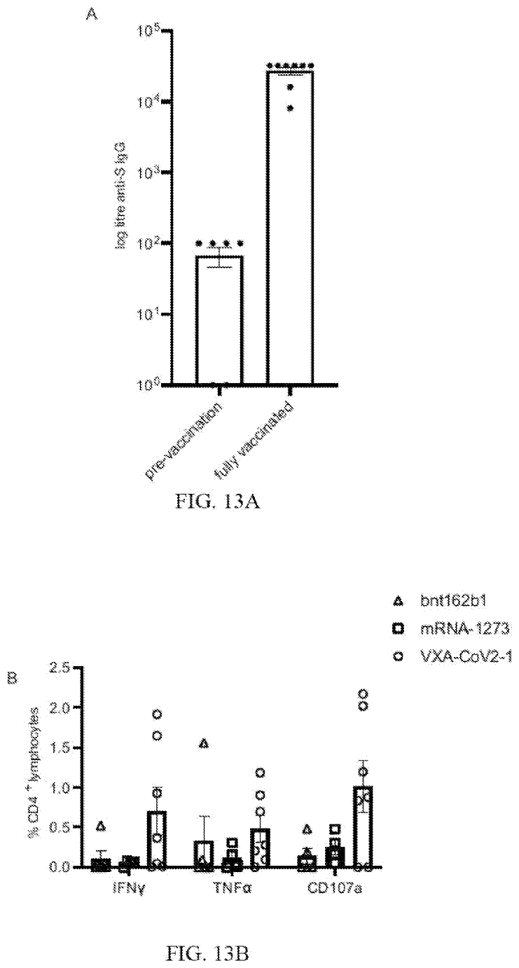

As only a small subset was tested in the same assay as the other vaccines, to account for potential bias in subject selection the whole cohort previously measured is graphed alongside for comparison, with significance (p=0.0066) still seen against the combined mRNA vaccines (FIG. 11B). The average of the whole cohort, including the non-responders, is 1.5% S specific IFNγ⁺ CD8 T cells, a >3.5-fold increase seen over the IM vaccines. For a comparison, we also measured the response of subjects previously infected with SARS-CoV-2 (convalescent), in which the average IFNγ response of the 4 convalescent subjects was 0.8% S specific IFNγ⁺ CD8 T cells. As the reported T cell measurements from the IM vaccines were taken 7 days post-second dose vaccine (Sahin, et al., Nature 2021), PBMCs were also measured at 7 days post second dose in the same assay and found to have responses of equal magnitude at both timepoints with the exception of one subject that had particularly good responses (FIG. 11C). The magnitude of the T cell responses post vaccination with bnt162b is similar to the data that was reported by Sahin and colleagues at 7 days post second dose (Nature 2021). A small cohort (n=5) in the Vaxart trial received 2 doses of VXA-CoV2-1, and CD8⁺ T cell IFNγ percentages maintained or increased at 7 days post second dose (FIG. 12B). CD4 T cell responses in the subjects that received VXA-CoV2-1 were also significantly higher when compared to the mRNA-1273 and bnt162b subjects (FIG. 13B).

As subjects can have varying immune responses and the study relied on self-reporting of vaccination status, we measured the anti-S antibody responses via ELISA and saw robust anti-S antibody responses from all bnt162b1 and mRNA-1273 (FIG. 13A) confirming vaccination status of these individuals and verifying that the lower T cell responses observed were not because of lack of vaccine administration.

### Summary of T Cell Analyses Examples 6 and 7

In the study presented in Example 7, substantial antiviral T cell responses were reported after oral immunization at frequencies higher than observed with the mRNA vaccines. Gene-based vaccines, such as VXA-CoV2-1 and mRNAs, are expected to induce substantial T cell activation because of the presentation of the antigen with MHC-I and MHC-II in vivo. The oral vaccine performed better in our study, however. One notable difference is that the N protein is present in VXA-CoV2-1, but not in either mRNA vaccine. Though it has not previously been associated with enhanced antigen presentation, N is known to have multiple biological functions including impacting the interferon induction pathways and activating TRIM21 (Caddy, et al. EMBO J 40, e106228, 2021); Mu, et al. Cell Discov 6, 65, 2020). Not to be bound by theory, the TLR-3 agonist used in VXA-CoV2-1 may improve T cell activation by maturing dendritic cells, promoting cross-presentation and driving anti-viral responses by cytotoxic T cells (Weck et al, Blood 109:3890-3894, 2007) although we have not seen T cell responses of this magnitude for other indications with this platform.

T cell responses to SARS-CoV-2 after vaccination have been measured in multiple different studies. Upon vaccination with the Bnt162b2 vaccine, activation and mobilization of T cells expressing CD38, CD39, and PD-1 were observed (Oberhardt et al, Nature. 2021). Our vaccine generated similar results, with an increase in those markers observed as well as an increase in HLA-DR⁺ CD38⁺ T cell populations. CD38⁺ HLA-DR⁺ T cells are observed in viral infection and are needed for optimal recall of memory responses upon secondary challenge, as seen in influenza (Jia et al, Clin Transl Immunology 10:e1336, 2021). In SARS-CoV-2, CD38⁺ HLA-DR⁺ CD8 T cells correlated with IFNγ responses and were associated with survival in COVID-19 patients with hematologic cancer (Bange el al, Nat Med 27:1280-1289, 2021).

In terms of cross-reactivity, T cell responses were found to be robust even against different species of HCoV, showing a substantial increase in the number of HCoV cross-reactive T cells. Because antibody responses may not adequately cross-react against all variants that appear, T cell responses could play an increasingly important role in this pandemic, where the injected licensed vaccines are potent inducers of serum antibodies. Due to the nature of T cell immunodominance hierarchies, in which responses are made to a broad range of epitopes, creating both public and private clonotypes (Shomuradova et at Immunity 53:1245-1257 e1245, 2020); Snyder, et al., medRxiv, 2020.2007.2031.20165647 (2020). T cells are also more likely to be resistant to variants and be cross-protective (Johnson, et al. J Immunol 194:1755-1762, 2015); da Silva et al, medRxiv, 2021; Tarke, et al., Cell Rep Med 2, 100204, 2021). As neutralizing antibodies target a narrow range of epitopes, mutations that arise due to selective pressure tend to decrease vaccine efficacy, but do not escape T cell responses. This has been demonstrated with SARS-CoV-2, showing that there is little impact on T cell immunity with variant strains Tarke et al, 2021, supra; Alter, et al. Nature 596:268-272, 2021; Tarke, et al. bioRxiv, 2021).

In a prospective study of first responders performed early during the pandemic, before vaccines and widespread infections had occurred, the most important correlate for clinical outcome was the pre-existing magnitude of T cell responses rather than antibody levels (Wylie et at, medRxiv, 2020.2011.2002.20222778, 2021. While antibodies are likely the correlate for the substantial efficacy of the mRNA vaccines (Gilber et al, medRxiv, 2021), studies on the Bnt162b2 vaccine showed that one dose elicited some protection as early as 12 days, despite neutralizing antibodies being undetectable at that timepoint, suggesting that T cell-mediated immunity may have played an early role in protection (Kalimuddin et al. Med (N Y) 2:682-688 e684, 2021). The S-specific T cells elicited by Bnt162b2 were shown to increase expression of CD38, which concurs with the data we obtained via mass cytometry phenotypic analysis. Lastly, people with the inability to make antibodies or B cells appear to be capable of mounting successful immune responses against SARS-CoV-2 and have a normal course of infection (Meyts et al. J. Allergy Clin Immunol 147:520-531, 2021) It was also shown that in patients with hematologic cancer, CD8 T cells correlated with survival (Huang et al, Res Sq, 2021)

The rise of multiple variants of SARS-CoV-2 that can infect even fully vaccinated people creates a scenario where new approaches are needed, potentially with heterologous boosting strategies, to complement existing protection (Barros-Martins et al, Nat Med 27:1525-1529, 2021). Any vaccine that is needed every 6-8 months would be difficult to implement in wealthy countries, and nearly impossible for the majority in the world. Further, as variants circumvent serum responses, the ability to create mucosal homing T cells might become more critical to decrease shedding and transmission. An oral tablet that creates a cross-protective response against variants and is easy to distribute can solve the key issues that impact global access.

In conclusion, oral immunization with VXA-CoV2-1 elicits antiviral SARS-CoV-2 specific T cells. The level of IFNγ-producing CD8⁺ T cells induced are of higher magnitude than the IM mRNA vaccines currently in use against COVID-19. These T cells are also cross-reactive to the four endemic human coronaviruses, indicating this vaccine could be cross-protective against a wide array of emerging pandemic coronaviruses. Because T cells may be important in protecting against death and severe infection, our vaccine candidate could offer an easy-to-administer global vaccine strategy to combat a pandemic; the current one and those of the future.

### Methods—Examples 6 and 7

### T Cell Immunogenicity Analysis

PBMCs were thawed, rested overnight, and cultured in Immunocult media (Stemcell Technologies) at a concentration of 1×10\hat{ }7 cells/ml in a 96 well round bottom plate for 5 hours at 37° C. with either the S or N peptide libraries of SARS-CoV-2 (Miltenyi) or the endemic human coronaviruses (JPT) in the presence of Brefeldin A (Invitrogen), Monensin (Biolegend), and CD107a-Alexa488 (clone H4A3) (Thermo Fisher Scientific). Cells were harvested and surface stained with CD4-BV605 (clone OKT4), CD8-BV785 (clone RPA-T8), and zombie near-IR viability dye (Biolegend). After fixing with 4% PFA (Biotium) and permeabilising with Cytoperm (BD Biosciences), antibodies to the cytokines IFNg-BV510 (clone B27) (Biolegend), TNFa-e450 (clone Mab11) (Thermo Fisher Scientific), IL-2-APC (clone MQ1-17H12) (Thermo Fisher Scientific), IL-4-PerCP5.5 (clone 8D4-8) (Biolegend), IL-5-PE (clone JES1-39D10) (Biolegend), and IL-13-PE-Cy7 (clone JES10-5A2) (Biolegend) were used to assess the intracellular cytokine response and analysed using an Attune (Thermo Fisher Scientific) flow cytometer. Data analysis was performed in Flowjo, Excel, and Graphpad Prism.

### Clinical Protocol

A phase 1 clinical study clinical trials.org NCT04563702) was designed to evaluate the safety and immunogenicity of the vaccine (termed VXA-CoV2-1) in 35 subjects at two different dose levels (1 10¹ IU and 5×10¹⁰). 5 sentinels were dosed first and after a week of monitoring for vaccine induced toxicities, the remaining subjects in the treated cohort were randomized with 4 placebo controls. Only 5 subjects in the low dose group were boosted, all other subjects were given 1 dose of VXA-CoV2-1.

### PBMC Isolation, Cryopreservation, and Thawing

PBMCs for the VXA-CoV2-101 trial were isolated from trial subject's blood and extracted on site at WCCT. PBMCs for the comparator study were extracted from blood taken by a trained phlebotomist. Blood was collected in heparin Vacutainer® tubes (BD, Franklin Lakes, NJ) and PBMCs were isolated the same day using leucosep tubes (Greinier bio one) and ficoll paque plus (Cytiva). PBMCs were frozen down in FBS with 10% DMSO in a Cool Cell (Corning) at −80° C. before being stored in liquid nitrogen until time of analysis. Cells were thawed using serum free reagents according to the manufacturer's instructions (Cellular Technology Ltd [CTL], Shaker Heights, OH).

### Vaccine

VXA-CoV2-1 is a rAd5 vector containing full-length SARS-CoV-2 S gene under control of the CMV promoter, and full-length SARS-CoV-2 N gene under control of the human beta-actin promoter. rAd5 vaccine constructs were created based on the published DNA sequence of SARS-CoV-2 publicly available as Genbank Accession No. MN908947.3. The published amino acid sequences of the SARS-CoV-2 S and the SARS-CoV-2 N were used to create recombinant plasmids containing transgenes cloned into the E1 region of Adenovirus Type 5, using the same vector backbone used in prior clinical trials for oral rAd tablets 2. All vaccines were grown in the Expi293F suspension cell-line (Thermo Fisher Scientific) and purified by CsCl density centrifugation.

### Comparator Study

For comparator studies, PBMCs were collected from healthy individuals scheduled to receive either the bnt162b1 (BioNT-Pfizer) or mRNA-1273 (Moderna) mRNA vaccines, prior to vaccination (d0), 7 days post first dose (d7), and 7 days post second dose (post boost). All subjects signed an informed consent and agreed to donate blood prior to receiving the vaccine and at 2 other timepoints: 7 days post first dose and 7 days post second dose. To confirm vaccination status, sera from mRNA vaccinated subjects were collected on d0 and day 28.

### Mass Cytometry

For initial processing, 750 uL of heparinized blood was mixed with 1050 uL of Smart Tube Proteomic Stabilizer (Smart Tube Inc., cat #PROT1), incubated for 11 minutes at room temperature, then flash-frozen on dry ice and stored at −80° C. Frozen samples were shipped on dry ice for subsequent analysis.

Two samples from each donor were transferred from −80° C. storage in an ice water bath and resuspended and washed twice in 1× Thaw-Lyse Buffer (Smart Tube Inc., cat #THAWLYSE1) for red blood cell lysis. Following red blood cell lysis, cells were counted, and 1.5×10⁶ cells were manually arranged in a 96-well block. Samples were barcoded in a 20-plex scheme with palladium metal by a robotics system that was previously described^{38 39}. Subsequent staining steps on the barcoded samples were performed manually. Barcoded cells were treated at room temperature with Fc-block (Human TruStain FcX, Biolegend cat #422302), washed once with cell staining medium (PBS with 0.5% BSA and 0.02% sodium azide), and stained with surface antibodies for 30 minutes in cell staining media. Following surface antibody staining, cells were permeabilized with ice-cold 100% methanol (Thermo Fisher, cat #A412-4), washed, and stained with intracellular antibodies for 60 minutes. After intracellular staining, cells were washed and resuspended in a solution containing iridium intercalator (Fluidigm, cat #201192B) and 1.6% paraformaldehyde (Thermo Fisher, cat #50-980-487). Prior to sample analysis on the mass cytometer, samples were washed and resuspended in 1× four-element normalization beads (140/142Ce, 151/153Eu, 165Ho, 175/176Lu) (Fluidigm, cat #201078). Collected data were normalized across all barcoded samples and debarcoded as previously described⁴⁰.

### S1 ELISA

S1 specific antibodies were measured using BioLegend Legend Max Human IgG ELISA kit. The elisa was run according to the manufacturer's instructions.

### Statistics

Statistical analyses were performed using GraphPad Prism v9 software. Each specific test is indicated in the figure legends. P values of ≤0.05 were considered significant. Bar graphs are presented as means and standard error of the mean (SEM).

### REFERENCES CITED IN EXAMPLES 6-7

Ejemel, M., Li, Q., Hou, S., Schiller, Z. A., Tree, J. A., Wallace, A., Amcheslavsky, A., Kurt Yilmaz, N., Buttigieg, K. R., Elmore, M. J., et al. (2020). A cross-reactive human IgA monoclonal antibody blocks SARS-CoV-2 spike-ACE2 interaction. Nat Commun 11, 4198.
Grifoni, A., Weiskopf, D., Ramirez, S. I., Mateus, J., Dan, J. M., Moderbacher, C. R., Rawlings, S. A., Sutherland, A., Premkumar, L., Jadi, R. S., et al. (2020). Targets of T Cell Responses to SARS-CoV-2 Coronavirus in Humans with COVID-19 Disease and Unexposed Individuals. Cell 181, 1489-1501 e1415.
He, X. S., Sasaki, S., Narvaez, C. F., Zhang, C., Liu, H., Woo, J. C., Kemble, G. W., Dekker, C. L., Davis, M. M., and Greenberg, H. B. (2011). Plasmablast-derived polyclonal antibody response after influenza vaccination. J Immunol Methods 365, 67-75.
Jeyanathan, M., Afkhami, S., Smaill, F., Miller, M. S., Lichty, B. D., and Xing, Z. (2020). Immunological considerations for COVID-19 vaccine strategies. Nat Rev Immunol 20, 615-632.
Ledford, H. (2021). How ‘killer’ T cells could boost COVID immunity in face of new variants. Nature 590, 374-375.
Lee, W. S., Wheatley, A. K., Kent, S. J., and DeKosky, B. J. (2020). Antibody-dependent enhancement and SARS-CoV-2 vaccines and therapies. Nat Microbiol 5, 1185-1191.
Liebowitz, D., Gottlieb, K., Kolhatkar, N. S., Garg, S. J., Asher, J. M., Nazareno, J., Kim, K., McIlwain, D. R., and Tucker, S. N. (2020). Efficacy, immunogenicity, and safety of an oral influenza vaccine: a placebo-controlled and active-controlled phase 2 human challenge study. Lancet Infect Dis 20, 435-444.
Mora, J. R., and von Andrian, U. H. (2008). Differentiation and homing of IgA-secreting cells. Mucosal immunology 1, 96-109.
Russell, M. W., Moldoveanu, Z., Ogra, P. L., and Mestecky, J. (2020). Mucosal Immunity in COVID-19. A Neglected but Critical Aspect of SARS-CoV-2 Infection. Front Immunol 11, 611337.
Sterlin, D., Mathian, A., Miyara, M., Mohr, A., Anna, F., Claer, L., Quentric, P., Fadlallah, J., Devilliers, H., Ghillani, P., et al. (2021). IgA dominates the early neutralizing antibody response to SARS-CoV-2. Sci Transl Med 13.
Makedonas, G. & Betts, M. R. Polyfunctional analysis of human t cell responses: importance in vaccine immunogenicity and natural infection. Springer Semin Immunopathol 28, 209-219 (2006).
Precopio, M. L., et al. Immunization with vaccinia virus induces polyfunctional and phenotypically distinctive CD8(+) T cell responses. J Exp Med 204, 1405-1416 (2007).
Soghoian, D. Z., et al. HIV-specific cytolytic CD4 T cell responses during acute HIV infection predict disease outcome. Sci Transl Med 4, 123ra125 (2012).
Johnson, S., et al. Cooperativity of HIV-Specific Cytolytic CD4 T Cells and CD8 T Cells in Control of HIV Viremia. J Viral 89, 7494-7505 (2015).
Bendall, S. C., et al. Single-cell mass cytometry of differential immune and drug responses across a human hematopoietic continuum. Science 332, 687-696 (2011).
Willinger, T., Freeman, T., Hasegawa, H., McMichael, A. J. & Callan, M. F. Molecular signatures distinguish human central memory from effector memory CD8 T cell subsets. J Immunol 175, 5895-5903 (2005).
Neidleman, J., et al. SARS-CoV-2-Specific T Cells Exhibit Phenotypic Features of Helper Function, Lack of Terminal Differentiation, and High Proliferation Potential. Cell Rep Med 1, 100081 (2020).
Rha, M. S., et al. PD-1-Expressing SARS-CoV-2-Specific CD8(+) T Cells Are Not Exhausted, but Functional in Patients with COVID-19. Immunity 54, 44-52 e43 (2021).
Chen, Y., et al. CXCR5(+)PD-1(+) follicular helper CD8 T cells control B cell tolerance. Nat Commun 10, 4415 (2019).
Mylvaganam, G. H., et al. Dynamics of SIV-specific CXCR5+CD8 T cells during chronic SIV infection. Proc Natl Acad Sci USA 114, 1976-1981 (2017).
Miller, J. D., et al. Human effector and memory CD8+ T cell responses to smallpox and yellow fever vaccines. Immunity 28, 710-722 (2008).
Christiaansen, A. F., et al. CD11a and CD49d enhance the detection of antigen-specific T cells following human vaccination. Vaccine 35, 4255-4261 (2017).
Dey, A., et al. Human Circulating Antibody-Producing B Cell as a Predictive Measure of Mucosal Immunity to Poliovirus. PloS one 11, e0146010 (2016).
Sahin, U., et a. BNT162b2 vaccine induces neutralizing antibodies and poly-specific T cells in humans. Nature (2021).
Caddy, S. L., et al. Viral nucleoprotein antibodies activate TRIM21 and induce T cell immunity. EMBO J 40, e106228 (2021).
Mu, J., et al. SARS-CoV-2 N protein antagonizes type I interferon signaling by suppressing phosphorylation and nuclear translocation of STAT1 and STAT2. Cell Discov 6, 65 (2020).
Weck, M. M., et al. TLR ligands differentially affect uptake and presentation of cellular antigens. Blood 109, 3890-3894 (2007).
Oberhardt, V., et al. Rapid and stable mobilization of CD8+ T cells by SARS-CoV-2 mRNA vaccine. Nature (2021).
Jia, X., et al. High expression of CD38 and MHC class II on CD8(+) T cells during severe influenza disease reflects bystander activation and trogocytosis. Clin Transl Immunology 10, e1336 (2021).
Bange, E. M., et al. CD8(+) T cells contribute to survival in patients with COVID-19 and hematologic cancer. Nat Med 27, 1280-1289 (2021).
Shomuradova, A. S., et al. SARS-CoV-2 Epitopes Are Recognized by a Public and Diverse Repertoire of Human T Cell Receptors. Immunity 53, 1245-1257 e1245 (2020).
Snyder, T. M., et al. Magnitude and Dynamics of the T-Cell Response to SARS-CoV-2 Infection at Both Individual and Population Levels. medRxiv, 2020.2007.2031.20165647 (2020).
Johnson, S., et al. Protective efficacy of individual CD8+ T cell specificities in chronic viral infection. J Immunol 194, 1755-1762 (2015).
da Silva Antunes, R., et al. Differential T cell reactivity to seasonal coronaviruses and SARS-CoV-2 in community and health care workers. medRxiv (2021).
Tarke, A., et al. Comprehensive analysis of T cell immunodominance and immunoprevalence of SARS-CoV-2 epitopes in COVID-19 cases. Cell Rep Med 2, 100204 (2021).
Alter, G., el al. Immunogenicity of Ad26.COV2.S vaccine against SARS-CoV-2 variants in humans. Nature 596, 268-272 (2021).
Tarke, A., et al. Negligible impact of SARS-CoV-2 variants on CD4 (+) and CD8 (+)
Wyllie, D., et al. SARS-CoV-2 responsive T cell numbers and anti-Spike IgG levels are both associated with protection from COVID-19: A prospective cohort study in keyworkers. medRxiv, 2020.2011.2002.20222778 (2021).
Gilbert, P. B., et al. Immune Correlates Analysis of the mRNA-1273 COVID-19 Vaccine Efficacy Trial. medRxiv (2021).
Kalimuddin, S., et al. Early T cell and binding antibody responses are associated with COVID-19 RNA vaccine efficacy onset. Med (N Y) 2, 682-688 e684 (2021).
Meyts, I., et al. Coronavirus disease 2019 in patients with inborn errors of immunity: An international study. J Allergy Clin Immunol 147, 520-531 (2021).
Huang, A., et al. CD8 T cells compensate for impaired humoral immunity in COVID-19 patients with hematologic cancer. Res Sq (2021).
Barros-Martins, J., et al. Immune responses against SARS-CoV-2 variants after heterologous and homologous ChAdOx1 nCoV-19/BNT162b2 vaccination. Nat Med 27, 1525-1529 (2021).

### Example 8

To test whether the N protein could enhance T cell responses, an experiment was performed in mice. Two vaccine constructs were used in this study: JL82 is a pAd5 vector encompassing the full adenovirus type 5 genome deleted for E1/E3 and containing a transgene cassette in the delE1 location under control of the CMV promoter/enhancer and followed by a bovine growth hormone polyadenylation signal. The transgene insert encodes the HPV16 E6/E7 transgene expressed as a fusion protein. To generate ED107.58 which expresses both HPV16 E6/E7 and the N protein, the transgene sequence of E6/E7 from JL82 without a stop codon was linked via a T2A sequence (doi:10.1038/s41598-017-02460-2) to the full length SARS-CoV-2 N gene (Genbank accession MN908947.3). Both HPV16 and SARS-CoV-2 sequences were codon optimized for expression in H. sapiens. The full insert sequence was synthesized in-house and cloned into pAd via recombination. See SEQ ID NOS:21-24.

Fifteen-week old female C57BL/6J mice were vaccinated via intranasal route. Seven days post vaccination, mice were sacrificed and the cells from the spleens were isolated. Splenocytes were then stimulated for approximately 18 hours with pools of 15 mer overlapping peptides derived from the HPV E6 and E7 proteins. After approximately 18 hrs, release of interferon gamma was measured via ELISpot as a measure of T cell functionality.

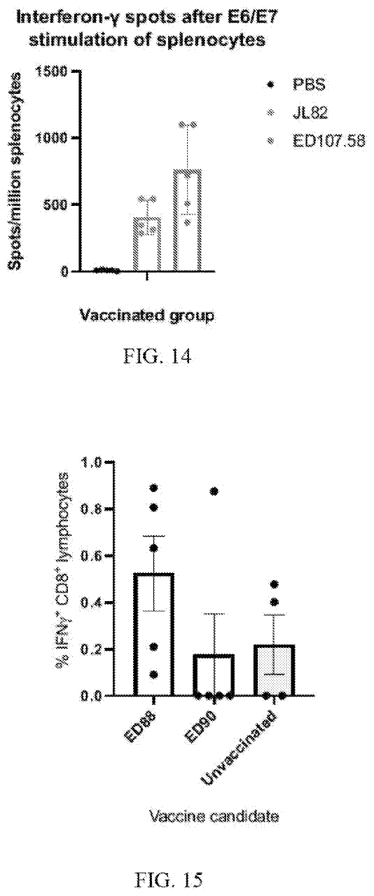

There was an increase in secreted interferon gamma from mice vaccinated with ED107 compared to JL82 in response HPV16 E6 and E7 peptides (FIG. 14). This suggests that presence of SARS-CoV-2 N protein in our vaccine construct enhanced the ability of T cells to respond to HPV16.

### Example 9

This example provides data illustrating that a construct that expresses S and N illicits a cytotoxic anti-spike T cells response that was higher than a corresponding vaccine that expresses S alone.

African green monkeys were vaccinated intranasally with a construct that expresses S and N (ED88) or S alone (ED90). To measure the response of T cells from these monkeys we took PBMCs on the day before vaccination and 7 days post vaccination. PBMCs were then stimulated for 5 hours in the presence of golgi blocking reagents with pools of 15 mer overlapping peptides from the SARS-CoV-2 Spike protein. As a measure of cytotoxic functionality, IFN-γ release by CD8 T cells was measured.

A significant increase in IFN-γ from CD8 T cells was observed in response to Spike peptides from the monkeys vaccinated with ED88 compared to ED90 (FIG. 15). Only 1 monkey vaccinated with ED90 showed an increase in IFN-γ above pre-vaccination levels whereas all 5 monkeys vaccinated with ED88 showed an increase in IFN-γ above baseline. 2 of 4 monkeys had a response above baseline in the unvaccinated group that was below the average of the monkeys vaccinated with ED88. We can conclude from this data that ED88, the vaccine that expresses both N and S proteins, had a cytotoxic anti-spike T cell response that was higher than the vaccine that expresses S alone.

FIG. 15 shows the percentage of CD8 T cells at day 8 post vaccination that are IFN-γ positive in response to spike peptides above baseline pre-vaccination samples.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

TABLE OF SEQUENCES

SEQ ID NO: 1: SARS-CoV-2 S Protein
(surface glycoprotein) amino acid sequence
MFVFLVLLPLVSSQCVNLTTRTQLPPAYTNSFTRGVYYPDKVFRS
SVLHSTQDLFLPFFSNVTWFHAIHVSGTNGTKRFDNPVLPENDGV
YFASTEKSNURGWIFGTTLDSKTQSLLIVNNATNVVIKVCEFQFC
NDPFLGVYYHKNNKSWMESEFRVYSSANNCTFEYVSQPFLMDLEG
KQGNFKNLREFVFKNIDGYFKIYSKHTPINLVRDLPQGFSALEPL
VDLPIGINITRFQTLLALHRSYLTPGDSSSGWTAGAAAYYVGYLQ
PRTFLLKYNENGTITDAVDCALDPLSETKCTLKSFTVEKGIYQTS
NFRVQPTESIVRFPNITNLCPFGEVFNATRFASVYAWNRKRISN
CVADYSVLYNSASFSTFKCYGVSPTKLNDLCFTNVYADSFVIRGD
EVRQIAPGQTGKIADYNYKLPDDFTGCVIAWNSNNLDSKVGGNYN
YLYRLFRKSNLKPFERDISTEIYQAGSTPCNGVEGENCYFPLQSY
GFQPTNGVGYQPYRVVVLSFELLHAPATVCGPKKSTNLVKNKCVN
FNFNGLTGTGVLTESNKKELPFQQFGRDIADTTDAVRDPQTLEIL
DITPCSFGGVSVITPGTNTSNQVAVLYQDVNCTEVPVAIHADQLT
PTWRVYSTGSNVFQTRAGCLIGAEHVNNSYECDIPIGAGICASYQ
TQTNSPRRARSVASQSIIAYTMSLGAENSVAYSNNSIAIPTNFTI
SVTTEILPVSMTKTSVDCTMYICGDSTECSNLLLQYGSFCTQLNR
ALTGIAVEQDKNTQEVFAQVKQIYKTPPIKDFGGFNFSQILPDP
SKPSKRSFIEDLLFNKVTLADAGFIKQYGDCLGDIAARDLICAQK
FNGLTVLPPLLTDEMIAQYTSALLAGTITSGWTFGAGAALQIPFA
MQMAYRENGIGVTQNVLYENQKLIANQFNSAIGKIQDSLSSTASA
LGKLQDVVNQNAQALNTLVKQLSSNFGAISSVLNDILSRLDKVEA
EVQIDRLITGRLQSLQTYVTQQLIRAAEIRASANLAATKMSECVL
GQSKRVDFCGKGYHLMSFPQSAPHGVVFLHVTYVPAQEKNFTTAP
AICHDGKAHFPREGVFVSNGTHWFVTQRNFYEPQIITTDNTFVSG
NCDVVIGIVNNTVYDPLQPELDSFKEELDKYFKNHTSPDVDLGDI
SGINASVVNIQKEIDRLNEVAKNLNESLIDLQELGKYEQYIKWPW
YIWLGFIAGLIAIVMVTIMLCCMTSCCSCLKGCCSCGSCCKFDED
DSEPVLKGVKLHYT*
SEQ ID NO: 2: SARS-CoV-2 N Protein
(nucleocapsid phosphoprotein) amino acid
sequence
MSDNGPQNQRNAPRITFGGPSDSTGSNQNGERSGARSKQRRPQGL
PNNTASWFTALTQHGKEDLKFPRGQGVPINTNSSPDDQIGYYRRA
TRRIRGGDGKMKDLSPRWYFYYLGTGPEAGLPYGANKDGIIWVAT
EGALNTPKDHIGTRNPANNAAIVLQLPQGTTLPKGFYAEGSRGGS
QASSRSSSRSRNSSRNSTPGSSRGTSPARMAGNGGDAALALLLLD
RLNQLESKMSGKGQQQQGQTVTKKSAAEASKKPRQKRTATKAYNV
TQAFGRRGPEQTQGNFGDQELIRQGTDYKHWPQIAQFAPSASAFF
GMSRIGMEVTPSGTWLTYTGAIKLDDKDPNFKDQVILLNKHIDAY
KTFPPTEPKKDKKKKADETQALPQRQKKQQTVTLLPAADLDDFSK
QLQQSMSSADSTQA*
SEQ ID NO: 3: SARS-CoV-2 S Protein
(surface glycoprotein) nucleic acid sequence
ggtaccgccaccATGTTTGTTTTTCTCGTACTCCTGCCCCTGGTT
TCCTCCCAATGTGTCAATCTGACTACCCGGACCCAACTTCCTCCC
GCCTACACCAATTCCTTTACCCGAGGTGTTTACTACCCAGACAAA
GTGTTCAGGTCATCCGTCCTCCATAGTACCCAAGACCTCTTCCTC
CCTTTTTTTTCTAACGTTACCTGGTTTCACGCTATTCACGTTAGC
GGCACCAACGGCACCAAAAGATTCGATAACCCCGTACTGCCGTTC
AACGACGGGGTATATTTTGCCTCTACTGAAAAATCAAACATCATA
CGCGGATGGATCTTTGGGACTACCCTGGACTCAAAAACTCAGTCC
CTGCTGATTGTGAATAACGCTACCAACGTGGTGATCAAAGTCTGT
GAATTCCAGTTTTGCAACGATCCTTTTCTCGGCGTTTATTATCAC
AAAAATAACAAATCCTGGATGGAGAGCGAGTTCCGGGTGTACTCC
TCCGCGAATAATTGCACCTTCGAATATGTGTCTCAGCCATTCCTC
ATGGACCTCGAGGGGAAGCAGGGCAATTTTAAGAATCTGCGAGAA
TTCGTGTTCAAGAATATAGACGGTTACTTCAAGATTTACTCCAAA
CACACCCCGATTAACCTGGTTAGGGACTTGCCTCAGGGCTTTTCT
GCATTGGAGCCCCTCGTGGACCTCCCAATCGGCATAAACATTACA
AGATTTCAGACTTTGCTTGCATTGCACAGGAGCTATTTGACACCC
GGCGATTCTTCTTCCGGATGGACCGCTGGAGCAGCTGCTTATTAC
GTGGGCTATCTGCAGCCTCGAACCTTTCTTTTGAAGTACAACGAA
AATGGAACTATCACCGATGCAGTTGACTGCGCCCTGGACCCCCTG
TCCGAAACTAAGTGCACGCTCAAAAGTTTCACAGTAGAGAAGGGG
ATATACCAGACTAGCAATTTCCGCGTTCAGCCAACCGAAAGTATA
GTGCGCTTTCCTAATATAACTAACCTGTGTCCTTTCGGGGAAGTG
TTTAACGCCACTAGATTCGCTTCCGTCTACGCCTGGAATAGAAAG
AGGATCTCAAATTGCGTTGCTGACTATAGTGTTTTGTACAATTCC
GCCTCTTTCTCAACCTTCAAATGTTACGGGGTGAGCCCTACCAAA
CTGAACGACCTGTGCTTTACAAACGTATACGCCGACAGCTTTGTT
ATCAGAGGAGACGAGGTTCGCCAGATTGCTCCGGGTCAGACAGGC
AAGATTGCTGATTATAATTACAAACTGCCCGACGACTTTACAGGA
TGTGTGATCGCGTGGAACAGTAACAATCTTGACTCAAAGGTTGGG
GGTAATTATAATTATCTTTACCGGCTGTTCAGAAAAAGCAATTTG
AAACCCTTCGAAAGGGACATATCCACCGAGATCTATCAGGCCGGG
TCCACTCCATGCAATGGTGTGGAAGGTTTTAATTGCTACTTCCCA
TTGCAGTCTTATGGATTCCAACCAACCAATGGCGTAGGCTACCAG
CCGTATCGCGTTGTCGTGCTCAGCTTCGAGCTGCTCCACGCCCCC
GCGACCGTATGCGGTCCTAAGAAGTCCACCAATCTTGTTAAGAAC
AAGTGTGTAAACTTTAACTTTAACGGGCTGACCGGGACCGGCGTT
CTGACTGAATCTAACAAAAAATTCCTGCCTTTCCAGCAGTTCGGC
CGCGATATTGCTGACACCACTGACGCTGTAAGAGACCCTCAGACC
CTTGAAATTCTCGATATCACACCTTGCAGCTTTGGGGGCGTGTCC
GTCATCACTCCAGGAACTAACACAAGCAACCAGGTGGCAGTGTTG
TACCAGGATGTTAATTGTACCGAGGTGCCAGTGGCCATCCACGCC
GATCAATTGACACCTACCTGGAGGGTTTACAGCACAGGGTCCAAT
GTTTTTCAGACAAGAGCCGGATGTCTGATCGGTGCCGAGCATGTC
AACAATTCCTACGAGTGTGATATCCCCATTGGTGCGGGAATTTGT
GCATCATATCAGACCCAGACTAATAGCCCAAGAAGAGCTAGATCC
GTCGCTAGTCAATCCATCATTGCATATACAATGTCCCTGGGAGCT
GAGAATTCAGTCGCGTATTCAAACAATTCCATTGCTATTCCTACT
AATTTCACTATCTCCGTCACGACCGAGATCCTGCCAGTTTCCATG
ACTAAGACTTCTGTTGACTGCACCATGTATATCTGTGGCGATAGC
ACCGAGTGCAGTAATCTGCTTCTGCAGTACGGCTCCTTCTGCACA
CAACTCAATCGAGCACTGACCGGTATTGCAGTTGAGCAGGACAAG
AACACACAGGAGGTCTTTGCACAGGTCAAACAAATTTACAAAACC
CCCCCCATAAAAGACTTTGGTGGGTTCAACTTCAGCCAAATCCTC
CCAGATCCCAGCAAGCCCTCCAAAAGATCCTTCATCGAAGACCTT
TTGTTCAATAAGGTAACCCTGGCCGACGCAGGCTTCATCAAACAA
TATGGCGATTGCCTTGGAGACATTGCTGCGCGCGATTTGATCTGT
GCTCAGAAATTTAACGGTTTGACCGTGCTGCCCCCACTTCTGACT
GATGAGATGATAGCACAGTATACTTCTGCTCTTCTGGCAGGAACA
ATCACTTCCGGGTGGACCTTTGGCGCTGGTGCAGCACTGCAAATC
CCCTTCGCAATGCAAATGGCCTACCGATTCAATGGTATTGGTGTT
ACCCAGAACGTGCTCTATGAGAATCAGAAACTCATCGCCAATCAG
TTCAATAGCGCTATTGGCAAGATTCAGGATTCCCTCAGCTCTACC
GCCAGCGCTCTGGGGAAGCTCCAGGACGTGGTGAACCAAAATGCT
CAAGCGCTCAATACCCTTGTGAAACAGCTCAGCTCCAATTTTGGC
GCAATTAGCAGCGTTCTGAATGATATTCTGTCCCGGCTGGACAAG
GTAGAAGCAGAAGTCCAGATCGACAGGCTGATCACCGGGCGGTTG
CAGAGTCTCCAGACCTATGTCACACAACAGCTGATCCGCGCCGCC
GAGATCAGGGCTTCCGCTAACCTGGCCGCCACTAAGATGTCCGAA
TGCGTGTTGGGGCAGAGTAAGCGGGTCGACTTTTGCGGGAAGGGA
TACCATCTGATGAGCTTCCCTCAGTCTGCACCCCACGGAGTAGTG
TTCCTCCACGTCACATATGTGCCCGCTCAGGAAAAGAATTTCACA
ACCGCACCTGCTATCTGTCACGACGGCAAGGCCCACTTTCCTAGA
GAAGGAGTTTTCGTATCTAACGGCACCCACTGGTTCGTGACACAG
CGGAACTTTTACGAGCCTCAGATTATAACTACGGACAACACTTTC
GTGTCAGGCAACTGTGACGTGGTGATTGGGATCGTGAACAACACA
GTCTACGACCCATTGCAGCCCGAGTTGGACTCCTTCAAAGAGGAG
CTTGATAAGTATTTCAAGAACCATACCTCTCCCGACGTGGACCTG
GGGGACATTAGCGGCATCAATGCATCCGTTGTGAATATCCAGAAA
GAAATCGATAGGCTGAATGAGGTCGCAAAAAATCTTAATGAGTCA
CTGATTGATCTGCAGGAACTCGGCAAATATGAGCAGTATATTAAG
TGGCCGTGGTACATATGGCTCGGCTTTATCGCCGGTCTGATTGCC
ATCGTGATGGTGACCATTATGCTGTGTTGTATGACAAGCTGCTGT
TCATGTCTCAAAGGATGCTGCTCCTGCGGTAGCTGCTGTAAGTTC
GATGAAGACGACAGTGAGCCCGTGCTCAAAGGAGTGAAACTCCAC
TACACATAAcgatcg
SEQ ID NO: 4: SARS-CoV-2 N Protein
(nucleocapsid phosphoprotein) nucleic acid
sequence
ggtaccgccaccATGTCCGATAACGGCCCCCAGAATCAGAGAAAC
GCTCCCCGCATCACGTTCGGCGGACCAAGTGACAGCACAGGCAGT
AACCAGAACGGAGAACGCTCCGGTGCTCGCTCCAAGCAGCGACGG
CCGCAAGGGCTTCCCAACAATACCGCCAGCTGGTTTACGGCTCTG
ACCCAACACGGGAAAGAAGATCTTAAATTCCCCAGGGGCCAGGGC
GTCCCTATCAATACTAACTCCAGCCCGGATGATCAGATAGGCTAC
TATAGACGCGCTACCCGACGGATACGAGGGGGGGACGGCAAAATG
AAGGACCTTTCCCCCCGGTGGTATTTCTATTACTTGGGCACCGGA
CCAGAAGCCGGACTGCCTTACGGCGCTAACAAAGACGGAATAATC
TGGGTTGCGACGGAGGGCGCCCTGAATACACCTAAAGACCATATC
GGCACAAGAAATCCTGCTAACAATGCCGCGATTGTGCTCCAGCTG
CCTCAGGGAACCACGCTGCCTAAAGGGTTTTACGCTGAGGGGTCA
AGGGGGGGGAGTCAAGCGTCTAGTAGGTCATCCTCTCGCTCTCGC
AATAGTTCCCGGAACTCAACCCCAGGCAGCAGCAGAGGAACCTCT
CCCGCACGGATGGCTGGCAATGGGGGAGATGCTGCCCTTGCTCTC
CTTCTGCTGGATCGCCTTAACCAGCTCGAATCAAAGATGTCTGGA
AAAGGTCAGCAGCAGCAAGGCCAGACCGTGACAAAGAAGAGTGCA
GCTGAAGCTAGTAAAAAGCCACGCCAAAAACGGACCGCAACTAAG
GCATATAACGTAACACAGGCCTTCGGCAGAAGAGGTCCAGAACAA
ACACAGGGAAACTTTGGCGATCAAGAGCTGATTAGACAGGGCACA
GATTACAAACACTGGCCACAGATCGCGCAGTTTGCACCAAGCGCC
TCTGCATTCTTCGGGATGAGTCGGATTGGGATGGAAGTCACTCCA
TCCGGGACCTGGCTTACCTACACAGGGGCAATAAAACTCGACGAC
AAAGACCCAAACTTTAAAGATCAGGTCATCCTGCTGAATAAACAC
ATCGATGCCTACAAAACTTTCCCCCCAACCGAACCAAAGAAAGAC
AAGAAAAAAAAGGCAGACGAAACGCAAGCGCTCCCTCAGCGCCAG
AAGAAGCAGCAGACCGTTACACTGTTGCCAGCAGCAGATCTGGAT
GATTTTTCCAAGCAGCTTCAACAGAGTATGTCAAGCGCTGACAGC
ACTCAGGCTTGAcgatcg
SEQ ID NO: 5: SARS-CoV-2 Fusion:
S1-Furin-N nucleic acid sequence
ggtaccgccaccATGTTTGTTTTTCTCGTACTCCTGCCCCTGGTT
TCCTCCCAATGTGTCAATCTGACTACCCGGACCCAACTTCCTCCC
GCCTACACCAATTCCTTTACCCGAGGTGTTTACTACCCAGACAAA
GTGTTCAGGTCATCCGTCCTCCATAGTACCCAAGACCTCTTCCTC
CCTTTTTTTTCTAACGTTACCTGGTTTCACGCTATTCACGTTAGC
GGCACCAACGGCACCAAAAGATTCGATAACCCCGTACTGCCGTTC
AACGACGGGGTATATTTTGCCTCTACTGAAAAATCAAACATCATA
CGCGGATGGATCTTTGGGACTACCCTGGACTCAAAAACTCAGTCC
CTGCTGATTGTGAATAACGCTACCAACGTGGTGATCAAAGTCTGT
GAATTCCAGTTTTGCAACGATCCTTTTCTCGGCGTTTATTATCAC
AAAAATAACAAATCCTGGATGGAGAGCGAGTTCCGGGTGTACTCC
TCCGCGAATAATTGCACCTTCGAATATGTGTCTCAGCCATTCCTC
ATGGACCTCGAGGGGAAGCAGGGCAATTTTAAGAATCTGCGAGAA
TTCGTGTTCAAGAATATAGACGGTTACTTCAAGATTTACTCCAAA
CACACCCCGATTAACCTGGTTAGGGACTTGCCTCAGGGCTTTTCT
GCATTGGAGCCCCTCGTGGACCTCCCAATCGGCATAAACATTACA
AGATTTCAGACTTTGCTTGCATTGCACAGGAGCTATTTGACACCC
GGCGATTCTTCTTCCGGATGGACCGCTGGAGCAGCTGCTTATTAC
GTGGGCTATCTGCAGCCTCGAACCTTTCTTTTGAAGTACAACGAA
AATGGAACTATCACCGATGCAGTTGACTGCGCCCTGGACCCCCTG
TCCGAAACTAAGTGCACGCTCAAAAGTTTCACAGTAGAGAAGGGG
ATATACCAGACTAGCAATTTCCGCGTTCAGCCAACCGAAAGTATA
GTGCGCTTTCCTAATATAACTAACCTGTGTCCTTTCGGGGAAGTG
TTTAACGCCACTAGATTCGCTTCCGTCTACGCCTGGAATAGAAAG
AGGATCTCAAATTGCGTTGCTGACTATAGTGTTTTGTACAATTCC
GCCTCTTTCTCAACCTTCAAATGTTACGGGGTGAGCCCTACCAAA
CTGAACGACCTGTGCTTTACAAACGTATACGCCGACAGCTTTGTT
ATCAGAGGAGACGAGGTTCGCCAGATTGCTCCGGGTCAGACAGGC
AAGATTGCTGATTATAATTACAAACTGCCCGACGACTTTACAGGA
TGTGTGATCGCGTGGAACAGTAACAATCTTGACTCAAAGGTTGGG
GGTAATTATAATTATCTTTACCGGCTGTTCAGAAAAAGCAATTTG
AAACCCTTCGAAAGGGACATATCCACCGAGATCTATCAGGCCGGG
TCCACTCCATGCAATGGTGTGGAAGGTTTTAATTGCTACTTGCCA
TTGCAGTCTTATGGATTCCAACCAACCAATGGCGTAGGCTACCAG
CCGTATCGCGTTGTCGTGCTCAGCTTCGAGCTGCTCCACGCCCCC
GCGACCGTATGCGGTCCTAAGAAGTCCACCAATCTTGTTAAGAAC
AAGTGTGTAAACTTTAACTTTAACGGGCTGACCGGGACCGGCGTT
CTGACTGAATCTAACAAAAAATTCCTGCCTTTCCAGCAGTTCGGC
CGCGATATTGCTGACACCACTGACGCTGTAAGAGACCCTCAGACC
CTTGAAATTCTCGATATCACACCTTGCAGCTTTGGGGGCGTGTCC
GTCATCACTCCAGGAACTAACACAAGCAACCAGGTGGCAGTGTTG
TACCAGGATGTTAATTGTACCGAGGTGCCAGTGGCCATCCACGCC
GATCAATTGACACCTACCTGGAGGGTTTACAGCACAGGGTCCAAT
GTTTTTCAGACAAGAGCCGGATGTCTGATCGGTGCCGAGCATGTC
AACAATTCCTACGAGTGTGATATCCCCATTGGTGCGGGAATTTGT
GCATCATATCAGACCCAGACTAATAGCCCAAGAAGAGCTAGATCC
GTCGCTAGTCAATCCATCATTGCATATACAATGATGTCCGATAAC
GGCCCCCAGAATCAGAGAAACGCTCCCCGCATCACGTTCGGCGGA
CCAAGTGACAGCACAGGCAGTAACCAGAACGGAGAACGCTCCGGT
GCTCGCTCCAAGCAGCGACGGCCGCAAGGGCTTCCCAACAATACC
GCCAGCTGGTTTACGGCTCTGACCCAACACGGGAAAGAAGATCTT
AAATTCCCCAGGGGCCAGGGCGTCCCTATCAATACTAACTCCAGC
CCGGATGATCAGATAGGCTACTATAGACGCGCTACCCGACGGATA
CGAGGGGGGGACGGCAAAATGAAGGACCTTTCCCCCCGGTGGTAT
TTCTATTACTTGGGCACCGGACCAGAAGCCGGACTGCCTTACGGC
GCTAACAAAGACGGAATAATCTGGGTTGCGACGGAGGGCGCCCTG
AATACACCTAAAGACCATATCGGCACAAGAAATCCTGCTAACAAT
GCCGCGATTGTGCTCCAGCTGCCTCAGGGAACCACGCTGCCTAAA
GGGTTTTACGCTGAGGGGTCAAGGGGGGGGAGTCAAGCGTCTAGT
AGGTCATCCTCTCGCTCTCGCAATAGTTCCCGGAACTCAACCCCA
GGCAGCAGCAGAGGAACCTCTCCCGCACGGATGGCTGGCAATGGG
GGAGATGCTGCCCTTGCTCTCCTTCTGCTGGATCGCCTTAACCAG
CTCGAATCAAAGATGTCTGGAAAAGGTCAGCAGCAGCAAGGCCAG
ACCGTGACAAAGAAGAGTGCAGCTGAAGCTAGTAAAAAGCCACGC
CAAAAACGGACCGCAACTAAGGCATATAACGTAACACAGGCCTTC
GGCAGAAGAGGTCCAGAACAAACACAGGGAAACTTTGGCGATCAA
GAGCTGATTAGACAGGGCACAGATTACAAACACTGGCCACAGATC
GCGCAGTTTGCACCAAGCGCCTCTGCATTCTTCGGGATGAGTCGG
ATTGGGATGGAAGTCACTCCATCCGGGACCTGGCTTACCTACACA
GGGGCAATAAAACTCGACGACAAAGACCCAAACTTTAAAGATCAG
GTCATCCTGCTGAATAAACACATCGATGCCTACAAAACTTTCCCC
CCAACCGAACCAAAGAAAGACAAGAAAAAAAAGGCAGACGAAACG
CAAGCGCTCCCTCAGCGCCAGAAGAAGCAGCAGACCGTTACACTG
TTGCCAGCAGCAGATCTGGATGATTTTTCCAAGCAGCTTCAACAG
AGTATGTCAAGCGCTGACAGCACTCAGGCTTGAcgatcg
SEQ ID NO: 6: CMV-SARS-CoV-2-S-BGH-
bActin-SARS-CoV-2-N-SPA-BGH-CMV-
dsRNA-SPA
TAGTAATCAATTACGGGGTCATTAGTTCATAGCCCATATATGGAG
TTCCGCGTTACATAACTTACGGTAAATGGCCCGCCTGGCTGACCG
CCCAACGACCCCCGCCCATTGACGTCAATAATGACGTATGTTCCC
ATAGTAACGCCAATAGGGACTTTCCATTGACGTCAATGGGTGGAG
TATTTACGGTAAACTGCCCACTTGGCAGTACATCAAGTGTATCAT
ATGCCAAGTACGCCCCCTATTGACGTCAATGACGGTAAATGGCCC
GCCTGGCATTATGCCCAGTACATGACCTTATGGGACTTTCCTACT
TGGCAGTACATCTACGTATTAGTCATCGCTATTACCATGGTGATG
CGGTTTTGGCAGTACATCAATGGGCGTGGATAGCGGTTTGACTCA
CGGGGATTTCCAAGTCTCCACCCCATTGACGTCAATGGGAGTTTG
TTTTGGCACCAAAATCAACGGGACTTTCCAAAATGTCGTAACAAC
TCCGCCCCATTGACGCAAATGGGCGGTAGGCGTGTACGGTGGGAG
GTCTATATAAGCAGAGCTCGTTTAGTGAACCGTCAGATCGCCTGG
AGACGCCATCCACGCTGTTTTGACCTCCATAGAAGACACCGGGAC
CGATCCAGCCTGACTCTAGCCTAGCTCTGAAGTTGGTGGTGAGGC
CCTGGGCAGGTTGGTATCAAGGTTacaagacaggtttaaggagac
caatagaaactgggcatgtggagacagagaagactcttgggtttc
tgataggcactgactctctctgcctattggtctattttcccaccc
ttaggctgctggtctgagcctagGAGATCTCTCGAGGTCGACGGT
ATCGATGggtaccgccaccATGTTTGTTTTTCTCGTACTCCTGCC
CCTGGTTTCCTCCCAATGTGTCAATCTGACTACCCGGACCCAACT
TCCTCCCGCCTACACCAATTCCTTTACCCGAGGTGTTTACTACCC
AGACAAAGTGTTCAGGTCATCCGTCCTCCATAGTACCCAAGACCT
CTTCCTCCCTTTTTTTTCTAACGTTACCTGGTTTCACGCTATTCA
CGTTAGCGGCACCAACGGCACCAAAAGATTCGATAACCCCGTACT
GCCGTTCAACGACGGGGTATATTTTGCCTCTACTGAAAAATCAAA
CATCATACGCGGATGGATCTTTGGGACTACCCTGGACTCAAAAAC
TCAGTCCCTGCTGATTGTGAATAACGCTACCAACGTGGTGATCAA
AGTCTGTGAATTCCAGTTTTGCAACGATCCTTTTCTCGGCGTTTA
TTATCACAAAAATAACAAATCCTGGATGGAGAGCGAGTTCCGGGT
GTACTCCTCCGCGAATAATTGCACCTTCGAATATGTGTCTCAGCC
ATTCCTCATGGACCTCGAGGGGAAGCAGGGCAATTTTAAGAATCT
GCGAGAATTCGTGTTCAAGAATATAGACGGTTACTTCAAGATTTA
CTCCAAACACACCCCGATTAACCTGGTTAGGGACTTGCCTCAGGG
CTTTTCTGCATTGGAGCCCCTCGTGGACCTCCCAATCGGCATAAA
CATTACAAGATTTCAGACTTTGCTTGCATTGCACAGGAGCTATTT
GACACCCGGCGATTCTTCTTCCGGATGGACCGCTGGAGCAGCTGC
TTATTACGTGGGCTATCTGCAGCCTCGAACCTTTCTTTTGAAGTA
CAACGAAAATGGAACTATCACCGATGCAGTTGACTGCGCCCTGGA
CCCCCTGTCCGAAACTAAGTGCACGCTCAAAAGTTTCACAGTAGA
GAAGGGGATATACCAGACTAGCAATTTCCGCGTTCAGCCAACCGA
AAGTATAGTGCGCTTTCCTAATATAACTAACCTGTGTCCTTTCGG
GGAAGTGTTTAACGCCACTAGATTCGCTTCCGTCTACGCCTGGAA
TAGAAAGAGGATCTCAAATTGCGTTGCTGACTATAGTGTTTTGTA
CAATTCCGCCTCTTTCTCAACCTTCAAATGTTACGGGGTGAGCCC
TACCAAACTGAACGACCTGTGCTTTACAAACGTATACGCCGACAG
CTTTGTTATCAGAGGAGACGAGGTTCGCCAGATTGCTCCGGGTCA
GACAGGCAAGATTGCTGATTATAATTACAAACTGCCCGACGACTT
TACAGGATGTGTGATCGCGTGGAACAGTAACAATCTTGACTCAAA
GGTTGGGGGTAATTATAATTATCTTTACCGGCTGTTCAGAAAAAG
CAATTTGAAACCCTTCGAAAGGGACATATCCACCGAGATCTATCA
GGCCGGGTCCACTCCATGCAATGGTGTGGAAGGTTTTAATTGCTA
CTTCCCATTGCAGTCTTATGGATTCCAACCAACCAATGGCGTAGG
CTACCAGCCGTATCGCGTTGTCGTGCTCAGCTTCGAGCTGCTCCA
CGCCCCCGCGACCGTATGCGGTCCTAAGAAGTCCACCAATCTTGT
TAAGAACAAGTGTGTAAACTTTAACTTTAACGGGCTGACCGGGAC
CGGCGTTCTGACTGAATCTAACAAAAAATTCCTGCCTTTCCAGCA
GTTCGGCCGCGATATTGCTGACACCACTGACGCTGTAAGAGACCC
TCAGACCCTTGAAATTCTCGATATCACACCTTGCAGCTTTGGGGG
CGTGTCCGTCATCACTCCAGGAACTAACACAAGCAACCAGGTGGC
AGTGTTGTACCAGGATGTTAATTGTACCGAGGTGCCAGTGGCCAT
CCACGCCGATCAATTGACACCTACCTGGAGGGTTTACAGCACAGG
GTCCAATGTTTTTCAGACAAGAGCCGGATGTCTGATCGGTGCCGA
GCATGTCAACAATTCCTACGAGTGTGATATCCCCATTGGTGCGGG
AATTTGTGCATCATATCAGACCCAGACTAATAGCCCAAGAAGAGC
TAGATCCGTCGCTAGTCAATCCATCATTGCATATACAATGTCCCT
GGGAGCTGAGAATTCAGTCGCGTATTCAAACAATTCCATTGCTAT
TCCTACTAATTTCACTATCTCCGTCACGACCGAGATCCTGCCAGT
TTCCATGACTAAGACTTCTGTTGACTGCACCATGTATATCTGTGG
CGATAGCACCGAGTGCAGTAATCTGCTTCTGCAGTACGGCTCCTT
CTGCACACAACTCAATCGAGCACTGACCGGTATTGCAGTTGAGCA
GGACAAGAACACACAGGAGGTCTTTGCACAGGTCAAACAAATTTA
CAAAACCCCCCCCATAAAAGACTTTGGTGGGTTCAACTTCAGCCA
AATCCTCCCAGATCCCAGCAAGCCCTCCAAAAGATCCTTCATCGA
AGACCTTTTGTTCAATAAGGTAACCCTGGCCGACGCAGGCTTCAT
CAAACAATATGGCGATTGCCTTGGAGACATTGCTGCGCGCGATTT
GATCTGTGCTCAGAAATTTAACGGTTTGACCGTGCTGCCCCCACT
TCTGACTGATGAGATGATAGCACAGTATACTTCTGCTCTTCTGGC
AGGAACAATCACTTCCGGGTGGACCTTTGGCGCTGGTGCAGCACT
GCAAATCCCCTTCGCAATGCAAATGGCCTACCGATTCAATGGTAT
TGGTGTTACCCAGAACGTGCTCTATGAGAATCAGAAACTCATCGC
CAATCAGTTCAATAGCGCTATTGGCAAGATTCAGGATTCCCTCAG
CTCTACCGCCAGCGCTCTGGGGAAGCTCCAGGACGTGGTGAACCA
AAATGCTCAAGCGCTCAATACCCTTGTGAAACAGCTCAGCTCCAA
TTTTGGCGCAATTAGCAGCGTTCTGAATGATATTCTGTCCCGGCT
GGACAAGGTAGAAGCAGAAGTCCAGATCGACAGGCTGATCACCGG
GCGGTTGCAGAGTCTCCAGACCTATGTCACACAACAGCTGATCCG
CGCCGCCGAGATCAGGGCTTCCGCTAACCTGGCCGCCACTAAGAT
GTCCGAATGCGTGTTGGGGCAGAGTAAGCGGGTCGACTTTTGCGG
GAAGGGATACCATCTGATGAGCTTCCCTCAGTCTGCACCCCACGG
AGTAGTGTTCCTCCACGTCACATATGTGCCCGCTCAGGAAAAGAA
TTTCACAACCGCACCTGCTATCTGTCACGACGGCAAGGCCCACTT
TCCTAGAGAAGGAGTTTTCGTATCTAACGGCACCCACTGGTTCGT
GACACAGCGGAACTTTTACGAGCCTCAGATTATAACTACGGACAA
CACTTTCGTGTCAGGCAACTGTGACGTGGTGATTGGGATCGTGAA
CAACACAGTCTACGACCCATTGCAGCCCGAGTTGGACTCCTTCAA
AGAGGAGCTTGATAAGTATTTCAAGAACCATACCTCTCCCGACGT
GGACCTGGGGGACATTAGCGGCATCAATGCATCCGTTGTGAATAT
CCAGAAAGAAATCGATAGGCTGAATGAGGTCGCAAAAAATCTTAA
TGAGTCACTGATTGATCTGCAGGAACTCGGCAAATATGAGCAGTA
TATTAAGTGGCCGTGGTACATATGGCTCGGCTTTATCGCCGGTCT
GATTGCCATCGTGATGGTGACCATTATGCTGTGTTGTATGACAAG
CTGCTGTTCATGTCTCAAAGGATGCTGCTCCTGCGGTAGCTGCTG
TAAGTTCGATGAAGACGACAGTGAGCCCGTGCTCAAAGGAGTGAA
ACTCCACTACACATAAcgatcgagcgtAGAGCTCGCTGATCAGCC
TCGACTGTGCCTTCTAGTTGCCAGCCATCTGTTGTTTGCCCCTCC
CCCGTGCCTTCCTTGACCCTGGAAGGTGCCACTCCCACTGTCCTT
TCCTAATAAAATGAGGAAATTGCATCGCATTGTCTGAGTAGGTGT
CATTCTATTCTGGGGGGTGGGGTGGGGCAGGACAGCAAGGGGGAG
GATTGGGAAGACAATAGCAGGCATGCTGGGGATGCGGTGGGCTCT
ATGGCTTCTGAGGCGGAAAGAACCAaagcttgcggccgcGCCCAG
CACCCCAAGGCGGCCAACGCCAAAACTCTCCCTCCTCCTCTTCCT
CAATCTCGCTCTCGCTCTTTTTTTTTTTCGCAAAAGGAGGGGAGA
GGGGGTAAAAAAATGCTGCACTGTGCGGCGAAGCCGGTGAGTGAG
CGGCGCGGGGCCAATCAGCGTGCGCCGTTCCGAAAGTTGCCTTTT
ATGGCTCGAGCGGCCGCGGCGGCGCCCTATAAAACCCAGCGGCGC
GACGCGCCACCACCGCCGAGACcctgcaggccgccaccATGTCCG
ATAACGGCCCCCAGAATCAGAGAAACGCTCCCCGCATCACGTTCG
GCGGACCAAGTGACAGCACAGGCAGTAACCAGAACGGAGAACGCT
CCGGTGCTCGCTCCAAGCAGCGACGGCCGCAAGGGCTTCCCAACA
ATACCGCCAGCTGGTTTACGGCTCTGACCCAACACGGGAAAGAAG
ATCTTAAATTCCCCAGGGGCCAGGGCGTCCCTATCAATACTAACT
CCAGCCCGGATGATCAGATAGGCTACTATAGACGCGCTACCCGAC
GGATACGAGGGGGGGACGGCAAAATGAAGGACCTTTCCCCCCGGT
GGTATTTCTATTACTTGGGCACCGGACCAGAAGCCGGACTGCCTT
ACGGCGCTAACAAAGACGGAATAATCTGGGTTGCGACGGAGGGCG
CCCTGAATACACCTAAAGACCATATCGGCACAAGAAATCCTGCTA
ACAATGCCGCGATTGTGCTCCAGCTGCCTCAGGGAACCACGCTGC
CTAAAGGGTTTTACGCTGAGGGGTCAAGGGGGGGGAGTCAAGCGT
CTAGTAGGTCATCCTCTCGCTCTCGCAATAGTTCCCGGAACTCAA
CCCCAGGCAGCAGCAGAGGAACCTCTCCCGCACGGATGGCTGGCA
ATGGGGGAGATGCTGCCCTTGCTCTCCTTCTGCTGGATCGCCTTA
ACCAGCTCGAATCAAAGATGTCTGGAAAAGGTCAGCAGCAGCAAG
GCCAGACCGTGACAAAGAAGAGTGCAGCTGAAGCTAGTAAAAAGC
CACGCCAAAAACGGACCGCAACTAAGGCATATAACGTAACACAGG
CCTTCGGCAGAAGAGGTCCAGAACAAACACAGGGAAACTTTGGCG
ATCAAGAGCTGATTAGACAGGGCACAGATTACAAACACTGGCCAC
AGATCGCGCAGTTTGCACCAAGCGCCTCTGCATTCTTCGGGATGA
GTCGGATTGGGATGGAAGTCACTCCATCCGGGACCTGGCTTACCT
ACACAGGGGCAATAAAACTCGACGACAAAGACCCAAACTTTAAAG
ATCAGGTCATCCTGCTGAATAAACACATCGATGCCTACAAAACTT
TCCCCCCAACCGAACCAAAGAAAGACAAGAAAAAAAAGGCAGACG
AAACGCAAGCGCTCCCTCAGCGCCAGAAGAAGCAGCAGACCGTTA
CACTGTTGCCAGCAGCAGATCTGGATGATTTTTCCAAGCAGCTTC
AACAGAGTATGTCAAGCGCTGACAGCACTCAGGCTTGAggcgcgc
cgctgaccgatAAATAAAATATCTTTATTTTCATTACATCTGTGT
GTTGGTTTTTTGTGTGacgcgttagttattaataGTAATCAATTA
CGGGGTCATTAGTTCATAGCCCATATATGGAGTTCCGCGTTACAT
AACTTACGGTAAATGGCCCGCCTGGCTGACCGCCCAACGACCCCC
GCCCATTGACGTCAATAATGACGTATGTTCCCATAGTAACGCCAA
TAGGGACTTTCCATTGACGTCAATGGGTGGAGTATTTACGGTAAA
CTGCCCACTTGGCAGTACATCAAGTGTATCATATGCCAAGTACGC
CCCCTATTGACGTCAATGACGGTAAATGGCCCGCCTGGCATTATG
CCCAGTACATGACCTTATGGGACTTTCCTACTTGGCAGTACATCT
ACGTATTAGTCATCGCTATTACCATGGTGATGCGGTTTTGGCAGT
ACATCAATGGGCGTGGATAGCGGTTTGACTCACGGGGATTTCCAA
GTCTCCACCCCATTGACGTCAATGGGAGTTTGTTTTGGCACCAAA
ATCAACGGGACTTTCCAAAATGTCGTAACAACTCCGCCCCATTGA
CGCAAATGGGCGGTAGGCGTGTACGGTGGGAGGTCTATATAAGCA
GAGCTGGTTTAGTGAACCGTCAGATCCGCTAGAGATATCGGGCCA
CTGCAGGAAACGATATGGGCTGAATACGGATCCGTATTCAGCCCA
TATCGTTTCTCTAGAAATAAAATATCTTTATTTTCATTACATCTG
TGTGTTGGTTTTTTGTGTG
SEQ ID NO: 7:
CMV-SARS-CoV-2-S1-Furin-N-BGH-CMV-dsRNA-SPA
TAGTAATCAATTACGGGGTCATTAGTTCATAGCCCATATATGGAG
TTCCGCGTTACATAACTTACGGTAAATGGCCCGCCTGGCTGACCG
CCCAACGACCCCCGCCCATTGACGTCAATAATGACGTATGTTCCC
ATAGTAACGCCAATAGGGACTTTCCATTGACGTCAATGGGTGGAG
TATTTACGGTAAACTGCCCACTTGGCAGTACATCAAGTGTATCAT
ATGCCAAGTACGCCCCCTATTGACGTCAATGACGGTAAATGGCCC
GCCTGGCATTATGCCCAGTACATGACCTTATGGGACTTTCCTACT
TGGCAGTACATCTACGTATTAGTCATCGCTATTACCATGGTGATG
CGGTTTTGGCAGTACATCAATGGGCGTGGATAGCGGTTTGACTCA
CGGGGATTTCCAAGTCTCCACCCCATTGACGTCAATGGGAGTTTG
TTTTGGCACCAAAATCAACGGGACTTTCCAAAATGTCGTAACAAC
TCCGCCCCATTGACGCAAATGGGCGGTAGGCGTGTACGGTGGGAG
GTCTATATAAGCAGAGCTCGTTTAGTGAACCGTCAGatcgcctgg
agacgccatccacgctgttttgacctccatagaagacaccgggac
cgatccagcctgactctagCctAGCTCtgaagttggtggtgaggc
cctgggcaggttggtatcaaggttacaagacaggtttaaggagac
caatagaaactgggcatgtggagacagagaagactcttgggtttc
tgataggcactgactctctctgcctattggtctattttcccaccc
ttaggctgctggtctgagcctagGAGATCTCTCGAGGTCGACGGT
ATCGATGggtaccgccaccATGTTTGTTTTTCTCGTACTCCTGCC
CCTGGTTTCCTCCCAATGTGTCAATCTGACTACCCGGACCCAACT
TCCTCCCGCCTACACCAATTCCTTTACCCGAGGTGTTTACTACCC
AGACAAAGTGTTCAGGTCATCCGTCCTCCATAGTACCCAAGACCT
CTTCCTCCCTTTTTTTTCTAACGTTACCTGGTTTCACGCTATTCA
CGTTAGCGGCACCAACGGCACCAAAAGATTCGATAACCCCGTACT
GCCGTTCAACGACGGGGTATATTTTGCCTCTACTGAAAAATCAAA
CATCATACGCGGATGGATCTTTGGGACTACCCTGGACTCAAAAAC
TCAGTCCCTGCTGATTGTGAATAACGCTACCAACGTGGTGATCAA
AGTCTGTGAATTCCAGTTTTGCAACGATCCTTTTCTCGGCGTTTA
TTATCACAAAAATAACAAATCCTGGATGGAGAGCGAGTTCCGGGT
GTACTCCTCCGCGAATAATTGCACCTTCGAATATGTGTCTCAGCC
ATTCCTCATGGACCTCGAGGGGAAGCAGGGCAATTTTAAGAATCT
GCGAGAATTCGTGTTCAAGAATATAGACGGTTACTTCAAGATTTA
CTCCAAACACACCCCGATTAACCTGGTTAGGGACTTGCCTCAGGG
CTTTTCTGCATTGGAGCCCCTCGTGGACCTCCCAATCGGCATAAA
CATTACAAGATTTCAGACTTTGCTTGCATTGCACAGGAGCTATTT
GACACCCGGCGATTCTTCTTCCGGATGGACCGCTGGAGCAGCTGC
TTATTACGTGGGCTATCTGCAGCCTCGAACCTTTCTTTTGAAGTA
CAACGAAAATGGAACTATCACCGATGCAGTTGACTGCGCCCTGGA
CCCCCTGTCCGAAACTAAGTGCACGCTCAAAAGTTTCACAGTAGA
GAAGGGGATATACCAGACTAGCAATTTCCGCGTTCAGCCAACCGA
AAGTATAGTGCGCTTTCCTAATATAACTAACCTGTGTCCTTTCGG
GGAAGTGTTTAACGCCACTAGATTCGCTTCCGTCTACGCCTGGAA
TAGAAAGAGGATCTCAAATTGCGTTGCTGACTATAGTGTTTTGTA
CAATTCCGCCTCTTTCTCAACCTTCAAATGTTACGGGGTGAGCCC
TACCAAACTGAACGACCTGTGCTTTACAAACGTATACGCCGACAG
CTTTGTTATCAGAGGAGACGAGGTTCGCCAGATTGCTCCGGGTCA
GACAGGCAAGATTGCTGATTATAATTACAAACTGCCCGACGACTT
TACAGGATGTGTGATCGCGTGGAACAGTAACAATCTTGACTCAAA
GGTTGGGGGTAATTATAATTATCTTTACCGGCTGTTCAGAAAAAG
CAATTTGAAACCCTTCGAAAGGGACATATCCACCGAGATCTATCA
GGCCGGGTCCACTCCATGCAATGGTGTGGAAGGTTTTAATTGCTA
CTTCCCATTGCAGTCTTATGGATTCCAACCAACCAATGGCGTAGG
CTACCAGCCGTATCGCGTTGTCGTGCTCAGCTTCGAGCTGCTCCA
CGCCCCCGCGACCGTATGCGGTCCTAAGAAGTCCACCAATCTTGT
TAAGAACAAGTGTGTAAACTTTAACTTTAACGGGCTGACCGGGAC
CGGCGTTCTGACTGAATCTAACAAAAAATTCCTGCCTTTCCAGCA
GTTCGGCCGCGATATTGCTGACACCACTGACGCTGTAAGAGACCC
TCAGACCCTTGAAATTCTCGATATCACACCTTGCAGCTTTGGGGG
CGTGTCCGTCATCACTCCAGGAACTAACACAAGCAACCAGGTGGC
AGTGTTGTACCAGGATGTTAATTGTACCGAGGTGCCAGTGGCCAT
CCACGCCGATCAATTGACACCTACCTGGAGGGTTTACAGCACAGG
GTCCAATGTTTTTCAGACAAGAGCCGGATGTCTGATCGGTGCCGA
GCATGTCAACAATTCCTACGAGTGTGATATCCCCATTGGTGCGGG
AATTTGTGCATCATATCAGACCCAGACTAATAGCCCAAGAAGAGC
TAGATCCGTCGCTAGTCAATCCATCATTGCATATACAATGATGTC
CGATAACGGCCCCCAGAATCAGAGAAACGCTCCCCGCATCACGTT
CGGCGGACCAAGTGACAGCACAGGCAGTAACCAGAACGGAGAACG
CTCCGGTGCTCGCTCCAAGCAGCGACGGCCGCAAGGGCTTCCCAA
CAATACCGCCAGCTGGTTTACGGCTCTGACCCAACACGGGAAAGA
AGATCTTAAATTCCCCAGGGGCCAGGGCGTCCCTATCAATACTAA
CTCCAGCCCGGATGATCAGATAGGCTACTATAGACGCGCTACCCG
ACGGATACGAGGGGGGGACGGCAAAATGAAGGACCTTTCCCCCCG
GTGGTATTTCTATTACTTGGGCACCGGACCAGAAGCCGGACTGCC
TTACGGCGCTAACAAAGACGGAATAATCTGGGTTGCGACGGAGGG
CGCCCTGAATACACCTAAAGACCATATCGGCACAAGAAATCCTGC
TAACAATGCCGCGATTGTGCTCCAGCTGCCTCAGGGAACCACGCT
GCCTAAAGGGTTTTACGCTGAGGGGTCAAGGGGGGGGAGTCAAGC
GTCTAGTAGGTCATCCTCTCGCTCTCGCAATAGTTCCCGGAACTC
AACCCCAGGCAGCAGCAGAGGAACCTCTCCCGCACGGATGGCTGG
CAATGGGGGAGATGCTGCCCTTGCTCTCCTTCTGCTGGATCGCCT
TAACCAGCTCGAATCAAAGATGTCTGGAAAAGGTCAGCAGCAGCA
AGGCCAGACCGTGACAAAGAAGAGTGCAGCTGAAGCTAGTAAAAA
GCCACGCCAAAAACGGACCGCAACTAAGGCATATAACGTAACACA
GGCCTTCGGCAGAAGAGGTCCAGAACAAACACAGGGAAACTTTGG
CGATCAAGAGCTGATTAGACAGGGCACAGATTACAAACACTGGCC
ACAGATCGCGCAGTTTGCACCAAGCGCCTCTGCATTCTTCGGGAT
GAGTCGGATTGGGATGGAAGTCACTCCATCCGGGACCTGGCTTAC
CTACACAGGGGCAATAAAACTCGACGACAAAGACCCAAACTTTAA
AGATCAGGTCATCCTGCTGAATAAACACATCGATGCCTACAAAAC
TTTCCCCCCAACCGAACCAAAGAAAGACAAGAAAAAAAAGGCAGA
CGAAACGCAAGCGCTCCCTCAGCGCCAGAAGAAGCAGCAGACCGT
TACACTGTTGCCAGCAGCAGATCTGGATGATTTTTCCAAGCAGCT
TCAACAGAGTATGTCAAGCGCTGACAGCACTCAGGCTTGAcgatc
gGATATCGCTAGCGTACCGGCGGCCGCCCTATTCTATAGTGTCAC
CTAAATGCTAGAGCTCGCTGATCAGCCTCGACTGTGCCTTCTAGT
TGCCAGCCATCTGTTGTTTGCCCCTCCCCCGTGCCTTCCTTGACC
CTGGAAGGTGCCACTCCCACTGTCCTTTCCTAATAAAATGAGGAA
ATTGCATCGCATTGTCTGAGTAGGTGTCATTCTATTCTGGGGGGT
GGGGTGGGGCAGGACAGCAAGGGGGAGGATTGGGAAGACAATAGC
AGGCATGCTGGGGATGCGGTGGGCTCTATGGCTTCTGAGGCGGAA
AGAACCAAAGCTTAcgcgttagttattaataGTAATCAATTACGG
GGTCATTAGTTCATAGCCCATATATGGAGTTCCGCGTTACATAAC
TTACGGTAAATGGCCCGCCTGGCTGACCGCCCAACGACCCCCGCC
CATTGACGTCAATAATGACGTATGTTCCCATAGTAACGCCAATAG
GGACTTTCCATTGACGTCAATGGGTGGAGTATTTACGGTAAACTG
CCCACTTGGCAGTACATCAAGTGTATCATATGCCAAGTACGCCCC
CTATTGACGTCAATGACGGTAAATGGCCCGCCTGGCATTATGCCC
AGTACATGACCTTATGGGACTTTCCTACTTGGCAGTACATCTACG
TATTAGTCATCGCTATTACCATGGTGATGCGGTTTTGGCAGTACA
TCAATGGGCGTGGATAGCGGTTTGACTCACGGGGATTTCCAAGTC
TCCACCCCATTGACGTCAATGGGAGTTTGTTTTGGCACCAAAATC
AACGGGACTTTCCAAAATGTCGTAACAACTCCGCCCCATTGACGC
AAATGGGCGGTAGGCGTGTACGGTGGGAGGTCTATATAAGCAGAG
CTGGTTTAGTGAACCGTCAGATCCGCTAGAGATATCGGGCCACTG
CAGGAAACGATATGGGCTGAATACGGATCCGTATTCAGCCCATAT
CGTTTCTCTAGAAATAAAATATCTTTATTTTCATTACATCTGTGT
GTTGGTTTTTTGTGTG
SEQ ID NO: 8
rAd-CMV-SARS-CoV-2-S-BGH-bActin-
SARS-CoV-2-N-SPA-BGH-CMV-dsRNA-SPA
TAAGGATCCCATCATCAATAATATACCTTATTTTGGATTGAAGCC
AATATGATAATGAGGGGGTGGAGTTTGTGACGTGGCGCGGGGCGT
GGGAACGGGGGGGGTGACGTAGTAGTGTGGCGGAAGTGTGATGTT
GCAAGTGTGGCGGAACACATGTAAGCGACGGATGTGGCAAAAGTG
ACGTTTTTGGTGTGCGCCGGTGTACACAGGAAGTGACAATTTTCG
CGCGGTTTTAGGCGGATGTTGTAGTAAATTTGGGCGTAACCGAGT
AAGATTTGGCCATTTTCGCGGGAAAACTGAATAAGAGGAAGTGAA
ATCTGAATAATTTTGTGTTACTCATAGCGCGTAATACTGCTAGAG
ATCTGGCGAAAGGGGGATGTGCTGCAAGGCGATTAAGTTGGGTAA
CGCCAGGGTTTTCCCAGTCACGACGTTGTAAAACGACGGCCAGTG
AATTGTAATACGACTCACTATAGGGCGAATTGGGTACTGGCCACA
GGAGCTTGGCCCATTGCATACGTTGTATCCATATCATAATATGTA
CATTTATATTGGCTCATGTCCAACATTACCGCCATGTTGACATTG
ATTATTGACTAGTTATTAATAGTAATCAATTACGGGGTCATTAGT
TCATAGCCCATATATGGAGTTCCGCGTTACATAACTTACGGTAAA
TGGCCCGCCTGGCTGACCGCCCAACGACCCCCGCCCATTGACGTC
AATAATGACGTATGTTCCCATAGTAACGCCAATAGGGACTTTCCA
TTGACGTCAATGGGTGGAGTATTTACGGTAAACTGCCCACTTGGC
AGTACATCAAGTGTATCATATGCCAAGTACGCCCCCTATTGACGT
CAATGACGGTAAATGGCCCGCCTGGCATTATGCCCAGTACATGAC
CTTATGGGACTTTCCTACTTGGCAGTACATCTACGTATTAGTCAT
CGCTATTACCATGGTGATGCGGTTTTGGCAGTACATCAATGGGCG
TGGATAGCGGTTTGACTCACGGGGATTTCCAAGTCTCCACCCCAT
TGACGTCAATGGGAGTTTGTTTTGGCACCAAAATCAACGGGACTT
TCCAAAATGTCGTAACAACTCCGCCCCATTGACGCAAATGGGCGG
TAGGCGTGTACGGTGGGAGGTCTATATAAGCAGAGCTCGTTTAGT
GAACCGTCAGATCGCCTGGAGACGCCATCCACGCTGTTTTGACCT
CCATAGAAGACACCGGGACCGATCCAGCCTGACTCTAGCCTAGCT
CTGAAGTTGGTGGTGAGGCCCTGGGCAGGTTGGTATCAAGGTTac
aagacaggtttaaggagaccaatagaaactgggcatgtggagaca
gagaagactcttgggtttctgataggcactgactctctctgccta
ttggtctattttcccacccttaggctgctggtctgagcctagGAG
ATCTCTCGAGGTCGACGGTATCGATGggtaccgccaccATGTTTG
TTTTTCTCGTACTCCTGCCCCTGGTTTCCTCCCAATGTGTCAATC
TGACTACCCGGACCCAACTTCCTCCCGCCTACACCAATTCCTTTA
CCCGAGGTGTTTACTACCCAGACAAAGTGTTCAGGTCATCCGTCC
TCCATAGTACCCAAGACCTCTTCCTCCCTTTTTTTTCTAACGTTA
CCTGGTTTCACGCTATTCACGTTAGCGGCACCAACGGCACCAAAA
GATTCGATAACCCCGTACTGCCGTTCAACGACGGGGTATATTTTG
CCTCTACTGAAAAATCAAACATCATACGCGGATGGATCTTTGGGA
CTACCCTGGACTCAAAAACTCAGTCCCTGCTGATTGTGAATAACG
CTACCAACGTGGTGATCAAAGTCTGTGAATTCCAGTTTTGCAACG
ATCCTTTTCTCGGCGTTTATTATCACAAAAATAACAAATCCTGGA
TGGAGAGCGAGTTCCGGGTGTACTCCTCCGCGAATAATTGCACCT
TCGAATATGTGTCTCAGCCATTCCTCATGGACCTCGAGGGGAAGC
AGGGCAATTTTAAGAATCTGCGAGAATTCGTGTTCAAGAATATAG
ACGGTTACTTCAAGATTTACTCCAAACACACCCCGATTAACCTGG
TTAGGGACTTGCCTCAGGGCTTTTCTGCATTGGAGCCCCTCGTGG
ACCTCCCAATCGGCATAAACATTACAAGATTTCAGACTTTGCTTG
CATTGCACAGGAGCTATTTGACACCCGGCGATTCTTCTTCCGGAT
GGACCGCTGGAGCAGCTGCTTATTACGTGGGCTATCTGCAGCCTC
GAACCTTTCTTTTGAAGTACAACGAAAATGGAACTATCACCGATG
CAGTTGACTGCGCCCTGGACCCCCTGTCCGAAACTAAGTGCACGC
TCAAAAGTTTCACAGTAGAGAAGGGGATATACCAGACTAGCAATT
TCCGCGTTCAGCCAACCGAAAGTATAGTGCGCTTTCCTAATATAA
CTAACCTGTGTCCTTTCGGGGAAGTGTTTAACGCCACTAGATTCG
CTTCCGTCTACGCCTGGAATAGAAAGAGGATCTCAAATTGCGTTG
CTGACTATAGTGTTTTGTACAATTCCGCCTCTTTCTCAACCTTCA
AATGTTACGGGGTGAGCCCTACCAAACTGAACGACCTGTGCTTTA
CAAACGTATACGCCGACAGCTTTGTTATCAGAGGAGACGAGGTTC
GCCAGATTGCTCCGGGTCAGACAGGCAAGATTGCTGATTATAATT
ACAAACTGCCCGACGACTTTACAGGATGTGTGATCGCGTGGAACA
GTAACAATCTTGACTCAAAGGTTGGGGGTAATTATAATTATCTTT
ACCGGCTGTTCAGAAAAAGCAATTTGAAACCCTTCGAAAGGGACA
TATCCACCGAGATCTATCAGGCCGGGTCCACTCCATGCAATGGTG
TGGAAGGTTTTAATTGCTACTTCCCATTGCAGTCTTATGGATTCC
AACCAACCAATGGCGTAGGCTACCAGCCGTATCGCGTTGTCGTGC
TCAGCTTCGAGCTGCTCCACGCCCCCGCGACCGTATGCGGTCCTA
AGAAGTCCACCAATCTTGTTAAGAACAAGTGTGTAAACTTTAACT
TTAACGGGCTGACCGGGACCGGCGTTCTGACTGAATCTAACAAAA
AATTCCTGCCTTTCCAGCAGTTCGGCCGCGATATTGCTGACACCA
CTGACGCTGTAAGAGACCCTCAGACCCTTGAAATTCTCGATATCA
CACCTTGCAGCTTTGGGGGCGTGTCCGTCATCACTCCAGGAACTA
ACACAAGCAACCAGGTGGCAGTGTTGTACCAGGATGTTAATTGTA
CCGAGGTGCCAGTGGCCATCCACGCCGATCAATTGACACCTACCT
GGAGGGTTTACAGCACAGGGTCCAATGTTTTTCAGACAAGAGCCG
GATGTCTGATCGGTGCCGAGCATGTCAACAATTCCTACGAGTGTG
ATATCCCCATTGGTGCGGGAATTTGTGCATCATATCAGACCCAGA
CTAATAGCCCAAGAAGAGCTAGATCCGTCGCTAGTCAATCCATCA
TTGCATATACAATGTCCCTGGGAGCTGAGAATTCAGTCGCGTATT
CAAACAATTCCATTGCTATTCCTACTAATTTCACTATCTCCGTCA
CGACCGAGATCCTGCCAGTTTCCATGACTAAGACTTCTGTTGACT
GCACCATGTATATCTGTGGCGATAGCACCGAGTGCAGTAATCTGC
TTCTGCAGTACGGCTCCTTCTGCACACAACTCAATCGAGCACTGA
CCGGTATTGCAGTTGAGCAGGACAAGAACACACAGGAGGTCTTTG
CACAGGTCAAACAAATTTACAAAACCCCCCCCATAAAAGACTTTG
GTGGGTTCAACTTCAGCCAAATCCTCCCAGATCCCAGCAAGCCCT
CCAAAAGATCCTTCATCGAAGACCTTTTGTTCAATAAGGTAACCC
TGGCCGACGCAGGCTTCATCAAACAATATGGCGATTGCCTTGGAG
ACATTGCTGCGCGCGATTTGATCTGTGCTCAGAAATTTAACGGTT
TGACCGTGCTGCCCCCACTTCTGACTGATGAGATGATAGCACAGT
ATACTTCTGCTCTTCTGGCAGGAACAATCACTTCCGGGTGGACCT
TTGGCGCTGGTGCAGCACTGCAAATCCCCTTCGCAATGCAAATGG
CCTACCGATTCAATGGTATTGGTGTTACCCAGAACGTGCTCTATG
AGAATCAGAAACTCATCGCCAATCAGTTCAATAGCGCTATTGGCA
AGATTCAGGATTCCCTCAGCTCTACCGCCAGCGCTCTGGGGAAGC
TCCAGGACGTGGTGAACCAAAATGCTCAAGCGCTCAATACCCTTG
TGAAACAGCTCAGCTCCAATTTTGGCGCAATTAGCAGCGTTCTGA
ATGATATTCTGTCCCGGCTGGACAAGGTAGAAGCAGAAGTCCAGA
TCGACAGGCTGATCACCGGGCGGTTGCAGAGTCTCCAGACCTATG
TCACACAACAGCTGATCCGCGCCGCCGAGATCAGGGCTTCCGCTA
ACCTGGCCGCCACTAAGATGTCCGAATGCGTGTTGGGGCAGAGTA
AGCGGGTCGACTTTTGCGGGAAGGGATACCATCTGATGAGCTTCC
CTCAGTCTGCACCCCACGGAGTAGTGTTCCTCCACGTCACATATG
TGCCCGCTCAGGAAAAGAATTTCACAACCGCACCTGCTATCTGTC
ACGACGGCAAGGCCCACTTTCCTAGAGAAGGAGTTTTCGTATCTA
ACGGCACCCACTGGTTCGTGACACAGCGGAACTTTTACGAGCCTC
AGATTATAACTACGGACAACACTTTCGTGTCAGGCAACTGTGACG
TGGTGATTGGGATCGTGAACAACACAGTCTACGACCCATTGCAGC
CCGAGTTGGACTCCTTCAAAGAGGAGCTTGATAAGTATTTCAAGA
ACCATACCTCTCCCGACGTGGACCTGGGGGACATTAGCGGCATCA
ATGCATCCGTTGTGAATATCCAGAAAGAAATCGATAGGCTGAATG
AGGTCGCAAAAAATCTTAATGAGTCACTGATTGATCTGCAGGAAC
TCGGCAAATATGAGCAGTATATTAAGTGGCCGTGGTACATATGGC
TCGGCTTTATCGCCGGTCTGATTGCCATCGTGATGGTGACCATTA
TGCTGTGTTGTATGACAAGCTGCTGTTCATGTCTCAAAGGATGCT
GCTCCTGCGGTAGCTGCTGTAAGTTCGATGAAGACGACAGTGAGC
CCGTGCTCAAAGGAGTGAAACTCCACTACACATAAcgatcgacgc
gtAGAGCTCGCTGATCAGCCTCGACTGTGCCTTCTAGTTGCCAGC
CATCTGTTGTTTGCCCCTCCCCCGTGCCTTCCTTGACCCTGGAAG
GTGCCACTCCCACTGTCCTTTCCTAATAAAATGAGGAAATTGCAT
CGCATTGTCTGAGTAGGTGTCATTCTATTCTGGGGGGTGGGGTGG
GGCAGGACAGCAAGGGGGAGGATTGGGAAGACAATAGCAGGCATG
CTGGGGATGCGGTGGGCTCTATGGCTTCTGAGGCGGAAAGAACCA
aagcttgcggccgcGCCCAGCACCCCAAGGCGGCCAACGCCAAAA
CTCTCCCTCCTCCTCTTCCTCAATCTCGCTCTCGCTCTTTTTTTT
TTTCGCAAAAGGAGGGGAGAGGGGGTAAAAAAATGCTGCACTGTG
CGGCGAAGCCGGTGAGTGAGCGGCGCGGGGCCAATCAGCGTGCGC
CGTTCCGAAAGTTGCCTTTTATGGCTCGAGCGGCCGCGGCGGCGC
CCTATAAAACCCAGCGGCGCGACGCGCCACCACCGCCGAGACcct
gcaggccgccaccATGTCCGATAACGGCCCCCAGAATCAGAGAAA
CGCTCCCCGCATCACGTTCGGCGGACCAAGTGACAGCACAGGCAG
TAACCAGAACGGAGAACGCTCCGGTGCTCGCTCCAAGCAGCGACG
GCCGCAAGGGCTTCCCAACAATACCGCCAGCTGGTTTACGGCTCT
GACCCAACACGGGAAAGAAGATCTTAAATTCCCCAGGGGCCAGGG
CGTCCCTATCAATACTAACTCCAGCCCGGATGATCAGATAGGCTA
CTATAGACGCGCTACCCGACGGATACGAGGGGGGGACGGCAAAAT
GAAGGACCTTTCCCCCCGGTGGTATTTCTATTACTTGGGCACCGG
ACCAGAAGCCGGACTGCCTTACGGCGCTAACAAAGACGGAATAAT
CTGGGTTGCGACGGAGGGCGCCCTGAATACACCTAAAGACCATAT
CGGCACAAGAAATCCTGCTAACAATGCCGCGATTGTGCTCCAGCT
GCCTCAGGGAACCACGCTGCCTAAAGGGTTTTACGCTGAGGGGTC
AAGGGGGGGGAGTCAAGCGTCTAGTAGGTCATCCTCTCGCTCTCG
CAATAGTTCCCGGAACTCAACCCCAGGCAGCAGCAGAGGAACCTC
TCCCGCACGGATGGCTGGCAATGGGGGAGATGCTGCCCTTGCTCT
CCTTCTGCTGGATCGCCTTAACCAGCTCGAATCAAAGATGTCTGG
AAAAGGTCAGCAGCAGCAAGGCCAGACCGTGACAAAGAAGAGTGC
AGCTGAAGCTAGTAAAAAGCCACGCCAAAAACGGACCGCAACTAA
GGCATATAACGTAACACAGGCCTTCGGCAGAAGAGGTCCAGAACA
AACACAGGGAAACTTTGGCGATCAAGAGCTGATTAGACAGGGCAC
AGATTACAAACACTGGCCACAGATCGCGCAGTTTGCACCAAGCGC
CTCTGCATTCTTCGGGATGAGTCGGATTGGGATGGAAGTCACTCC
ATCCGGGACCTGGCTTACCTACACAGGGGCAATAAAACTCGACGA
CAAAGACCCAAACTTTAAAGATCAGGTCATCCTGCTGAATAAACA
CATCGATGCCTACAAAACTTTCCCCCCAACCGAACCAAAGAAAGA
CAAGAAAAAAAAGGCAGACGAAACGCAAGCGCTCCCTCAGCGCCA
GAAGAAGCAGCAGACCGTTACACTGTTGCCAGCAGCAGATCTGGA
TGATTTTTCCAAGCAGCTTCAACAGAGTATGTCAAGCGCTGACAG
CACTCAGGCTTGAggcgcgccgctgaccgatAAATAAAATATCTT
TATTTTCATTACATCTGTGTGTTGGTTTTTTGTGTGacgcgttag
ttattaataGTAATCAATTACGGGGTCATTAGTTCATAGCCCATA
TATGGAGTTCCGCGTTACATAACTTACGGTAAATGGCCCGCCTGG
CTGACCGCCCAACGACCCCCGCCCATTGACGTCAATAATGACGTA
TGTTCCCATAGTAACGCCAATAGGGACTTTCCATTGACGTCAATG
GGTGGAGTATTTACGGTAAACTGCCCACTTGGCAGTACATCAAGT
GTATCATATGCCAAGTACGCCCCCTATTGACGTCAATGACGGTAA
ATGGCCCGCCTGGCATTATGCCCAGTACATGACCTTATGGGACTT
TCCTACTTGGCAGTACATCTACGTATTAGTCATCGCTATTACCAT
GGTGATGCGGTTTTGGCAGTACATCAATGGGCGTGGATAGCGGTT
TGACTCACGGGGATTTCCAAGTCTCCACCCCATTGACGTCAATGG
GAGTTTGTTTTGGCACCAAAATCAACGGGACTTTCCAAAATGTCG
TAACAACTCCGCCCCATTGACGCAAATGGGCGGTAGGCGTGTACG
GTGGGAGGTCTATATAAGCAGAGCTGGTTTAGTGAACCGTCAGAT
CCGCTAGAGATATCGGGCCACTGCAGGAAACGATATGGGCTGAAT
ACGGATCCGTATTCAGCCCATATCGTTTCTCTAGAAATAAAATAT
CTTTATTTTCATTACATCTGTGTGTTGGTTTTTTGTGTGAATCGA
TAGTACTAACATACGCTCTCCATCTCGAGCCTAAGCTTGTCGACT
CGAAGATCTGGGCGTGGTTAAGGGTGGGAAAGAATATATAAGGTG
GGGGTCTTATGTAGTTTTGTATCTGTTTTGCAGCAGCCGCCGCCG
CCATGAGCACCAACTCGTTTGATGGAAGCATTGTGAGCTCATATT
TGACAACGCGCATGCCCCCATGGGCCGGGGTGCGTCAGAATGTGA
TGGGCTCCAGCATTGATGGTCGCCCCGTCCTGCCCGCAAACTCTA
CTACCTTGACCTACGAGACCGTGTCTGGAACGCCGTTGGAGACTG
CAGCCTCCGCCGCCGCTTCAGCCGCTGCAGCCACCGCCCGCGGGA
TTGTGACTGACTTTGCTTTCCTGAGCCCGCTTGCAAGCAGTGCAG
CTTCCCGTTCATCCGCCCGCGATGACAAGTTGACGGCTCTTTTGG
CACAATTGGATTCTTTGACCCGGGAACTTAATGTCGTTTCTCAGC
AGCTGTTGGATCTGCGCCAGCAGGTTTCTGCCCTGAAGGCTTCCT
CCCCTCCCAATGCGGTTTAAAACATAAATAAAAAACCAGACTCTG
TTTGGATTTGGATCAAGCAAGTGTCTTGCTGTCTTTATTTAGGGG
TTTTGCGCGCGCGGTAGGCCCGGGACCAGCGGTCTCGGTCGTTGA
GGGTCCTGTGTATTTTTTCCAGGACGTGGTAAAGGTGACTCTGGA
TGTTCAGATACATGGGCATAAGCCCGTCTCTGGGGTGGAGGTAGC
ACCACTGCAGAGCTTCATGCTGCGGGGTGGTGTTGTAGATGATCC
AGTCGTAGCAGGAGCGCTGGGCGTGGTGCCTAAAAATGTCTTTCA
GTAGCAAGCTGATTGCCAGGGGCAGGCCCTTGGTGTAAGTGTTTA
CAAAGCGGTTAAGCTGGGATGGGTGCATACGTGGGGATATGAGAT
GCATCTTGGACTGTATTTTTAGGTTGGCTATGTTCCCAGCCATAT
CCCTCCGGGGATTCATGTTGTGCAGAACCACCAGCACAGTGTATC
CGGTGCACTTGGGAAATTTGTCATGTAGCTTAGAAGGAAATGCGT
GGAAGAACTTGGAGACGCCCTTGTGACCTCCAAGATTTTCCATGC
ATTCGTCCATAATGATGGCAATGGGCCCACGGGCGGCGGCCTGGG
CGAAGATATTTCTGGGATCACTAACGTCATAGTTGTGTTCCAGGA
TGAGATCGTCATAGGCCATTTTTACAAAGCGCGGGCGGAGGGTGC
CAGACTGCGGTATAATGGTTCCATCCGGCCCAGGGGCGTAGTTAC
CCTCACAGATTTGCATTTCCCACGCTTTGAGTTCAGATGGGGGGA
TCATGTCTACCTGCGGGGCGATGAAGAAAACGGTTTCCGGGGTAG
GGGAGATCAGCTGGGAAGAAAGCAGGTTCCTGAGCAGCTGCGACT
TACCGCAGCCGGTGGGCCCGTAAATCACACCTATTACCGGCTGCA
ACTGGTAGTTAAGAGAGCTGCAGCTGCCGTCATCCCTGAGCAGGG
GGGCCACTTCGTTAAGCATGTCCCTGACTCGCATGTTTTCCCTGA
CCAAATCCGCCAGAAGGCGCTCGCCGCCCAGCGATAGCAGTTCTT
GCAAGGAAGCAAAGTTTTTCAACGGTTTGAGACCGTCCGCCGTAG
GCATGCTTTTGAGCGTTTGACCAAGCAGTTCCAGGCGGTCCCACA
GCTCGGTCACCTGCTCTACGGCATCTCGATCCAGCATATCTCCTC
GTTTCGCGGGTTGGGGCGGCTTTCGCTGTACGGCAGTAGTCGGTG
CTCGTCCAGACGGGCCAGGGTCATGTCTTTCCACGGGCGCAGGGT
CCTCGTCAGCGTAGTCTGGGTCACGGTGAAGGGGTGCGCTCCGGG
CTGCGCGCTGGCCAGGGTGCGCTTGAGGCTGGTCCTGCTGGTGCT
GAAGCGCTGCCGGTCTTCGCCCTGCGCGTCGGCCAGGTAGCATTT
GACCATGGTGTCATAGTCCAGCCCCTCCGCGGCGTGGCCCTTGGC
GCGCAGCTTGCCCTTGGAGGAGGCGCCGCACGAGGGGCAGTGCAG
ACTTTTGAGGGCGTAGAGCTTGGGCGCGAGAAATACCGATTCCGG
GGAGTAGGCATCCGCGCCGCAGGCCCCGCAGACGGTCTCGCATTC
CACGAGCCAGGTGAGCTCTGGCCGTTCGGGGTCAAAAACCAGGTT
TCCCCCATGCTTTTTGATGCGTTTCTTACCTCTGGTTTCCATGAG
CCGGTGTCCACGCTCGGTGACGAAAAGGCTGTCCGTGTCCCCGTA
TACAGACTTGAGAGGCCTGTCCTCGAGCGGTGTTCCGCGGTCCTC
CTCGTATAGAAACTCGGACCACTCTGAGACAAAGGCTCGCGTCCA
GGCCAGCACGAAGGAGGCTAAGTGGGAGGGGTAGCGGTCGTTGTC
CACTAGGGGGTCCACTCGCTCCAGGGTGTGAAGACACATGTCGCC
CTCTTCGGCATCAAGGAAGGTGATTGGTTTGTAGGTGTAGGCCAC
GTGACCGGGTGTTCCTGAAGGGGGGCTATAAAAGGGGGTGGGGGC
GCGTTCGTCCTCACTCTCTTCCGCATCGCTGTCTGCGAGGGCCAG
CTGTTGGGGTGAGTACTCCCTCTGAAAAGCGGGCATGACTTCTGC
GCTAAGATTGTCAGTTTCCAAAAACGAGGAGGATTTGATATTCAC
CTGGCCCGCGGTGATGCCTTTGAGGGTGGCCGCATCCATCTGGTC
AGAAAAGACAATCTTTTTGTTGTCAAGCTTGGTGGCAAACGACCC
GTAGAGGGCGTTGGACAGCAACTTGGCGATGGAGCGCAGGGTTTG
GTTTTTGTCGCGATCGGCGCGCTCCTTGGCCGCGATGTTTAGCTG
CACGTATTCGCGCGCAACGCACCGCCATTCGGGAAAGACGGTGGT
GCGCTCGTCGGGCACCAGGTGCACGCGCCAACCGCGGTTGTGCAG
GGTGACAAGGTCAACGCTGGTGGCTACCTCTCCGCGTAGGCGCTC
GTTGGTCCAGCAGAGGCGGCCGCCCTTGCGCGAGCAGAATGGCGG
TAGGGGGTCTAGCTGCGTCTCGTCCGGGGGGTCTGCGTCCACGGT
AAAGACCCCGGGCAGCAGGCGCGCGTCGAAGTAGTCTATCTTGCA
TCCTTGCAAGTCTAGCGCCTGCTGCCATGCGCGGGCGGCAAGCGC
GCGCTCGTATGGGTTGAGTGGGGGACCCCATGGCATGGGGTGGGT
GAGCGCGGAGGCGTACATGCCGCAAATGTCGTAAACGTAGAGGGG
CTCTCTGAGTATTCCAAGATATGTAGGGTAGCATCTTCCACCGCG
GATGCTGGCGCGCACGTAATCGTATAGTTCGTGCGAGGGAGCGAG
GAGGTCGGGACCGAGGTTGCTACGGGCGGGCTGCTCTGCTCGGAA
GACTATCTGCCTGAAGATGGCATGTGAGTTGGATGATATGGTTGG
ACGCTGGAAGACGTTGAAGCTGGCGTCTGTGAGACCTACCGCGTC
ACGCACGAAGGAGGCGTAGGAGTCGCGCAGCTTGTTGACCAGCTC
GGCGGTGACCTGCACGTCTAGGGCGCAGTAGTCCAGGGTTTCCTT
GATGATGTCATACTTATCCTGTCCCTTTTTTTTCCACAGCTCGCG
GTTGAGGACAAACTCTTCGCGGTCTTTCCAGTACTCTTGGATCGG
AAACCCGTCGGCCTCCGAACGGTAAGAGCCTAGCATGTAGAACTG
GTTGACGGCCTGGTAGGCGCAGCATCCCTTTTCTACGGGTAGCGC
GTATGCCTGCGCGGCCTTCCGGAGCGAGGTGTGGGTGAGCGCAAA
GGTGTCCCTGACCATGACTTTGAGGTACTGGTATTTGAAGTCAGT
GTCGTCGCATCCGCCCTGCTCCCAGAGCAAAAAGTCCGTGCGCTT
TTTGGAACGCGGATTTGGCAGGGCGAAGGTGACATCGTTGAAGAG
TATCTTTCCCGCGCGAGGCATAAAGTTGCGTGTGATGCGGAAGGG
TCCCGGCACCTCGGAACGGTTGTTAATTACCTGGGCGGCGAGCAC
GATCTCGTCAAAGCCGTTGATGTTGTGGCCCACAATGTAAAGTTC
CAAGAAGCGCGGGATGCCCTTGATGGAAGGCAATTTTTTAAGTTC
CTCGTAGGTGAGCTCTTCAGGGGAGCTGAGCCCGTGCTCTGAAAG
GGCCCAGTCTGCAAGATGAGGGTTGGAAGCGACGAATGAGCTCCA
CAGGTCACGGGCCATTAGCATTTGCAGGTGGTCGCGAAAGGTCCT
AAACTGGCGACCTATGGCCATTTTTTCTGGGGTGATGCAGTAGAA
GGTAAGCGGGTCTTGTTCCCAGCGGTCCCATCCAAGGTTCGCGGC
TAGGTCTCGCGCGGCAGTCACTAGAGGCTCATCTCCGCCGAACTT
CATGACCAGCATGAAGGGCACGAGCTGCTTCCCAAAGGCCCCCAT
CCAAGTATAGGTCTCTACATCGTAGGTGACAAAGAGACGCTCGGT
GCGAGGATGCGAGCCGATCGGGAAGAACTGGATCTCCCGCCACCA
ATTGGAGGAGTGGCTATTGATGTGGTGAAAGTAGAAGTCCCTGCG
ACGGGCCGAACACTCGTGCTGGCTTTTGTAAAAACGTGCGCAGTA
CTGGCAGCGGTGCACGGGCTGTACATCCTGCACGAGGTTGACCTG
ACGACCGCGCACAAGGAAGCAGAGTGGGAATTTGAGCCCCTCGCC
TGGCGGGTTTGGCTGGTGGTCTTCTACTTCGGCTGCTTGTCCTTG
ACCGTCTGGCTGCTCGAGGGGAGTTACGGTGGATCGGACCACCAC
GCCGCGCGAGCCCAAAGTCCAGATGTCCGCGCGCGGCGGTCGGAG
CTTGATGACAACATCGCGCAGATGGGAGCTGTCCATGGTCTGGAG
CTCCCGCGGCGTCAGGTCAGGCGGGAGCTCCTGCAGGTTTACCTC
GCATAGACGGGTCAGGGCGCGGGCTAGATCCAGGTGATACCTAAT
TTCCAGGGGCTGGTTGGTGGCGGCGTCGATGGCTTGCAAGAGGCC
GCATCCCCGCGGCGCGACTACGGTACCGCGCGGCGGGCGGTGGGC
CGCGGGGGTGTCCTTGGATGATGCATCTAAAAGCGGTGACGCGGG
CGAGCCCCCGGAGGTAGGGGGGGCTCCGGACCCGCCGGGAGAGGG
GGCAGGGGCACGTCGGCGCCGCGCGCGGGCAGGAGCTGGTGCTGC
GCGCGTAGGTTGCTGGCGAACGCGACGACGCGGCGGTTGATCTCC
TGAATCTGGCGCCTCTGCGTGAAGACGACGGGCCCGGTGAGCTTG
AACCTGAAAGAGAGTTCGACAGAATCAATTTCGGTGTCGTTGACG
GCGGCCTGGCGCAAAATCTCCTGCACGTCTCCTGAGTIGTCTTGA
TAGGCGATCTCGGCCATGAACTGCTCGATCTCTTCCTCCTGGAGA
TCTCCGCGTCCGGCTCGCTCCACGGTGGCGGCGAGGTCGTTGGAA
ATGCGGGCCATGAGCTGCGAGAAGGCGTTGAGGCCTCCCTCGTTC
CAGACGCGGCTGTAGACCACGCCCCCTTCGGCATCGCGGGCGCGC
ATGACCACCTGCGCGAGATTGAGCTCCACGTGCCGGGCGAAGACG
GCGTAGTTTCGCAGGCGCTGAAAGAGGTAGTTGAGGGTGGTGGCG
GTGTGTTCTGCCACGAAGAAGTACATAACCCAGCGTCGCAACGTG
GATTCGTTGATATCCCCCAAGGCCTCAAGGCGCTCCATGGCCTCG
TAGAAGTCCACGGCGAAGTTGAAAAACTGGGAGTTGCGCGCCGAC
ACGGTTAACTCCTCCTCCAGAAGACGGATGAGCTCGGCGACAGTG
TCGCGCACCTCGCGCTCAAAGGCTACAGGGGCCTCTTCTTCTTCT
TCAATCTCCTCTTCCATAAGGGCCTCCCCTTCTTCTTCTTCTGGC
GGCGGTGGGGGAGGGGGGACACGGCGGCGACGACGGCGCACCGGG
AGGCGGTCGACAAAGCGCTCGATCATCTCCCCGCGGCGACGGCGC
ATGGTCTCGGTGACGGCGCGGCCGTTCTCGCGGGGGCGCAGTTGG
AAGACGCCGCCCGTCATGTCCCGGTTATGGGTTGGCGGGGGGCTG
CCATGCGGCAGGGATACGGCGCTAACGATGCATCTCAACAATTGT
TGTGTAGGTACTCCGCCGCCGAGGGACCTGAGCGAGTCCGCATCG
ACCGGATCGGAAAACCTCTCGAGAAAGGCGTCTAACCAGTCACAG
TCGCAAGGTAGGCTGAGCACCGTGGCGGGCGGCAGCGGGCGGCGG
TCGGGGTTGTTTCTGGCGGAGGTGCTGCTGATGATGTAATTAAAG
TAGGCGGTCTTGAGACGGCGGATGGTCGACAGAAGCACCATGTCC
TTGGGTCCGGCCTGCTGAATGCGCAGGCGGTCGGCCATGCCCCAG
GCTTCGTTTTGACATCGGCGCAGGTCTTTGTAGTAGTCTTGCATG
AGCCTTTCTACCGGCACTTCTTCTTCTCCTTCCTCTTGTCCTGCA
TCTCTTGCATCTATCGCTGCGGCGGCGGCGGAGTTTGGCCGTAGG
TGGCGCCCTCTTCCTCCCATGCGTGTGACCCCGAAGCCCCTCATC
GGCTGAAGCAGGGCTAGGTCGGCGACAACGCGCTCGGCTAATATG
GCCTGCTGCACCTGCGTGAGGGTAGACTGGAAGTCATCCATGTCC
ACAAAGCGGTGGTATGCGCCCGTGTTGATGGTGTAAGTGCAGTTG
GCCATAACGGACCAGTTAACGGTCTGGTGACCCGGCTGCGAGAGC
TCGGTGTACCTGAGACGCGAGTAAGCCCTCGAGTCAAATACGTAG
TCGTTGCAAGTCCGCACCAGGTACTGGTATCCCACCAAAAAGTGC
GGCGGCGGCTGGCGGTAGAGGGGCCAGCGTAGGGTGGCCGGGGCT
CCGGGGGCGAGATCTTCCAACATAAGGCGATGATATCCGTAGATG
TACCTGGACATCCAGGTGATGCCGGCGGCGGTGGTGGAGGCGCGC
GGAAAGTCGCGGACGCGGTTCCAGATGTTGCGCAGCGGCAAAAAG
TGCTCCATGGTCGGGACGCTCTGGCCGGTCAGGCGCGCGCAATCG
TTGACGCTCTAGCGTGCAAAAGGAGAGCCTGTAAGCGGGCACTCT
TCCGTGGTCTGGTGGATAAATTCGCAAGGGTATCATGGCGGACGA
CCGGGGTTCGAGCCCCGTATCCGGCCGTCCGCCGTGATCCATGCG
GTTACCGCCCGCGTGTCGAACCCAGGTGTGCGACGTCAGACAACG
GGGGAGTGCTCCTTTTGGCTTCCTTCCAGGCGCGGCGGCTGCTGC
GCTAGCTTTTTTGGCCACTGGCCGCGCGCAGCGTAAGCGGTTAGG
CTGGAAAGCGAAAGCATTAAGTGGCTCGCTCCCTGTAGCCGGAGG
GTTATTTTCCAAGGGTTGAGTCGCGGGACCCCCGGTTCGAGTCTC
GGACCGGCCGGACTGCGGCGAACGGGGGTTTGCCTCCCCGTCATG
CAAGACCCCGCTTGCAAATTCCTCCGGAAACAGGGACGAGCCCCT
TTTTTGCTTTTCCCAGATGCATCCGGTGCTGCGGCAGATGCGCCC
CCCTCCTCAGCAGCGGCAAGAGCAAGAGCAGCGGCAGACATGCAG
GGCACCCTCCCCTCCTCCTACCGCGTCAGGAGGGGCGACATCCGC
GGTTGACGCGGCAGCAGATGGTGATTACGAACCCCCGCGGCGCCG
GGCCCGGCACTACCTGGACTTGGAGGAGGGCGAGGGCCTGGCGCG
GCTAGGAGCGCCCTCTCCTGAGCGGCACCCAAGGGTGCAGCTGAA
GCGTGATACGCGTGAGGCGTACGTGCCGCGGCAGAACCTGTTTCG
CGACCGCGAGGGAGAGGAGCCCGAGGAGATGCGGGATCGAAAGTT
CCACGCAGGGCGCGAGCTGCGGCATGGCCTGAATCGCGAGCGGTT
GCTGCGCGAGGAGGACTTTGAGCCCGACGCGCGAACCGGGATTAG
TCCCGCGCGCGCACACGTGGCGGCCGCCGACCTGGTAACCGCATA
CGAGCAGACGGTGAACCAGGAGATTAACTTTCAAAAAAGCTTTAA
CAACCACGTGCGTACGCTTGTGGCGCGCGAGGAGGTGGCTATAGG
ACTGATGCATCTGTGGGACTTTGTAAGCGCGCTGGAGCAAAACCC
AAATAGCAAGCCGCTCATGGCGCAGCTGTTCCTTATAGTGCAGCA
CAGCAGGGACAACGAGGCATTCAGGGATGCGCTGCTAAACATAGT
AGAGCCCGAGGGCCGCTGGCTGCTCGATTTGATAAACATCCTGCA
GAGCATAGTGGTGCAGGAGCGCAGCTTGAGCCTGGCTGACAAGGT
GGCCGCCATCAACTATTCCATGCTTAGCCTGGGCAAGTTTTACGC
CCGCAAGATATACCATACCCCTTACGTTCCCATAGACAAGGAGGT
AAAGATCGAGGGGTTCTACATGCGCATGGCGCTGAAGGTGCTTAC
CTTGAGCGACGACCTGGGCGTTTATCGCAACGAGCGCATCCACAA
GGCCGTGAGCGTGAGCCGGCGGCGCGAGCTCAGCGACCGCGAGCT
GATGCACAGCCTGCAAAGGGCCCTGGCTGGCACGGGCAGCGGCGA
TAGAGAGGCCGAGTCCTACTTTGACGCGGGCGCTGACCTGCGCTG
GGCCCCAAGCCGACGCGCCCTGGAGGCAGCTGGGGCCGGACCTGG
GCTGGCGGTGGCACCCGCGCGCGCTGGCAACGTCGGCGGCGTGGA
GGAATATGACGAGGACGATGAGTACGAGCCAGAGGACGGCGAGTA
CTAAGCGGTGATGTTTCTGATCAGATGATGCAAGACGCAACGGAC
CCGGCGGTGCGGGCGGCGCTGCAGAGCCAGCCGTCCGGCCTTAAC
TCCACGGACGACTGGCGCCAGGTCATGGACCGCATCATGTCGCTG
ACTGCGCGCAATCCTGACGCGTTCCGGCAGCAGCCGCAGGCCAAC
CGGCTCTCCGCAATTCTGGAAGCGGTGGTCCCGGCGCGCGCAAAC
CCCACGCACGAGAAGGTGCTGGCGATCGTAAACGCGCTGGCCGAA
AACAGGGCCATCCGGCCCGACGAGGCCGGCCTGGTCTACGACGCG
CTGCTTCAGCGCGTGGCTCGTTACAACAGCGGCAACGTGCAGACC
AACCTGGACCGGCTGGTGGGGGATGTGCGCGAGGCCGTGGCGCAG
CGTGAGCGCGCGCAGCAGCAGGGCAACCTGGGCTCCATGGTTGCA
CTAAACGCCTTCCTGAGTACACAGCCCGCCAACGTGCCGCGGGGA
CAGGAGGACTACACCAACTTTGTGAGCGCACTGCGGCTAATGGTG
ACTGAGACACCGCAAAGTGAGGTGTACCAGTCTGGGCCAGACTAT
TTTTTCCAGACCAGTAGACAAGGCCTGCAGACCGTAAACCTGAGC
CAGGCTTTCAAAAACTTGCAGGGGCTGTGGGGGGTGCGGGCTCCC
ACAGGCGACCGCGCGACCGTGTCTAGCTTGCTGACGCCCAACTCG
CGCCTGTTGCTGCTGCTAATAGCGCCCTTCACGGACAGTGGCAGC
GTGTCCCGGGACACATACCTAGGTCACTTGCTGACACTGTACCGC
GAGGCCATAGGTCAGGCGCATGTGGACGAGCATACTTTCCAGGAG
ATTACAAGTGTCAGCCGCGCGCTGGGGCAGGAGGACACGGGCAGC
CTGGAGGCAACCCTAAACTACCTGCTGACCAACCGGCGGCAGAAG
ATCCCCTCGTTGCACAGTTTAAACAGCGAGGAGGAGCGCATTTTG
CGCTACGTGCAGCAGAGCGTGAGCCTTAACCTGATGCGCGACGGG
GTAACGCCCAGCGTGGCGCTGGACATGACCGCGCGCAACATGGAA
CCGGGCATGTATGCCTCAAACCGGCCGTTTATCAACCGCCTAATG
GACTACTTGCATCGCGCGGCCGCCGTGAACCCCGAGTATTTCACC
AATGCCATCTTGAACCCGCACTGGCTACCGCCCCCTGGTTTCTAC
ACCGGGGGATTCGAGGTGCCCGAGGGTAACGATGGATTCCTCTGG
GACGACATAGACGACAGCGTGTTTTCCCCGCAACCGCAGACCCTG
CTAGAGTTGCAACAGCGCGAGCAGGCAGAGGCGGCGCTGCGAAAG
GAAAGCTTCCGCAGGCCAAGCAGCTTGTCCGATCTAGGCGCTGCG
GCCCCGCGGTCAGATGCTAGTAGCCCATTTCCAAGCTTGATAGGG
TCTCTTACCAGCACTCGCACCACCCGCCCGCGCCTGCTGGGCGAG
GAGGAGTACCTAAACAACTCGCTGCTGCAGCCGCAGCGCGAAAAA
AACCTGCCTCCGGCATTTCCCAACAACGGGATAGAGAGCCTAGTG
GACAAGATGAGTAGATGGAAGACGTACGCGCAGGAGCACAGGGAC
GTGCCAGGCCCGCGCCCGCCCACCCGTCGTCAAAGGCACGACCGT
CAGCGGGGTCTGGTGTGGGAGGACGATGACTCGGCAGACGACAGC
AGCGTCCTGGATTTGGGAGGGAGTGGCAACCCGTTTGCGCACCTT
CGCCCCAGGCTGGGGAGAATGTTTTAAAAAAAAAAAAGCATGATG
CAAAATAAAAAACTCACCAAGGCCATGGCACCGAGCGTTGGTTTT
CTTGTATTCCCCTTAGTATGCGGCGCGCGGCGATGTATGAGGAAG
GTCCTCCTCCCTCCTACGAGAGTGTGGTGAGCGCGGCGCCAGTGG
CGGCGGCGCTGGGTTCTCCCTTCGATGCTCCCCTGGACCCGCCGT
TTGTGCCTCCGCGGTACCTGCGGCCTACCGGGGGGAGAAACAGCA
TCCGTTACTCTGAGTTGGCACCCCTATTCGACACCACCCGTGTGT
ACCTGGTGGACAACAAGTCAACGGATGTGGCATCCCTGAACTACC
AGAACGACCACAGCAACTTTCTGACCACGGTCATTCAAAACAATG
ACTACAGCCCGGGGGAGGCAAGCACACAGACCATCAATCTTGACG
ACCGGTCGCACTGGGGCGGCGACCTGAAAACCATCCTGCATACCA
ACATGCCAAATGTGAACGAGTTCATGTTTACCAATAAGTTTAAGG
CGCGGGTGATGGTGTCGCGCTTGCCTACTAAGGACAATCAGGTGG
AGCTGAAATACGAGTGGGTGGAGTTCACGCTGCCCGAGGGCAACT
ACTCCGAGACCATGACCATAGACCTTATGAACAACGCGATCGTGG
AGCACTACTTGAAAGTGGGCAGACAGAACGGGGTTCTGGAAAGCG
ACATCGGGGTAAAGTTTGACACCCGCAACTTCAGACTGGGGTTTG
ACCCCGTCACTGGTCTTGTCATGCCTGGGGTATATACAAACGAAG
CCTTCCATCCAGACATCATTTTGCTGCCAGGATGCGGGGTGGACT
TCACCCACAGCCGCCTGAGCAACTTGTTGGGCATCCGCAAGCGGC
AACCCTTCCAGGAGGGCTTTAGGATCACCTACGATGATCTGGAGG
GTGGTAACATTCCCGCACTGTTGGATGTGGACGCCTACCAGGCGA
GCTTGAAAGATGACACCGAACAGGGCGGGGGTGGCGCAGGCGGCA
GCAACAGCAGTGGCAGCGGCGCGGAAGAGAACTCCAACGCGGCAG
CCGCGGCAATGCAGCCGGTGGAGGACATGAACGATCATGCCATTC
GCGGCGACACCTTTGCCACACGGGCTGAGGAGAAGCGCGCTGAGG
CCGAAGCAGCGGCCGAAGCTGCCGCCCCCGCTGCGCAACCCGAGG
TCGAGAAGCCTCAGAAGAAACCGGTGATCAAACCCCTGACAGAGG
ACAGCAAGAAACGCAGTTACAACCTAATAAGCAATGACAGCACCT
TCACCCAGTACCGCAGCTGGTACCTTGCATACAACTACGGCGACC
CTCAGACCGGAATCCGCTCATGGACCCTGCTTTGCACTCCTGACG
TAACCTGCGGCTCGGAGCAGGTCTACTGGTCGTTGCCAGACATGA
TGCAAGACCCCGTGACCTTCCGCTCCACGCGCCAGATCAGCAACT
TTCCGGTGGTGGGCGCCGAGCTGTTGCCCGTGCACTCCAAGAGCT
TCTACAACGACCAGGCCGTCTACTCCCAACTCATCCGCCAGTTTA
CCTCTCTGACCCACGTGTTCAATCGCTTTCCCGAGAACCAGATTT
TGGCGCGCCCGCCAGCCCCCACCATCACCACCGTCAGTGAAAACG
TTCCTGCTCTCACAGATCACGGGACGCTACCGCTGCGCAACAGCA
TCGGAGGAGTCCAGCGAGTGACCATTACTGACGCCAGACGCCGCA
CCTGCCCCTACGTTTACAAGGCCCTGGGCATAGTCTCGCCGCGCG
TCCTATCGAGCCGCACTTTTTGAGCAAGCATGTCCATCCTTATAT
CGCCCAGCAATAACACAGGCTGGGGCCTGCGCTTCCCAAGCAAGA
TGTTTGGCGGGGCCAAGAAGCGCTCCGACCAACACCCAGTGCGCG
TGCGCGGGCACTACCGCGCGCCCTGGGGCGCGCACAAACGCGGCC
GCACTGGGCGCACCACCGTCGATGACGCCATCGACGCGGTGGTGG
AGGAGGCGCGCAACTACACGCCCACGCCGCCACCAGTGTCCACAG
TGGACGCGGCCATTCAGACCGTGGTGCGCGGAGCCCGGCGCTATG
CTAAAATGAAGAGACGGCGGAGGCGCGTAGCACGTCGCCACCGCC
GCCGACCCGGCACTGCCGCCCAACGCGCGGCGGCGGCCCTGCTTA
ACCGCGCACGTCGCACCGGCCGACGGGCGGCCATGCGGGCCGCTC
GAAGGCTGGCCGCGGGTATTGTCACTGTGCCCCCCAGGTCCAGGC
GACGAGCGGCCGCCGCAGCAGCCGCGGCCATTAGTGCTATGACTC
AGGGTCGCAGGGGCAACGTGTATTGGGTGCGCGACTCGGTTAGCG
GCCTGCGCGTGCCCGTGCGCACCCGCCCCCCGCGCAACTAGATTG
CAAGAAAAAACTACTTAGACTCGTACTGTTGTATGTATCCAGCGG
CGGCGGCGCGCAACGAAGCTATGTCCAAGCGCAAAATCAAAGAAG
AGATGCTCCAGGTCATCGCGCCGGAGATCTATGGCCCCCCGAAGA
AGGAAGAGCAGGATTACAAGCCCCGAAAGCTAAAGCGGGTCAAAA
AGAAAAAGAAAGATGATGATGATGAACTTGACGACGAGGTGGAAC
TGCTGCACGCTACCGCGCCCAGGCGACGGGTACAGTGGAAAGGTC
GACGCGTAAAACGTGTTTTGCGACCCGGCACCACCGTAGTCTTTA
CGCCCGGTGAGCGCTCCACCCGCACCTACAAGCGCGTGTATGATG
AGGTGTACGGCGACGAGGACCTGCTTGAGCAGGCCAACGAGCGCC
TCGGGGAGTTTGCCTACGGAAAGCGGCATAAGGACATGCTGGCGT
TGCCGCTGGACGAGGGCAACCCAACACCTAGCCTAAAGCCCGTAA
CACTGCAGCAGGTGCTGCCCGCGCTTGCACCGTCCGAAGAAAAGC
GCGGCCTAAAGCGCGAGTCTGGTGACTTGGCACCCACCGTGCAGC
TGATGGTACCCAAGCGCCAGCGACTGGAAGATGTCTTGGAAAAAA
TGACCGTGGAACCTGGGCTGGAGCCCGAGGTCCGCGTGCGGCCAA
TCAAGCAGGTGGCGCCGGGACTGGGCGTGCAGACCGTGGACGTTC
AGATACCCACTACCAGTAGCACCAGTATTGCCACCGCCACAGAGG
GCATGGAGACACAAACGTCCCCGGTTGCCTCAGCGGTGGCGGATG
CCGCGGTGCAGGCGGTCGCTGCGGCCGCGTCCAAGACCTCTACGG
AGGTGCAAACGGACCCGTGGATGTTTCGCGTTTCAGCCCCCCGGC
GCCCGCGCCGTTCGAGGAAGTACGGCGCCGCCAGCGCGCTACTGC
CCGAATATGCCCTACATCCTTCCATTGCGCCTACCCCCGGCTATC
GTGGCTACACCTACCGCCCCAGAAGACGAGCAACTACCCGACGCC
GAACCACCACTGGAACCCGCCGCCGCCGTCGCCGTCGCCAGCCCG
TGCTGGCCCCGATTTCCGTGCGCAGGGTGGCTCGCGAAGGAGGCA
GGACCCTGGTGCTGCCAACAGCGCGCTACCACCCCAGCATCGTTT
AAAAGCCGGTCTTTGTGGTTCTTGCAGATATGGCCCTCACCTGCC
GCCTCCGTTTCCCGGTGCCGGGATTCCGAGGAAGAATGCACCGTA
GGAGGGGCATGGCCGGCCACGGCCTGACGGGCGGCATGCGTCGTG
CGCACCACCGGCGGCGGCGCGCGTCGCACCGTCGCATGCGCGGCG
GTATCCTGCCCCTCCTTATTCCACTGATCGCCGCGGCGATTGGCG
CCGTGCCCGGAATTGCATCCGTGGCCTTGCAGGCGCAGAGACACT
GATTAAAAACAAGTTGCATGTGGAAAAATCAAAATAAAAAGTCTG
GACTCTCACGCTCGCTTGGTCCTGTAACTATTTTGTAGAATGGAA
GACATCAACTTTGCGTCTCTGGCCCCGCGACACGGCTCGCGCCCG
TTCATGGGAAACTGGCAAGATATCGGCACCAGCAATATGAGCGGT
GGCGCCTTCAGCTGGGGCTCGCTGTGGAGCGGCATTAAAAATTTC
GGTTCCACCGTTAAGAACTATGGCAGCAAGGCCTGGAACAGCAGC
ACAGGCCAGATGCTGAGGGATAAGTTGAAAGAGCAAAATTTCCAA
CAAAAGGTGGTAGATGGCCTGGCCTCTGGCATTAGCGGGGTGGTG
GACCTGGCCAACCAGGCAGTGCAAAATAAGATTAACAGTAAGCTT
GATCCCCGCCCTCCCGTAGAGGAGCCTCCACCGGCCGTGGAGACA
GTGTCTCCAGAGGGGCGTGGCGAAAAGCGTCCGCGCCCCGACAGG
GAAGAAACTCTGGTGACGCAAATAGACGAGCCTCCCTCGTACGAG
GAGGCACTAAAGCAAGGCCTGCCCACCACCCGTCCCATCGCGCCC
ATGGCTACCGGAGTGCTGGGCCAGCACACACCCGTAACGCTGGAC
CTGCCTCCCCCCGCCGACACCCAGCAGAAACCTGTGCTGCCAGGC
CCGACCGCCGTTGTTGTAACCCGTCCTAGCCGCGCGTCCCTGCGC
CGCGCCGCCAGCGGTCCGCGATCGTTGCGGCCCGTAGCCAGTGGC
AACTGGCAAAGCACACTGAACAGCATCGTGGGTCTGGGGGTGCAA
TCCCTGAAGCGCCGACGATGCTTCTGATAGCTAACGTGTCGTATG
TGTGTCATGTATGCGTCCATGTCGCCGCCAGAGGAGCTGCTGAGC
CGCCGCGCGCCCGCTTTCCAAGATGGCTACCCCTTCGATGATGCC
GCAGTGGTCTTACATGCACATCTCGGGCCAGGACGCCTCGGAGTA
CCTGAGCCCCGGGCTGGTGCAGTTTGCCCGCGCCACCGAGACGTA
CTTCAGCCTGAATAACAAGTTTAGAAACCCCACGGTGGCGCCTAC
GCACGACGTGACCACAGACCGGTCCCAGCGTTTGACGCTGCGGTT
CATCCCTGTGGACCGTGAGGATACTGCGTACTCGTACAAGGCGCG
GTTCACCCTAGCTGTGGGTGATAACCGTGTGCTGGACATGGCTTC
CACGTACTTTGACATCCGCGGCGTGCTGGACAGGGGCCCTACTTT
TAAGCCCTACTCTGGCACTGCCTACAACGCCCTGGCTCCCAAGGG
TGCCCCAAATCCTTGCGAATGGGATGAAGCTGCTACTGCTCTTGA
AATAAACCTAGAAGAAGAGGACGATGACAACGAAGACGAAGTAGA
CGAGCAAGCTGAGCAGCAAAAAACTCACGTATTTGGGCAGGCGCC
TTATTCTGGTATAAATATTACAAAGGAGGGTATTCAAATAGGTGT
CGAAGGTCAAACACCTAAATATGCCGATAAAACATTTCAACCTGA
ACCTCAAATAGGAGAATCTCAGTGGTACGAAACAGAAATTAATCA
TGCAGCTGGGAGAGTCCTAAAAAAGACTACCCCAATGAAACCATG
TTACGGTTCATATGCAAAACCCACAAATGAAAATGGAGGGCAAGG
CATTCTTGTAAAGCAACAAAATGGAAAGCTAGAAAGTCAAGTGGA
AATGCAATTTTTCTCAACTACTGAGGCAGCCGCAGGCAATGGTGA
TAACTTGACTCCTAAAGTGGTATTGTACAGTGAAGATGTAGATAT
AGAAACCCCAGACACTCATATTTCTTACATGCCCACTATTAAGGA
AGGTAACTCACGAGAACTAATGGGCCAACAATCTATGCCCAACAG
GCCTAATTACATTGCTTTTAGGGACAATTTTATTGGTCTAATGTA
TTACAACAGCACGGGTAATATGGGTGTTCTGGCGGGCCAAGCATC
GCAGTTGAATGCTGTTGTAGATTTGCAAGACAGAAACACAGAGCT
TTCATACCAGCTTTTGCTTGATTCCATTGGTGATAGAACCAGGTA
CTTTTCTATGTGGAATCAGGCTGTTGACAGCTATGATCCAGATGT
TAGAATTATTGAAAATCATGGAACTGAAGATGAACTTCCAAATTA
CTGCTTTCCACTGGGAGGTGTGATTAATACAGAGACTCTTACCAA
GGTAAAACCTAAAACAGGTCAGGAAAATGGATGGGAAAAAGATGC
TACAGAATTTTCAGATAAAAATGAAATAAGAGTTGGAAATAATTT
TGCCATGGAAATCAATCTAAATGCCAACCTGTGGAGAAATTTCCT
GTACTCCAACATAGCGCTGTATTTGCCCGACAAGCTAAAGTACAG
TCCTTCCAACGTAAAAATTTCTGATAACCCAAACACCTACGACTA
CATGAACAAGCGAGTGGTGGCTCCCGGGCTAGTGGACTGCTACAT
TAACCTTGGAGCACGCTGGTCCCTTGACTATATGGACAACGTCAA
CCCATTTAACCACCACCGCAATGCTGGCCTGCGCTACCGCTCAAT
GTTGCTGGGCAATGGTCGCTATGTGCCCTTCCACATCCAGGTGCC
TCAGAAGTTCTTTGCCATTAAAAACCTCCTTCTCCTGCCGGGCTC
ATACACCTACGAGTGGAACTTCAGGAAGGATGTTAACATGGTTCT
GCAGAGCTCCCTAGGAAATGACCTAAGGGTTGACGGAGCCAGCAT
TAAGTTTGATAGCATTTGCCTTTACGCCACCTTCTTCCCCATGGC
CCACAACACCGCCTCCACGCTTGAGGCCATGCTTAGAAACGACAC
CAACGACCAGTCCTTTAACGACTATCTCTCCGCCGCCAACATGCT
CTACCCTATACCCGCCAACGCTACCAACGTGCCCATATCCATCCC
CTCCCGCAACTGGGGGCTTTCCGCGGCTGGGCCTTCACGCGCCTT
AAGACTAAGGAAACCCCATCACTGGGCTCGGGCTACGACCCTTAT
TACACCTACTCTGGCTCTATACCCTACCTAGATGGAACCTTTTAC
CTCAACCACACCTTTAAGAAGGTGGCCATTACCTTTGACTCTTCT
GTCAGCTGGCCTGGCAATGACCGCCTGCTTACCCCCAACGAGTTT
GAAATTAAGCGCTCAGTTGACGGGGAGGGTTACAACGTTGCCCAG
TGTAACATGACCAAAGACTGGTTCCTGGTACAAATGCTAGCTAAC
TATAACATTGGCTACCAGGGCTTCTATATCCCAGAGAGCTACAAG
GACCGCATGTACTCCTTCTTTAGAAACTTCCAGCCCATGAGCCGT
CAGGTGGTGGATGATACTAAATACAAGGACTACCAACAGGTGGGC
ATCCTACACCAACACAACAACTCTGGATTTGTTGGCTACCTTGCC
CCCACCATGCGCGAAGGACAGGCCTACCCTGCTAACTTCCCCTAT
CCGCTTATAGGCAAGACCGCAGTTGACAGCATTACCCAGAAAAAG
TTTCTTTGCGATCGCACCCTTTGGCGCATCCCATTCTCCAGTAAC
TTTATGTCCATGGGCGCACTCACAGACCTGGGCCAAAACCTTCTC
TACGCCAACTCCGCCCACGCGCTAGACATGACTTTTGAGGTGGAT
CCCATGGACGAGCCCACCCTTCTTTATGTTTTGTTTGAAGTCTTT
GACGTGGTCCGTGTGCACCAGCCGCACCGCGGCGTCATCGAAACC
GTGTACCTGCGCACGCCCTTCTCGGCCGGCAACGCCACAACATAA
AGAAGCAAGCAACATCAACAACAGCTGCCGCCATGGGCTCCAGTG
AGCAGGAACTGAAAGCCATTGTCAAAGATCTTGGTTGTGGGCCAT
ATTTTTTGGGCACCTATGACAAGCGCTTTCCAGGCTTTGTTTCTC
CACACAAGCTCGCCTGCGCCATAGTCAATACGGCCGGTCGCGAGA
CTGGGGGCGTACACTGGATGGCCTTTGCCTGGAACCCGCACTCAA
AAACATGCTACCTCTTTGAGCCCTTTGGCTTTTCTGACCAGCGAC
TCAAGCAGGTTTACCAGTTTGAGTACGAGTCACTCCTGCGCCGTA
GCGCCATTGCTTCTTCCCCCGACCGCTGTATAACGCTGGAAAAGT
CCACCCAAAGCGTACAGGGGCCCAACTCGGCCGCCTGTGGACTAT
TCTGCTGCATGTTTCTCCACGCCTTTGCCAACTGGCCCCAAACTC
CCATGGATCACAACCCCACCATGAACCTTATTACCGGGGTACCCA
ACTCCATGCTCAACAGTCCCCAGGTACAGCCCACCCTGCGTCGCA
ACCAGGAACAGCTCTACAGCTTCCTGGAGCGCCACTCGCCCTACT
TCCGCAGCCACAGTGCGCAGATTAGGAGCGCCACTTCTTTTTGTC
ACTTGAAAAACATGTAAAAATAATGTACTAGAGACACTTTCAATA
AAGGCAAATGCTTTTATTTGTACACTCTCGGGTGATTATTTACCC
CCACCCTTGCCGTCTGCGCCGTTTAAAAATCAAAGGGGTTCTGCC
GCGCATCGCTATGCGCCACTGGCAGGGACACGTTGCGATACTGGT
GTTTAGTGCTCCACTTAAACTCAGGCACAACCATCCGCGGCAGCT
CGGTGAAGTTTTCACTCCACAGGCTGCGCACCATCACCAACGCGT
TTAGCAGGTCGGGCGCCGATATCTTGAAGTCGCAGTTGGGGCCTC
CGCCCTGCGCGCGCGAGTTGCGATACACAGGGTTGCAGCACTGGA
ACACTATCAGCGCCGGGTGGTGCACGCTGGCCAGCACGCTCTTGT
CGGAGATCAGATCCGCGTCCAGGTCCTCCGCGTTGCTCAGGGCGA
ACGGAGTCAACTTTGGTAGCTGCCTTCCCAAAAAGGGCGCGTGCC
CAGGCTTTGAGTTGCACTCGCACCGTAGTGGCATCAAAAGGTGAC
CGTGCCCGGTCTGGGCGTTAGGATACAGCGCCTGCATAAAAGCCT
TGATCTGCTTAAAAGCCACCTGAGCCTTTGCGCCTTCAGAGAAGA
ACATGCCGCAAGACTTGCCGGAAAACTGATTGGCCGGACAGGCCG
CGTCGTGCACGCAGCACCTTGCGTCGGTGTTGGAGATCTGCACCA
CATTTCGGCCCCACCGGTTCTTCACGATCTTGGCCTTGCTAGACT
GCTCCTTCAGCGCGCGCTGCCCGTTTTCGCTCGTCACATCCATTT
CAATCACGTGCTCCTTATTTATCATAATGCTTCCGTGTAGACACT
TAAGCTCGCCTTCGATCTCAGCGCAGCGGTGCAGCCACAACGCGC
AGCCCGTGGGCTCGTGATGCTTGTAGGTCACCTCTGCAAACGACT
GCAGGTACGCCTGCAGGAATCGCCCCATCATCGTCACAAAGGTCT
TGTTGCTGGTGAAGGTCAGCTGCAACCCGCGGTGCTCCTCGTTCA
GCCAGGTCTTGCATACGGCCGCCAGAGCTTCCACTTGGTCAGGCA
GTAGTTTGAAGTTCGCCTTTAGATCGTTATCCACGTGGTACTTGT
CCATCAGCGCGCGCGCAGCCTCCATGCCCTTCTCCCACGCAGACA
CGATCGGCACACTCAGCGGGTTCATCACCGTAATTTCACTTTCCG
CTTCGCTGGGCTCTTCCTCTTCCTCTTGCGTCCGCATACCACGCG
CCACTGGGTCGTCTTCATTCAGCCGCCGCACTGTGCGCTTACCTC
CTTTGCCATGCTTGATTAGCACCGGTGGGTTGCTGAAACCCACCA
TTTGTAGCGCCACATCTTCTCTTTCTTCCTCGCTGTCCACGATTA
CCTCTGGTGATGGCGGGCGCTCGGGCTTGGGAGAAGGGCGCTTCT
TTTTCTTCTTGGGCGCAATGGCCAAATCCGCCGCCGAGGTCGATG
GCCGCGGGCTGGGTGTGCGCGGCACCAGCGCGTCTTGTGATGAGT
CTTCCTCGTCCTCGGACTCGATACGCCGCCTCATCCGCTTTTTTG
GGGGCGCCCGGGGAGGCGGCGGCGACGGGGACGGGGACGACACGT
CCTCCATGGTTGGGGGACGTCGCGCCGCACCGCGTCCGCGCTCGG
GGGTGGTTTCGCGCTGCTCCTCTTCCCGACTGGCCATTTCCTTCT
CCTATAGGCAGAAAAAGATCATGGAGTCAGTCGAGAAGAAGGACA
GCCTAACCGCCCCCTCTGAGTTCGCCACCACCGCCTCCACCGATG
CCGCCAACGCGCCTACCACCTTCCCCGTCGAGGCACCCCCGCTTG
AGGAGGAGGAAGTGATTATCGAGCAGGACCCAGGTTTTGTAAGCG
AAGACGACGAGGACCGCTCAGTACCAACAGAGGATAAAAAGCAAG
ACCAGGACAACGCAGAGGCAAACGAGGAACAAGTCGGGCGGGGGG
ACGAAAGGCATGGCGACTACCTAGATGTGGGAGACGACGTGCTGT
TGAAGCATCTGCAGCGCCAGTGCGCCATTATCTGCGACGCGTTGC
AAGAGCGCAGCGATGTGCCCCTCGCCATAGCGGATGTCAGCCTTG
CCTACGAACGCCACCTATTCTCACCGCGCGTACCCCCCAAACGCC
AAGAAAACGGCACATGCGAGCCCAACCCGCGCCTCAACTTCTACC
CCGTATTTGCCGTGCCAGAGGTGCTTGCCACCTATCACATCTTTT
TCCAAAACTGCAAGATACCCCTATCCTGCCGTGCCAACCGCAGCC
GAGCGGACAAGCAGCTGGCCTTGCGGCAGGGCGCTGTCATACCTG
ATATCGCCTCGCTCAACGAAGTGCCAAAAATCTTTGAGGGTCTTG
GACGCGACGAGAAGCGCGCGGCAAACGCTCTGCAACAGGAAAACA
GCGAAAATGAAAGTCACTCTGGAGTGTTGGTGGAACTCGAGGGTG
ACAACGCGCGCCTAGCCGTACTAAAACGCAGCATCGAGGTCACCC
ACTTTGCCTACCCGGCACTTAACCTACCCCCCAAGGTCATGAGCA
CAGTCATGAGTGAGCTGATCGTGCGCCGTGCGCAGCCCCTGGAGA
GGGATGCAAATTTGCAAGAACAAACAGAGGAGGGCCTACCCGCAG
TTGGCGACGAGCAGCTAGCGCGCTGGCTTCAAACGCGCGAGCCTG
CCGACTTGGAGGAGCGACGCAAACTAATGATGGCCGCAGTGCTCG
TTACCGTGGAGCTTGAGTGCATGCAGCGGTTCTTTGCTGACCCGG
AGATGCAGCGCAAGCTAGAGGAAACATTGCACTACACCTTTCGAC
AGGGCTACGTACGCCAGGCCTGCAAGATCTCCAACGTGGAGCTCT
GCAACCTGGTCTCCTACCTTGGAATTTTGCACGAAAACCGCCTTG
GGCAAAACGTGCTTCATTCCACGCTCAAGGGCGAGGCGCGCCGCG
ACTACGTCCGCGACTGCGTTTACTTATTTCTATGCTACACCTGGC
AGACGGCCATGGGCGTTTGGCAGCAGTGCTTGGAGGAGTGCAACC
TCAAGGAGCTGCAGAAACTGCTAAAGCAAAACTTGAAGGACCTAT
GGACGGCCTTCAACGAGCGCTCCGTGGCCGCGCACCTGGCGGACA
TCATTTTCCCCGAACGCCTGCTTAAAACCCTGCAACAGGGTCTGC
CAGACTTCACCAGTCAAAGCATGTTGCAGAACTTTAGGAACTTTA
TCCTAGAGCGCTCAGGAATCTTGCCCGCCACCTGCTGTGCACTTC
CTAGCGACTTTGTGCCCATTAAGTACCGCGAATGCCCTCCGCCGC
TTTGGGGCCACTGCTACCTTCTGCAGCTAGCCAACTACCTTGCCT
ACCACTCTGACATAATGGAAGACGTGAGCGGTGACGGTCTACTGG
AGTGTCACTGTCGCTGCAACCTATGCACCCCGCACCGCTCCCTGG
TTTGCAATTCGCAGCTGCTTAACGAAAGTCAAATTATCGGTACCT
TTGAGCTGCAGGGTCCCTCGCCTGACGAAAAGTCCGCGGCTCCGG
GGTTGAAACTCACTCCGGGGCTGTGGACGTCGGCTTACCTTCGCA
AATTTGTACCTGAGGACTACCACGCCCACGAGATTAGGTTCTACG
AAGACCAATCCCGCCCGCCTAATGCGGAGCTTACCGCCTGCGTCA
TTACCCAGGGCCACATTCTTGGCCAATTGCAAGCCATCAACAAAG
CCCGCCAAGAGTTTCTGCTACGAAAGGGACGGGGGGTTTACTTGG
ACCCCCAGTCCGGCGAGGAGCTCAACCCAATCCCCCCGCCGCCGC
AGCCCTATCAGCAGCAGCCGCGGGCCCTTGCTTCCCAGGATGGCA
CCCAAAAAGAAGCTGCAGCTGCCGCCGCCACCCACGGACGAGGAG
GAATACTGGGACAGTCAGGCAGAGGAGGTTTTGGACGAGGAGGAG
GAGGACATGATGGAAGACTGGGAGAGCCTAGACGAGGAAGCTTCC
GAGGTCGAAGAGGTGTCAGACGAAACACCGTCACCCTCGGTCGCA
TTCCCCTCGCCGGCGCCCCAGAAATCGGCAACCGGTTCCAGCATG
GCTACAACCTCCGCTCCTCAGGCGCCGCCGGCACTGCCCGTTCGC
CGACCCAACCGTAGATGGGACACCACTGGAACCAGGGCCGGTAAG
TCCAAGCAGCCGCCGCCGTTAGCCCAAGAGCAACAACAGCGCCAA
GGCTACCGCTCATGGCGCGGGCACAAGAACGCCATAGTTGCTTGC
TTGCAAGACTGTGGGGGCAACATCTCCTTCGCCCGCCGCTTTCTT
CTCTACCATCACGGCGTGGCCTTCCCCCGTAACATCCTGCATTAC
TACCGTCATCTCTACAGCCCATACTGCACCGGCGGCAGCGGCAGC
AACAGCAGCGGCCACACAGAAGCAAAGGCGACCGGATAGCAAGAC
TCTGACAAAGCCCAAGAAATCCACAGCGGCGGCAGCAGCAGGAGG
AGGAGCGCTGCGTCTGGCGCCCAACGAACCCGTATCGACCCGCGA
GCTTAGAAACAGGATTTTTCCCACTCTGTATGCTATATTTCAACA
GAGCAGGGGCCAAGAACAAGAGCTGAAAATAAAAAACAGGTCTCT
GCGATCCCTCACCCGCAGCTGCCTGTATCACAAAAGCGAAGATCA
GCTTCGGCGCACGCTGGAAGACGCGGAGGCTCTCTTCAGTAAATA
CTGCGCGCTGACTCTTAAGGACTAGTTTCGCGCCCTTTCTCAAAT
TTAAGCGCGAAAACTACGTCATCTCCAGCGGCCACACCCGGCGCC
AGCACCTGTTGTCAGCGCCATTATGAGCAAGGAAATTCCCACGCC
CTACATGTGGAGTTACCAGCCACAAATGGGACTTGCGGCTGGAGC
TGCCCAAGACTACTCAACCCGAATAAACTACATGAGCGCGGGACC
CCACATGATATCCCGGGTCAACGGAATACGCGCCCACCGAAACCG
AATTCTCCTGGAACAGGCGGCTATTACCACCACACCTCGTAATAA
CCTTAATCCCCGTAGTTGGCCCGCTGCCCTGGTGTACCAGGAAAG
TCCCGCTCCCACCACTGTGGTACTTCCCAGAGACGCCCAGGCCGA
AGTTCAGATGACTAACTCAGGGGCGCAGCTTGCGGGCGGCTTTCG
TCACAGGGTGCGGTCGCCCGGGCAGGGTATAACTCACCTGACAAT
CAGAGGGCGAGGTATTCAGCTCAACGACGAGTCGGTGAGCTCCTC
GCTTGGTCTCCGTCCGGACGGGACATTTCAGATCGGCGGCGCCGG
CCGCTCTTCATTCACGCCTCGTCAGGCAATCCTAACTCTGCAGAC
CTCGTCCTCTGAGCCGCGCTCTGGAGGCATTGGAACTCTGCAATT
TATTGAGGAGTTTGTGCCATCGGTCTACTTTAACCCCTTCTCGGG
ACCTCCCGGCCACTATCCGGATCAATTTATTCCTAACTTTGACGC
GGTAAAGGACTCGGCGGACGGCTACGACTGAATGTTAAGTGGAGA
GGCAGAGCAACTGCGCCTGAAACACCTGGTCCACTGTCGCCGCCA
CAAGTGCTTTGCCCGCGACTCCGGTGAGTTTTGCTACTTTGAATT
GCCCGAGGATCATATCGAGGGCCCGGCGCACGGCGTCCGGCTTAC
CGCCCAGGGAGAGCTTGCCCGTAGCCTGATTCGGGAGTTTACCCA
GCGCCCCCTGCTAGITGAGCGGGACAGGGGACCCTGTGTTCTCAC
TGTGATTTGCAACTGTCCTAACCCTGGATTACATCAAGATCCTCT
AGTTAATGTCAGGTCGCCTAAGTCGATTAACTAGAGTACCCGGGG
ATCTTATTCCCTTTAACTAATAAAAAAAAATAATAAAGCATCACT
TACTTAAAATCAGTTAGCAAATTTCTGTCCAGTTTATTCAGCAGC
ACCTCCTTGCCCTCCTCCCAGCTCTGGTATTGCAGCTTCCTCCTG
GCTGCAAACTTTCTCCACAATCTAAATGGAATGTCAGTTTCCTCC
TGTTCCTGTCCATCCGCACCCACTATCTTCATGTTGTTGCAGATG
AAGCGCGCAAGACCGTCTGAAGATACCTTCAACCCCGTGTATCCA
TATGACACGGAAACCGGTCCTCCAACTGTGCCTTTTCTTACTCCT
CCCTTTGTATCCCCCAATGGGTTTCAAGAGAGTCCCCCTGGGGTA
CTCTCTTTGCGCCTATCCGAACCTCTAGTTACCTCCAATGGCATG
CTTGCGCTCAAAATGGGCAACGGCCTCTCTCTGGACGAGGCCGGC
AACCTTACCTCCCAAAATGTAACCACTGTGAGCCCACCTCTCAAA
AAAACCAAGTCAAACATAAACCTGGAAATATCTGCACCCCTCACA
GTTACCTCAGAAGCCCTAACTGTGGCTGCCGCCGCACCTCTAATG
GTCGCGGGCAACACACTCACCATGCAATCACAGGCCCCGCTAACC
GTGCACGACTCCAAACTTAGCATTGCCACCCAAGGACCCCTCACA
GTGTCAGAAGGAAAGCTAGCCCTGCAAACATCAGGCCCCCTCACC
ACCACCGATAGCAGTACCCTTACTATCACTGCCTCACCCCCTCTA
ACTACTGCCACTGGTAGCTTGGGCATTGACTTGAAAGAGCCCATT
TATACACAAAATGGAAAACTAGGACTAAAGTACGGGGCTCCTTTG
CATGTAACAGACGACCTAAACACTTTGACCGTAGCAACTGGTCCA
GGTGTGACTATTAATAATACTTCCTTGCAAACTAAAGTTACTGGA
GCCTTGGGTTTTGATTCACAAGGCAATATGCAACTTAATGTAGCA
GGAGGACTAAGGATTGATTCTCAAAACAGACGCCTTATACTTGAT
GTTAGTTATCCGTTTGATGCTCAAAACCAACTAAATCTAAGACTA
GGACAGGGCCCTCTTTTTATAAACTCAGCCCACAACTTGGATATT
AACTACAACAAAGGCCTTTACTTGTTTACAGCTTCAAACAATTCC
AAAAAGCTTGAGGTTAACCTAAGCACTGCCAAGGGGTTGATGTTT
GACGCTACAGCCATAGCCATTAATGCAGGAGATGGGCTTGAATTT
GGTTCACCTAATGCACCAAACACAAATCCCCTCAAAACAAAAATT
GGCCATGGCCTAGAATTTGATTCAAACAAGGCTATGGTTCCTAAA
CTAGGAACTGGCCTTAGTTTTGACAGCACAGGTGCCATTACAGTA
GGAAACAAAAATAATGATAAGCTAACTTTGTGGACCACACCAGCT
CCATCTCCTAACTGTAGACTAAATGCAGAGAAAGATGCTAAACTC
ACTTTGGTCTTAACAAAATGTGGCAGTCAAATACTTGCTACAGTT
TCAGTTTTGGCTGTTAAAGGCAGTTTGGCTCCAATATCTGGAACA
GTTCAAAGTGCTCATCTTATTATAAGATTTGACGAAAATGGAGTG
CTACTAAACAATTCCTTCCTGGACCCAGAATATTGGAACTTTAGA
AATGGAGATCTTACTGAAGGCACAGCCTATACAAACGCTGTTGGA
TTTATGCCTAACCTATCAGCTTATCCAAAATCTCACGGTAAAACT
GCCAAAAGTAACATTGTCAGTCAAGTTTACTTAAACGGAGACAAA
ACTAAACCTGTAACACTAACCATTACACTAAACGGTACACAGGAA
ACAGGAGACACAACTCCAAGTGCATACTCTATGTCATTTTCATGG
GACTGGTCTGGCCACAACTACATTAATGAAATATTTGCCACATCC
TCTTACACTTTTTCATACATTGCCCAAGAATAAAGAATCGTTTGT
GTTATGTTTCAACGTGTTTATTTTTCAATTGCAGAAAATTTCAAG
TCATTTTTCATTCAGTAGTATAGCCCCACCACCACATAGCTTATA
CAGATCACCGTACCTTAATCAAACTCACAGAACCCTAGTATTCAA
CCTGCCACCTCCCTCCCAACACACAGAGTACACAGTCCTTTCTCC
CCGGCTGGCCTTAAAAAGCATCATATCATGGGTAACAGACATATT
CTTAGGTGTTATATTCCACACGGTTTCCTGTCGAGCCAAACGCTC
ATCAGTGATATTAATAAACTCCCCGGGCAGCTCACTTAAGTTCAT
GTCGCTGTCCAGCTGCTGAGCCACAGGCTGCTGTCCAACTTGCGG
TTGCTTAACGGGCGGCGAAGGAGAAGTCCACGCCTACATGGGGGT
AGAGTCATAATCGTGCATCAGGATAGGGCGGTGGTGCTGCAGCAG
CGCGCGAATAAACTGCTGCCGCCGCCGCTCCGTCCTGCAGGAATA
CAACATGGCAGTGGTCTCCTCAGCGATGATTCGCACCGCCCGCAG
CATAAGGCGCCTTGTCCTCCGGGCACAGCAGCGCACCCTGATCTC
ACTTAAATCAGCACAGTAACTGCAGCACAGCACCACAATATTGTT
CAAAATCCCACAGTGCAAGGCGCTGTATCCAAAGCTCATGGGGGG
ACCACAGAACCCACGTGGCCATCATACCACAAGCGCAGGTAGATT
AAGTGGCGACCCCTCATAAACACGCTGGACATAAACATTACCTCT
TTTGGCATGTTGTAATTCACCACCTCCCGGTACCATATAAACCTC
TGATTAAACATGGCGCCATCCACCACCATCCTAAACCAGCTGGCC
AAAACCTGCCCGCCGGCTATACACTGCAGGGAACCGGGACTGGAA
CAATGACAGTGGAGAGCCCAGGACTCGTAACCATGGATCATCATG
CTCGTCATGATATCAATGTTGGCACAACACAGGCACACGTGCATA
CACTTCCTCAGGATTACAAGCTCCTCCCGCGTTAGAACCATATCC
CAGGGAACAACCCATTCCTGAATCAGCGTAAATCCCACACTGCAG
GGAAGACCTCGCACGTAACTCACGTTGTGCATTGTCAAAGTGTTA
CATTCGGGCAGCAGCGGATGATCCTCCAGTATGGTAGCGCGGGTT
TCTGTCTCAAAAGGAGGTAGACGATCCCTACTGTACGGAGTGCGC
CGAGACAACCGAGATCGTGTTGGTCGTAGTGTCATGCCAAATGGA
ACGCCGGACGTAGTCATATTTCCTGAAGCAAAACCAGGTGCGGGC
GTGACAAACAGATCTGCGTCTCCGGTCTCGCCGCTTAGATCGCTC
TGTGTAGTAGTTGTAGTATATCCACTCTCTCAAAGCATCCAGGCG
CCCCCTGGCTTCGGGTTCTATGTAAACTCCTTCATGCGCCGCTGC
CCTGATAACATCCACCACCGCAGAATAAGCCACACCCAGCCAACC
TACACATTCGTTCTGCGAGTCACACACGGGAGGAGCGGGAAGAGC
TGGAAGAACCATGTTTTTTTTTTTATTCCAAAAGATTATCCAAAA
CCTCAAAATGAAGATCTATTAAGTGAACGCGCTCCCCTCCGGTGG
CGTGGTCAAACTCTACAGCCAAAGAACAGATAATGGCATTTGTAA
GATGTTGCACAATGGCTTCCAAAAGGCAAACGGCCCTCACGTCCA
AGTGGACGTAAAGGCTAAACCCTTCAGGGTGAATCTCCTCTATAA
ACATTCCAGCACCTTCAACCATGCCCAAATAATTCTCATCTCGCC
ACCTTCTCAATATATCTCTAAGCAAATCCCGAATATTAAGTCCGG
CCATTGTAAAAATCTGCTCCAGAGCGCCCTCCACCTTCAGCCTCA
AGCAGCGAATCATGATTGCAAAAATTCAGGTTCCTCACAGACCTG
TATAAGATTCAAAAGCGGAACATTAACAAAAATACCGCGATCCCG
TAGGTCCCTTCGCAGGGCCAGCTGAACATAATCGTGCAGGTCTGC
ACGGACCAGCGCGGCCACTTCCCCGCCAGGAACCATGACAAAAGA
ACCCACACTGATTATGACACGCATACTCGGAGCTATGCTAACCAG
CGTAGCCCCGATGTAAGCTTGTTGCATGGGCGGCGATATAAAATG
CAAGGTGCTGCTCAAAAAATCAGGCAAAGCCTCGCGCAAAAAAGA
AAGCACATCGTAGTCATGCTCATGCAGATAAAGGCAGGTAAGCTC
CGGAACCACCACAGAAAAAGACACCATTTTTCTCTCAAACATGTC
TGCGGGTTTCTGCATAAACACAAAATAAAATAACAAAAAAACATT
TAAACATTAGAAGCCTGTCTTACAACAGGAAAAACAACCCTTATA
AGCATAAGACGGACTACGGCCATGCCGGCGTGACCGTAAAAAAAC
TGGTCACCGTGATTAAAAAGCACCACCGACAGCTCCTCGGTCATG
TCCGGAGTCATAATGTAAGACTCGGTAAACACATCAGGTTGATTC
ACATCGGTCAGTGCTAAAAAGCGACCGAAATAGCCCGGGGGAATA
CATACCCGCAGGCGTAGAGACAACATTACAGCCCCCATAGGAGGT
ATAACAAAATTAATAGGAGAGAAAAACACATAAACACCTGAAAAA
CCCTCCTGCCTAGGCAAAATAGCACCCTCCCGCTCCAGAACAACA
TACAGCGCTTCCACAGCGGCAGCCATAACAGTCAGCCTTACCAGT
AAAAAAGAAAACCTATTAAAAAAACACCACTCGACACGGCACCAG
CTCAATCAGTCACAGTGTAAAAAAGGGCCAAGTGCAGAGCGAGTA
TATATAGGACTAAAAAATGACGTAACGGTTAAAGTCCACAAAAAA
CACCCAGAAAACCGCACGCGAACCTACGCCCAGAAACGAAAGCCA
AAAAACCCACAACTTCCTCAAATCGTCACTTCCGTTTTCCCACGT
TACGTCACTTCCCATTTTAAGAAAACTACAATTCCCAACACATAC
AAGTTACTCCGCCCTAAAACCTACGTCACCCGCCCCGTTCCCACG
CCCCGCGCCACGTCACAAACTCCACCCCCTCATTATCATATTGGC
TTCAATCCAAAATAAGGTATATT
SEQ ID NO: 9:
rAd-CMV-SARS-CoV-2-S1-Furin-N-BGH-CMV-dsRNA-SPA
TAAGGATCCCATCATCAATAATATACCTTATTTTGGATTGAAGCC
AATATGATAATGAGGGGGTGGAGTTTGTGACGTGGCGCGGGGCGT
GGGAACGGGGGGGGTGACGTAGTAGTGTGGCGGAAGTGTGATGTT
GCAAGTGTGGCGGAACACATGTAAGCGACGGATGTGGCAAAAGTG
ACGTTTTTGGTGTGCGCCGGTGTACACAGGAAGTGACAATTTTCG
CGCGGTTTTAGGCGGATGTTGTAGTAAATTTGGGCGTAACCGAGT
AAGATTTGGCCATTTTCGCGGGAAAACTGAATAAGAGGAAGTGAA
ATCTGAATAATTTTGTGTTACTCATAGCGCGTAATACTGCTAGAG
ATCTGGCGAAAGGGGGATGTGCTGCAAGGCGATTAAGITGGGTAA
CGCCAGGGTTTTCCCAGTCACGACGTTGTAAAACGACGGCCAGTG
AATTGTAATACGACTCACTATAGGGCGAATTGGGTACTGGCCACA
GGAGCTTGGCCCATTGCATACGTTGTATCCATATCATAATATGTA
CATTTATATTGGCTCATGTCCAACATTACCGCCATGTTGACATTG
ATTATTGACTAGTTATTAATAGTAATCAATTACGGGGTCATTAGT
TCATAGCCCATATATGGAGTTCCGCGTTACATAACTTACGGTAAA
TGGCCCGCCTGGCTGACCGCCCAACGACCCCCGCCCATTGACGTC
AATAATGACGTATGTTCCCATAGTAACGCCAATAGGGACTTTCCA
TTGACGTCAATGGGTGGAGTATTTACGGTAAACTGCCCACTTGGC
AGTACATCAAGTGTATCATATGCCAAGTACGCCCCCTATTGACGT
CAATGACGGTAAATGGCCCGCCTGGCATTATGCCCAGTACATGAC
CTTATGGGACTTTCCTACTTGGCAGTACATCTACGTATTAGTCAT
CGCTATTACCATGGTGATGCGGTTTTGGCAGTACATCAATGGGCG
TGGATAGCGGTTTGACTCACGGGGATTTCCAAGTCTCCACCCCAT
TGACGTCAATGGGAGTTTGTTTTGGCACCAAAATCAACGGGACTT
TCCAAAATGTCGTAACAACTCCGCCCCATTGACGCAAATGGGCGG
TAGGCGTGTACGGTGGGAGGTCTATATAAGCAGAGCTCGTTTAGT
GAACCGTCAGatcgcctggagacgccatccacgctgttttgacct
ccatagaagacaccgggaccgatccagcctgactctagCctAGCT
Ctgaagttggtggtgaggccctgggcaggttggtatcaaggttac
aagacaggtttaaggagaccaatagaaactgggcatgtggagaca
gagaagactcttgggtttctgataggcactgactctctctgccta
ttggtctattttcccacccttaggctgctggtctgagcctagGAG
ATCTCTCGAGGTCGACGGTATCGATGggtaccgccaccATGTTTG
TTTTTCTCGTACTCCTGCCCCTGGTTTCCTCCCAATGTGTCAATC
TGACTACCCGGACCCAACTTCCTCCCGCCTACACCAATTCCTTTA
CCCGAGGTGTTTACTACCCAGACAAAGTGTTCAGGTCATCCGTCC
TCCATAGTACCCAAGACCTCTTCCTCCCTTTTTTTTCTAACGTTA
CCTGGTTTCACGCTATTCACGTTAGCGGCACCAACGGCACCAAAA
GATTCGATAACCCCGTACTGCCGTTCAACGACGGGGTATATTTTG
CCTCTACTGAAAAATCAAACATCATACGCGGATGGATCTTTGGGA
CTACCCTGGACTCAAAAACTCAGTCCCTGCTGATTGTGAATAACG
CTACCAACGTGGTGATCAAAGTCTGTGAATTCCAGTTTTGCAACG
ATCCTTTTCTCGGCGTTTATTATCACAAAAATAACAAATCCTGGA
TGGAGAGCGAGTTCCGGGTGTACTCCTCCGCGAATAATTGCACCT
TCGAATATGTGTCTCAGCCATTCCTCATGGACCTCGAGGGGAAGC
AGGGCAATTTTAAGAATCTGCGAGAATTCGTGTTCAAGAATATAG
ACGGTTACTTCAAGATTTACTCCAAACACACCCCGATTAACCTGG
TTAGGGACTTGCCTCAGGGCTTTTCTGCATTGGAGCCCCTCGTGG
ACCTCCCAATCGGCATAAACATTACAAGATTTCAGACTTTGCTTG
CATTGCACAGGAGCTATTTGACACCCGGCGATTCTTCTTCCGGAT
GGACCGCTGGAGCAGCTGCTTATTACGTGGGCTATCTGCAGCCTC
GAACCTTTCTTTTGAAGTACAACGAAAATGGAACTATCACCGATG
CAGTTGACTGCGCCCTGGACCCCCTGTCCGAAACTAAGTGCACGC
TCAAAAGTTTCACAGTAGAGAAGGGGATATACCAGACTAGCAATT
TCCGCGTTCAGCCAACCGAAAGTATAGTGCGCTTTCCTAATATAA
CTAACCTGTGTCCTTTCGGGGAAGTGTTTAACGCCACTAGATTCG
CTTCCGTCTACGCCTGGAATAGAAAGAGGATCTCAAATTGCGTTG
CTGACTATAGTGTTTTGTACAATTCCGCCTCTTTCTCAACCTTCA
AATGTTACGGGGTGAGCCCTACCAAACTGAACGACCTGTGCTTTA
CAAACGTATACGCCGACAGCTTTGTTATCAGAGGAGACGAGGTTC
GCCAGATTGCTCCGGGTCAGACAGGCAAGATTGCTGATTATAATT
ACAAACTGCCCGACGACTTTACAGGATGTGTGATCGCGTGGAACA
GTAACAATCTTGACTCAAAGGTTGGGGGTAATTATAATTATCTTT
ACCGGCTGTTCAGAAAAAGCAATTTGAAACCCTTCGAAAGGGACA
TATCCACCGAGATCTATCAGGCCGGGTCCACTCCATGCAATGGTG
TGGAAGGTTTTAATTGCTACTTCCCATTGCAGTCTTATGGATTCC
AACCAACCAATGGCGTAGGCTACCAGCCGTATCGCGTTGTCGTGC
TCAGCTTCGAGCTGCTCCACGCCCCCGCGACCGTATGCGGTCCTA
AGAAGTCCACCAATCTTGTTAAGAACAAGTGTGTAAACTTTAACT
TTAACGGGCTGACCGGGACCGGCGTTCTGACTGAATCTAACAAAA
AATTCCTGCCTTTCCAGCAGTTCGGCCGCGATATTGCTGACACCA
CTGACGCTGTAAGAGACCCTCAGACCCTTGAAATTCTCGATATCA
CACCTTGCAGCTTTGGGGGCGTGTCCGTCATCACTCCAGGAACTA
ACACAAGCAACCAGGTGGCAGTGTTGTACCAGGATGTTAATTGTA
CCGAGGTGCCAGTGGCCATCCACGCCGATCAATTGACACCTACCT
GGAGGGTTTACAGCACAGGGTCCAATGTTTTTCAGACAAGAGCCG
GATGTCTGATCGGTGCCGAGCATGTCAACAATTCCTACGAGTGTG
ATATCCCCATTGGTGCGGGAATTTGTGCATCATATCAGACCCAGA
CTAATAGCCCAAGAAGAGCTAGATCCGTCGCTAGTCAATCCATCA
TTGCATATACAATGATGTCCGATAACGGCCCCCAGAATCAGAGAA
ACGCTCCCCGCATCACGTTCGGCGGACCAAGTGACAGCACAGGCA
GTAACCAGAACGGAGAACGCTCCGGTGCTCGCTCCAAGCAGCGAC
GGCCGCAAGGGCTTCCCAACAATACCGCCAGCTGGTTTACGGCTC
TGACCCAACACGGGAAAGAAGATCTTAAATTCCCCAGGGGCCAGG
GCGTCCCTATCAATACTAACTCCAGCCCGGATGATCAGATAGGCT
ACTATAGACGCGCTACCCGACGGATACGAGGGGGGGACGGCAAAA
TGAAGGACCTTTCCCCCCGGTGGTATTTCTATTACTTGGGCACCG
GACCAGAAGCCGGACTGCCTTACGGCGCTAACAAAGACGGAATAA
TCTGGGTTGCGACGGAGGGCGCCCTGAATACACCTAAAGACCATA
TCGGCACAAGAAATCCTGCTAACAATGCCGCGATTGTGCTCCAGC
TGCCTCAGGGAACCACGCTGCCTAAAGGGTTTTACGCTGAGGGGT
CAAGGGGGGGGAGTCAAGCGTCTAGTAGGTCATCCTCTCGCTCTC
GCAATAGTTCCCGGAACTCAACCCCAGGCAGCAGCAGAGGAACCT
CTCCCGCACGGATGGCTGGCAATGGGGGAGATGCTGCCCTTGCTC
TCCTTCTGCTGGATCGCCTTAACCAGCTCGAATCAAAGATGTCTG
GAAAAGGTCAGCAGCAGCAAGGCCAGACCGTGACAAAGAAGAGTG
CAGCTGAAGCTAGTAAAAAGCCACGCCAAAAACGGACCGCAACTA
AGGCATATAACGTAACACAGGCCTTCGGCAGAAGAGGTCCAGAAC
AAACACAGGGAAACTTTGGCGATCAAGAGCTGATTAGACAGGGCA
CAGATTACAAACACTGGCCACAGATCGCGCAGTTTGCACCAAGCG
CCTCTGCATTCTTCGGGATGAGTCGGATTGGGATGGAAGTCACTC
CATCCGGGACCTGGCTTACCTACACAGGGGCAATAAAACTCGACG
ACAAAGACCCAAACTTTAAAGATCAGGTCATCCTGCTGAATAAAC
ACATCGATGCCTACAAAACTTTCCCCCCAACCGAACCAAAGAAAG
ACAAGAAAAAAAAGGCAGACGAAACGCAAGCGCTCCCTCAGCGCC
AGAAGAAGCAGCAGACCGTTACACTGTTGCCAGCAGCAGATCTGG
ATGATTTTTCCAAGCAGCTTCAACAGAGTATGTCAAGCGCTGACA
GCACTCAGGCTTGAcgatcgGATATCGCTAGCGTACCGGCGGCCG
CCCTATTCTATAGTGTCACCTAAATGCTAGAGCTCGCTGATCAGC
CTCGACTGTGCCTTCTAGTTGCCAGCCATCTGTTGTTTGCCCCTC
CCCCGTGCCTTCCTTGACCCTGGAAGGTGCCACTCCCACTGTCCT
TTCCTAATAAAATGAGGAAATTGCATCGCATTGTCTGAGTAGGTG
TCATTCTATTCTGGGGGGTGGGGTGGGGCAGGACAGCAAGGGGGA
GGATTGGGAAGACAATAGCAGGCATGCTGGGGATGCGGTGGGCTC
TATGGCTTCTGAGGCGGAAAGAACCAAAGCTTAcgcgttagttat
taataGTAATCAATTACGGGGTCATTAGTTCATAGCCCATATATG
GAGTTCCGCGTTACATAACTTACGGTAAATGGCCCGCCTGGCTGA
CCGCCCAACGACCCCCGCCCATTGACGTCAATAATGACGTATGTT
CCCATAGTAACGCCAATAGGGACTTTCCATTGACGTCAATGGGTG
GAGTATTTACGGTAAACTGCCCACTTGGCAGTACATCAAGTGTAT
CATATGCCAAGTACGCCCCCTATTGACGTCAATGACGGTAAATGG
CCCGCCTGGCATTATGCCCAGTACATGACCTTATGGGACTTTCCT
ACTTGGCAGTACATCTACGTATTAGTCATCGCTATTACCATGGTG
ATGCGGTTTTGGCAGTACATCAATGGGCGTGGATAGCGGTTTGAC
TCACGGGGATTTCCAAGTCTCCACCCCATTGACGTCAATGGGAGT
TTGTTTTGGCACCAAAATCAACGGGACTTTCCAAAATGTCGTAAC
AACTCCGCCCCATTGACGCAAATGGGCGGTAGGCGTGTACGGTGG
GAGGTCTATATAAGCAGAGCTGGTTTAGTGAACCGTCAGATCCGC
TAGAGATATCGGGCCACTGCAGGAAACGATATGGGCTGAATACGG
ATCCGTATTCAGCCCATATCGTTTCTCTAGAAATAAAATATCTTT
ATTTTCATTACATCTGTGTGTTGGTTTTTTGTGTGAATCGATAGT
ACTAACATACGCTCTCCATCTCGAGCCTAAGCTTGTCGACTCGAA
GATCTGGGCGTGGTTAAGGGTGGGAAAGAATATATAAGGTGGGGG
TCTTATGTAGTTTTGTATCTGTTTTGCAGCAGCCGCCGCCGCCAT
GAGCACCAACTCGTTTGATGGAAGCATTGTGAGCTCATATTTGAC
AACGCGCATGCCCCCATGGGCCGGGGTGCGTCAGAATGTGATGGG
CTCCAGCATTGATGGTCGCCCCGTCCTGCCCGCAAACTCTACTAC
CTTGACCTACGAGACCGTGTCTGGAACGCCGTTGGAGACTGCAGC
CTCCGCCGCCGCTTCAGCCGCTGCAGCCACCGCCCGCGGGATTGT
GACTGACTTTGCTTTCCTGAGCCCGCTTGCAAGCAGTGCAGCTTC
CCGTTCATCCGCCCGCGATGACAAGTTGACGGCTCTTTTGGCACA
ATTGGATTCTTTGACCCGGGAACTTAATGTCGTTTCTCAGCAGCT
GTTGGATCTGCGCCAGCAGGTTTCTGCCCTGAAGGCTTCCTCCCC
TCCCAATGCGGTTTAAAACATAAATAAAAAACCAGACTCTGTTTG
GATTTGGATCAAGCAAGTGTCTTGCTGTCTTTATTTAGGGGTTTT
GCGCGCGCGGTAGGCCCGGGACCAGCGGTCTCGGTCGTTGAGGGT
CCTGTGTATTTTTTCCAGGACGTGGTAAAGGTGACTCTGGATGTT
CAGATACATGGGCATAAGCCCGTCTCTGGGGTGGAGGTAGCACCA
CTGCAGAGCTTCATGCTGCGGGGTGGTGTTGTAGATGATCCAGTC
GTAGCAGGAGCGCTGGGCGTGGTGCCTAAAAATGTCTTTCAGTAG
CAAGCTGATTGCCAGGGGCAGGCCCTTGGTGTAAGTGTTTACAAA
GCGGTTAAGCTGGGATGGGTGCATACGTGGGGATATGAGATGCAT
CTTGGACTGTATTTTTAGGTTGGCTATGTTCCCAGCCATATCCCT
CCGGGGATTCATGTTGTGCAGAACCACCAGCACAGTGTATCCGGT
GCACTTGGGAAATTTGTCATGTAGCTTAGAAGGAAATGCGTGGAA
GAACTTGGAGACGCCCTTGTGACCTCCAAGATTTTCCATGCATTC
GTCCATAATGATGGCAATGGGCCCACGGGCGGCGGCCTGGGCGAA
GATATTTCTGGGATCACTAACGTCATAGTTGTGTTCCAGGATGAG
ATCGTCATAGGCCATTTTTACAAAGCGCGGGCGGAGGGTGCCAGA
CTGCGGTATAATGGTTCCATCCGGCCCAGGGGCGTAGTTACCCTC
ACAGATTTGCATTTCCCACGCTTTGAGTTCAGATGGGGGGATCAT
GTCTACCTGCGGGGCGATGAAGAAAACGGTTTCCGGGGTAGGGGA
GATCAGCTGGGAAGAAAGCAGGTTCCTGAGCAGCTGCGACTTACC
GCAGCCGGTGGGCCCGTAAATCACACCTATTACCGGCTGCAACTG
GTAGTTAAGAGAGCTGCAGCTGCCGTCATCCCTGAGCAGGGGGGC
CACTTCGTTAAGCATGTCCCTGACTCGCATGTTTTCCCTGACCAA
ATCCGCCAGAAGGCGCTCGCCGCCCAGCGATAGCAGTTCTTGCAA
GGAAGCAAAGTTTTTCAACGGTTTGAGACCGTCCGCCGTAGGCAT
GCTTTTGAGCGTTTGACCAAGCAGTTCCAGGCGGTCCCACAGCTC
GGTCACCTGCTCTACGGCATCTCGATCCAGCATATCTCCTCGTTT
CGCGGGTTGGGGCGGCTTTCGCTGTACGGCAGTAGTCGGTGCTCG
TCCAGACGGGCCAGGGTCATGTCTTTCCACGGGCGCAGGGTCCTC
GTCAGCGTAGTCTGGGTCACGGTGAAGGGGTGCGCTCCGGGCTGC
GCGCTGGCCAGGGTGCGCTTGAGGCTGGTCCTGCTGGTGCTGAAG
CGCTGCCGGTCTTCGCCCTGCGCGTCGGCCAGGTAGCATTTGACC
ATGGTGTCATAGTCCAGCCCCTCCGCGGCGTGGCCCTTGGCGCGC
AGCTTGCCCTTGGAGGAGGCGCCGCACGAGGGGCAGTGCAGACTT
TTGAGGGCGTAGAGCTTGGGCGCGAGAAATACCGATTCCGGGGAG
TAGGCATCCGCGCCGCAGGCCCCGCAGACGGTCTCGCATTCCACG
AGCCAGGTGAGCTCTGGCCGTTCGGGGTCAAAAACCAGGTTTCCC
CCATGCTTTTTGATGCGTTTCTTACCTCTGGTTTCCATGAGCCGG
TGTCCACGCTCGGTGACGAAAAGGCTGTCCGTGTCCCCGTATACA
GACTTGAGAGGCCTGTCCTCGAGCGGTGTTCCGCGGTCCTCCTCG
TATAGAAACTCGGACCACTCTGAGACAAAGGCTCGCGTCCAGGCC
AGCACGAAGGAGGCTAAGTGGGAGGGGTAGCGGTCGTTGTCCACT
AGGGGGTCCACTCGCTCCAGGGTGTGAAGACACATGTCGCCCTCT
TCGGCATCAAGGAAGGTGATTGGTTTGTAGGTGTAGGCCACGTGA
CCGGGTGTTCCTGAAGGGGGGCTATAAAAGGGGGTGGGGGCGCGT
TCGTCCTCACTCTCTTCCGCATCGCTGTCTGCGAGGGCCAGCTGT
TGGGGTGAGTACTCCCTCTGAAAAGCGGGCATGACTTCTGCGCTA
AGATTGTCAGTTTCCAAAAACGAGGAGGATTTGATATTCACCTGG
CCCGCGGTGATGCCTTTGAGGGTGGCCGCATCCATCTGGTCAGAA
AAGACAATCTTTTTGTTGTCAAGCTTGGTGGCAAACGACCCGTAG
AGGGCGTTGGACAGCAACTTGGCGATGGAGCGCAGGGTTTGGTTT
TTGTCGCGATCGGCGCGCTCCTTGGCCGCGATGTTTAGCTGCACG
TATTCGCGCGCAACGCACCGCCATTCGGGAAAGACGGTGGTGCGC
TCGTCGGGCACCAGGTGCACGCGCCAACCGCGGTTGTGCAGGGTG
ACAAGGTCAACGCTGGTGGCTACCTCTCCGCGTAGGCGCTCGTTG
GTCCAGCAGAGGCGGCCGCCCTTGCGCGAGCAGAATGGCGGTAGG
GGGTCTAGCTGCGTCTCGTCCGGGGGGTCTGCGTCCACGGTAAAG
ACCCCGGGCAGCAGGCGCGCGTCGAAGTAGTCTATCTTGCATCCT
TGCAAGTCTAGCGCCTGCTGCCATGCGCGGGCGGCAAGCGCGCGC
TCGTATGGGTTGAGTGGGGGACCCCATGGCATGGGGTGGGTGAGC
GCGGAGGCGTACATGCCGCAAATGTCGTAAACGTAGAGGGGCTCT
CTGAGTATTCCAAGATATGTAGGGTAGCATCTTCCACCGCGGATG
CTGGCGCGCACGTAATCGTATAGTTCGTGCGAGGGAGCGAGGAGG
TCGGGACCGAGGTTGCTACGGGCGGGCTGCTCTGCTCGGAAGACT
ATCTGCCTGAAGATGGCATGTGAGTTGGATGATATGGTTGGACGC
TGGAAGACGTTGAAGCTGGCGTCTGTGAGACCTACCGCGTCACGC
ACGAAGGAGGCGTAGGAGTCGCGCAGCTTGTTGACCAGCTCGGCG
GTGACCTGCACGTCTAGGGCGCAGTAGTCCAGGGTTTCCTTGATG
ATGTCATACTTATCCTGTCCCTTTTTTTTCCACAGCTCGCGGTTG
AGGACAAACTCTTCGCGGTCTTTCCAGTACTCTTGGATCGGAAAC
CCGTCGGCCTCCGAACGGTAAGAGCCTAGCATGTAGAACTGGTTG
ACGGCCTGGTAGGCGCAGCATCCCTTTTCTACGGGTAGCGCGTAT
GCCTGCGCGGCCTTCCGGAGCGAGGTGTGGGTGAGCGCAAAGGTG
TCCCTGACCATGACTTTGAGGTACTGGTATTTGAAGTCAGTGTCG
TCGCATCCGCCCTGCTCCCAGAGCAAAAAGTCCGTGCGCTTTTTG
GAACGCGGATTTGGCAGGGCGAAGGTGACATCGTTGAAGAGTATC
TTTCCCGCGCGAGGCATAAAGTTGCGTGTGATGCGGAAGGGTCCC
GGCACCTCGGAACGGTTGTTAATTACCTGGGCGGCGAGCACGATC
TCGTCAAAGCCGTTGATGTTGTGGCCCACAATGTAAAGTTCCAAG
AAGCGCGGGATGCCCTTGATGGAAGGCAATTTTTTAAGTTCCTCG
TAGGTGAGCTCTTCAGGGGAGCTGAGCCCGTGCTCTGAAAGGGCC
CAGTCTGCAAGATGAGGGTTGGAAGCGACGAATGAGCTCCACAGG
TCACGGGCCATTAGCATTTGCAGGTGGTCGCGAAAGGTCCTAAAC
TGGCGACCTATGGCCATTTTTTCTGGGGTGATGCAGTAGAAGGTA
AGCGGGTCTTGTTCCCAGCGGTCCCATCCAAGGTTCGCGGCTAGG
TCTCGCGCGGCAGTCACTAGAGGCTCATCTCCGCCGAACTTCATG
ACCAGCATGAAGGGCACGAGCTGCTTCCCAAAGGCCCCCATCCAA
GTATAGGTCTCTACATCGTAGGTGACAAAGAGACGCTCGGTGCGA
GGATGCGAGCCGATCGGGAAGAACTGGATCTCCCGCCACCAATTG
GAGGAGTGGCTATTGATGTGGTGAAAGTAGAAGTCCCTGCGACGG
GCCGAACACTCGTGCTGGCTTTTGTAAAAACGTGCGCAGTACTGG
CAGCGGTGCACGGGCTGTACATCCTGCACGAGGTTGACCTGACGA
CCGCGCACAAGGAAGCAGAGTGGGAATTTGAGCCCCTCGCCTGGC
GGGTTTGGCTGGTGGTCTTCTACTTCGGCTGCTTGTCCTTGACCG
TCTGGCTGCTCGAGGGGAGTTACGGTGGATCGGACCACCACGCCG
CGCGAGCCCAAAGTCCAGATGTCCGCGCGCGGCGGTCGGAGCTTG
ATGACAACATCGCGCAGATGGGAGCTGTCCATGGTCTGGAGCTCC
CGCGGCGTCAGGTCAGGCGGGAGCTCCTGCAGGTTTACCTCGCAT
AGACGGGTCAGGGCGCGGGCTAGATCCAGGTGATACCTAATTTCC
AGGGGCTGGTTGGTGGCGGCGTCGATGGCTTGCAAGAGGCCGCAT
CCCCGCGGCGCGACTACGGTACCGCGCGGCGGGCGGTGGGCCGCG
GGGGTGTCCTTGGATGATGCATCTAAAAGCGGTGACGCGGGCGAG
CCCCCGGAGGTAGGGGGGGCTCCGGACCCGCCGGGAGAGGGGGCA
GGGGCACGTCGGCGCCGCGCGCGGGCAGGAGCTGGTGCTGCGCGC
GTAGGTTGCTGGCGAACGCGACGACGCGGCGGTTGATCTCCTGAA
TCTGGCGCCTCTGCGTGAAGACGACGGGCCCGGTGAGCTTGAACC
TGAAAGAGAGTTCGACAGAATCAATTTCGGTGTCGTTGACGGCGG
CCTGGCGCAAAATCTCCTGCACGTCTCCTGAGTIGTCTTGATAGG
CGATCTCGGCCATGAACTGCTCGATCTCTTCCTCCTGGAGATCTC
CGCGTCCGGCTCGCTCCACGGTGGCGGCGAGGTCGTTGGAAATGC
GGGCCATGAGCTGCGAGAAGGCGTTGAGGCCTCCCTCGTTCCAGA
CGCGGCTGTAGACCACGCCCCCTTCGGCATCGCGGGCGCGCATGA
CCACCTGCGCGAGATTGAGCTCCACGTGCCGGGCGAAGACGGCGT
AGTTTCGCAGGCGCTGAAAGAGGTAGTTGAGGGTGGTGGCGGTGT
GTTCTGCCACGAAGAAGTACATAACCCAGCGTCGCAACGTGGATT
CGTTGATATCCCCCAAGGCCTCAAGGCGCTCCATGGCCTCGTAGA
AGTCCACGGCGAAGTTGAAAAACTGGGAGTTGCGCGCCGACACGG
TTAACTCCTCCTCCAGAAGACGGATGAGCTCGGCGACAGTGTCGC
GCACCTCGCGCTCAAAGGCTACAGGGGCCTCTTCTTCTTCTTCAA
TCTCCTCTTCCATAAGGGCCTCCCCTTCTTCTTCTTCTGGCGGCG
GTGGGGGAGGGGGGACACGGCGGCGACGACGGCGCACCGGGAGGC
GGTCGACAAAGCGCTCGATCATCTCCCCGCGGCGACGGCGCATGG
TCTCGGTGACGGCGCGGCCGTTCTCGCGGGGGCGCAGTTGGAAGA
CGCCGCCCGTCATGTCCCGGTTATGGGTTGGCGGGGGGCTGCCAT
GCGGCAGGGATACGGCGCTAACGATGCATCTCAACAATTGTTGTG
TAGGTACTCCGCCGCCGAGGGACCTGAGCGAGTCCGCATCGACCG
GATCGGAAAACCTCTCGAGAAAGGCGTCTAACCAGTCACAGTCGC
AAGGTAGGCTGAGCACCGTGGCGGGCGGCAGCGGGCGGCGGTCGG
GGTTGTTTCTGGCGGAGGTGCTGCTGATGATGTAATTAAAGTAGG
CGGTCTTGAGACGGCGGATGGTCGACAGAAGCACCATGTCCTTGG
GTCCGGCCTGCTGAATGCGCAGGCGGTCGGCCATGCCCCAGGCTT
CGTTTTGACATCGGCGCAGGTCTTTGTAGTAGTCTTGCATGAGCC
TTTCTACCGGCACTTCTTCTTCTCCTTCCTCTTGTCCTGCATCTC
TTGCATCTATCGCTGCGGCGGCGGCGGAGTTTGGCCGTAGGTGGC
GCCCTCTTCCTCCCATGCGTGTGACCCCGAAGCCCCTCATCGGCT
GAAGCAGGGCTAGGTCGGCGACAACGCGCTCGGCTAATATGGCCT
GCTGCACCTGCGTGAGGGTAGACTGGAAGTCATCCATGTCCACAA
AGCGGTGGTATGCGCCCGTGTTGATGGTGTAAGTGCAGTTGGCCA
TAACGGACCAGTTAACGGTCTGGTGACCCGGCTGCGAGAGCTCGG
TGTACCTGAGACGCGAGTAAGCCCTCGAGTCAAATACGTAGTCGT
TGCAAGTCCGCACCAGGTACTGGTATCCCACCAAAAAGTGCGGCG
GCGGCTGGCGGTAGAGGGGCCAGCGTAGGGTGGCCGGGGCTCCGG
GGGCGAGATCTTCCAACATAAGGCGATGATATCCGTAGATGTACC
TGGACATCCAGGTGATGCCGGCGGCGGTGGTGGAGGCGCGCGGAA
AGTCGCGGACGCGGTTCCAGATGTTGCGCAGCGGCAAAAAGTGCT
CCATGGTCGGGACGCTCTGGCCGGTCAGGCGCGCGCAATCGTTGA
CGCTCTAGCGTGCAAAAGGAGAGCCTGTAAGCGGGCACTCTTCCG
TGGTCTGGTGGATAAATTCGCAAGGGTATCATGGCGGACGACCGG
GGTTCGAGCCCCGTATCCGGCCGTCCGCCGTGATCCATGCGGTTA
CCGCCCGCGTGTCGAACCCAGGTGTGCGACGTCAGACAACGGGGG
AGTGCTCCTTTTGGCTTCCTTCCAGGCGCGGCGGCTGCTGCGCTA
GCTTTTTTGGCCACTGGCCGCGCGCAGCGTAAGCGGTTAGGCTGG
AAAGCGAAAGCATTAAGTGGCTCGCTCCCTGTAGCCGGAGGGTTA
TTTTCCAAGGGTTGAGTCGCGGGACCCCCGGTTCGAGTCTCGGAC
CGGCCGGACTGCGGCGAACGGGGGTTTGCCTCCCCGTCATGCAAG
ACCCCGCTTGCAAATTCCTCCGGAAACAGGGACGAGCCCCTTTTT
TGCTTTTCCCAGATGCATCCGGTGCTGCGGCAGATGCGCCCCCCT
CCTCAGCAGCGGCAAGAGCAAGAGCAGCGGCAGACATGCAGGGCA
CCCTCCCCTCCTCCTACCGCGTCAGGAGGGGCGACATCCGCGGTT
GACGCGGCAGCAGATGGTGATTACGAACCCCCGCGGCGCCGGGCC
CGGCACTACCTGGACTTGGAGGAGGGCGAGGGCCTGGCGCGGCTA
GGAGCGCCCTCTCCTGAGCGGCACCCAAGGGTGCAGCTGAAGCGT
GATACGCGTGAGGCGTACGTGCCGCGGCAGAACCTGTTTCGCGAC
CGCGAGGGAGAGGAGCCCGAGGAGATGCGGGATCGAAAGTTCCAC
GCAGGGCGCGAGCTGCGGCATGGCCTGAATCGCGAGCGGTTGCTG
CGCGAGGAGGACTTTGAGCCCGACGCGCGAACCGGGATTAGTCCC
GCGCGCGCACACGTGGCGGCCGCCGACCTGGTAACCGCATACGAG
CAGACGGTGAACCAGGAGATTAACTTTCAAAAAAGCTTTAACAAC
CACGTGCGTACGCTTGTGGCGCGCGAGGAGGTGGCTATAGGACTG
ATGCATCTGTGGGACTTTGTAAGCGCGCTGGAGCAAAACCCAAAT
AGCAAGCCGCTCATGGCGCAGCTGTTCCTTATAGTGCAGCACAGC
AGGGACAACGAGGCATTCAGGGATGCGCTGCTAAACATAGTAGAG
CCCGAGGGCCGCTGGCTGCTCGATTTGATAAACATCCTGCAGAGC
ATAGTGGTGCAGGAGCGCAGCTTGAGCCTGGCTGACAAGGTGGCC
GCCATCAACTATTCCATGCTTAGCCTGGGCAAGTTTTACGCCCGC
AAGATATACCATACCCCTTACGTTCCCATAGACAAGGAGGTAAAG
ATCGAGGGGTTCTACATGCGCATGGCGCTGAAGGTGCTTACCTTG
AGCGACGACCTGGGCGTTTATCGCAACGAGCGCATCCACAAGGCC
GTGAGCGTGAGCCGGCGGCGCGAGCTCAGCGACCGCGAGCTGATG
CACAGCCTGCAAAGGGCCCTGGCTGGCACGGGCAGCGGCGATAGA
GAGGCCGAGTCCTACTTTGACGCGGGCGCTGACCTGCGCTGGGCC
CCAAGCCGACGCGCCCTGGAGGCAGCTGGGGCCGGACCTGGGCTG
GCGGTGGCACCCGCGCGCGCTGGCAACGTCGGCGGCGTGGAGGAA
TATGACGAGGACGATGAGTACGAGCCAGAGGACGGCGAGTACTAA
GCGGTGATGTTTCTGATCAGATGATGCAAGACGCAACGGACCCGG
CGGTGCGGGCGGCGCTGCAGAGCCAGCCGTCCGGCCTTAACTCCA
CGGACGACTGGCGCCAGGTCATGGACCGCATCATGTCGCTGACTG
CGCGCAATCCTGACGCGTTCCGGCAGCAGCCGCAGGCCAACCGGC
TCTCCGCAATTCTGGAAGCGGTGGTCCCGGCGCGCGCAAACCCCA
CGCACGAGAAGGTGCTGGCGATCGTAAACGCGCTGGCCGAAAACA
GGGCCATCCGGCCCGACGAGGCCGGCCTGGTCTACGACGCGCTGC
TTCAGCGCGTGGCTCGTTACAACAGCGGCAACGTGCAGACCAACC
TGGACCGGCTGGTGGGGGATGTGCGCGAGGCCGTGGCGCAGCGTG
AGCGCGCGCAGCAGCAGGGCAACCTGGGCTCCATGGTTGCACTAA
ACGCCTTCCTGAGTACACAGCCCGCCAACGTGCCGCGGGGACAGG
AGGACTACACCAACTTTGTGAGCGCACTGCGGCTAATGGTGACTG
AGACACCGCAAAGTGAGGTGTACCAGTCTGGGCCAGACTATTTTT
TCCAGACCAGTAGACAAGGCCTGCAGACCGTAAACCTGAGCCAGG
CTTTCAAAAACTTGCAGGGGCTGTGGGGGGTGCGGGCTCCCACAG
GCGACCGCGCGACCGTGTCTAGCTTGCTGACGCCCAACTCGCGCC
TGTTGCTGCTGCTAATAGCGCCCTTCACGGACAGTGGCAGCGTGT
CCCGGGACACATACCTAGGTCACTTGCTGACACTGTACCGCGAGG
CCATAGGTCAGGCGCATGTGGACGAGCATACTTTCCAGGAGATTA
CAAGTGTCAGCCGCGCGCTGGGGCAGGAGGACACGGGCAGCCTGG
AGGCAACCCTAAACTACCTGCTGACCAACCGGCGGCAGAAGATCC
CCTCGTTGCACAGTTTAAACAGCGAGGAGGAGCGCATTTTGCGCT
ACGTGCAGCAGAGCGTGAGCCTTAACCTGATGCGCGACGGGGTAA
CGCCCAGCGTGGCGCTGGACATGACCGCGCGCAACATGGAACCGG
GCATGTATGCCTCAAACCGGCCGTTTATCAACCGCCTAATGGACT
ACTTGCATCGCGCGGCCGCCGTGAACCCCGAGTATTTCACCAATG
CCATCTTGAACCCGCACTGGCTACCGCCCCCTGGTTTCTACACCG
GGGGATTCGAGGTGCCCGAGGGTAACGATGGATTCCTCTGGGACG
ACATAGACGACAGCGTGTTTTCCCCGCAACCGCAGACCCTGCTAG
AGTTGCAACAGCGCGAGCAGGCAGAGGCGGCGCTGCGAAAGGAAA
GCTTCCGCAGGCCAAGCAGCTTGTCCGATCTAGGCGCTGCGGCCC
CGCGGTCAGATGCTAGTAGCCCATTTCCAAGCTTGATAGGGTCTC
TTACCAGCACTCGCACCACCCGCCCGCGCCTGCTGGGCGAGGAGG
AGTACCTAAACAACTCGCTGCTGCAGCCGCAGCGCGAAAAAAACC
TGCCTCCGGCATTTCCCAACAACGGGATAGAGAGCCTAGTGGACA
AGATGAGTAGATGGAAGACGTACGCGCAGGAGCACAGGGACGTGC
CAGGCCCGCGCCCGCCCACCCGTCGTCAAAGGCACGACCGTCAGC
GGGGTCTGGTGTGGGAGGACGATGACTCGGCAGACGACAGCAGCG
TCCTGGATTTGGGAGGGAGTGGCAACCCGTTTGCGCACCTTCGCC
CCAGGCTGGGGAGAATGTTTTAAAAAAAAAAAAGCATGATGCAAA
AAAAAAACTCACCAAGGCCATGGCACCGAGCGTTGGTTTTCTTGT
ATTCCCCTTAGTATGCGGCGCGCGGCGATGTATGAGGAAGGTCCT
CCTCCCTCCTACGAGAGTGTGGTGAGCGCGGCGCCAGTGGCGGCG
GCGCTGGGTTCTCCCTTCGATGCTCCCCTGGACCCGCCGTTTGTG
CCTCCGCGGTACCTGCGGCCTACCGGGGGGAGAAACAGCATCCGT
TACTCTGAGTTGGCACCCCTATTCGACACCACCCGTGTGTACCTG
GTGGACAACAAGTCAACGGATGTGGCATCCCTGAACTACCAGAAC
GACCACAGCAACTTTCTGACCACGGTCATTCAAAACAATGACTAC
AGCCCGGGGGAGGCAAGCACACAGACCATCAATCTTGACGACCGG
TCGCACTGGGGCGGCGACCTGAAAACCATCCTGCATACCAACATG
CCAAATGTGAACGAGTTCATGTTTACCAATAAGTTTAAGGCGCGG
GTGATGGTGTCGCGCTTGCCTACTAAGGACAATCAGGTGGAGCTG
AAATACGAGTGGGTGGAGTTCACGCTGCCCGAGGGCAACTACTCC
GAGACCATGACCATAGACCTTATGAACAACGCGATCGTGGAGCAC
TACTTGAAAGTGGGCAGACAGAACGGGGTTCTGGAAAGCGACATC
GGGGTAAAGTTTGACACCCGCAACTTCAGACTGGGGTTTGACCCC
GTCACTGGTCTTGTCATGCCTGGGGTATATACAAACGAAGCCTTC
CATCCAGACATCATTTTGCTGCCAGGATGCGGGGTGGACTTCACC
CACAGCCGCCTGAGCAACTTGTTGGGCATCCGCAAGCGGCAACCC
TTCCAGGAGGGCTTTAGGATCACCTACGATGATCTGGAGGGTGGT
AACATTCCCGCACTGTTGGATGTGGACGCCTACCAGGCGAGCTTG
AAAGATGACACCGAACAGGGCGGGGGTGGCGCAGGCGGCAGCAAC
AGCAGTGGCAGCGGCGCGGAAGAGAACTCCAACGCGGCAGCCGCG
GCAATGCAGCCGGTGGAGGACATGAACGATCATGCCATTCGCGGC
GACACCTTTGCCACACGGGCTGAGGAGAAGCGCGCTGAGGCCGAA
GCAGCGGCCGAAGCTGCCGCCCCCGCTGCGCAACCCGAGGTCGAG
AAGCCTCAGAAGAAACCGGTGATCAAACCCCTGACAGAGGACAGC
AAGAAACGCAGTTACAACCTAATAAGCAATGACAGCACCTTCACC
CAGTACCGCAGCTGGTACCTTGCATACAACTACGGCGACCCTCAG
ACCGGAATCCGCTCATGGACCCTGCTTTGCACTCCTGACGTAACC
TGCGGCTCGGAGCAGGTCTACTGGTCGTTGCCAGACATGATGCAA
GACCCCGTGACCTTCCGCTCCACGCGCCAGATCAGCAACTTTCCG
GTGGTGGGCGCCGAGCTGTTGCCCGTGCACTCCAAGAGCTTCTAC
AACGACCAGGCCGTCTACTCCCAACTCATCCGCCAGTTTACCTCT
CTGACCCACGTGTTCAATCGCTTTCCCGAGAACCAGATTTTGGCG
CGCCCGCCAGCCCCCACCATCACCACCGTCAGTGAAAACGTTCCT
GCTCTCACAGATCACGGGACGCTACCGCTGCGCAACAGCATCGGA
GGAGTCCAGCGAGTGACCATTACTGACGCCAGACGCCGCACCTGC
CCCTACGTTTACAAGGCCCTGGGCATAGTCTCGCCGCGCGTCCTA
TCGAGCCGCACTTTTTGAGCAAGCATGTCCATCCTTATATCGCCC
AGCAATAACACAGGCTGGGGCCTGCGCTTCCCAAGCAAGATGTTT
GGCGGGGCCAAGAAGCGCTCCGACCAACACCCAGTGCGCGTGCGC
GGGCACTACCGCGCGCCCTGGGGCGCGCACAAACGCGGCCGCACT
GGGCGCACCACCGTCGATGACGCCATCGACGCGGTGGTGGAGGAG
GCGCGCAACTACACGCCCACGCCGCCACCAGTGTCCACAGTGGAC
GCGGCCATTCAGACCGTGGTGCGCGGAGCCCGGCGCTATGCTAAA
ATGAAGAGACGGCGGAGGCGCGTAGCACGTCGCCACCGCCGCCGA
CCCGGCACTGCCGCCCAACGCGCGGCGGCGGCCCTGCTTAACCGC
GCACGTCGCACCGGCCGACGGGCGGCCATGCGGGCCGCTCGAAGG
CTGGCCGCGGGTATTGTCACTGTGCCCCCCAGGTCCAGGCGACGA
GCGGCCGCCGCAGCAGCCGCGGCCATTAGTGCTATGACTCAGGGT
CGCAGGGGCAACGTGTATTGGGTGCGCGACTCGGTTAGCGGCCTG
CGCGTGCCCGTGCGCACCCGCCCCCCGCGCAACTAGATTGCAAGA
AAAAACTACTTAGACTCGTACTGTTGTATGTATCCAGCGGCGGCG
GCGCGCAACGAAGCTATGTCCAAGCGCAAAATCAAAGAAGAGATG
CTCCAGGTCATCGCGCCGGAGATCTATGGCCCCCCGAAGAAGGAA
GAGCAGGATTACAAGCCCCGAAAGCTAAAGCGGGTCAAAAAGAAA
AAGAAAGATGATGATGATGAACTTGACGACGAGGTGGAACTGCTG
CACGCTACCGCGCCCAGGCGACGGGTACAGTGGAAAGGTCGACGC
GTAAAACGTGTTTTGCGACCCGGCACCACCGTAGTCTTTACGCCC
GGTGAGCGCTCCACCCGCACCTACAAGCGCGTGTATGATGAGGTG
TACGGCGACGAGGACCTGCTTGAGCAGGCCAACGAGCGCCTCGGG
GAGTTTGCCTACGGAAAGCGGCATAAGGACATGCTGGCGTTGCCG
CTGGACGAGGGCAACCCAACACCIAGCCTAAAGCCCGTAACACTG
CAGCAGGTGCTGCCCGCGCTTGCACCGTCCGAAGAAAAGCGCGGC
CTAAAGCGCGAGTCTGGTGACTTGGCACCCACCGTGCAGCTGATG
GTACCCAAGCGCCAGCGACTGGAAGATGTCTTGGAAAAAATGACC
GTGGAACCTGGGCTGGAGCCCGAGGTCCGCGTGCGGCCAATCAAG
CAGGTGGCGCCGGGACTGGGCGTGCAGACCGTGGACGTTCAGATA
CCCACTACCAGTAGCACCAGTATTGCCACCGCCACAGAGGGCATG
GAGACACAAACGTCCCCGGTTGCCTCAGCGGTGGCGGATGCCGCG
GTGCAGGCGGTCGCTGCGGCCGCGTCCAAGACCTCTACGGAGGTG
CAAACGGACCCGTGGATGTTTCGCGTTTCAGCCCCCCGGCGCCCG
CGCCGTTCGAGGAAGTACGGCGCCGCCAGCGCGCTACTGCCCGAA
TATGCCCTACATCCTTCCATTGCGCCTACCCCCGGCTATCGTGGC
TACACCTACCGCCCCAGAAGACGAGCAACTACCCGACGCCGAACC
ACCACTGGAACCCGCCGCCGCCGTCGCCGTCGCCAGCCCGTGCTG
GCCCCGATTTCCGTGCGCAGGGTGGCTCGCGAAGGAGGCAGGACC
CTGGTGCTGCCAACAGCGCGCTACCACCCCAGCATCGTTTAAAAG
CCGGTCTTTGTGGTTCTTGCAGATATGGCCCTCACCTGCCGCCTC
CGTTTCCCGGTGCCGGGATTCCGAGGAAGAATGCACCGTAGGAGG
GGCATGGCCGGCCACGGCCTGACGGGCGGCATGCGTCGTGCGCAC
CACCGGCGGCGGCGCGCGTCGCACCGTCGCATGCGCGGCGGTATC
CTGCCCCTCCTTATTCCACTGATCGCCGCGGCGATTGGCGCCGTG
CCCGGAATTGCATCCGTGGCCTTGCAGGCGCAGAGACACTGATTA
AAAACAAGTTGCATGTGGAAAAATCAAAATAAAAAGTCTGGACTC
TCACGCTCGCTTGGTCCTGTAACTATTTTGTAGAATGGAAGACAT
CAACTTTGCGTCTCTGGCCCCGCGACACGGCTCGCGCCCGTTCAT
GGGAAACTGGCAAGATATCGGCACCAGCAATATGAGCGGTGGCGC
CTTCAGCTGGGGCTCGCTGTGGAGCGGCATTAAAAATTTCGGTTC
CACCGTTAAGAACTATGGCAGCAAGGCCTGGAACAGCAGCACAGG
CCAGATGCTGAGGGATAAGTTGAAAGAGCAAAATTTCCAACAAAA
GGTGGTAGATGGCCTGGCCTCTGGCATTAGCGGGGTGGTGGACCT
GGCCAACCAGGCAGTGCAAAATAAGATTAACAGTAAGCTTGATCC
CCGCCCTCCCGTAGAGGAGCCTCCACCGGCCGTGGAGACAGTGTC
TCCAGAGGGGCGTGGCGAAAAGCGTCCGCGCCCCGACAGGGAAGA
AACTCTGGTGACGCAAATAGACGAGCCTCCCTCGTACGAGGAGGC
ACTAAAGCAAGGCCTGCCCACCACCCGTCCCATCGCGCCCATGGC
TACCGGAGTGCTGGGCCAGCACACACCCGTAACGCTGGACCTGCC
TCCCCCCGCCGACACCCAGCAGAAACCTGTGCTGCCAGGCCCGAC
CGCCGTTGTTGTAACCCGTCCTAGCCGCGCGTCCCTGCGCCGCGC
CGCCAGCGGTCCGCGATCGTTGCGGCCCGTAGCCAGTGGCAACTG
GCAAAGCACACTGAACAGCATCGTGGGTCTGGGGGTGCAATCCCT
GAAGCGCCGACGATGCTTCTGATAGCTAACGTGTCGTATGTGTGT
CATGTATGCGTCCATGTCGCCGCCAGAGGAGCTGCTGAGCCGCCG
CGCGCCCGCTTTCCAAGATGGCTACCCCTTCGATGATGCCGCAGT
GGTCTTACATGCACATCTCGGGCCAGGACGCCTCGGAGTACCTGA
GCCCCGGGCTGGTGCAGTTTGCCCGCGCCACCGAGACGTACTTCA
GCCTGAATAACAAGTTTAGAAACCCCACGGTGGCGCCTACGCACG
ACGTGACCACAGACCGGTCCCAGCGTTTGACGCTGCGGTTCATCC
CTGTGGACCGTGAGGATACTGCGTACTCGTACAAGGCGCGGTTCA
CCCTAGCTGTGGGTGATAACCGTGTGCTGGACATGGCTTCCACGT
ACTTTGACATCCGCGGCGTGCTGGACAGGGGCCCTACTTTTAAGC
CCTACTCTGGCACTGCCTACAACGCCCTGGCTCCCAAGGGTGCCC
CAAATCCTTGCGAATGGGATGAAGCTGCTACTGCTCTTGAAATAA
ACCTAGAAGAAGAGGACGATGACAACGAAGACGAAGTAGACGAGC
AAGCTGAGCAGCAAAAAACTCACGTATTTGGGCAGGCGCCTTATT
CTGGTATAAATATTACAAAGGAGGGTATTCAAATAGGTGTCGAAG
GTCAAACACCTAAATATGCCGATAAAACATTTCAACCTGAACCTC
AAATAGGAGAATCTCAGTGGTACGAAACAGAAATTAATCATGCAG
CTGGGAGAGTCCTAAAAAAGACTACCCCAATGAAACCATGTTACG
GTTCATATGCAAAACCCACAAATGAAAATGGAGGGCAAGGCATTC
TTGTAAAGCAACAAAATGGAAAGCTAGAAAGTCAAGTGGAAATGC
AATTTTTCTCAACTACTGAGGCAGCCGCAGGCAATGGTGATAACT
TGACTCCTAAAGTGGTATTGTACAGTGAAGATGTAGATATAGAAA
CCCCAGACACTCATATTTCTTACATGCCCACTATTAAGGAAGGTA
ACTCACGAGAACTAATGGGCCAACAATCTATGCCCAACAGGCCTA
ATTACATTGCTTTTAGGGACAATTTTATTGGTCTAATGTATTACA
ACAGCACGGGTAATATGGGTGTTCTGGCGGGCCAAGCATCGCAGT
TGAATGCTGTTGTAGATTTGCAAGACAGAAACACAGAGCTTTCAT
ACCAGCTTTTGCTTGATTCCATTGGTGATAGAACCAGGTACTTTT
CTATGTGGAATCAGGCTGTTGACAGCTATGATCCAGATGTTAGAA
TTATTGAAAATCATGGAACTGAAGATGAACTTCCAAATTACTGCT
TTCCACTGGGAGGTGTGATTAATACAGAGACTCTTACCAAGGTAA
AACCTAAAACAGGTCAGGAAAATGGATGGGAAAAAGATGCTACAG
AATTTTCAGATAAAAATGAAATAAGAGTTGGAAATAATTTTGCCA
TGGAAATCAATCTAAATGCCAACCTGTGGAGAAATTTCCTGTACT
CCAACATAGCGCTGTATTTGCCCGACAAGCTAAAGTACAGTCCTT
CCAACGTAAAAATTTCTGATAACCCAAACACCTACGACTACATGA
ACAAGCGAGTGGTGGCTCCCGGGCTAGTGGACTGCTACATTAACC
TTGGAGCACGCTGGTCCCTTGACTATATGGACAACGTCAACCCAT
TTAACCACCACCGCAATGCTGGCCTGCGCTACCGCTCAATGTTGC
TGGGCAATGGTCGCTATGTGCCCTTCCACATCCAGGTGCCTCAGA
AGTTCTTTGCCATTAAAAACCTCCTTCTCCTGCCGGGCTCATACA
CCTACGAGTGGAACTTCAGGAAGGATGTTAACATGGTTCTGCAGA
GCTCCCTAGGAAATGACCTAAGGGTTGACGGAGCCAGCATTAAGT
TTGATAGCATTTGCCTTTACGCCACCTTCTTCCCCATGGCCCACA
ACACCGCCTCCACGCTTGAGGCCATGCTTAGAAACGACACCAACG
ACCAGTCCTTTAACGACTATCTCTCCGCCGCCAACATGCTCTACC
CTATACCCGCCAACGCTACCAACGTGCCCATATCCATCCCCTCCC
GCAACTGGGCGGCTTTCCGCGGCTGGGCCTTCACGCGCCTTAAGA
CTAAGGAAACCCCATCACTGGGCTCGGGCTACGACCCTTATTACA
CCTACTCTGGCTCTATACCCTACCTAGATGGAACCTTTTACCTCA
ACCACACCTTTAAGAAGGTGGCCATTACCTTTGACTCTTCTGTCA
GCTGGCCTGGCAATGACCGCCTGCTTACCCCCAACGAGTTTGAAA
TTAAGCGCTCAGTTGACGGGGAGGGTTACAACGTTGCCCAGTGTA
ACATGACCAAAGACTGGTTCCTGGTACAAATGCTAGCTAACTATA
ACATTGGCTACCAGGGCTTCTATATCCCAGAGAGCTACAAGGACC
GCATGTACTCCTTCTTTAGAAACTTCCAGCCCATGAGCCGTCAGG
TGGTGGATGATACTAAATACAAGGACTACCAACAGGTGGGCATCC
TACACCAACACAACAACTCTGGATTTGTTGGCTACCTTGCCCCCA
CCATGCGCGAAGGACAGGCCTACCCTGCTAACTTCCCCTATCCGC
TTATAGGCAAGACCGCAGTTGACAGCATTACCCAGAAAAAGTTTC
TTTGCGATCGCACCCTTTGGCGCATCCCATTCTCCAGTAACTTTA
TGTCCATGGGCGCACTCACAGACCTGGGCCAAAACCTTCTCTACG
CCAACTCCGCCCACGCGCTAGACATGACTTTTGAGGTGGATCCCA
TGGACGAGCCCACCCTTCTTTATGTTTTGTTTGAAGTCTTTGACG
TGGTCCGTGTGCACCAGCCGCACCGCGGCGTCATCGAAACCGTGT
ACCTGCGCACGCCCTTCTCGGCCGGCAACGCCACAACATAAAGAA
GCAAGCAACATCAACAACAGCTGCCGCCATGGGCTCCAGTGAGCA
GGAACTGAAAGCCATTGTCAAAGATCTTGGTTGTGGGCCATATTT
TTTGGGCACCTATGACAAGCGCTTTCCAGGCTTTGTTTCTCCACA
CAAGCTCGCCTGCGCCATAGTCAATACGGCCGGTCGCGAGACTGG
GGGCGTACACTGGATGGCCTTTGCCTGGAACCCGCACTCAAAAAC
ATGCTACCTCTTTGAGCCCTTTGGCTTTTCTGACCAGCGACTCAA
GCAGGTTTACCAGTTTGAGTACGAGTCACTCCTGCGCCGTAGCGC
CATTGCTTCTTCCCCCGACCGCTGTATAACGCTGGAAAAGTCCAC
CCAAAGCGTACAGGGGCCCAACTCGGCCGCCTGTGGACTATTCTG
CTGCATGTTTCTCCACGCCTTTGCCAACTGGCCCCAAACTCCCAT
GGATCACAACCCCACCATGAACCTTATTACCGGGGTACCCAACTC
CATGCTCAACAGTCCCCAGGTACAGCCCACCCTGCGTCGCAACCA
GGAACAGCTCTACAGCTTCCTGGAGCGCCACTCGCCCTACTTCCG
CAGCCACAGTGCGCAGATTAGGAGCGCCACTTCTTTTTGTCACTT
GAAAAACATGTAAAAATAATGTACTAGAGACACTTTCAATAAAGG
CAAATGCTTTTATTTGTACACTCTCGGGTGATTATTTACCCCCAC
CCTTGCCGTCTGCGCCGTTTAAAAATCAAAGGGGTTCTGCCGCGC
ATCGCTATGCGCCACTGGCAGGGACACGTTGCGATACTGGTGTTT
AGTGCTCCACTTAAACTCAGGCACAACCATCCGCGGCAGCTCGGT
GAAGTTTTCACTCCACAGGCTGCGCACCATCACCAACGCGTTTAG
CAGGTCGGGCGCCGATATCTTGAAGTCGCAGTTGGGGCCTCCGCC
CTGCGCGCGCGAGTTGCGATACACAGGGTTGCAGCACTGGAACAC
TATCAGCGCCGGGTGGTGCACGCTGGCCAGCACGCTCTTGTCGGA
GATCAGATCCGCGTCCAGGTCCTCCGCGTTGCTCAGGGCGAACGG
AGTCAACTTTGGTAGCTGCCTTCCCAAAAAGGGCGCGTGCCCAGG
CTTTGAGTTGCACTCGCACCGTAGTGGCATCAAAAGGTGACCGTG
CCCGGTCTGGGCGTTAGGATACAGCGCCTGCATAAAAGCCTTGAT
CTGCTTAAAAGCCACCTGAGCCTTTGCGCCTTCAGAGAAGAACAT
GCCGCAAGACTTGCCGGAAAACTGATTGGCCGGACAGGCCGCGTC
GTGCACGCAGCACCTTGCGTCGGTGTTGGAGATCTGCACCACATT
TCGGCCCCACCGGTTCTTCACGATCTTGGCCTTGCTAGACTGCTC
CTTCAGCGCGCGCTGCCCGTTTTCGCTCGTCACATCCATTTCAAT
CACGTGCTCCTTATTTATCATAATGCTTCCGTGTAGACACTTAAG
CTCGCCTTCGATCTCAGCGCAGCGGTGCAGCCACAACGCGCAGCC
CGTGGGCTCGTGATGCTTGTAGGTCACCTCTGCAAACGACTGCAG
GTACGCCTGCAGGAATCGCCCCATCATCGTCACAAAGGTCTTGTT
GCTGGTGAAGGTCAGCTGCAACCCGCGGTGCTCCTCGTTCAGCCA
GGTCTTGCATACGGCCGCCAGAGCTTCCACTTGGTCAGGCAGTAG
TTTGAAGTTCGCCTTTAGATCGTTATCCACGTGGTACTTGTCCAT
CAGCGCGCGCGCAGCCTCCATGCCCTTCTCCCACGCAGACACGAT
CGGCACACTCAGCGGGTTCATCACCGTAATTTCACTTTCCGCTTC
GCTGGGCTCTTCCTCTTCCTCTTGCGTCCGCATACCACGCGCCAC
TGGGTCGTCTTCATTCAGCCGCCGCACTGTGCGCTTACCTCCTTT
GCCATGCTTGATTAGCACCGGTGGGTTGCTGAAACCCACCATTTG
TAGCGCCACATCTTCTCTTTCTTCCTCGCTGTCCACGATTACCTC
TGGTGATGGCGGGCGCTCGGGCTTGGGAGAAGGGCGCTTCTTTTT
CTTCTTGGGCGCAATGGCCAAATCCGCCGCCGAGGTCGATGGCCG
CGGGCTGGGTGTGCGCGGCACCAGCGCGTCTTGTGATGAGTCTTC
CTCGTCCTCGGACTCGATACGCCGCCTCATCCGCTTTTTTGGGGG
CGCCCGGGGAGGCGGCGGCGACGGGGACGGGGACGACACGTCCTC
CATGGTTGGGGGACGTCGCGCCGCACCGCGTCCGCGCTCGGGGGT
GGTTTCGCGCTGCTCCTCTTCCCGACTGGCCATTTCCTTCTCCTA
TAGGCAGAAAAAGATCATGGAGTCAGTCGAGAAGAAGGACAGCCT
AACCGCCCCCTCTGAGTTCGCCACCACCGCCTCCACCGATGCCGC
CAACGCGCCTACCACCTTCCCCGTCGAGGCACCCCCGCTTGAGGA
GGAGGAAGTGATTATCGAGCAGGACCCAGGTTTTGTAAGCGAAGA
CGACGAGGACCGCTCAGTACCAACAGAGGATAAAAAGCAAGACCA
GGACAACGCAGAGGCAAACGAGGAACAAGTCGGGCGGGGGGACGA
AAGGCATGGCGACTACCTAGATGTGGGAGACGACGTGCTGTTGAA
GCATCTGCAGCGCCAGTGCGCCATTATCTGCGACGCGTTGCAAGA
GCGCAGCGATGTGCCCCTCGCCATAGCGGATGTCAGCCTTGCCTA
CGAACGCCACCTATTCTCACCGCGCGTACCCCCCAAACGCCAAGA
AAACGGCACATGCGAGCCCAACCCGCGCCTCAACTTCTACCCCGT
ATTTGCCGTGCCAGAGGTGCTTGCCACCTATCACATCTTTTTCCA
AAACTGCAAGATACCCCTATCCTGCCGTGCCAACCGCAGCCGAGC
GGACAAGCAGCTGGCCTTGCGGCAGGGCGCTGTCATACCTGATAT
CGCCTCGCTCAACGAAGTGCCAAAAATCTTTGAGGGTCTTGGACG
CGACGAGAAGCGCGCGGCAAACGCTCTGCAACAGGAAAACAGCGA
AAATGAAAGTCACTCTGGAGTGTTGGTGGAACTCGAGGGTGACAA
CGCGCGCCTAGCCGTACTAAAACGCAGCATCGAGGTCACCCACTT
TGCCTACCCGGCACTTAACCTACCCCCCAAGGTCATGAGCACAGT
CATGAGTGAGCTGATCGTGCGCCGTGCGCAGCCCCTGGAGAGGGA
TGCAAATTTGCAAGAACAAACAGAGGAGGGCCTACCCGCAGTTGG
CGACGAGCAGCTAGCGCGCTGGCTTCAAACGCGCGAGCCTGCCGA
CTTGGAGGAGCGACGCAAACTAATGATGGCCGCAGTGCTCGTTAC
CGTGGAGCTTGAGTGCATGCAGCGGTTCTTTGCTGACCCGGAGAT
GCAGCGCAAGCTAGAGGAAACATTGCACTACACCTTTCGACAGGG
CTACGTACGCCAGGCCTGCAAGATCTCCAACGTGGAGCTCTGCAA
CCTGGTCTCCTACCTTGGAATTTTGCACGAAAACCGCCTTGGGCA
AAACGTGCTTCATTCCACGCTCAAGGGCGAGGCGCGCCGCGACTA
CGTCCGCGACTGCGTTTACTTATTTCTATGCTACACCTGGCAGAC
GGCCATGGGCGTTTGGCAGCAGTGCTTGGAGGAGTGCAACCTCAA
GGAGCTGCAGAAACTGCTAAAGCAAAACTTGAAGGACCTATGGAC
GGCCTTCAACGAGCGCTCCGTGGCCGCGCACCTGGCGGACATCAT
TTTCCCCGAACGCCTGCTTAAAACCCTGCAACAGGGTCTGCCAGA
CTTCACCAGTCAAAGCATGTTGCAGAACTTTAGGAACTTTATCCT
AGAGCGCTCAGGAATCTTGCCCGCCACCTGCTGTGCACTTCCTAG
CGACTTTGTGCCCATTAAGTACCGCGAATGCCCTCCGCCGCTTTG
GGGCCACTGCTACCTTCTGCAGCTAGCCAACTACCTTGCCTACCA
CTCTGACATAATGGAAGACGTGAGCGGTGACGGTCTACTGGAGTG
TCACTGTCGCTGCAACCTATGCACCCCGCACCGCTCCCTGGTTTG
CAATTCGCAGCTGCTTAACGAAAGTCAAATTATCGGTACCTTTGA
GCTGCAGGGTCCCTCGCCTGACGAAAAGTCCGCGGCTCCGGGGTT
GAAACTCACTCCGGGGCTGTGGACGTCGGCTTACCTTCGCAAATT
TGTACCTGAGGACTACCACGCCCACGAGATTAGGTTCTACGAAGA
CCAATCCCGCCCGCCTAATGCGGAGCTTACCGCCTGCGTCATTAC
CCAGGGCCACATTCTTGGCCAATTGCAAGCCATCAACAAAGCCCG
CCAAGAGTTTCTGCTACGAAAGGGACGGGGGGTTTACTTGGACCC
CCAGTCCGGCGAGGAGCTCAACCCAATCCCCCCGCCGCCGCAGCC
CTATCAGCAGCAGCCGCGGGCCCTTGCTTCCCAGGATGGCACCCA
AAAAGAAGCTGCAGCTGCCGCCGCCACCCACGGACGAGGAGGAAT
ACTGGGACAGTCAGGCAGAGGAGGTTTTGGACGAGGAGGAGGAGG
ACATGATGGAAGACTGGGAGAGCCTAGACGAGGAAGCTTCCGAGG
TCGAAGAGGTGTCAGACGAAACACCGTCACCCTCGGTCGCATTCC
CCTCGCCGGCGCCCCAGAAATCGGCAACCGGTTCCAGCATGGCTA
CAACCTCCGCTCCTCAGGCGCCGCCGGCACTGCCCGTTCGCCGAC
CCAACCGTAGATGGGACACCACTGGAACCAGGGCCGGTAAGTCCA
AGCAGCCGCCGCCGTTAGCCCAAGAGCAACAACAGCGCCAAGGCT
ACCGCTCATGGCGCGGGCACAAGAACGCCATAGTTGCTTGCTTGC
AAGACTGTGGGGGCAACATCTCCTTCGCCCGCCGCTTTCTTCTCT
ACCATCACGGCGTGGCCTTCCCCCGTAACATCCTGCATTACTACC
GTCATCTCTACAGCCCATACTGCACCGGCGGCAGCGGCAGCAACA
GCAGCGGCCACACAGAAGCAAAGGCGACCGGATAGCAAGACTCTG
ACAAAGCCCAAGAAATCCACAGCGGCGGCAGCAGCAGGAGGAGGA
GCGCTGCGTCTGGCGCCCAACGAACCCGTATCGACCCGCGAGCTT
AGAAACAGGATTTTTCCCACTCTGTATGCTATATTICAACAGAGC
AGGGGCCAAGAACAAGAGCTGAAAATAAAAAACAGGTCTCTGCGA
TCCCTCACCCGCAGCTGCCTGTATCACAAAAGCGAAGATCAGCTT
CGGCGCACGCTGGAAGACGCGGAGGCTCTCTTCAGTAAATACTGC
GCGCTGACTCTTAAGGACTAGTTTCGCGCCCTTTCTCAAATTTAA
GCGCGAAAACTACGTCATCTCCAGCGGCCACACCCGGCGCCAGCA
CCTGTTGTCAGCGCCATTATGAGCAAGGAAATTCCCACGCCCTAC
ATGTGGAGTTACCAGCCACAAATGGGACTTGCGGCTGGAGCTGCC
CAAGACTACTCAACCCGAATAAACTACATGAGCGCGGGACCCCAC
ATGATATCCCGGGTCAACGGAATACGCGCCCACCGAAACCGAATT
CTCCTGGAACAGGCGGCTATTACCACCACACCTCGTAATAACCTT
AATCCCCGTAGTTGGCCCGCTGCCCTGGTGTACCAGGAAAGTCCC
GCTCCCACCACTGTGGTACTTCCCAGAGACGCCCAGGCCGAAGTT
CAGATGACTAACTCAGGGGCGCAGCTTGCGGGCGGCTTTCGTCAC
AGGGTGCGGTCGCCCGGGCAGGGTATAACTCACCTGACAATCAGA
GGGCGAGGTATTCAGCTCAACGACGAGTCGGTGAGCTCCTCGCTT
GGTCTCCGTCCGGACGGGACATTTCAGATCGGCGGCGCCGGCCGC
TCTTCATTCACGCCTCGTCAGGCAATCCTAACTCTGCAGACCTCG
TCCTCTGAGCCGCGCTCTGGAGGCATTGGAACTCTGCAATTTATT
GAGGAGTTTGTGCCATCGGTCTACTTTAACCCCTTCTCGGGACCT
CCCGGCCACTATCCGGATCAATTTATTCCTAACTTTGACGCGGTA
AAGGACTCGGCGGACGGCTACGACTGAATGTTAAGTGGAGAGGCA
GAGCAACTGCGCCTGAAACACCTGGTCCACTGTCGCCGCCACAAG
TGCTTTGCCCGCGACTCCGGTGAGTTTTGCTACTTTGAATTGCCC
GAGGATCATATCGAGGGCCCGGCGCACGGCGTCCGGCTTACCGCC
CAGGGAGAGCTTGCCCGTAGCCTGATTCGGGAGTTTACCCAGCGC
CCCCTGCTAGTTGAGCGGGACAGGGGACCCTGTGTTCTCACTGTG
ATTTGCAACTGTCCTAACCCTGGATTACATCAAGATCCTCTAGTT
AATGTCAGGTCGCCTAAGTCGATTAACTAGAGTACCCGGGGATCT
TATTCCCTTTAACTAATAAAAAAAAATAATAAAGCATCACTTACT
TAAAATCAGTTAGCAAATTTCTGTCCAGTTTATTCAGCAGCACCT
CCTTGCCCTCCTCCCAGCTCTGGTATTGCAGCTTCCTCCTGGCTG
CAAACTTTCTCCACAATCTAAATGGAATGTCAGTTTCCTCCTGTT
CCTGTCCATCCGCACCCACTATCTTCATGTTGTTGCAGATGAAGC
GCGCAAGACCGTCTGAAGATACCTTCAACCCCGTGTATCCATATG
ACACGGAAACCGGTCCTCCAACTGTGCCTTTTCTTACTCCTCCCT
TTGTATCCCCCAATGGGTTTCAAGAGAGTCCCCCTGGGGTACTCT
CTTTGCGCCTATCCGAACCTCTAGTTACCTCCAATGGCATGCTTG
CGCTCAAAATGGGCAACGGCCTCTCTCTGGACGAGGCCGGCAACC
TTACCTCCCAAAATGTAACCACTGTGAGCCCACCTCTCAAAAAAA
CCAAGTCAAACATAAACCTGGAAATATCTGCACCCCTCACAGTTA
CCTCAGAAGCCCTAACTGTGGCTGCCGCCGCACCTCTAATGGTCG
CGGGCAACACACTCACCATGCAATCACAGGCCCCGCTAACCGTGC
ACGACTCCAAACTTAGCATTGCCACCCAAGGACCCCTCACAGTGT
CAGAAGGAAAGCTAGCCCTGCAAACATCAGGCCCCCTCACCACCA
CCGATAGCAGTACCCTTACTATCACTGCCTCACCCCCTCTAACTA
CTGCCACTGGTAGCTTGGGCATTGACTTGAAAGAGCCCATTTATA
CACAAAATGGAAAACTAGGACTAAAGTACGGGGCTCCTTTGCATG
TAACAGACGACCTAAACACTTTGACCGTAGCAACTGGTCCAGGTG
TGACTATTAATAATACTTCCTTGCAAACTAAAGTTACTGGAGCCT
TGGGTTTTGATTCACAAGGCAATATGCAACTTAATGTAGCAGGAG
GACTAAGGATTGATTCTCAAAACAGACGCCTTATACTTGATGTTA
GTTATCCGTTTGATGCTCAAAACCAACTAAATCTAAGACTAGGAC
AGGGCCCTCTTTTTATAAACTCAGCCCACAACTTGGATATTAACT
ACAACAAAGGCCTTTACTTGTTTACAGCTTCAAACAATTCCAAAA
AGCTTGAGGTTAACCTAAGCACTGCCAAGGGGTTGATGTTTGACG
CTACAGCCATAGCCATTAATGCAGGAGATGGGCTTGAATTTGGTT
CACCTAATGCACCAAACACAAATCCCCTCAAAACAAAAATTGGCC
ATGGCCTAGAATTTGATTCAAACAAGGCTATGGTTCCTAAACTAG
GAACTGGCCTTAGTTTTGACAGCACAGGTGCCATTACAGTAGGAA
ACAAAAATAATGATAAGCTAACTTTGTGGACCACACCAGCTCCAT
CTCCTAACTGTAGACTAAATGCAGAGAAAGATGCTAAACTCACTT
TGGTCTTAACAAAATGTGGCAGTCAAATACTTGCTACAGTTTCAG
TTTTGGCTGTTAAAGGCAGTTTGGCTCCAATATCTGGAACAGTTC
AAAGTGCTCATCTTATTATAAGATTTGACGAAAATGGAGTGCTAC
TAAACAATTCCTTCCTGGACCCAGAATATTGGAACTTTAGAAATG
GAGATCTTACTGAAGGCACAGCCTATACAAACGCTGTTGGATTTA
TGCCTAACCTATCAGCTTATCCAAAATCTCACGGTAAAACTGCCA
AAAGTAACATTGTCAGTCAAGTTTACTTAAACGGAGACAAAACTA
AACCTGTAACACTAACCATTACACTAAACGGTACACAGGAAACAG
GAGACACAACTCCAAGTGCATACTCTATGTCATTTTCATGGGACT
GGTCTGGCCACAACTACATTAATGAAATATTTGCCACATCCTCTT
ACACTTTTTCATACATTGCCCAAGAATAAAGAATCGTTTGTGTTA
TGTTTCAACGTGTTTATTTTTCAATTGCAGAAAATTTCAAGTCAT
TTTTCATTCAGTAGTATAGCCCCACCACCACATAGCTTATACAGA
TCACCGTACCTTAATCAAACTCACAGAACCCTAGTATTCAACCTG
CCACCTCCCTCCCAACACACAGAGTACACAGTCCTTTCTCCCCGG
CTGGCCTTAAAAAGCATCATATCATGGGTAACAGACATATTCTTA
GGTGTTATATTCCACACGGTTTCCTGTCGAGCCAAACGCTCATCA
GTGATATTAATAAACTCCCCGGGCAGCTCACTTAAGTTCATGTCG
CTGTCCAGCTGCTGAGCCACAGGCTGCTGTCCAACTTGCGGTTGC
TTAACGGGCGGCGAAGGAGAAGTCCACGCCTACATGGGGGTAGAG
TCATAATCGTGCATCAGGATAGGGCGGTGGTGCTGCAGCAGCGCG
CGAATAAACTGCTGCCGCCGCCGCTCCGTCCTGCAGGAATACAAC
ATGGCAGTGGTCTCCTCAGCGATGATTCGCACCGCCCGCAGCATA
AGGCGCCTTGTCCTCCGGGCACAGCAGCGCACCCTGATCTCACTT
AAATCAGCACAGTAACTGCAGCACAGCACCACAATATTGTTCAAA
ATCCCACAGTGCAAGGCGCTGTATCCAAAGCTCATGGCGGGGACC
ACAGAACCCACGTGGCCATCATACCACAAGCGCAGGTAGATTAAG
TGGCGACCCCTCATAAACACGCTGGACATAAACATTACCTCTTTT
GGCATGTTGTAATTCACCACCTCCCGGTACCATATAAACCTCTGA
TTAAACATGGCGCCATCCACCACCATCCTAAACCAGCTGGCCAAA
ACCTGCCCGCCGGCTATACACTGCAGGGAACCGGGACTGGAACAA
TGACAGTGGAGAGCCCAGGACTCGTAACCATGGATCATCATGCTC
GTCATGATATCAATGTTGGCACAACACAGGCACACGTGCATACAC
TTCCTCAGGATTACAAGCTCCTCCCGCGTTAGAACCATATCCCAG
GGAACAACCCATTCCTGAATCAGCGTAAATCCCACACTGCAGGGA
AGACCTCGCACGTAACTCACGTTGTGCATTGTCAAAGTGTTACAT
TCGGGCAGCAGCGGATGATCCTCCAGTATGGTAGCGCGGGTTTCT
GTCTCAAAAGGAGGTAGACGATCCCTACTGTACGGAGTGCGCCGA
GACAACCGAGATCGTGTTGGTCGTAGTGTCATGCCAAATGGAACG
CCGGACGTAGTCATATTTCCTGAAGCAAAACCAGGTGCGGGCGTG
ACAAACAGATCTGCGTCTCCGGTCTCGCCGCTTAGATCGCTCTGT
GTAGTAGTTGTAGTATATCCACTCTCTCAAAGCATCCAGGCGCCC
CCTGGCTTCGGGTTCTATGTAAACTCCTTCATGCGCCGCTGCCCT
GATAACATCCACCACCGCAGAATAAGCCACACCCAGCCAACCTAC
ACATTCGTTCTGCGAGTCACACACGGGAGGAGCGGGAAGAGCTGG
AAGAACCATGTTTTTTTTTTTATTCCAAAAGATTATCCAAAACCT
CAAAATGAAGATCTATTAAGTGAACGCGCTCCCCTCCGGTGGCGT
GGTCAAACTCTACAGCCAAAGAACAGATAATGGCATTTGTAAGAT
GTTGCACAATGGCTTCCAAAAGGCAAACGGCCCTCACGTCCAAGT
GGACGTAAAGGCTAAACCCTTCAGGGTGAATCTCCTCTATAAACA
TTCCAGCACCTTCAACCATGCCCAAATAATTCTCATCTCGCCACC
TTCTCAATATATCTCTAAGCAAATCCCGAATATTAAGTCCGGCCA
TTGTAAAAATCTGCTCCAGAGCGCCCTCCACCTTCAGCCTCAAGC
AGCGAATCATGATTGCAAAAATTCAGGTTCCTCACAGACCTGTAT
AAGATTCAAAAGCGGAACATTAACAAAAATACCGCGATCCCGTAG
GTCCCTTCGCAGGGCCAGCTGAACATAATCGTGCAGGTCTGCACG
GACCAGCGCGGCCACTTCCCCGCCAGGAACCATGACAAAAGAACC
CACACTGATTATGACACGCATACTCGGAGCTATGCTAACCAGCGT
AGCCCCGATGTAAGCTTGTTGCATGGGCGGCGATATAAAATGCAA
GGTGCTGCTCAAAAAATCAGGCAAAGCCTCGCGCAAAAAAGAAAG
CACATCGTAGTCATGCTCATGCAGATAAAGGCAGGTAAGCTCCGG
AACCACCACAGAAAAAGACACCATTTTTCTCTCAAACATGTCTGC
GGGTTTCTGCATAAACACAAAATAAAATAACAAAAAAACATTTAA
ACATTAGAAGCCTGTCTTACAACAGGAAAAACAACCCTTATAAGC
ATAAGACGGACTACGGCCATGCCGGCGTGACCGTAAAAAAACTGG
TCACCGTGATTAAAAAGCACCACCGACAGCTCCTCGGTCATGTCC
GGAGTCATAATGTAAGACTCGGTAAACACATCAGGTTGATTCACA
TCGGTCAGTGCTAAAAAGCGACCGAAATAGCCCGGGGGAATACAT
ACCCGCAGGCGTAGAGACAACATTACAGCCCCCATAGGAGGTATA
ACAAAATTAATAGGAGAGAAAAACACATAAACACCTGAAAAACCC
TCCTGCCTAGGCAAAATAGCACCCTCCCGCTCCAGAACAACATAC
AGCGCTTCCACAGCGGCAGCCATAACAGTCAGCCTTACCAGTAAA
AAAGAAAACCTATTAAAAAAACACCACTCGACACGGCACCAGCTC
AATCAGTCACAGTGTAAAAAAGGGCCAAGTGCAGAGCGAGTATAT
ATAGGACTAAAAAATGACGTAACGGTTAAAGTCCACAAAAAACAC
CCAGAAAACCGCACGCGAACCTACGCCCAGAAACGAAAGCCAAAA
AACCCACAACTTCCTCAAATCGTCACTTCCGTTTTCCCACGTTAC
GTCACTTCCCATTTTAAGAAAACTACAATTCCCAACACATACAAG
TTACTCCGCCCTAAAACCTACGTCACCCGCCCCGTTCCCACGCCC
CGCGCCACGTCACAAACTCCACCCCCTCATTATCATATTGGCTTC
AATCCAAAATAAGGTATATT
SEQ ID NO: 10: Amino Sequence of SI-N
MFVFLVLLPLVSSQCVNLTTRTQLPPAYTNSFTRGVYYPDKVFRS
SVLHSTQDLFLPFFSNVTWFHAIHVSGTNGTKRFDNPVLPENDGV
YFASTEKSNIIRGWIFGTTLDSKTQSLLIVNNATNVVIKVCEFQF
CNDPFLGVYYHKNNKSWMESEFRVYSSANNCTFEYVSQPFLMDLE
GKQGNFKNLREFVFKNIDGYFKIYSKHTPINLVRDLPQGFSALEP
LVDLPIGINITRFQTLLALHRSYLTPGDSSSGWTAGAAAYYVGYL
QPRTFLLKYNENGTITDAVDCALDPLSETKCTLKSFTVEKGIYQT
SNFRVQPTESIVRFPNITNLCPFGEVENATRFASVYAWNRKRISN
CVADYSVLYNSASFSTFKCYGVSPTKLNDLCFTNVYADSFVIRGD
EVRQIAPGQTGKIADYNYKLPDDFTGCVIAWNSNNLDSKVGGNYN
YLYRLFRKSNLKPFERDISTEIYQAGSTPCNGVEGENCYFPLQSY
GFQPTNGVGYQPYRVVVLSFELLHAPATVCGPKKSTNLVKNKCVN
FNFNGLTGTGVLTESNKKELPFQQFGRDIADTTDAVRDPQTLEIL
DITPCSFGGVSVITPGTNTSNQVAVLYQDVNCTEVPVAIHADQLT
PTWRVYSTGSNVFQTRAGCLIGAEHVNNSYECDIPIGAGICASYQ
TQTNSPRRARSVASQSIIAYTMMSDNGPQNQRNAPRITFGGPSDS
TGSNQNGERSGARSKQRRPQGLPNNTASWFTALTQHGKEDLKFPR
GQGVPINTNSSPDDQIGYYRRATRRIRGGDGKMKDLSPRWYFYYL
GTGPEAGLPYGANKDGIIWVATEGALNTPKDHIGTRNPANNAAIV
LQLPQGTTLPKGFYAEGSRGGSQASSRSSSRSRNSSRNSTPGSSR
GTSPARMAGNGGDAALALLLLDRLNQLESKMSGKGQQQQGQTVTK
KSAAEASKKPRQKRTATKAYNVTQAFGRRGPEQTQGNFGDQELIR
QGTDYKHWPQIAQFAPSASAFFGMSRIGMEVTPSGTWLTYTGAIK
LDDKDPNFKDQVILLNKHIDAYKTFPPTEPKKDKKKKADETQALP
QRQKKQQTVTLLPAADLDDFSKQLQQSMSSADSTQA
SEQ ID NO: 11: TLR-3 agonist sequence
GAAACGATATGGGCTGAATACTTAAGTATTCAGCCCATATCGTTT
C
SEQ ID NO: 12: TLR-3 agonist sequence
CGGGCCCCCCCTCGAGGTCGACGGTATCGATAAGCTTGATATCGA
ATTCGCCCTTAGATATCGTCGACGCCCAGCACCCCAAGGCGGCCA
ACGCCAAAACTCTCCCTCCTCCTCTTCCTCAATCTCGCTCTCGCT
CTTTTTTTTTTTCGCAAAAGGAGGGGAGAGGGGGTAAAAAAATGC
TGCACTGTGCGGCGAAGCCGGTGAGTGAGCGGCGCGGGGCCAATC
AGCGTGCGCCGTTCCGAAAGTTGCCTTTTATGGCTCGAGCGGCCG
CGGCGGCGCCCTATAAAACCCAGCGGCGCGACGCGCCACCACCGC
CGAGACATCGATGATATCTAAAGGGCGAATTCCTGCAGCCCGGGG
GATCCACTAGTCTAGATGCATGCTCGAGCGGCCGCCAGTGTGATG
GATATCTGCAGAATTCGCCCTTCAGCTGCGGATCCATTCGCCATT
CAGGCTGCGCAACTGTTGGGAAGGGCGATCGGTGCGGGCCTCTTC
GCTATTACGCCAGCTGGCGAAAGGGGGATGTGCTGCAAGGCGATT
AAGTTGGGTAACGCCAGGGTTTTCCCAGTCACGACGTTGTAAAAC
GACGGCCAGTGAATTGTAATACGACTCACTATAGGGCGAATTGGG
TACCGGGCCCCCCCTCGAGGTCGACGGTATCGATAAGCTTGATAT
CGAATTCCTGCAGCCCGGGGGATCCACTAGTTTCTAGAAATAAAA
TATCTTTATTTTCATTACATCTGTGTGTTGGTTTTTTGTGTGGCG
GCCGCCACCGCGGTGGAGCTATCGAATTCAAGCTTGTCGACTCGA
AGATCCTAGACTAGTGGATCCCCCGGGCTGCAGGAATTCGCCCTT
TAGATATCATCGATGTCTCGGCGGTGGTGGCGCGTCGCGCCGCTG
GGTTTTATAGGGCGCCGCCGCGGCCGCTCGAGCCATAAAAGGCAA
CTTTCGGAACGGCGCACGCTGATTGGCCCCGCGCCGCTCACTCAC
CGGCTTCGCCGCACAGTGCAGCATTTTTTTACCCCCTCTCCCCTC
CTTTTGCGAAAAAAAAAAAGAGCGAGAGCGAGATTGAGGAAGAGG
AGGAGGGAGAGTTTTGGCGTTGGCCGCCTTGGGGTGCTGGGCGTC
GACGATATCTAAGGGCGAATTCGATATCAAGCTTATCGATACCGT
CGACCTCGAGGGGGGGCCCG
SEQ ID NO: 13: TLR-3 agonist sequence
CGGGCCCCCCCTCGAGGTCGACGGTATCGATAAGCTTGATATCGA
ATTCGCCCTTAGATATCGTCGACGCCCAGCACCCCAAGGCGGCCA
ACGCCAAAACTCTCCCTCCTCCTCTTCCTCAATCTCGCTCTCGCT
CTTTTTTTTTTTCGCAAAAGGAGGGGAGAGGGGGTAAAAAAATGC
TGCACTGTGCGGCGAAGCCGGTGAGTGAGCGGCGCGGGGCCAATC
AGCGTGCGCCGTTCCGAAAGTTGCCTTTTATGGCTCGAGCGGCCG
CGGCGGCGCCCTATAAAACCCAGCGGCGCGACGCGCCACCACCGC
CGAGACATCGATGATATCTAAAGGGCGAATTCCTGCAGCCCGGGG
GATCCACTAGTCTAGATGCATGCTCGAGCGGCCGCCAGTGTGATG
GATATCTGCAGAATTCGCCCTTCAGCTGCGGATCCATTCGCCATT
CAGGCTGCGCAACTGTTGGGAAGGGCGATCGGTGCGGGCCTCTTC
GCTATTACGCCAGCTGGCGAAAGGGGGATGTGCTGCAAGGCGATT
AAGTTGGGTAACGCCAGGGTTTTCCCAGTCACGACGTTGTAAAAC
GACGGCCAGTGAATTGTAATACGACTCACTATAGGGCGAATTGGG
TACCGGGCCCCCCCTCGAGGTCGACGGTATCGATAAGCTTGATAT
CGAATTCCTGCAGCCCGGGGGATCCACTAGTTTCTAGAAATAAAA
TATCTTTATTTTCATTACATCTGTGTGTTGGTTTTTTGTGTGGCG
GCCGCCACCGCGGTGGAGCTATCGAATTCAAGCTTGTCGACTCGA
AGATCGTACACAGGAAGTGACAATTTTCGCGCGGTTTTAGGCGGA
TGTTGTAGTAAATTTGGGCGTAACCGAGTAAGATTTGGCCATTTT
CGCGGGAAAACTGAATAAGAGGAAGTGAAATCTGAATAATTTTGT
GTTACTCATAGCGCGTAATACTGGTACCGGGCCCCCCCTCGAGGT
CGACGGTATCGATAAGCTTGATATCGAATTCGCCCTTAGATATCG
TCGACGCCCAGCACCCCAAGGCGGCCAACGCCAAAACTCTCCCTC
CTCCTCTTCCTCAATCTCGCTCTCGCTCTTTTTTTTTTTCGCAAA
AGGAGGGGAGAGGGGGTAAAAAAATGCTGCACTGTGCGGCGAAGC
CGGTGAGTGAGCGGCGCGGGGCCAATCAGCGTGCGCCGTTCCGAA
AGTTGCCTTTTATGGCTCGAGCGGCCGCGGCGGCGCCCTATAAAA
CCCAGCGGCGCGACGCGCCACCACCGCCGAGACATCGATGATATC
TAAAGGGCGAATTCCTGCAGCCCGGGGGATCCACTAGTCTAGAAC
TAGTGGATCCCCCGGGCTGCAGGAATTCGATATCAAGCTTATCGA
TACCGTCGACCTCGAGGGGGGGCCCGGTACCCAATTCGCCCTATA
GTGAGTCGTATTACAATTCACTGGCCGTCGTTTTACAACGTCGTG
ACTGGGAAAACCCTGGCGTTACCCAACTTAATCGCCTTGCAGCAC
ATCCCCCTTTCGCCAGCTGGCGTAATAGCGAAGAGGCCCGCACCG
ATCGCCCTTCCCAACAGTTGCGCAGCCTGAATGGCGAATGGATCC
GCAGCTGAAGGGCGAATTCTGCAGATATCCATCACACTGGCGGCC
GCTCGAGCATGCATCTAGAAATAAAATATCTTTATTTTCATTACA
TCTGTGTGTTGGTTTTTTGTGTGGCGGCCGCCACCGCGGTGGAGC
TA
SEQ ID NO: 14: TLR-3 agonist sequence
GATGGTGCTTCAAGCTAGTACTTAAGTACTAGCTTGAAGCACCAT
C
SEQ ID NO: 15: TLR-3 agonist sequence
GATGGTGCTTCAAGCTAGTACGGATCCGTACTAGCTTGAAGCACC
ATC
SEQ ID NO: 16: TLR-3 agonist sequence
GAAACGATATGGGCTGAATACGGATCCGTATTCAGCCCATATCGT
TTC
SEQ ID NO: 17: TLR-3 agonist sequence
CCTAATAATTATCAAAATGTGGATCCACATTTTGATAATTATTAG
G
SEQ ID NO: 18: TLR-3 agonist sequence
CCTAATAATTATCAAAATGTAATTACATTTTGATAATTATTAGG
SEQ ID NO: 19
UK B.1.1.7 S Protein Variant
GISAID Accession #EPI_ISL_601443
MFVFLVLLPLVSSQCVNLTTRTQLPPAYTNSFTRGVYYPDKVFRS
SVLHSTQDLFLPFFSNVTWFHAISGTNGTKRFDNPVLPFNDGVYF
ASTEKSNIIRGWIFGTTLDSKTQSLLIVNNATNVVIKVCEFQFCN
DPFLGVYHKNNKSWMESEFRVYSSANNCTFEYVSQPFLMDLEGKQ
GNFKNLREFVFKNIDGYFKIYSKHTPINLVRDLPQGFSALEPLVD
LPIGINITRFQTLLALHRSYLTPGDSSSGWTAGAAAYYVGYLQPR
TFLLKYNENGTITDAVDCALDPLSETKCTLKSFTVEKGIYQTSNF
RVQPTESIVRFPNITNLCPFGEVENATRFASVYAWNRKRISNCVA
DYSVLYNSASFSTFKCYGVSPTKLNDLCFTNVYADSFVIRGDEVR
QIAPGQTGKIADYNYKLPDDFTGCVIAWNSNNLDSKVGGNYNYLY
RLFRKSNLKPFERDISTEIYQAGSTPCNGVEGENCYFPLQSYGFQ
PTYGVGYQPYRVVVLSFELLHAPATVCGPKKSTNLVKNKCVNFNF
NGLTGTGVLTESNKKFLPFQQFGRDIDDTTDAVRDPQTLEILDIT
PCSFGGVSVITPGTNTSNQVAVLYQGVNCTEVPVAIHADQLTPTW
RVYSTGSNVFQTRAGCLIGAEHVNNSYECDIPIGAGICASYQTQT
NSHRRARSVASQSIIAYTMSLGAENSVAYSNNSIAIPINFTISVT
TEILPVSMTKTSVDCTMYICGDSTECSNLLLQYGSFCTQLNRALT
GIAVEQDKNTQEVFAQVKQIYKTPPIKDFGGFNFSQILPDPSKPS
KRSFIEDLLFNKVTLADAGFIKQYGDCLGDIAARDLICAQKFNGL
TVLPPLLTDEMIAQYTSALLAGTITSGWTFGAGAALQIPFAMQMA
YRENGIGVTQNVLYENQKLIANQFNSAIGKIQDSLSSTASALGKL
QDVVNQNAQALNTLVKQLSSNFGAISSVLNDILARLDKVEAEVQI
DRLITGRLQSLQTYVTQQLIRAAEIRASANLAATKMSECVLGQSK
RVDFCGKGYHLMSFPQSAPHGVVFLHVTYVPAQEKNFTTAPAICH
DGKAHFPREGVFVSNGTHWFVTQRNFYEPQIITTHNTFVSGNCDV
VIGIVNNTVYDPLQPELDSFKEELDKYFKNHTSPDVDLGDISGIN
ASVVNIQKEIDRLNEVAKNLNESLIDLQELGKYEQYIKWPWYIWL
GFIAGLIAIVMVTIMLCCMTSCCSCLKGCCSCGSCCKFDEDDSEP
VLKGVKLHYT*
SEQ ID NO: 20
S. African B.1.351 501Y.V2 S Protein Variant
GISAID Accession #EPI_ISL_678597
MFVFLVLLPLVSSQCVNLTTRTQLPPAYTNSFTRGVYYPDKVFRS
SVLHSTQDLFLPFFSNVTWFHAIHVSGTNGTKRFANPVLPFNDGV
YFASTEKSNIIRG
WIFGTTLDSKTQSLLIVNNATNVVIKVCEFQFCNDPFLGVYYHKN
NKSWMESEFRVYSSANNCTFEYVSQPFLMDLEGKQGNFKNLREFV
FKNIDGYFKIYSKHTPINLVRGLPQGFSALEPLVDLPIGINITRF
QTLHRSYLTPGDSSSGWTAGAAAYYVGYLQPRTFLLKYNENGTIT
DAVDCALDPLSETKCTLKSFTVEKGIYQTSNFRVQPTESIVRFPN
ITNLCPFGEVENATRFASVYAWNRKRISNCVADYSVLYNSASFST
FKCYGVSPTKLNDLCFTNVYADSFVIRGDEVRQIAPGQTGNIADY
NYKLPDDFTGCVIAWNSNNLDSKVGGNYNYLYRLFRKSNLKPFER
DISTEIYQAGSTPCNGVKGFNCYFPLQSYGFQPTYGVGYQPYRVV
VLSFELLHAPATVCGPKKSTNLVKNKCVNFNFNGLTGTGVLTESN
KKFLPFQQFGRDIADTTDAVRDPQTLEILDITPCSFGGVSVITPG
TNTSNQVAVLYQGVNCTEVPVAIHADQLTPTWRVYSTGSNVFQTR
AGCLIGAEHVNNSYECDIPIGAGICASYQTQTNSPRRARSVASQS
IIAYTMSLGVENSVAYSNNSIAIPTNFTISVTTEILPVSMTKTSV
DCTMYICGDSTECSNLLLQYGSFCTQLNRALTGIAVEQDKNTQEV
FAQVKQIYKTPPIKDFGGFNFSQILPDPSKPSKRSFIEDLLFNKV
TLADAGFIKQYGDCLGDIAARDLICAQKFNGLTVLPPLLTDEMIA
QYTSALLAGTITSGWTFGAGAALQIPFAMQMAYRENGIGVTQNVL
YENQKLIANQFNSAIGKIQDSLSSTASALGKLQDVVNQNAQALNT
LVKQLSSNFGAISSVLNDILSRLDKVEAEVQIDRLITGRLQSLQT
YVTQQLIRAAEIRASANLAATKMSECVLGQSKRVDFCGKGYHLMS
FPQSAPHGVVFLHVTYVPAQEKNFTTAPAICHDGKAHFPREGVFV
SNGTHWFVTQRNFYEPQIITTDNTFVSGNCDVVIGIVNNTVYDPL
QPELDSFKEELDKYFKNHTSPDVDLGDISGINASVVNIQKEIDRL
NEVAKNLNESLIDLQELGKYEQYIKWPWYIWLGFIAGLIAIVMVT
IMLCCMTSCCSCLKGCCSCGSCCKFDEDDSEPVLKGVKLHYT*
SEQ ID NO: 21
JL82 Insert DNA sequence encoding
SEQ ID NO: 22:
ATGGACGCCATGAAACGAGGCCTGTGCTGCGTCCTCCTGCTGTGT
GGGGCAGTGTTCGTTAGTCACCAGAAGCGAACCGCAATGTTTCAG
GACCCCCAGGAAAGGCCCCGAAAATTGCCGCAGCTGTGCACCGAG
CTCCAGACAACCATTCATGACATCATTCTGGAGTGTGTGTATTGT
AAGCAGCAGCTGTTGAGGAGGGAGGTGTATGACTTCGCTTTTCGG
GACGGTTGCATTGTTTACCGGGATGGAAACCCTTACGCCGTTTGC
GATAAATGTCTGAAGTTCTATAGCAAAATTAGTGAATATAGGCAT
TATTGCTACTCACTGTACGGAACCACACTGGAACAGCAGTATAAC
AAACCCCTGTGCGACCTTCTGATTAGGTGCATTAATTGTCAGAAA
CCGCTGTGCCCAGAGGAAAAGCAGCGCCATCTTGACAAGAAACAG
AGATTCCATAACATCCGGGGCAGATGGACTGGACGCTGCATGTCT
TGTTGTCGCTCCTCAAGGACGAGACGGGCCGCGGCTGCCAGGAAG
AAACGTAGGATGCCCGGCGATACCCCGACACTGCACGAATATATG
CIGGACCTCCAACCCGAGACGACAGATCTGTACGGTTACGAGCAA
CTGAACGACTCCTCCGAGGAAGAAGACGAAATCGACGGGCCCGCA
GGTCAGGCAGCACCTGACCGCGCCCACTACAATATTGTCACCTTT
TGCTGCAAATGTGACTCCACACTCCGAcgTTGTGTTCAATCAACC
CACGTGGATATTCGAACTCTGGAGGATCTTCTGATGGGAACCCTG
GGTATTGTATGCCCCATCTGCAGCCAAAAACCATAG
SEQ ID NO: 22
JL82 insert amino acid sequence (HPV16E6E7)
encoded by SEQ ID NO: 21
MDAMKRGLCCVLLLCGAVEVSHQKRTAMFQDPQERPRKLPQLCTE
LQTTIHDIILECVYCKQQLLRREVYDFAFRDGCIVYRDGNPYAVC
DKCLKFYSKISEYRHYCYSLYGTTLEQQYNKPLCDLLIRCINCQK
PLCPEEKQRHLDKKQRFHNIRGRWTGRCMSCCRSSRTRRAAAARK
KRRMPGDTPTLHEYMLDLQPETTDLYGYEQLNDSSEEEDEIDGPA
GQAAPDRAHYNIVTFCCKCDSTLRRCVQSTHVDIRTLEDLLMGTL
GIVCPICSQKP*
SEQ ID NO: 23:
HPV16E6E7-T2A-SARS-CoV-2 N insert DNA
sequence. Sequence encoding T2A
peptide is underlined
ATGGACGCCATGAAACGAGGCCTGTGCTGCGTCCTCCTGCTGTGT
GGGGCAGTGTTCGTTAGTCACCAGAAGCGAACCGCAATGTTTCAG
GACCCCCAGGAAAGGCCCCGAAAATTGCCGCAGCTGTGCACCGAG
CTCCAGACAACCATTCATGACATCATTCTGGAGTGTGTGTATTGT
AAGCAGCAGCTGTTGAGGAGGGAGGTGTATGACTTCGCTTTTCGG
GACGGTTGCATTGTTTACCGGGATGGAAACCCTTACGCCGTTTGC
GATAAATGTCTGAAGTTCTATAGCAAAATTAGTGAATATAGGCAT
TATTGCTACTCACTGTACGGAACCACACTGGAACAGCAGTATAAC
AAACCCCTGTGCGACCTTCTGATTAGGTGCATTAATTGTCAGAAA
CCGCTGTGCCCAGAGGAAAAGCAGCGCCATCTTGACAAGAAACAG
AGATTCCATAACATCCGGGGCAGATGGACTGGACGCTGCATGTCT
TGTTGTCGCTCCTCAAGGACGAGACGGGCCGCGGCTGCCAGGAAG
AAACGTAGGATGCCCGGCGATACCCCGACACTGCACGAATATATG
CTGGACCTCCAACCCGAGACGACAGATCTGTACGGTTACGAGCAA
CTGAACGACTCCTCCGAGGAAGAAGACGAAATCGACGGGCCCGCA
GGTCAGGCAGCACCTGACCGCGCCCACTACAATATTGTCACCTTT
TGCTGCAAATGTGACTCCACACTCCGAcgTTGTGTTCAATCAACC
CACGTGGATATTCGAACTCTGGAGGATCTTCTGATGGGAACCCTG
GGTATTGTATGCCCCATCTGCAGCCAAAAACCAGGCTCCGGCGAG
GGCAGGGGAAGTCTTCTAACATGCGGGGACGTGGAGGAAAATCCC
GGCCCAATGTCCGATAACGGCCCCCAGAATCAGAGAAACGCTCCC
CGCATCACGTTCGGCGGACCAAGTGACAGCACAGGCAGTAACCAG
AACGGAGAACGCTCCGGTGCTCGCTCCAAGCAGCGACGGCCGCAA
GGGCTTCCCAACAATACCGCCAGCTGGTTTACGGCTCTGACCCAA
CACGGGAAAGAAGATCTTAAATTCCCCAGGGGCCAGGGCGTCCCT
ATCAATACTAACTCCAGCCCGGATGATCAGATAGGCTACTATAGA
CGCGCTACCCGACGGATACGAGGGGGGGACGGCAAAATGAAGGAC
CTTTCCCCCCGGTGGTATTTCTATTACTTGGGCACCGGACCAGAA
GCCGGACTGCCTTACGGCGCTAACAAAGACGGAATAATCTGGGTT
GCGACGGAGGGCGCCCTGAATACACCTAAAGACCATATCGGCACA
AGAAATCCTGCTAACAATGCCGCGATTGTGCTCCAGCTGCCTCAG
GGAACCACGCTGCCTAAAGGGTTTTACGCTGAGGGGTCAAGGGGG
GGGAGTCAAGCGTCTAGTAGGTCATCCTCTCGCTCTCGCAATAGT
TCCCGGAACTCAACCCCAGGCAGCAGCAGAGGAACCTCTCCCGCA
CGGATGGCTGGCAATGGGGGAGATGCTGCCCTTGCTCTCCTTCTG
CTGGATCGCCTTAACCAGCTCGAATCAAAGATGTCTGGAAAAGGT
CAGCAGCAGCAAGGCCAGACCGTGACAAAGAAGAGTGCAGCTGAA
GCTAGTAAAAAGCCACGCCAAAAACGGACCGCAACTAAGGCATAT
AACGTAACACAGGCCTTCGGCAGAAGAGGTCCAGAACAAACACAG
GGAAACTTTGGCGATCAAGAGCTGATTAGACAGGGCACAGATTAC
AAACACTGGCCACAGATCGCGCAGTTTGCACCAAGCGCCTCTGCA
TTCTTCGGGATGAGTCGGATTGGGATGGAAGTCACTCCATCCGGG
ACCTGGCTTACCTACACAGGGGCAATAAAACTCGACGACAAAGAC
CCAAACTTTAAAGATCAGGTCATCCTGCTGAATAAACACATCGAT
GCCTACAAAACTTTCCCCCCAACCGAACCAAAGAAAGACAAGAAA
AAAAAGGCAGACGAAACGCAAGCGCTCCCTCAGCGCCAGAAGAAG
CAGCAGACCGTTACACTGTTGCCAGCAGCAGATCTGGATGATTTT
TCCAAGCAGCTTCAACAGAGTATGTCAAGCGCTGACAGCACTCAG
GCTTGA
Peptide sequences encoded by SEQ ID NO: 23:
HPV16E6E7 (SEQ ID NO: 22):
MDAMKRGLCCVLLLCGAVFVSHQKRTAMFQDPQERPRKLPQLCTE
LQTTIHDIILECVYCKQQLLRREVYDFAFRDGCIVYRDGNPYAVC
DKCLKFYSKISEYRHYCYSLYGTTLEQQYNKPLCDLLIRCINCQK
PLCPEEKQRHLDKKQRFHNIRGRWTGRCMSCCRSSRTRRAAAARK
KRRMPGDTPTLHEYMLDLQPETTDLYGYEQLNDSSEEEDEIDGPA
GQAAPDRAHYNIVTFCCKCDSTLRRCVQSTHVDIRTLEDLLMGTL
GIVCPICSQKP
SEQ ID NO: 24: T2A amino acid sequence:
GSGEGRGSLLTCGDVEENPGP
SARS-CoV-2 N protein (SEQ ID NO: 2)
MSDNGPQNQRNAPRITFGGPSDSTGSNQNGERSGARSKQRRPQGL
PNNTASWFTALTQHGKEDLKFPRGQGVPINTNSSPDDQIGYYRRA
TRRIRGGDGKMKDLSPRWYFYYLGTGPEAGLPYGANKDGIIWVAT
EGALNTPKDHIGTRNPANNAAIVLQLPQGTTLPKGFYAEGSRGGS
QASSRSSSRSRNSSRNSTPGSSRGTSPARMAGNGGDAALALLLLD
RINQLESKMSGKGQQQQGQTVTKKSAAEASKKPRQKRTATKAYNV
TQAFGRRGPEQTQGNFGDQELIRQGTDYKHWPQIAQFAPSASAFF
GMSRIGMEVTPSGTWLTYTGAIKLDDKDPNEKDQVILLNKHIDAY
KTFPPTEPKKDKKKKADETQALPQRQKKQQTVTLLPAADLDDFSK
QLQQSMSSADSTQA*

1. A chimeric adenoviral expression vector, comprising an expression cassette comprising:
a nucleic acid encoding an antigenic polypeptide; and
a nucleic acid encoding a SARS-CoV-2 N protein,
wherein the antigenic polypeptide is not a SARS-CoV2 protein.

2. The chimeric adenoviral expression vector of claim 1, wherein the SARS-CoV-2 N protein comprises an amino acid sequence having at least 95%, 96%, 97%, 98%, 99%, or 100% identity to the amino acid sequence of SEQ ID NO:2.

3. The chimeric adenoviral expression vector of claim 2, wherein the nucleic acid encoding the SARS-CoV-2 N protein comprises a sequence having at least 85%, 90%, 95%, 97%, 99%, or 100% identity to the sequence of SEQ ID NO:4.

4. The chimeric adenoviral expression vector of claim 1, wherein the antigenic polypeptide is (i) a cancer antigen: (ii) from a pathogen; (iii) from a virus, bacteria, fungus, or parasite: or (iv) from a human papilloma virus (HPV) antigen, optionally, wherein the antigenic polypeptide comprises SEQ ID NO:22.

5-7. (canceled)

8. The chimeric adenoviral expression vector of claim 1, wherein the expression cassette comprises a bicistronic or multicistronic construct comprising the nucleic acid encoding the antigenic polypeptide and the nucleic acid encoding the SARS-CoV-2 N protein operably linked to a promoter.

9. The chimeric adenoviral expression vector of claim 8, wherein:
(a) the nucleic acid encoding the antigenic polypeptide is positioned 5′ of the nucleic acid encoding the SARS-CoV2-N protein; or the nucleic acid encoding the SARS-CoV2-N protein is positioned 5′ of the nucleic acid encoding the antigenic polypeptide; and/or
(b) the expression cassette comprises: (i) an internal ribosome entry site (IRES), a ribosomal skipping element, or a furin cleavage site positioned between the nucleic acid encoding the antigenic polypeptide and the nucleic acid encoding the SARS-CoV-2 N protein: or (ii) a ribosomal skipping element, wherein the ribosomal skipping element is a sequence encoding a peptide selected from the group consisting of a 2A peptide (T2A), a porcine teschovirus-1 2A peptide (P2A), a foot-and-mouth disease virus 2A peptide (F2A), equine rhinitis A virus 2A peptide (E2A), a cytoplasmic polyhedrosis virus 2A peptide (BmCPV 2A), and a flacherie virus of B. mori 2A peptide (BmIFV 2A); and/or
(c) the promoter is a CMV promoter.

10-14. (canceled)

15. The chimeric adenoviral expression vector of claim 1, wherein the nucleic acid encoding the antigenic polypeptide is operably linked to a first promoter and the nucleic acid encoding the SARS-CoV-2 N protein is operably linked to a second promoter.

16. The chimeric adenoviral expression vector of claim 15, wherein the first promoter and the second promoter are each a CMV promoter; or the first promoter is a CMV promoter and the second promoter is a beta-actin promoter; or the first promoter is a beta-actin promoter and the second promoter is a CMV promoter.

17. (canceled)

18. The chimeric adenoviral expression vector of claim 1, wherein the expression cassette comprises a polyadenylation signal, optionally wherein the polyadenylation signal is a bovine growth hormone polyadenylation signal.

19. (canceled)

20. The chimeric adenoviral expression vector of claim 1, wherein the chimeric adenoviral expression vector further comprises a nucleic acid encoding a toll-like receptor-3 (TLR-3) agonist.

21. The chimeric adenoviral expression vector of claim 20, wherein the TLR-3 agonist comprises a nucleic acid encoding a dsRNA, optionally wherein the nucleic acid encoding the TLR-3 agonist comprises a sequence selected from the group consisting of: SEQ ID NOS:11-18.

22. (canceled)

23. A host cell comprising a chimeric adenoviral vector of claim 1.

24. An immunogenic composition comprising the chimeric adenoviral expression vector of claim 1 and a pharmaceutically acceptable carrier.

25. A method for eliciting an immune response towards an antigenic polypeptide in a subject, comprising administering to the subject an immunogenically effective amount of the chimeric adenoviral expression vector of claim 1 to a mammalian subject, optionally wherein the mammalian subject is a human.

26. The method of claim 25, wherein the route of administration is oral, intranasal, or mucosal.

27. (canceled)

28. The method of claim 25, wherein the immune response is elicited in an alveolar cell, an absorptive enterocyte, a ciliated cell, a goblet cell, a club cells, and/or an airway basal cell of the subject.

29. (canceled)

30. A chimeric polynucleotide, comprising an expression cassette comprising:
a nucleic acid encoding an antigenic polypeptide, with the proviso that the antigenic polypeptide is not a SARS-CoV-2 protein; and
a nucleic acid encoding a SARS-CoV-2 N protein.

31. The chimeric polynucleotide of claim 30, wherein the SARS-CoV-2 N protein comprises an amino acid sequence having at least 95%, 96%, 97%, 98%, 99%, or 100% identity to the amino acid sequence of SEQ ID NO:2, optionally wherein the nucleic acid encoding the SARS-CoV-2 N protein comprises a sequence having at least 85%, 90%, 95%, 97%, 99%, or 100% identity to the sequence of SEQ ID NO:4.

32. (canceled)

33. The chimeric polynucleotide of claim 30, wherein the antigenic polypeptide is (i) a cancer antigen; (ii) from a pathogen: (iii) from a virus, bacteria, fungus, or parasite: or (iv) from a human papilloma virus (HPV) antigen, optionally, wherein the polypeptide comprises SEQ ID NO:22.

34-36. (canceled)

37. The chimeric polynucleotide of claim 30, wherein the expression cassette comprises a bicistronic or multicistronic construct comprising the nucleic acid encoding the antigenic polypeptide and the nucleic acid encoding the SARS-CoV-2 N protein operably linked to a promoter.

38. The chimeric polynucleotide of claim 37, wherein:
(a) the nucleic acid encoding the antigenic protein is positioned 5′ of the nucleic acid encoding the SARS-CoV2-N protein: or the nucleic acid encoding the SARS-CoV2-N protein is positioned 5′ of the nucleic acid encoding the antigenic polypeptide; and/or
(b) the expression cassette comprises: (i) an internal ribosome entry site (IRES), a ribosomal skipping element, or a furin cleavage site positioned between the nucleic acid encoding the antigenic polypeptide and the nucleic acid encoding the SARS-CoV-2 N protein: or (ii) a ribosomal skipping element and the ribosomal skipping element is a sequence encoding a peptide selected from the group consisting of a 2A peptide (T2A), a porcine teschovirus-1 2A peptide (P2A), a foot-and-mouth disease virus 2A peptide (F2A), equine rhinitis A virus 2A peptide (E2A), a cytoplasmic polyhedrosis virus 2A peptide (BmCPV 2A), and a flacherie virus of B. mori 2A peptide (BmIFV 2A); and/or
(c) the promoter is a CMV promoter.

39-42. (canceled)

43. The chimeric polynucleotide of claim 30, wherein the nucleic acid encoding the antigenic polypeptide is operably linked to a first promoter and the nucleic acid encoding the SARS-CoV-2 N protein is operably linked to a second promoter.

44. The chimeric polynucleotide of claim 43, wherein the first promoter and the second promoter are each a CMV promoter: or the first promoter is a CMV promoter and the second promoter is a beta-actin promoter; or the first promoter is a beta-actin promoter and the second promoter is a CMV promoter.

45. (canceled)

46. The chimeric polynucleotide of claim 30, wherein the expression cassette comprises a polyadenylation signal, optionally wherein the polyadenylation signal is a bovine growth hormone polyadenylation signal.

47. (canceled)

48. The chimeric polynucleotide of claim 30, wherein the chimeric polynucleotide further comprises a nucleic acid encoding a a toll-like receptor-3 (TLR-3) agonist.

49. The chimeric polynucleotide of claim 48, wherein the TLR-3 agonist comprises a nucleic acid encoding a dsRNA, optionally wherein the nucleic acid encoding the TLR-3 agonist comprises a sequence selected from the group consisting of SEQ ID NOS:11-18.

50. (canceled)

51. An expression vector comprising the chimeric polynucleotide of claim 30.

52. A method of inducing an immune response in a subject, the method comprising administering the expression vector of claim 51 to the subject, optionally wherein the subject is a human.

53. (canceled)

54. A host cell comprising the chimeric polynucleotide of claim 30, optionally wherein the host cell is mammalian.

55. (canceled)

56. A chimeric adenoviral expression vector, comprising a bicistronic or multicistronic expression construct comprising:
a nucleic acid encoding a SARS-CoV-2 S protein; and
a nucleic acid encoding a SARS-CoV-2 N protein,
wherein the bicistronic construct is operably linked to a promoter.

57. The chimeric adenoviral expression vector of claim 56, wherein the SARS-CoV-2 N protein comprises an amino acid sequence having at least 95%, 96%, 97%, 98%, 99%, or 100% identity to the amino acid sequence of SEQ ID NO:2, optionally wherein the nucleic acid encoding the SARS-CoV-2 N protein comprises a sequence having at least 85%, 90%, 95%, 97%, 99%, or 100% identity to the sequence of SEQ ID NO:4; and/or the SARS-CoV-2 S protein comprises a sequence having at least 90% identity to SEQ ID NO:1, optionally wherein the nucleic acid encoding the SARS-CoV-2 S protein comprises a sequence having at least 85%, 90%, 95%, 97%, 99%, or 100% identity to the sequence of SEQ ID NO:3.

58-63. (canceled)

64. The chimeric adenoviral expression vector of claim 56, wherein the expression cassette comprises an internal ribosome entry site (IRES), a ribosomal skipping element, or a furin cleavage site positioned between the nucleic acid encoding the SARS-CoV-2 S protein and the nucleic acid encoding the SARS-CoV-2 N protein, optionally wherein the expression cassette comprises a ribosomal skipping element and the ribosomal skipping element is a sequence encoding a peptide selected from the group consisting of a 2A peptide (T2A), a porcine teschovirus-1 2A peptide (P2A), a foot-and-mouth disease virus 2A peptide (F2A), equine rhinitis A virus 2A peptide (E2A), a cytoplasmic polyhedrosis virus 2A peptide (BmCPV 2A), and a flacherie virus of B. mori 2A peptide (BmIFV 2A).

65-66. (canceled)

67. The chimeric adenoviral vector of claim 56, wherein:
the promoter is a CMV promoter, and/or
the expression cassette comprises a polyadenylation signal, optionally wherein the polyadenylation signal is a bovine growth hormone polyadenylation signal; and/or,
the chimeric adenoviral expression vector further comprises a nucleic acid encoding a toll-like receptor-3 (TLR-3) agonist, optionally wherein the nucleic acid encoding the TLR-3 agonist comprises a sequence selected from the group consisting of SEQ ID NOS:11-18.

68-72. (canceled)


SEQUENCE LISTING (25 sequences)

SEQ ID NO: 1 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 419 aa, Severe acute respiratory syndrome coronavirus 2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 3 - DNA, 3840 bp, Severe acute respiratory syndrome coronavirus 2
ggtaccgccaccatgtttgtttttctcgtactcctgcccctggtttcctcccaatgtgtcaatctgactacccggacccaacttcctcccgcctacaccaattcctttacccgaggtgtttactacccagacaaagtgttcaggtcatccgtcctccatagtacccaagacctcttcctcccttttttttctaacgttacctggtttcacgctattcacgttagcggcaccaacggcaccaaaagattcgataaccccgtactgccgttcaacgacggggtatattttgcctctactgaaaaatcaaacatcatacgcggatggatctttgggactaccctggactcaaaaactcagtccctgctgattgtgaataacgctaccaacgtggtgatcaaagtctgtgaattccagttttgcaacgatccttttctcggcgtttattatcacaaaaataacaaatcctggatggagagcgagttccgggtgtactcctccgcgaataattgcaccttcgaatatgtgtctcagccattcctcatggacctcgaggggaagcagggcaattttaagaatctgcgagaattcgtgttcaagaatatagacggttacttcaagatttactccaaacacaccccgattaacctggttagggacttgcctcagggcttttctgcattggagcccctcgtggacctcccaatcggcataaacattacaagatttcagactttgcttgcattgcacaggagctatttgacacccggcgattcttcttccggatggaccgctggagcagctgcttattacgtgggctatctgcagcctcgaacctttcttttgaagtacaacgaaaatggaactatcaccgatgcagttgactgcgccctggaccccctgtccgaaactaagtgcacgctcaaaagtttcacagtagagaaggggatataccagactagcaatttccgcgttcagccaaccgaaagtatagtgcgctttcctaatataactaacctgtgtcctttcggggaagtgtttaacgccactagattcgcttccgtctacgcctggaatagaaagaggatctcaaattgcgttgctgactatagtgttttgtacaattccgcctctttctcaaccttcaaatgttacggggtgagccctaccaaactgaacgacctgtgctttacaaacgtatacgccgacagctttgttatcagaggagacgaggttcgccagattgctccgggtcagacaggcaagattgctgattataattacaaactgcccgacgactttacaggatgtgtgatcgcgtggaacagtaacaatcttgactcaaaggttgggggtaattataattatctttaccggctgttcagaaaaagcaatttgaaacccttcgaaagggacatatccaccgagatctatcaggccgggtccactccatgcaatggtgtggaaggttttaattgctacttcccattgcagtcttatggattccaaccaaccaatggcgtaggctaccagccgtatcgcgttgtcgtgctcagcttcgagctgctccacgcccccgcgaccgtatgcggtcctaagaagtccaccaatcttgttaagaacaagtgtgtaaactttaactttaacgggctgaccgggaccggcgttctgactgaatctaacaaaaaattcctgcctttccagcagttcggccgcgatattgctgacaccactgacgctgtaagagaccctcagacccttgaaattctcgatatcacaccttgcagctttgggggcgtgtccgtcatcactccaggaactaacacaagcaaccaggtggcagtgttgtaccaggatgttaattgtaccgaggtgccagtggccatccacgccgatcaattgacacctacctggagggtttacagcacagggtccaatgtttttcagacaagagccggatgtctgatcggtgccgagcatgtcaacaattcctacgagtgtgatatccccattggtgcgggaatttgtgcatcatatcagacccagactaatagcccaagaagagctagatccgtcgctagtcaatccatcattgcatatacaatgtccctgggagctgagaattcagtcgcgtattcaaacaattccattgctattcctactaatttcactatctccgtcacgaccgagatcctgccagtttccatgactaagacttctgttgactgcaccatgtatatctgtggcgatagcaccgagtgcagtaatctgcttctgcagtacggctccttctgcacacaactcaatcgagcactgaccggtattgcagttgagcaggacaagaacacacaggaggtctttgcacaggtcaaacaaatttacaaaaccccccccataaaagactttggtgggttcaacttcagccaaatcctcccagatcccagcaagccctccaaaagatccttcatcgaagaccttttgttcaataaggtaaccctggccgacgcaggcttcatcaaacaatatggcgattgccttggagacattgctgcgcgcgatttgatctgtgctcagaaatttaacggtttgaccgtgctgcccccacttctgactgatgagatgatagcacagtatacttctgctcttctggcaggaacaatcacttccgggtggacctttggcgctggtgcagcactgcaaatccccttcgcaatgcaaatggcctaccgattcaatggtattggtgttacccagaacgtgctctatgagaatcagaaactcatcgccaatcagttcaatagcgctattggcaagattcaggattccctcagctctaccgccagcgctctggggaagctccaggacgtggtgaaccaaaatgctcaagcgctcaatacccttgtgaaacagctcagctccaattttggcgcaattagcagcgttctgaatgatattctgtcccggctggacaaggtagaagcagaagtccagatcgacaggctgatcaccgggcggttgcagagtctccagacctatgtcacacaacagctgatccgcgccgccgagatcagggcttccgctaacctggccgccactaagatgtccgaatgcgtgttggggcagagtaagcgggtcgacttttgcgggaagggataccatctgatgagcttccctcagtctgcaccccacggagtagtgttcctccacgtcacatatgtgcccgctcaggaaaagaatttcacaaccgcacctgctatctgtcacgacggcaaggcccactttcctagagaaggagttttcgtatctaacggcacccactggttcgtgacacagcggaacttttacgagcctcagattataactacggacaacactttcgtgtcaggcaactgtgacgtggtgattgggatcgtgaacaacacagtctacgacccattgcagcccgagttggactccttcaaagaggagcttgataagtatttcaagaaccatacctctcccgacgtggacctgggggacattagcggcatcaatgcatccgttgtgaatatccagaaagaaatcgataggctgaatgaggtcgcaaaaaatcttaatgagtcactgattgatctgcaggaactcggcaaatatgagcagtatattaagtggccgtggtacatatggctcggctttatcgccggtctgattgccatcgtgatggtgaccattatgctgtgttgtatgacaagctgctgttcatgtctcaaaggatgctgctcctgcggtagctgctgtaagttcgatgaagacgacagtgagcccgtgctcaaaggagtgaaactccactacacataacgatcg

SEQ ID NO: 4 - DNA, 1278 bp, Severe acute respiratory syndrome coronavirus 2
ggtaccgccaccatgtccgataacggcccccagaatcagagaaacgctccccgcatcacgttcggcggaccaagtgacagcacaggcagtaaccagaacggagaacgctccggtgctcgctccaagcagcgacggccgcaagggcttcccaacaataccgccagctggtttacggctctgacccaacacgggaaagaagatcttaaattccccaggggccagggcgtccctatcaatactaactccagcccggatgatcagataggctactatagacgcgctacccgacggatacgagggggggacggcaaaatgaaggacctttccccccggtggtatttctattacttgggcaccggaccagaagccggactgccttacggcgctaacaaagacggaataatctgggttgcgacggagggcgccctgaatacacctaaagaccatatcggcacaagaaatcctgctaacaatgccgcgattgtgctccagctgcctcagggaaccacgctgcctaaagggttttacgctgaggggtcaaggggggggagtcaagcgtctagtaggtcatcctctcgctctcgcaatagttcccggaactcaaccccaggcagcagcagaggaacctctcccgcacggatggctggcaatgggggagatgctgcccttgctctccttctgctggatcgccttaaccagctcgaatcaaagatgtctggaaaaggtcagcagcagcaaggccagaccgtgacaaagaagagtgcagctgaagctagtaaaaagccacgccaaaaacggaccgcaactaaggcatataacgtaacacaggccttcggcagaagaggtccagaacaaacacagggaaactttggcgatcaagagctgattagacagggcacagattacaaacactggccacagatcgcgcagtttgcaccaagcgcctctgcattcttcgggatgagtcggattgggatggaagtcactccatccgggacctggcttacctacacaggggcaataaaactcgacgacaaagacccaaactttaaagatcaggtcatcctgctgaataaacacatcgatgcctacaaaactttccccccaaccgaaccaaagaaagacaagaaaaaaaaggcagacgaaacgcaagcgctccctcagcgccagaagaagcagcagaccgttacactgttgccagcagcagatctggatgatttttccaagcagcttcaacagagtatgtcaagcgctgacagcactcaggcttgacgatcg

SEQ ID NO: 5 - DNA, 3369 bp, Artificial Sequence
  Description of Artificial Sequence Synthetic polynucleotide
ggtaccgccaccatgtttgtttttctcgtactcctgcccctggtttcctcccaatgtgtcaatctgactacccggacccaacttcctcccgcctacaccaattcctttacccgaggtgtttactacccagacaaagtgttcaggtcatccgtcctccatagtacccaagacctcttcctcccttttttttctaacgttacctggtttcacgctattcacgttagcggcaccaacggcaccaaaagattcgataaccccgtactgccgttcaacgacggggtatattttgcctctactgaaaaatcaaacatcatacgcggatggatctttgggactaccctggactcaaaaactcagtccctgctgattgtgaataacgctaccaacgtggtgatcaaagtctgtgaattccagttttgcaacgatccttttctcggcgtttattatcacaaaaataacaaatcctggatggagagcgagttccgggtgtactcctccgcgaataattgcaccttcgaatatgtgtctcagccattcctcatggacctcgaggggaagcagggcaattttaagaatctgcgagaattcgtgttcaagaatatagacggttacttcaagatttactccaaacacaccccgattaacctggttagggacttgcctcagggcttttctgcattggagcccctcgtggacctcccaatcggcataaacattacaagatttcagactttgcttgcattgcacaggagctatttgacacccggcgattcttcttccggatggaccgctggagcagctgcttattacgtgggctatctgcagcctcgaacctttcttttgaagtacaacgaaaatggaactatcaccgatgcagttgactgcgccctggaccccctgtccgaaactaagtgcacgctcaaaagtttcacagtagagaaggggatataccagactagcaatttccgcgttcagccaaccgaaagtatagtgcgctttcctaatataactaacctgtgtcctttcggggaagtgtttaacgccactagattcgcttccgtctacgcctggaatagaaagaggatctcaaattgcgttgctgactatagtgttttgtacaattccgcctctttctcaaccttcaaatgttacggggtgagccctaccaaactgaacgacctgtgctttacaaacgtatacgccgacagctttgttatcagaggagacgaggttcgccagattgctccgggtcagacaggcaagattgctgattataattacaaactgcccgacgactttacaggatgtgtgatcgcgtggaacagtaacaatcttgactcaaaggttgggggtaattataattatctttaccggctgttcagaaaaagcaatttgaaacccttcgaaagggacatatccaccgagatctatcaggccgggtccactccatgcaatggtgtggaaggttttaattgctacttcccattgcagtcttatggattccaaccaaccaatggcgtaggctaccagccgtatcgcgttgtcgtgctcagcttcgagctgctccacgcccccgcgaccgtatgcggtcctaagaagtccaccaatcttgttaagaacaagtgtgtaaactttaactttaacgggctgaccgggaccggcgttctgactgaatctaacaaaaaattcctgcctttccagcagttcggccgcgatattgctgacaccactgacgctgtaagagaccctcagacccttgaaattctcgatatcacaccttgcagctttgggggcgtgtccgtcatcactccaggaactaacacaagcaaccaggtggcagtgttgtaccaggatgttaattgtaccgaggtgccagtggccatccacgccgatcaattgacacctacctggagggtttacagcacagggtccaatgtttttcagacaagagccggatgtctgatcggtgccgagcatgtcaacaattcctacgagtgtgatatccccattggtgcgggaatttgtgcatcatatcagacccagactaatagcccaagaagagctagatccgtcgctagtcaatccatcattgcatatacaatgatgtccgataacggcccccagaatcagagaaacgctccccgcatcacgttcggcggaccaagtgacagcacaggcagtaaccagaacggagaacgctccggtgctcgctccaagcagcgacggccgcaagggcttcccaacaataccgccagctggtttacggctctgacccaacacgggaaagaagatcttaaattccccaggggccagggcgtccctatcaatactaactccagcccggatgatcagataggctactatagacgcgctacccgacggatacgagggggggacggcaaaatgaaggacctttccccccggtggtatttctattacttgggcaccggaccagaagccggactgccttacggcgctaacaaagacggaataatctgggttgcgacggagggcgccctgaatacacctaaagaccatatcggcacaagaaatcctgctaacaatgccgcgattgtgctccagctgcctcagggaaccacgctgcctaaagggttttacgctgaggggtcaaggggggggagtcaagcgtctagtaggtcatcctctcgctctcgcaatagttcccggaactcaaccccaggcagcagcagaggaacctctcccgcacggatggctggcaatgggggagatgctgcccttgctctccttctgctggatcgccttaaccagctcgaatcaaagatgtctggaaaaggtcagcagcagcaaggccagaccgtgacaaagaagagtgcagctgaagctagtaaaaagccacgccaaaaacggaccgcaactaaggcatataacgtaacacaggccttcggcagaagaggtccagaacaaacacagggaaactttggcgatcaagagctgattagacagggcacagattacaaacactggccacagatcgcgcagtttgcaccaagcgcctctgcattcttcgggatgagtcggattgggatggaagtcactccatccgggacctggcttacctacacaggggcaataaaactcgacgacaaagacccaaactttaaagatcaggtcatcctgctgaataaacacatcgatgcctacaaaactttccccccaaccgaaccaaagaaagacaagaaaaaaaaggcagacgaaacgcaagcgctccctcagcgccagaagaagcagcagaccgttacactgttgccagcagcagatctggatgatttttccaagcagcttcaacagagtatgtcaagcgctgacagcactcaggcttgacgatcg

SEQ ID NO: 6 - DNA, 7310 bp, Artificial Sequence
  Description of Artificial Sequence Synthetic polynucleotide
tagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctgactctagcctagctctgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtctgagcctaggagatctctcgaggtcgacggtatcgatgggtaccgccaccatgtttgtttttctcgtactcctgcccctggtttcctcccaatgtgtcaatctgactacccggacccaacttcctcccgcctacaccaattcctttacccgaggtgtttactacccagacaaagtgttcaggtcatccgtcctccatagtacccaagacctcttcctcccttttttttctaacgttacctggtttcacgctattcacgttagcggcaccaacggcaccaaaagattcgataaccccgtactgccgttcaacgacggggtatattttgcctctactgaaaaatcaaacatcatacgcggatggatctttgggactaccctggactcaaaaactcagtccctgctgattgtgaataacgctaccaacgtggtgatcaaagtctgtgaattccagttttgcaacgatccttttctcggcgtttattatcacaaaaataacaaatcctggatggagagcgagttccgggtgtactcctccgcgaataattgcaccttcgaatatgtgtctcagccattcctcatggacctcgaggggaagcagggcaattttaagaatctgcgagaattcgtgttcaagaatatagacggttacttcaagatttactccaaacacaccccgattaacctggttagggacttgcctcagggcttttctgcattggagcccctcgtggacctcccaatcggcataaacattacaagatttcagactttgcttgcattgcacaggagctatttgacacccggcgattcttcttccggatggaccgctggagcagctgcttattacgtgggctatctgcagcctcgaacctttcttttgaagtacaacgaaaatggaactatcaccgatgcagttgactgcgccctggaccccctgtccgaaactaagtgcacgctcaaaagtttcacagtagagaaggggatataccagactagcaatttccgcgttcagccaaccgaaagtatagtgcgctttcctaatataactaacctgtgtcctttcggggaagtgtttaacgccactagattcgcttccgtctacgcctggaatagaaagaggatctcaaattgcgttgctgactatagtgttttgtacaattccgcctctttctcaaccttcaaatgttacggggtgagccctaccaaactgaacgacctgtgctttacaaacgtatacgccgacagctttgttatcagaggagacgaggttcgccagattgctccgggtcagacaggcaagattgctgattataattacaaactgcccgacgactttacaggatgtgtgatcgcgtggaacagtaacaatcttgactcaaaggttgggggtaattataattatctttaccggctgttcagaaaaagcaatttgaaacccttcgaaagggacatatccaccgagatctatcaggccgggtccactccatgcaatggtgtggaaggttttaattgctacttcccattgcagtcttatggattccaaccaaccaatggcgtaggctaccagccgtatcgcgttgtcgtgctcagcttcgagctgctccacgcccccgcgaccgtatgcggtcctaagaagtccaccaatcttgttaagaacaagtgtgtaaactttaactttaacgggctgaccgggaccggcgttctgactgaatctaacaaaaaattcctgcctttccagcagttcggccgcgatattgctgacaccactgacgctgtaagagaccctcagacccttgaaattctcgatatcacaccttgcagctttgggggcgtgtccgtcatcactccaggaactaacacaagcaaccaggtggcagtgttgtaccaggatgttaattgtaccgaggtgccagtggccatccacgccgatcaattgacacctacctggagggtttacagcacagggtccaatgtttttcagacaagagccggatgtctgatcggtgccgagcatgtcaacaattcctacgagtgtgatatccccattggtgcgggaatttgtgcatcatatcagacccagactaatagcccaagaagagctagatccgtcgctagtcaatccatcattgcatatacaatgtccctgggagctgagaattcagtcgcgtattcaaacaattccattgctattcctactaatttcactatctccgtcacgaccgagatcctgccagtttccatgactaagacttctgttgactgcaccatgtatatctgtggcgatagcaccgagtgcagtaatctgcttctgcagtacggctccttctgcacacaactcaatcgagcactgaccggtattgcagttgagcaggacaagaacacacaggaggtctttgcacaggtcaaacaaatttacaaaaccccccccataaaagactttggtgggttcaacttcagccaaatcctcccagatcccagcaagccctccaaaagatccttcatcgaagaccttttgttcaataaggtaaccctggccgacgcaggcttcatcaaacaatatggcgattgccttggagacattgctgcgcgcgatttgatctgtgctcagaaatttaacggtttgaccgtgctgcccccacttctgactgatgagatgatagcacagtatacttctgctcttctggcaggaacaatcacttccgggtggacctttggcgctggtgcagcactgcaaatccccttcgcaatgcaaatggcctaccgattcaatggtattggtgttacccagaacgtgctctatgagaatcagaaactcatcgccaatcagttcaatagcgctattggcaagattcaggattccctcagctctaccgccagcgctctggggaagctccaggacgtggtgaaccaaaatgctcaagcgctcaatacccttgtgaaacagctcagctccaattttggcgcaattagcagcgttctgaatgatattctgtcccggctggacaaggtagaagcagaagtccagatcgacaggctgatcaccgggcggttgcagagtctccagacctatgtcacacaacagctgatccgcgccgccgagatcagggcttccgctaacctggccgccactaagatgtccgaatgcgtgttggggcagagtaagcgggtcgacttttgcgggaagggataccatctgatgagcttccctcagtctgcaccccacggagtagtgttcctccacgtcacatatgtgcccgctcaggaaaagaatttcacaaccgcacctgctatctgtcacgacggcaaggcccactttcctagagaaggagttttcgtatctaacggcacccactggttcgtgacacagcggaacttttacgagcctcagattataactacggacaacactttcgtgtcaggcaactgtgacgtggtgattgggatcgtgaacaacacagtctacgacccattgcagcccgagttggactccttcaaagaggagcttgataagtatttcaagaaccatacctctcccgacgtggacctgggggacattagcggcatcaatgcatccgttgtgaatatccagaaagaaatcgataggctgaatgaggtcgcaaaaaatcttaatgagtcactgattgatctgcaggaactcggcaaatatgagcagtatattaagtggccgtggtacatatggctcggctttatcgccggtctgattgccatcgtgatggtgaccattatgctgtgttgtatgacaagctgctgttcatgtctcaaaggatgctgctcctgcggtagctgctgtaagttcgatgaagacgacagtgagcccgtgctcaaaggagtgaaactccactacacataacgatcgacgcgtagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccaaagcttgcggccgcgcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagaccctgcaggccgccaccatgtccgataacggcccccagaatcagagaaacgctccccgcatcacgttcggcggaccaagtgacagcacaggcagtaaccagaacggagaacgctccggtgctcgctccaagcagcgacggccgcaagggcttcccaacaataccgccagctggtttacggctctgacccaacacgggaaagaagatcttaaattccccaggggccagggcgtccctatcaatactaactccagcccggatgatcagataggctactatagacgcgctacccgacggatacgagggggggacggcaaaatgaaggacctttccccccggtggtatttctattacttgggcaccggaccagaagccggactgccttacggcgctaacaaagacggaataatctgggttgcgacggagggcgccctgaatacacctaaagaccatatcggcacaagaaatcctgctaacaatgccgcgattgtgctccagctgcctcagggaaccacgctgcctaaagggttttacgctgaggggtcaaggggggggagtcaagcgtctagtaggtcatcctctcgctctcgcaatagttcccggaactcaaccccaggcagcagcagaggaacctctcccgcacggatggctggcaatgggggagatgctgcccttgctctccttctgctggatcgccttaaccagctcgaatcaaagatgtctggaaaaggtcagcagcagcaaggccagaccgtgacaaagaagagtgcagctgaagctagtaaaaagccacgccaaaaacggaccgcaactaaggcatataacgtaacacaggccttcggcagaagaggtccagaacaaacacagggaaactttggcgatcaagagctgattagacagggcacagattacaaacactggccacagatcgcgcagtttgcaccaagcgcctctgcattcttcgggatgagtcggattgggatggaagtcactccatccgggacctggcttacctacacaggggcaataaaactcgacgacaaagacccaaactttaaagatcaggtcatcctgctgaataaacacatcgatgcctacaaaactttccccccaaccgaaccaaagaaagacaagaaaaaaaaggcagacgaaacgcaagcgctccctcagcgccagaagaagcagcagaccgttacactgttgccagcagcagatctggatgatttttccaagcagcttcaacagagtatgtcaagcgctgacagcactcaggcttgaggcgcgccgctgaccgataaataaaatatctttattttcattacatctgtgtgttggttttttgtgtgacgcgttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatatcgggccactgcaggaaacgatatgggctgaatacggatccgtattcagcccatatcgtttctctagaaataaaatatctttattttcattacatctgtgtgttggttttttgtgtg

SEQ ID NO: 7 - DNA, 5281 bp, Artificial Sequence
  Description of Artificial Sequence Synthetic polynucleotide
tagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctgactctagcctagctctgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtctgagcctaggagatctctcgaggtcgacggtatcgatgggtaccgccaccatgtttgtttttctcgtactcctgcccctggtttcctcccaatgtgtcaatctgactacccggacccaacttcctcccgcctacaccaattcctttacccgaggtgtttactacccagacaaagtgttcaggtcatccgtcctccatagtacccaagacctcttcctcccttttttttctaacgttacctggtttcacgctattcacgttagcggcaccaacggcaccaaaagattcgataaccccgtactgccgttcaacgacggggtatattttgcctctactgaaaaatcaaacatcatacgcggatggatctttgggactaccctggactcaaaaactcagtccctgctgattgtgaataacgctaccaacgtggtgatcaaagtctgtgaattccagttttgcaacgatccttttctcggcgtttattatcacaaaaataacaaatcctggatggagagcgagttccgggtgtactcctccgcgaataattgcaccttcgaatatgtgtctcagccattcctcatggacctcgaggggaagcagggcaattttaagaatctgcgagaattcgtgttcaagaatatagacggttacttcaagatttactccaaacacaccccgattaacctggttagggacttgcctcagggcttttctgcattggagcccctcgtggacctcccaatcggcataaacattacaagatttcagactttgcttgcattgcacaggagctatttgacacccggcgattcttcttccggatggaccgctggagcagctgcttattacgtgggctatctgcagcctcgaacctttcttttgaagtacaacgaaaatggaactatcaccgatgcagttgactgcgccctggaccccctgtccgaaactaagtgcacgctcaaaagtttcacagtagagaaggggatataccagactagcaatttccgcgttcagccaaccgaaagtatagtgcgctttcctaatataactaacctgtgtcctttcggggaagtgtttaacgccactagattcgcttccgtctacgcctggaatagaaagaggatctcaaattgcgttgctgactatagtgttttgtacaattccgcctctttctcaaccttcaaatgttacggggtgagccctaccaaactgaacgacctgtgctttacaaacgtatacgccgacagctttgttatcagaggagacgaggttcgccagattgctccgggtcagacaggcaagattgctgattataattacaaactgcccgacgactttacaggatgtgtgatcgcgtggaacagtaacaatcttgactcaaaggttgggggtaattataattatctttaccggctgttcagaaaaagcaatttgaaacccttcgaaagggacatatccaccgagatctatcaggccgggtccactccatgcaatggtgtggaaggttttaattgctacttcccattgcagtcttatggattccaaccaaccaatggcgtaggctaccagccgtatcgcgttgtcgtgctcagcttcgagctgctccacgcccccgcgaccgtatgcggtcctaagaagtccaccaatcttgttaagaacaagtgtgtaaactttaactttaacgggctgaccgggaccggcgttctgactgaatctaacaaaaaattcctgcctttccagcagttcggccgcgatattgctgacaccactgacgctgtaagagaccctcagacccttgaaattctcgatatcacaccttgcagctttgggggcgtgtccgtcatcactccaggaactaacacaagcaaccaggtggcagtgttgtaccaggatgttaattgtaccgaggtgccagtggccatccacgccgatcaattgacacctacctggagggtttacagcacagggtccaatgtttttcagacaagagccggatgtctgatcggtgccgagcatgtcaacaattcctacgagtgtgatatccccattggtgcgggaatttgtgcatcatatcagacccagactaatagcccaagaagagctagatccgtcgctagtcaatccatcattgcatatacaatgatgtccgataacggcccccagaatcagagaaacgctccccgcatcacgttcggcggaccaagtgacagcacaggcagtaaccagaacggagaacgctccggtgctcgctccaagcagcgacggccgcaagggcttcccaacaataccgccagctggtttacggctctgacccaacacgggaaagaagatcttaaattccccaggggccagggcgtccctatcaatactaactccagcccggatgatcagataggctactatagacgcgctacccgacggatacgagggggggacggcaaaatgaaggacctttccccccggtggtatttctattacttgggcaccggaccagaagccggactgccttacggcgctaacaaagacggaataatctgggttgcgacggagggcgccctgaatacacctaaagaccatatcggcacaagaaatcctgctaacaatgccgcgattgtgctccagctgcctcagggaaccacgctgcctaaagggttttacgctgaggggtcaaggggggggagtcaagcgtctagtaggtcatcctctcgctctcgcaatagttcccggaactcaaccccaggcagcagcagaggaacctctcccgcacggatggctggcaatgggggagatgctgcccttgctctccttctgctggatcgccttaaccagctcgaatcaaagatgtctggaaaaggtcagcagcagcaaggccagaccgtgacaaagaagagtgcagctgaagctagtaaaaagccacgccaaaaacggaccgcaactaaggcatataacgtaacacaggccttcggcagaagaggtccagaacaaacacagggaaactttggcgatcaagagctgattagacagggcacagattacaaacactggccacagatcgcgcagtttgcaccaagcgcctctgcattcttcgggatgagtcggattgggatggaagtcactccatccgggacctggcttacctacacaggggcaataaaactcgacgacaaagacccaaactttaaagatcaggtcatcctgctgaataaacacatcgatgcctacaaaactttccccccaaccgaaccaaagaaagacaagaaaaaaaaggcagacgaaacgcaagcgctccctcagcgccagaagaagcagcagaccgttacactgttgccagcagcagatctggatgatttttccaagcagcttcaacagagtatgtcaagcgctgacagcactcaggcttgacgatcggatatcgctagcgtaccggcggccgccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccaaagcttacgcgttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatatcgggccactgcaggaaacgatatgggctgaatacggatccgtattcagcccatatcgtttctctagaaataaaatatctttattttcattacatctgtgtgttggttttttgtgtg

SEQ ID NO: 8 - DNA, 37735 bp, Artificial Sequence
  Description of Artificial Sequence Synthetic polynucleotide
taaggatcccatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatactgctagagatctggcgaaagggggatgtgctgcaaggcgattaagttgggtaacgccagggttttcccagtcacgacgttgtaaaacgacggccagtgaattgtaatacgactcactatagggcgaattgggtactggccacaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctgactctagcctagctctgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtctgagcctaggagatctctcgaggtcgacggtatcgatgggtaccgccaccatgtttgtttttctcgtactcctgcccctggtttcctcccaatgtgtcaatctgactacccggacccaacttcctcccgcctacaccaattcctttacccgaggtgtttactacccagacaaagtgttcaggtcatccgtcctccatagtacccaagacctcttcctcccttttttttctaacgttacctggtttcacgctattcacgttagcggcaccaacggcaccaaaagattcgataaccccgtactgccgttcaacgacggggtatattttgcctctactgaaaaatcaaacatcatacgcggatggatctttgggactaccctggactcaaaaactcagtccctgctgattgtgaataacgctaccaacgtggtgatcaaagtctgtgaattccagttttgcaacgatccttttctcggcgtttattatcacaaaaataacaaatcctggatggagagcgagttccgggtgtactcctccgcgaataattgcaccttcgaatatgtgtctcagccattcctcatggacctcgaggggaagcagggcaattttaagaatctgcgagaattcgtgttcaagaatatagacggttacttcaagatttactccaaacacaccccgattaacctggttagggacttgcctcagggcttttctgcattggagcccctcgtggacctcccaatcggcataaacattacaagatttcagactttgcttgcattgcacaggagctatttgacacccggcgattcttcttccggatggaccgctggagcagctgcttattacgtgggctatctgcagcctcgaacctttcttttgaagtacaacgaaaatggaactatcaccgatgcagttgactgcgccctggaccccctgtccgaaactaagtgcacgctcaaaagtttcacagtagagaaggggatataccagactagcaatttccgcgttcagccaaccgaaagtatagtgcgctttcctaatataactaacctgtgtcctttcggggaagtgtttaacgccactagattcgcttccgtctacgcctggaatagaaagaggatctcaaattgcgttgctgactatagtgttttgtacaattccgcctctttctcaaccttcaaatgttacggggtgagccctaccaaactgaacgacctgtgctttacaaacgtatacgccgacagctttgttatcagaggagacgaggttcgccagattgctccgggtcagacaggcaagattgctgattataattacaaactgcccgacgactttacaggatgtgtgatcgcgtggaacagtaacaatcttgactcaaaggttgggggtaattataattatctttaccggctgttcagaaaaagcaatttgaaacccttcgaaagggacatatccaccgagatctatcaggccgggtccactccatgcaatggtgtggaaggttttaattgctacttcccattgcagtcttatggattccaaccaaccaatggcgtaggctaccagccgtatcgcgttgtcgtgctcagcttcgagctgctccacgcccccgcgaccgtatgcggtcctaagaagtccaccaatcttgttaagaacaagtgtgtaaactttaactttaacgggctgaccgggaccggcgttctgactgaatctaacaaaaaattcctgcctttccagcagttcggccgcgatattgctgacaccactgacgctgtaagagaccctcagacccttgaaattctcgatatcacaccttgcagctttgggggcgtgtccgtcatcactccaggaactaacacaagcaaccaggtggcagtgttgtaccaggatgttaattgtaccgaggtgccagtggccatccacgccgatcaattgacacctacctggagggtttacagcacagggtccaatgtttttcagacaagagccggatgtctgatcggtgccgagcatgtcaacaattcctacgagtgtgatatccccattggtgcgggaatttgtgcatcatatcagacccagactaatagcccaagaagagctagatccgtcgctagtcaatccatcattgcatatacaatgtccctgggagctgagaattcagtcgcgtattcaaacaattccattgctattcctactaatttcactatctccgtcacgaccgagatcctgccagtttccatgactaagacttctgttgactgcaccatgtatatctgtggcgatagcaccgagtgcagtaatctgcttctgcagtacggctccttctgcacacaactcaatcgagcactgaccggtattgcagttgagcaggacaagaacacacaggaggtctttgcacaggtcaaacaaatttacaaaaccccccccataaaagactttggtgggttcaacttcagccaaatcctcccagatcccagcaagccctccaaaagatccttcatcgaagaccttttgttcaataaggtaaccctggccgacgcaggcttcatcaaacaatatggcgattgccttggagacattgctgcgcgcgatttgatctgtgctcagaaatttaacggtttgaccgtgctgcccccacttctgactgatgagatgatagcacagtatacttctgctcttctggcaggaacaatcacttccgggtggacctttggcgctggtgcagcactgcaaatccccttcgcaatgcaaatggcctaccgattcaatggtattggtgttacccagaacgtgctctatgagaatcagaaactcatcgccaatcagttcaatagcgctattggcaagattcaggattccctcagctctaccgccagcgctctggggaagctccaggacgtggtgaaccaaaatgctcaagcgctcaatacccttgtgaaacagctcagctccaattttggcgcaattagcagcgttctgaatgatattctgtcccggctggacaaggtagaagcagaagtccagatcgacaggctgatcaccgggcggttgcagagtctccagacctatgtcacacaacagctgatccgcgccgccgagatcagggcttccgctaacctggccgccactaagatgtccgaatgcgtgttggggcagagtaagcgggtcgacttttgcgggaagggataccatctgatgagcttccctcagtctgcaccccacggagtagtgttcctccacgtcacatatgtgcccgctcaggaaaagaatttcacaaccgcacctgctatctgtcacgacggcaaggcccactttcctagagaaggagttttcgtatctaacggcacccactggttcgtgacacagcggaacttttacgagcctcagattataactacggacaacactttcgtgtcaggcaactgtgacgtggtgattgggatcgtgaacaacacagtctacgacccattgcagcccgagttggactccttcaaagaggagcttgataagtatttcaagaaccatacctctcccgacgtggacctgggggacattagcggcatcaatgcatccgttgtgaatatccagaaagaaatcgataggctgaatgaggtcgcaaaaaatcttaatgagtcactgattgatctgcaggaactcggcaaatatgagcagtatattaagtggccgtggtacatatggctcggctttatcgccggtctgattgccatcgtgatggtgaccattatgctgtgttgtatgacaagctgctgttcatgtctcaaaggatgctgctcctgcggtagctgctgtaagttcgatgaagacgacagtgagcccgtgctcaaaggagtgaaactccactacacataacgatcgacgcgtagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccaaagcttgcggccgcgcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagaccctgcaggccgccaccatgtccgataacggcccccagaatcagagaaacgctccccgcatcacgttcggcggaccaagtgacagcacaggcagtaaccagaacggagaacgctccggtgctcgctccaagcagcgacggccgcaagggcttcccaacaataccgccagctggtttacggctctgacccaacacgggaaagaagatcttaaattccccaggggccagggcgtccctatcaatactaactccagcccggatgatcagataggctactatagacgcgctacccgacggatacgagggggggacggcaaaatgaaggacctttccccccggtggtatttctattacttgggcaccggaccagaagccggactgccttacggcgctaacaaagacggaataatctgggttgcgacggagggcgccctgaatacacctaaagaccatatcggcacaagaaatcctgctaacaatgccgcgattgtgctccagctgcctcagggaaccacgctgcctaaagggttttacgctgaggggtcaaggggggggagtcaagcgtctagtaggtcatcctctcgctctcgcaatagttcccggaactcaaccccaggcagcagcagaggaacctctcccgcacggatggctggcaatgggggagatgctgcccttgctctccttctgctggatcgccttaaccagctcgaatcaaagatgtctggaaaaggtcagcagcagcaaggccagaccgtgacaaagaagagtgcagctgaagctagtaaaaagccacgccaaaaacggaccgcaactaaggcatataacgtaacacaggccttcggcagaagaggtccagaacaaacacagggaaactttggcgatcaagagctgattagacagggcacagattacaaacactggccacagatcgcgcagtttgcaccaagcgcctctgcattcttcgggatgagtcggattgggatggaagtcactccatccgggacctggcttacctacacaggggcaataaaactcgacgacaaagacccaaactttaaagatcaggtcatcctgctgaataaacacatcgatgcctacaaaactttccccccaaccgaaccaaagaaagacaagaaaaaaaaggcagacgaaacgcaagcgctccctcagcgccagaagaagcagcagaccgttacactgttgccagcagcagatctggatgatttttccaagcagcttcaacagagtatgtcaagcgctgacagcactcaggcttgaggcgcgccgctgaccgataaataaaatatctttattttcattacatctgtgtgttggttttttgtgtgacgcgttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatatcgggccactgcaggaaacgatatgggctgaatacggatccgtattcagcccatatcgtttctctagaaataaaatatctttattttcattacatctgtgtgttggttttttgtgtgaatcgatagtactaacatacgctctccatctcgagcctaagcttgtcgactcgaagatctgggcgtggttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatcctctagttaatgtcaggtcgcctaagtcgattaactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatatt

SEQ ID NO: 9 - DNA, 35706 bp, Artificial Sequence
  Description of Artificial Sequence Synthetic polynucleotide
taaggatcccatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatactgctagagatctggcgaaagggggatgtgctgcaaggcgattaagttgggtaacgccagggttttcccagtcacgacgttgtaaaacgacggccagtgaattgtaatacgactcactatagggcgaattgggtactggccacaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctgactctagcctagctctgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtctgagcctaggagatctctcgaggtcgacggtatcgatgggtaccgccaccatgtttgtttttctcgtactcctgcccctggtttcctcccaatgtgtcaatctgactacccggacccaacttcctcccgcctacaccaattcctttacccgaggtgtttactacccagacaaagtgttcaggtcatccgtcctccatagtacccaagacctcttcctcccttttttttctaacgttacctggtttcacgctattcacgttagcggcaccaacggcaccaaaagattcgataaccccgtactgccgttcaacgacggggtatattttgcctctactgaaaaatcaaacatcatacgcggatggatctttgggactaccctggactcaaaaactcagtccctgctgattgtgaataacgctaccaacgtggtgatcaaagtctgtgaattccagttttgcaacgatccttttctcggcgtttattatcacaaaaataacaaatcctggatggagagcgagttccgggtgtactcctccgcgaataattgcaccttcgaatatgtgtctcagccattcctcatggacctcgaggggaagcagggcaattttaagaatctgcgagaattcgtgttcaagaatatagacggttacttcaagatttactccaaacacaccccgattaacctggttagggacttgcctcagggcttttctgcattggagcccctcgtggacctcccaatcggcataaacattacaagatttcagactttgcttgcattgcacaggagctatttgacacccggcgattcttcttccggatggaccgctggagcagctgcttattacgtgggctatctgcagcctcgaacctttcttttgaagtacaacgaaaatggaactatcaccgatgcagttgactgcgccctggaccccctgtccgaaactaagtgcacgctcaaaagtttcacagtagagaaggggatataccagactagcaatttccgcgttcagccaaccgaaagtatagtgcgctttcctaatataactaacctgtgtcctttcggggaagtgtttaacgccactagattcgcttccgtctacgcctggaatagaaagaggatctcaaattgcgttgctgactatagtgttttgtacaattccgcctctttctcaaccttcaaatgttacggggtgagccctaccaaactgaacgacctgtgctttacaaacgtatacgccgacagctttgttatcagaggagacgaggttcgccagattgctccgggtcagacaggcaagattgctgattataattacaaactgcccgacgactttacaggatgtgtgatcgcgtggaacagtaacaatcttgactcaaaggttgggggtaattataattatctttaccggctgttcagaaaaagcaatttgaaacccttcgaaagggacatatccaccgagatctatcaggccgggtccactccatgcaatggtgtggaaggttttaattgctacttcccattgcagtcttatggattccaaccaaccaatggcgtaggctaccagccgtatcgcgttgtcgtgctcagcttcgagctgctccacgcccccgcgaccgtatgcggtcctaagaagtccaccaatcttgttaagaacaagtgtgtaaactttaactttaacgggctgaccgggaccggcgttctgactgaatctaacaaaaaattcctgcctttccagcagttcggccgcgatattgctgacaccactgacgctgtaagagaccctcagacccttgaaattctcgatatcacaccttgcagctttgggggcgtgtccgtcatcactccaggaactaacacaagcaaccaggtggcagtgttgtaccaggatgttaattgtaccgaggtgccagtggccatccacgccgatcaattgacacctacctggagggtttacagcacagggtccaatgtttttcagacaagagccggatgtctgatcggtgccgagcatgtcaacaattcctacgagtgtgatatccccattggtgcgggaatttgtgcatcatatcagacccagactaatagcccaagaagagctagatccgtcgctagtcaatccatcattgcatatacaatgatgtccgataacggcccccagaatcagagaaacgctccccgcatcacgttcggcggaccaagtgacagcacaggcagtaaccagaacggagaacgctccggtgctcgctccaagcagcgacggccgcaagggcttcccaacaataccgccagctggtttacggctctgacccaacacgggaaagaagatcttaaattccccaggggccagggcgtccctatcaatactaactccagcccggatgatcagataggctactatagacgcgctacccgacggatacgagggggggacggcaaaatgaaggacctttccccccggtggtatttctattacttgggcaccggaccagaagccggactgccttacggcgctaacaaagacggaataatctgggttgcgacggagggcgccctgaatacacctaaagaccatatcggcacaagaaatcctgctaacaatgccgcgattgtgctccagctgcctcagggaaccacgctgcctaaagggttttacgctgaggggtcaaggggggggagtcaagcgtctagtaggtcatcctctcgctctcgcaatagttcccggaactcaaccccaggcagcagcagaggaacctctcccgcacggatggctggcaatgggggagatgctgcccttgctctccttctgctggatcgccttaaccagctcgaatcaaagatgtctggaaaaggtcagcagcagcaaggccagaccgtgacaaagaagagtgcagctgaagctagtaaaaagccacgccaaaaacggaccgcaactaaggcatataacgtaacacaggccttcggcagaagaggtccagaacaaacacagggaaactttggcgatcaagagctgattagacagggcacagattacaaacactggccacagatcgcgcagtttgcaccaagcgcctctgcattcttcgggatgagtcggattgggatggaagtcactccatccgggacctggcttacctacacaggggcaataaaactcgacgacaaagacccaaactttaaagatcaggtcatcctgctgaataaacacatcgatgcctacaaaactttccccccaaccgaaccaaagaaagacaagaaaaaaaaggcagacgaaacgcaagcgctccctcagcgccagaagaagcagcagaccgttacactgttgccagcagcagatctggatgatttttccaagcagcttcaacagagtatgtcaagcgctgacagcactcaggcttgacgatcggatatcgctagcgtaccggcggccgccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccaaagcttacgcgttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatatcgggccactgcaggaaacgatatgggctgaatacggatccgtattcagcccatatcgtttctctagaaataaaatatctttattttcattacatctgtgtgttggttttttgtgtgaatcgatagtactaacatacgctctccatctcgagcctaagcttgtcgactcgaagatctgggcgtggttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatcctctagttaatgtcaggtcgcctaagtcgattaactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatatt

SEQ ID NO: 10 - protein, 1116 aa, Artificial Sequence
  Description of Artificial Sequence Synthetic polypeptide
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 11 - DNA, 46 bp, Artificial Sequence
  Description of Artificial Sequence Synthetic oligonucleotide
gaaacgatatgggctgaatacttaagtattcagcccatatcgtttc

SEQ ID NO: 12 - DNA, 1190 bp, Artificial Sequence
  Description of Artificial Sequence Synthetic polynucleotide
cgggccccccctcgaggtcgacggtatcgataagcttgatatcgaattcgcccttagatatcgtcgacgcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagacatcgatgatatctaaagggcgaattcctgcagcccgggggatccactagtctagatgcatgctcgagcggccgccagtgtgatggatatctgcagaattcgcccttcagctgcggatccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccgggccccccctcgaggtcgacggtatcgataagcttgatatcgaattcctgcagcccgggggatccactagtttctagaaataaaatatctttattttcattacatctgtgtgttggttttttgtgtggcggccgccaccgcggtggagctatcgaattcaagcttgtcgactcgaagatcctagactagtggatcccccgggctgcaggaattcgccctttagatatcatcgatgtctcggcggtggtggcgcgtcgcgccgctgggttttatagggcgccgccgcggccgctcgagccataaaaggcaactttcggaacggcgcacgctgattggccccgcgccgctcactcaccggcttcgccgcacagtgcagcatttttttaccccctctcccctccttttgcgaaaaaaaaaaagagcgagagcgagattgaggaagaggaggagggagagttttggcgttggccgccttggggtgctgggcgtcgacgatatctaagggcgaattcgatatcaagcttatcgataccgtcgacctcgagggggggcccg

SEQ ID NO: 13 - DNA, 1757 bp, Artificial Sequence
  Description of Artificial Sequence Synthetic polynucleotide
cgggccccccctcgaggtcgacggtatcgataagcttgatatcgaattcgcccttagatatcgtcgacgcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagacatcgatgatatctaaagggcgaattcctgcagcccgggggatccactagtctagatgcatgctcgagcggccgccagtgtgatggatatctgcagaattcgcccttcagctgcggatccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccgggccccccctcgaggtcgacggtatcgataagcttgatatcgaattcctgcagcccgggggatccactagtttctagaaataaaatatctttattttcattacatctgtgtgttggttttttgtgtggcggccgccaccgcggtggagctatcgaattcaagcttgtcgactcgaagatcgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatactggtaccgggccccccctcgaggtcgacggtatcgataagcttgatatcgaattcgcccttagatatcgtcgacgcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagacatcgatgatatctaaagggcgaattcctgcagcccgggggatccactagtctagaactagtggatcccccgggctgcaggaattcgatatcaagcttatcgataccgtcgacctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggatccgcagctgaagggcgaattctgcagatatccatcacactggcggccgctcgagcatgcatctagaaataaaatatctttattttcattacatctgtgtgttggttttttgtgtggcggccgccaccgcggtggagcta

SEQ ID NO: 14 - DNA, 46 bp, Artificial Sequence
  Description of Artificial Sequence Synthetic oligonucleotide
gatggtgcttcaagctagtacttaagtactagcttgaagcaccatc

SEQ ID NO: 15 - DNA, 48 bp, Artificial Sequence
  Description of Artificial Sequence Synthetic oligonucleotide
gatggtgcttcaagctagtacggatccgtactagcttgaagcaccatc

SEQ ID NO: 16 - DNA, 48 bp, Artificial Sequence
  Description of Artificial Sequence Synthetic oligonucleotide
gaaacgatatgggctgaatacggatccgtattcagcccatatcgtttc

SEQ ID NO: 17 - DNA, 46 bp, Artificial Sequence
  Description of Artificial Sequence Synthetic oligonucleotide
cctaataattatcaaaatgtggatccacattttgataattattagg

SEQ ID NO: 18 - DNA, 44 bp, Artificial Sequence
  Description of Artificial Sequence Synthetic oligonucleotide
cctaataattatcaaaatgtaattacattttgataattattagg

SEQ ID NO: 19 - protein, 1270 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 20 - protein, 1270 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 21 - DNA, 846 bp, Artificial Sequence
  Description of Artificial Sequence Synthetic polynucleotide
atggacgccatgaaacgaggcctgtgctgcgtcctcctgctgtgtggggcagtgttcgttagtcaccagaagcgaaccgcaatgtttcaggacccccaggaaaggccccgaaaattgccgcagctgtgcaccgagctccagacaaccattcatgacatcattctggagtgtgtgtattgtaagcagcagctgttgaggagggaggtgtatgacttcgcttttcgggacggttgcattgtttaccgggatggaaacccttacgccgtttgcgataaatgtctgaagttctatagcaaaattagtgaatataggcattattgctactcactgtacggaaccacactggaacagcagtataacaaacccctgtgcgaccttctgattaggtgcattaattgtcagaaaccgctgtgcccagaggaaaagcagcgccatcttgacaagaaacagagattccataacatccggggcagatggactggacgctgcatgtcttgttgtcgctcctcaaggacgagacgggccgcggctgccaggaagaaacgtaggatgcccggcgataccccgacactgcacgaatatatgctggacctccaacccgagacgacagatctgtacggttacgagcaactgaacgactcctccgaggaagaagacgaaatcgacgggcccgcaggtcaggcagcacctgaccgcgcccactacaatattgtcaccttttgctgcaaatgtgactccacactccgacgttgtgttcaatcaacccacgtggatattcgaactctggaggatcttctgatgggaaccctgggtattgtatgccccatctgcagccaaaaaccatag

SEQ ID NO: 22 - protein, 281 aa, Artificial Sequence
  Description of Artificial Sequence Synthetic polypeptide
MDAMKRGLCCVLLLCGAVFVSHQKRTAMFQDPQERPRKLPQLCTELQTTIHDIILECVYCKQQLLRREVYDFAFRDGCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRAAAARKKRRMPGDTPTLHEYMLDLQPETTDLYGYEQLNDSSEEEDEIDGPAGQAAPDRAHYNIVTFCCKCDSTLRRCVQSTHVDIRTLEDLLMGTLGIVCPICSQKP

SEQ ID NO: 23 - DNA, 2166 bp, Artificial Sequence
  Description of Artificial Sequence Synthetic polynucleotide
atggacgccatgaaacgaggcctgtgctgcgtcctcctgctgtgtggggcagtgttcgttagtcaccagaagcgaaccgcaatgtttcaggacccccaggaaaggccccgaaaattgccgcagctgtgcaccgagctccagacaaccattcatgacatcattctggagtgtgtgtattgtaagcagcagctgttgaggagggaggtgtatgacttcgcttttcgggacggttgcattgtttaccgggatggaaacccttacgccgtttgcgataaatgtctgaagttctatagcaaaattagtgaatataggcattattgctactcactgtacggaaccacactggaacagcagtataacaaacccctgtgcgaccttctgattaggtgcattaattgtcagaaaccgctgtgcccagaggaaaagcagcgccatcttgacaagaaacagagattccataacatccggggcagatggactggacgctgcatgtcttgttgtcgctcctcaaggacgagacgggccgcggctgccaggaagaaacgtaggatgcccggcgataccccgacactgcacgaatatatgctggacctccaacccgagacgacagatctgtacggttacgagcaactgaacgactcctccgaggaagaagacgaaatcgacgggcccgcaggtcaggcagcacctgaccgcgcccactacaatattgtcaccttttgctgcaaatgtgactccacactccgacgttgtgttcaatcaacccacgtggatattcgaactctggaggatcttctgatgggaaccctgggtattgtatgccccatctgcagccaaaaaccaggctccggcgagggcaggggaagtcttctaacatgcggggacgtggaggaaaatcccggcccaatgtccgataacggcccccagaatcagagaaacgctccccgcatcacgttcggcggaccaagtgacagcacaggcagtaaccagaacggagaacgctccggtgctcgctccaagcagcgacggccgcaagggcttcccaacaataccgccagctggtttacggctctgacccaacacgggaaagaagatcttaaattccccaggggccagggcgtccctatcaatactaactccagcccggatgatcagataggctactatagacgcgctacccgacggatacgagggggggacggcaaaatgaaggacctttccccccggtggtatttctattacttgggcaccggaccagaagccggactgccttacggcgctaacaaagacggaataatctgggttgcgacggagggcgccctgaatacacctaaagaccatatcggcacaagaaatcctgctaacaatgccgcgattgtgctccagctgcctcagggaaccacgctgcctaaagggttttacgctgaggggtcaaggggggggagtcaagcgtctagtaggtcatcctctcgctctcgcaatagttcccggaactcaaccccaggcagcagcagaggaacctctcccgcacggatggctggcaatgggggagatgctgcccttgctctccttctgctggatcgccttaaccagctcgaatcaaagatgtctggaaaaggtcagcagcagcaaggccagaccgtgacaaagaagagtgcagctgaagctagtaaaaagccacgccaaaaacggaccgcaactaaggcatataacgtaacacaggccttcggcagaagaggtccagaacaaacacagggaaactttggcgatcaagagctgattagacagggcacagattacaaacactggccacagatcgcgcagtttgcaccaagcgcctctgcattcttcgggatgagtcggattgggatggaagtcactccatccgggacctggcttacctacacaggggcaataaaactcgacgacaaagacccaaactttaaagatcaggtcatcctgctgaataaacacatcgatgcctacaaaactttccccccaaccgaaccaaagaaagacaagaaaaaaaaggcagacgaaacgcaagcgctccctcagcgccagaagaagcagcagaccgttacactgttgccagcagcagatctggatgatttttccaagcagcttcaacagagtatgtcaagcgctgacagcactcaggcttga

SEQ ID NO: 24 - protein, 21 aa, Artificial Sequence
  Description of Artificial Sequence Synthetic peptide
GSGEGRGSLLTCGDVEENPGP

SEQ ID NO: 25 - protein, 27 aa, Artificial Sequence
  Description of Artificial Sequence Synthetic peptide
GYIPEAPRDGQAYVRKDGEWVLLSTFL